SEQUENCE LISTING (36 sequences)

SEQ ID NO: 1 - DNA, 27 bp, Fugu rubripes
aggtggatcccacaaaacgtcaaattc

SEQ ID NO: 2 - DNA, 27 bp, Tetraodon nigroviridis
aggtggacccctcgaaatgtcacattc

SEQ ID NO: 3 - DNA, 27 bp, Oreochromis niloticus
aagtggatcccactaaacgtcagatcc

SEQ ID NO: 4 - DNA, 27 bp, Gasterosteus aculeatus
aggtggaccccactgagcgccgcgtcc

SEQ ID NO: 5 - DNA, 27 bp, Oryzias latipes
aggtggacccctccaaacggcagattc

SEQ ID NO: 6 - DNA, 27 bp, Gadus morhua
aacttgatcccactaaacgcaagattc

SEQ ID NO: 7 - DNA, 27 bp, Danio rerio
aggtggacccaagcaaaaggcgaatcc

SEQ ID NO: 8 - DNA, 27 bp, Macropus eugenii
aggtggacccatccaagagaagagtcc

SEQ ID NO: 9 - DNA, 27 bp, Monodelphis domestica
aggttgacccatccaagagaaaagttc

SEQ ID NO: 10 - DNA, 27 bp, Sarcophilus harrisii
aggtggacccatccaagagcagggtcc

SEQ ID NO: 11 - DNA, 27 bp, Rattus norvegicus
aagtggacccatcccggcagcgtgtgc

SEQ ID NO: 12 - DNA, 27 bp, Mus musculus
aagtggacccatcccagcagcgtgtgc

SEQ ID NO: 13 - DNA, 27 bp, Dipodomys ordii
aggtggatgggtcccagcggcgtgtgc

SEQ ID NO: 14 - DNA, 27 bp, Spermophilus tridecemlineatus
aggtggacccatcccagcggcatgtgc

SEQ ID NO: 15 - DNA, 27 bp, Cavia porcellus
aggtggacccctcaaggcagcgcgtgc

SEQ ID NO: 16 - DNA, 27 bp, Ochotona princeps
ccgtggacccgtcccggcggcaggtgc

SEQ ID NO: 17 - DNA, 27 bp, Tupaia belangeri
aggtggacccatcccagaggcgcgtgc

SEQ ID NO: 18 - DNA, 27 bp, Homo sapiens
aggtggacccatcgcggatacatgtgc

SEQ ID NO: 19 - DNA, 27 bp, Pan troglodytes
aggtggacccatcgcggatacacgtgc

SEQ ID NO: 20 - DNA, 27 bp, Gorilla gorilla
aggtggacccatcgcggacacatgtgc

SEQ ID NO: 21 - DNA, 27 bp, Pongo abelii
aggtggacccatctcagacacatgtgc

SEQ ID NO: 22 - DNA, 28 bp, Nomascus leucogenys
aggtggaccccatcacggacacatgtgc

SEQ ID NO: 23 - DNA, 28 bp, Macaca mulatta
aggtggaccccgtcacagacacgtgtgc

SEQ ID NO: 24 - DNA, 27 bp, Callithrix jacchus
aggtggacccgtcggggaggcaggtgc

SEQ ID NO: 25 - DNA, 27 bp, Otolemur garnettii
aggtggacccctcccagaggcaggtgc

SEQ ID NO: 26 - DNA, 27 bp, Ailuropoda melanoleuca
aggtggacccatcccggagcagagtgc

SEQ ID NO: 27 - DNA, 27 bp, Myotis lucifugus
aggtggacccgtcccggaggcaggtgc

SEQ ID NO: 28 - DNA, 27 bp, Tursiops truncatus
aggtggacccgacccagggccgagtgc

SEQ ID NO: 29 - DNA, 27 bp, Equus caballus
aggtggacccatcccagagccgtgtgc

SEQ ID NO: 30 - DNA, 27 bp, Loxodonta africana
aggtggacccatcccggcgccaggtac

SEQ ID NO: 31 - DNA, 27 bp, Ornithorhynchus anatinus
aggtggacccgtcggcccggagagtcc

SEQ ID NO: 32 - DNA, 27 bp, Gallus gallus
aagttgactccaccaagaaaagggtac

SEQ ID NO: 33 - DNA, 27 bp, Meleagris gallopavo
aagttgactcaaccaagaaaagggtgc

SEQ ID NO: 34 - DNA, 27 bp, Taeniopygia guttata
aggttgacccaagtaagaaaaaggtgc

SEQ ID NO: 35 - DNA, 27 bp, Xenopus tropicalis
aggttgatttatctaaaagaaaagttc

SEQ ID NO: 36 - DNA, 27 bp, Latimeria chalumnae
aggttgatccatccaataggagggttc

(19) United States
(12) Patent Application Publication
(10) Pub. No.: US 2020/0097835 A1
(43) Pub. Date: Mar. 26, 2020

(54) DEVICE, SYSTEM AND METHOD FOR ASSESSING RISK OF VARIANT-SPECIFIC GENE DYSFUNCTION

(71) Applicant: Ancestry.com DNA, LLC, Lehi, UT (US)

(72) Inventors: Ari Julian Silver, Somerville, MA (US); Velina Kozareva, Cambridge, MA (US); Maxwell J. Silver, New York, NY (US); Nigel Delaney, San Francisco, CA (US); Lee M. Silver, New York, NY (US)

(21) Appl. No.: 16/579,700

(22) Filed: Sep. 23, 2019

Related U.S. Application Data
(63) Continuation-in-part of application No. 15/136,093, filed on Apr. 22, 2016.
Application No. 15/136,093 is a continuation-in-part of application No. 14/568,456, filed on Dec. 12, 2014.
(60) Provisional application No. 62/739,038, filed on Sep. 28, 2018.
(60) Provisional application No. 62/151,116, filed on Apr. 22, 2015.
(60) Provisional application No. 62/013,139, filed on Jun. 17, 2014.

(51) Int. Cl. G06N 3/12; G16H 50/70; G06N 3/08; G06F 17/11

(52) U.S. Cl. CPC G06N 3/126; G06F 17/11; G06N 3/08; G16H 50/70

(57) ABSTRACT

A method may include generating multiple virtual progenies from multiple first virtual gametes and multiple second virtual gametes. Each virtual progeny may combine one of the first virtual gametes and one of the second virtual gametes. A computing server may input, for each virtual progeny, data associated with the first virtual gamete of the virtual progeny to a machine learning model to determine a first variant-specific gene dysfunction score corresponding to a target allele site. The computing server may also input, for each virtual progeny, data associated with the second virtual gamete of the virtual progeny to the machine learning model to determine a second variant-specific gene dysfunction score corresponding to the target allele site. The computing server may derive, for each virtual progeny, a dysfunction likelihood score of the target allele site from the first variant-specific gene dysfunction score and the second variant-specific gene dysfunction score.

### CROSS-REFERENCE TO RELATED APPLICATIONS

This patent application claims the benefit of U.S. Provisional Patent Application No. 62/739,038, filed Sep. 28, 2018, and is also a continuation-in-part of U.S. patent application Ser. No. 15/136,093, filed Apr. 22, 2016, which claims the benefit of U.S. Provisional Patent Application No. 62/151,116, filed Apr. 22, 2015 and is also a continuation-in-part of U.S. patent application Ser. No. 14/568,456, filed Dec. 12, 2014, which claims the benefit of U.S. Provisional Patent Application No. 62/013,139 filed Jun. 17, 2014, all of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Dec. 3, 2019, is named 44064 US CFR SequenceListing.txt and is 5,945 bytes in size.

### FIELD OF THE INVENTION

Embodiments of the present invention relate generally to the field of genetics. Embodiments of the present invention relate to predicting the risk that one or more specific allele variants will cause gene dysfunction or deleterious mutations associated with disease or reduced likelihood of surviving or reproducing in an organism.

### BACKGROUND OF THE INVENTION

Every year thousands of babies are born with genetic diseases. Often, the parents of these children are both healthy, but each parent possesses genetic mutations that when passed in combination to the child, endow it from the time of conception with an unmitigated genetic defect. Children with such diseases may suffer, have diminished lifespans and can entail large emotional and financial costs, so many prospective parents attempt to minimize the chance that they pass on genetic elements that cause disease.

Carrier testing, in which both parents are genotyped at loci of their genomes that are known to cause disease, is a technique widely used to achieve this goal. Carrier testing is unique among medical diagnostics in that recessive disease is only predicted to occur in persons other than those actually being tested. Variants in a known disease gene are classified as “pathogenic” when observed in correlation with patients diagnosed with the corresponding disease. A panel of pathogenic variants from the same gene provides the basis for developing a specific test. Persons who carry any targeted clinically validated variant are scored “positive,” and two prospective parents who test positive for the same autosomal gene are assigned a 25% risk of conceiving a diseased child. Conventional carrier testing suffers from several limitations.

Firstly, carrier testing uses a binary classification system, defining an allele variant as having only either a “positive” (pathogenic) or a “negative” (benign) effect of causing a disease. This binary classification fails to identify any continuum or intermediate effects (e.g., a degree of disease or partial functionality of a phenotype) or to illuminate allele-specific or genotype-specific differences in predicted phenotypes from the same gene. In some cases, variants with partial functionality will express allele-specific or genotype-specific effects (e.g., associated with disease in some allelic combinations but not others). The binary classification system cannot differentiate between different phenotypes caused by different allele or genotype combinations of the same gene.

Binary classification is typically useful for patients with a known disease or phenotype to search for the variant that causes their disease or phenotype. A successful search distinguishes the patient's “pathogenic” variants from “benign” variants. If the patient's condition cannot be ascribed to previously characterized variants, a number of computational tools have been developed for filtering and ranking potential culprits. The performance of these discovery tools is typically measured by an area under a receiver operator characteristic (ROC) curve for benchmark sets of pathogenic and benign variants.

In recently published guidelines for scoring the pathogenicity of DNA sequence variants, the American College of Medical Genetics and Genomics encouraged clinical researchers to “arrive at a single conclusion” that is “determined by the entire body of evidence.” However, the assumption of all-or-none pathogenicity is inappropriate for variants in recessive disease genes. “Pathogenic” implies that a variant has an absolute or determinative causal relationship to a disease or phenotype, and yet, in molecular terms, a single recessive disease allele cannot independently cause a disease, but participates passively in a reduction or loss of function that is tolerated in the heterozygous presence of a fully functional gene copy. Recessive disease will only ensue in a homozygote or compound heterozygote where the molecular sum of functional products from both gene copies fails to rise above the threshold required for health.

A second limitation of conventional carrier testing is that it is very difficult to identify the disease-risk of variants of recessive diseases or traits because many of the patients carrying those variants are heterozygous and do not express the recessive disease or trait. Newly arising mutations in recessive disease genes will usually be transmitted silently from one generation of heterozygotes to the next, without appearing in diseased patients. In a recent analysis of the Cystic Fibrosis Transmembrane Conductance Regulator (CFTR) gene in 60,000 exomes from individuals not affected with cystic fibrosis, the number of likely disease-causing variants that had not been clinically validated was twice the number that were validated. The expanded use of Next Generation Sequencing (NGS) for genetic screening of all recessive disease genes will result in the detection of many more “untested” variants that are not available for informed reproductive decision making under the current testing regimen.

A third limitation of conventional carrier testing is that it typically only tests for variants validated to cause disease in clinical studies. Carrier testing typically relies on the curation of clinical reports as its primary source for variant inclusion. Such tests rely on a defined set of alleles known to cause diseases, and then screen for the presence of these alleles in one or both parents prior to conception. The alleles screened in such tests have been established to cause disease by examining pedigrees of patients with the disease, by using cellular or animal models of the effect of the particular allele, or alternate means. The incompleteness of these tests is evidenced by the fact that the number of alleles associated with disease in public databases such as ClinVar (http://www.ncbi.nlm.nih.gov/clinvar/) and OMIM (http://www.ncbi.nlm.nih.gov/omim) continues to grow every year, and in turn so do the number of loci tested by carrier screening. Similarly, many patients can present with pathologies which appear to have a genetic basis, but for which no specific underlying genetic mutation has yet been determined. In many of these cases, a novel pathogenic variant or variants is then later discovered by various means and added to the catalog of known disease associated mutations. For example, the genomes of many patients with similar pathologies can be sequenced and shared mutations found. Alternatively, mutations that occur in an individual patient's genome which appear damaging (missense, nonsense, etc.) and are present in genes known to be associated with a biological process related to the pathology, may be tested in a cellular or animal model.

While the steady increase of the catalog of variants known to cause disease implies that carrier testing will get better, it also evinces that it suffers from the limitation that it only screens for clinically validated mutations, and cannot assess the impact of novel or de novo mutations. If a variant is specific to an individual or family and has not been previously studied, carrier testing cannot determine what effect it may have on future offspring.

A fourth limitation of conventional carrier testing is that a diseased child must be born and diagnosed in order to find a new disease associated allele. In all cases, the correlation between alleles and genetic diseases are determined by studying one or more individuals that have already been born with the disease. In the case of recessive disease, the problem is compounded because novel variants usually initially only appear as one half of a heterozygote genotype which does not express disease, and will spread silently through populations before it is combined with itself or another recessive mutation as homozygotes to express the disease in patients. Thus, it is very difficult to resolve the effect of the mutation until children suffering from the disease are born, and from the perspective of a parent who wants to avoid passing on disease causing alleles, it is too late.

### SUMMARY OF THE INVENTION

A system, device and method are described to overcome the aforementioned longstanding issues inherent in the art.

In an embodiment, a method may include generating a plurality of virtual progenies from a plurality of first virtual gametes and a plurality of second virtual gametes. Each virtual progeny may combine one of the first virtual gametes and one of the second virtual gametes. The method may also include inputting, for each virtual progeny, data associated with the first virtual gamete of the virtual progeny to a machine learning model to determine a first variant-specific gene dysfunction score corresponding to a target allele site. The method may further include inputting, for each virtual progeny, data associated with the second virtual gamete of the virtual progeny to the machine learning model to determine a second variant-specific gene dysfunction score corresponding to the target allele site. The method may further include deriving, for each virtual progeny, a dysfunction likelihood score of the target allele site from the first variant-specific gene dysfunction score and the second variant-specific gene dysfunction score. The method may further include generating a distribution of dysfunction likelihood of the target allele site based on a plurality of the dysfunction likelihood scores determined from the plurality of virtual progenies.

In an embodiment of the invention, a device, system and method is provided for predicting gene-dysfunction caused by a defined genetic mutation in the genome of an organism. A neural network may be stored, for example in one or more memory units. The neural network may comprise multiple nodes respectively associated with multiple different gene-dysfunction metrics and multiple different confidence weights. One or more processors may process the neural network to combine the multiple gene-dysfunction metrics according to the respective associated confidence weights to generate one or more likelihoods that a genetic mutation causes gene-dysfunction in organisms. The one or more processors may process the neural network in a training-phase and a run-time phase. In a training-phase, the neural network may be trained using an input data set including one or more genetic mutations to generate new gene-dysfunction metrics and new associated confidence weights that optimize the neural network based on a cost factor. In a run-time phase, a genetic mutation may be identified and one or more likelihoods may be computed that the identified genetic mutation causes gene-dysfunction in the organism based on the new gene-dysfunction metrics and the associated new confidence weights of the neural network. The multiple different gene-dysfunction metrics may include combinations of one or more of a population selection component, an evolutionary selection component, a pathogenic predictor component, a mutation class component and/or a clinical classification component.

In an embodiment of the invention, a device, system and method is provided for predicting gene-dysfunction associated with a genetic mutation in an organism based on population-specific selection factors. Multiple population-specific sets of genetic sequences may be received each including multiple genetic sequences obtained from genetic samples of organisms from a different respective one of multiple populations. Each of multiple population-specific measures of homozygosity of the genetic mutation may be generated for each of the respective multiple populations by comparing the count of observed homozygotes of the genetic mutation measured on both chromosomes at a genetic locus in the population-specific set and an expected homozygote count based on a total observed count of the genetic mutation measured on either chromosome at the genetic locus in the population-specific set. One or more likelihoods may be computed that the genetic mutation causes gene-dysfunction in the organism based on one or more of the multiple population-specific measures of homozygosity.

In an embodiment of the invention, a device, system and method is provided for predicting gene-dysfunction associated with a genetic mutation in an organism based on the evolution of genetic variation of multiple organisms within one species (“single-species” or “intra-species” model) or across multiple different species (“multi-species” or “inter-species” model). Past evolutionary trends in allele mutations of extant or surviving (currently or once-living) organisms representative of one or more species or populations may be analyzed to predict the future fitness of a living organism or a potential hypothetical or virtual progeny simulated for two living potential parents. In some embodiments of the invention, a system, device and method may receive multiple aligned genetic sequences obtained from genetic samples of multiple organisms of one or more different species. Genetic loci may be aligned from different sequences for different organisms that are derived from one or more common ancestral genetic loci correlated with the same trait(s), disease(s), codon(s), that are positioned or sandwiched between other correlated marker loci, or that are otherwise related. A measure of evolutionary variation may be computed for one or more alleles at each of one or more aligned genetic loci of the multiple aligned sequences. The measure of evolutionary variation may be a function of variation in alleles at corresponding aligned genetic loci in the multiple aligned genetic sequences. One or more likelihoods may be computed that an allele, either a new mutation or one present in the alignment, at each of the one or more genetic loci in an organism will be deleterious based on the measure of evolutionary variation of alleles at the corresponding aligned genetic loci for the multiple organisms.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 schematically illustrates a system for assessing risk or likelihood of variant-specific gene dysfunction according to an embodiment of the invention;

FIG. 2 schematically illustrates a display visualizing a DNA image and/or sequence of an organism comprising one or more variants or mutations labeled in the DNA sequence together with a likelihood of variant-specific gene dysfunction for the organism according to an embodiment of the invention;

FIGS. 3A-3D schematically illustrate an example of (A) a neural network combining multiple gene dysfunction components for determining the risk of variant-specific gene dysfunction in an organism; (B) an exploded view of the population selection component; (C) an exploded view of the clinical classification component, mutation class component and pathogenic predictors component; and (D) optimized parameters for the components in FIGS. 3A-3C, according to an embodiment of the invention;

FIGS. 4A and 4B show violin plots of an example of variant-specific gene dysfunction (A) without and (B) with a clinical classification component according to an embodiment of the invention;

FIGS. 5A and 5B show violin plots of an example of variant-specific gene dysfunction (A) without and (B) with a mutation type component according to an embodiment of the invention;

FIGS. 6A-6D show violin plots of an example comparison of variant-specific gene dysfunction and (A) PolyPhen-2, (B) adjusted CADD, (C) adjusted PROVEAN, and (D) VEST values, stratified by clinical classification category according to an embodiment of the invention;

FIGS. 7A-7B are graphs of an example of (A) one-sided bootstrap confidence intervals per gene, (B) a distribution of the density of confidence interval thresholds versus variant-specific gene dysfunction (VGD^{-Cl}), according to some embodiments of the invention;

FIG. 8 is a graph of an example of Phred scaled CADD values versus adjusted CADD values (Rⱼ^{C}) and raw weights (wⱼ^{0C}) according to an embodiment of the invention;

FIG. 9 is a graph of an example of raw PROVEAN values versus adjusted PROVEAN values Rⱼ^{PR} and raw weights (wⱼ^{0PR}) according to an embodiment of the invention;

FIG. 10 is a graph of an example relationship between in-frame indel mutation class values Sⱼ^{M} vs. the number of inserted codons according to an embodiment of the invention;

FIGS. 11A-11B are plots of maximum population frequency for all variants in (A) Pore Forming Protein 1 (PRF1) and (B) Phosphorylase, Glycogen, Muscle (PYGM), according to some embodiments of the invention;

FIG. 12 shows a violin plot of an example of variant-specific gene dysfunction for each variant stratified by VariBench category according to an embodiment of the invention;

FIGS. 13A-13D show violin plots of an example comparison of variant-specific gene dysfunction and (A) PolyPhen-2, (B) adjusted CADD, (C) adjusted PROVEAN, and (D) VEST values, stratified by VariBench category according to an embodiment of the invention;

FIGS. 14A-14D show violin plots of an example comparison of variant-specific gene dysfunction and (A) PolyPhen-2, (B) adjusted CADD, (C) adjusted PROVEAN, and (D) VEST values, stratified by mutation type for all variants according to an embodiment of the invention;

FIG. 15 schematically illustrates an example of an alignment of multiple genetic sequences according to an embodiment of the invention;

FIG. 16 schematically illustrates an example of simulating a hypothetical mating of two potential parents for generating a virtual progeny according to an embodiment of the invention;

FIG. 17 schematically illustrates an example of a phylogenetic tree inferred from the multiple sequence alignment shown in FIG. 16 according to an embodiment of the invention;

FIGS. 18A and 18B are graphs of the likelihood of variant-specific gene dysfunction for 400 variants of an example disease gene, transglutaminase (TGM) 1, compared with clinically validated results in (A) 2012 and (B) 2016 according to an embodiment of the invention;

FIG. 19A is a flowchart of a method for assessing risk of variant-specific gene dysfunction according to an embodiment of the invention;

FIG. 19B is a flowchart of a method of predicting gene-dysfunction associated with a genetic mutation in an organism based on population-specific selection factors or nodes according to an embodiment of the invention; and

FIG. 19C is a flowchart of a method of predicting gene-dysfunction associated with a genetic mutation in an organism based on evolutionary selection factors or nodes according to an embodiment of the invention.

FIG. 20 is a flowchart depicting an example process of determining an allele dysfunction likelihood, in accordance with an embodiment.

FIG. 21 shows a ranking of genes by a proportion of ExAC LoF variants in ClinVar, in accordance with an embodiment.

FIG. 22 shows the results of some of the matches and genotypes predicted to be at preconception risk by the Paired Carrier Tests and Virtual Progeny Analytics test.

FIG. 23 is a plot of continuous trait model of biotinidase deficiency, in accordance with an embodiment.

FIG. 24 illustrates MEFV alleles, genotypes, and phenotypes in the Ashkenazi Jewish population.

FIG. 25 is a VGD score plot highlighting the distribution of DHCR7 variants by gene dysfunction effect and population frequency, in accordance with an embodiment.

FIG. 26 is a VGD score plot highlighting likely disease-causing genotypes for CFTR gene, in accordance with an embodiment.

FIG. 27 is a VGD score plot highlighting likely disease-causing genotypes and population frequency for IDUA gene, in accordance with an embodiment.

FIG. 28 is a VGD score plot highlighting likely disease-causing genotypes and population frequency for SMPD1 gene, in accordance with an embodiment.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OR EMBODIMENTS OF THE INVENTION

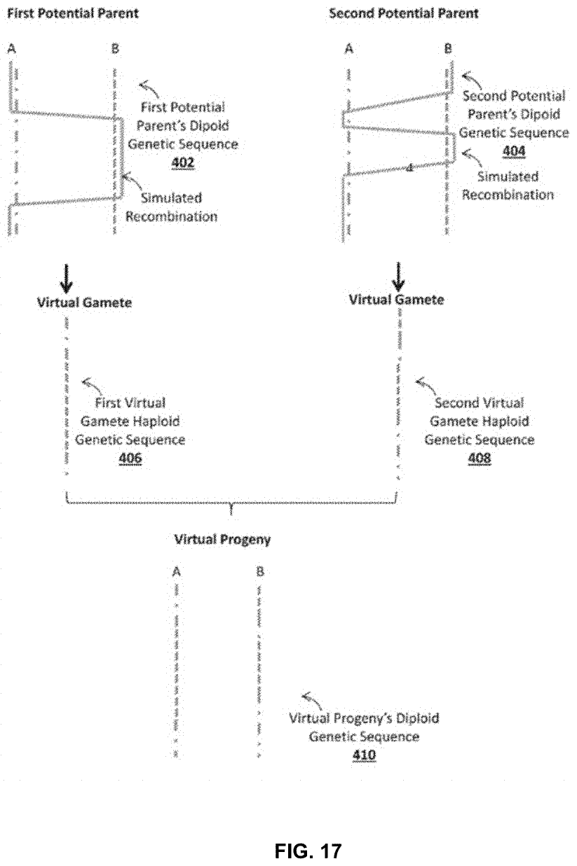

Embodiments of the invention provide a system, device and method for analyzing a DNA sequence to determine risk or probability of gene dysfunction associated with specific variants or allele combinations in the DNA sequence, for example, associated with disease or reduced likelihood of surviving or reproducing in an organism. The DNA sequence may be sequenced from a biological sample of a living organism (a “real” or “extant” organism) or may be simulated (e.g., simulating a mating) by combining at least a portion of genetic information representing genetic material obtained from biological DNA samples of two living potential parents (e.g. as shown in FIG. 17) (a “virtual” or “simulated” progeny). All genetic information that is genetically screened is derived or transformed from biological DNA samples of living human organisms.

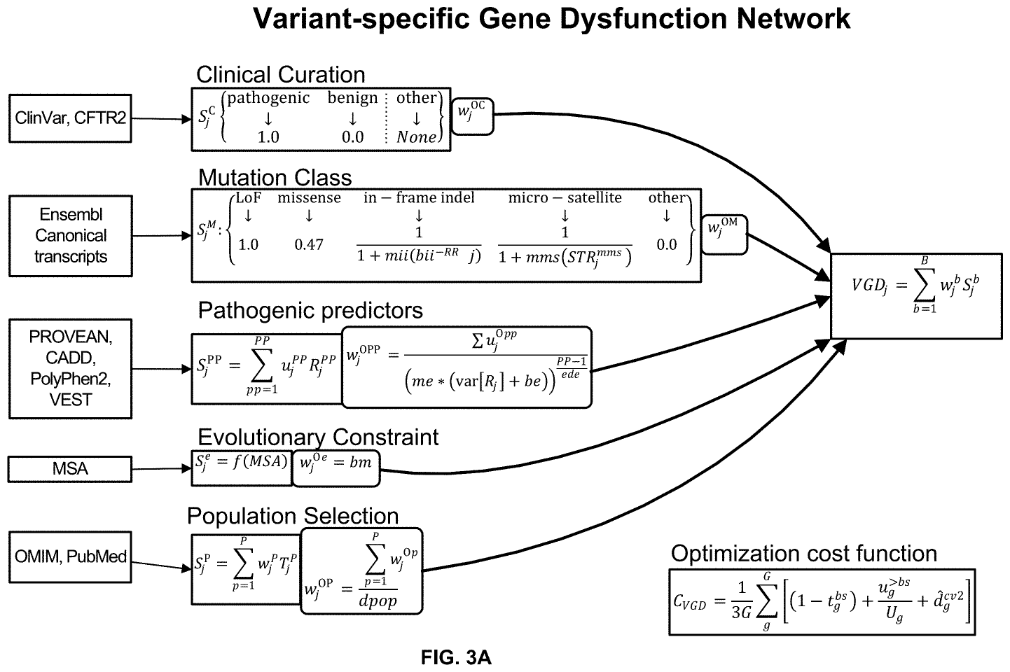

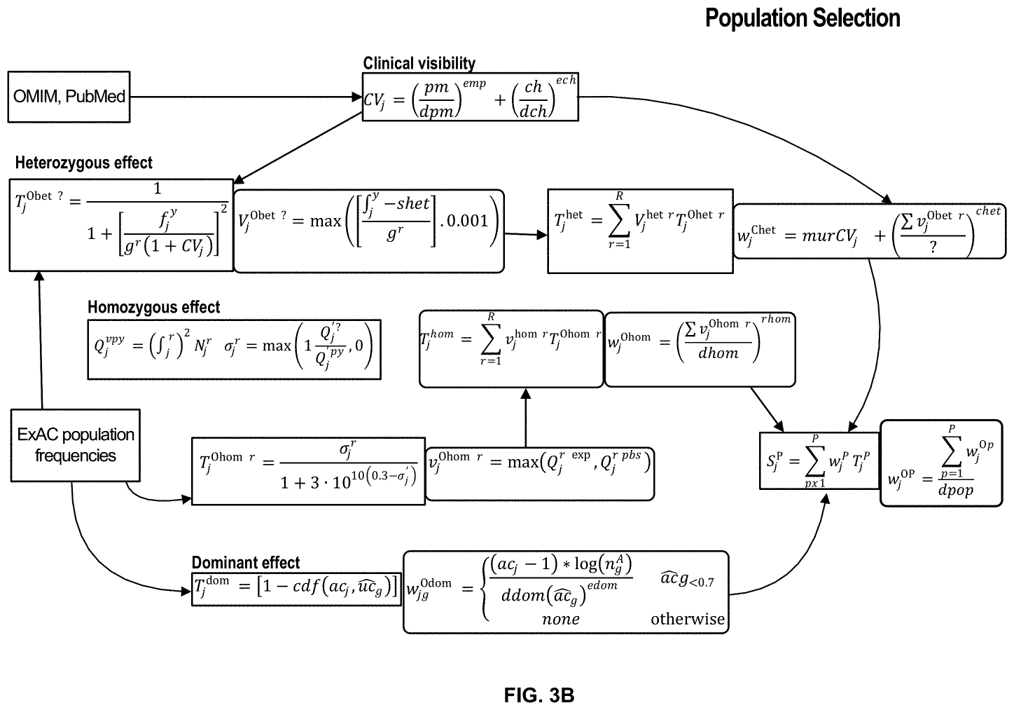

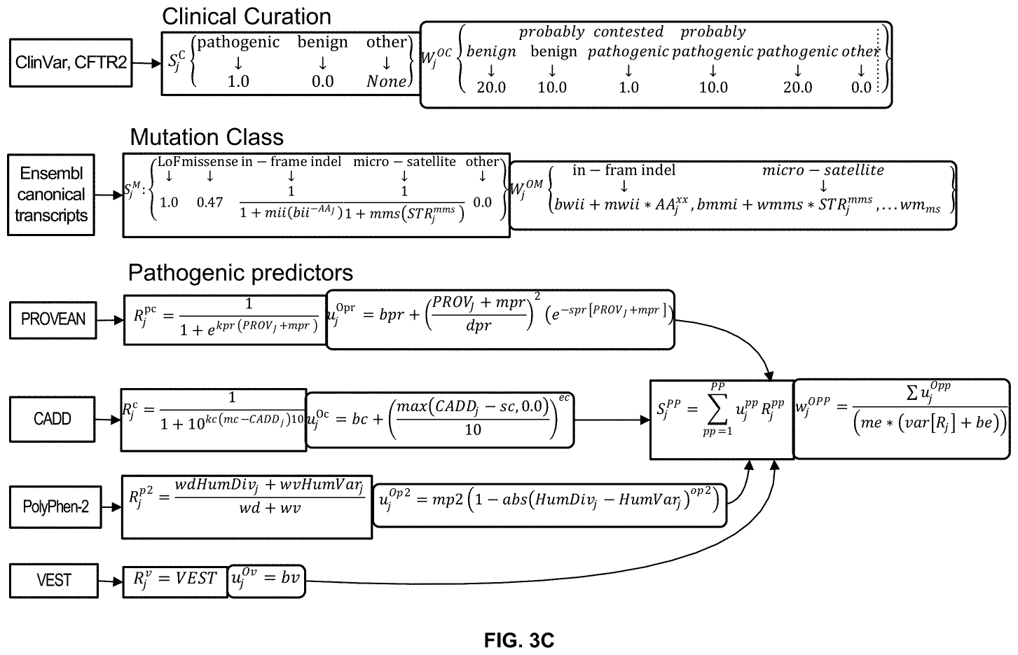

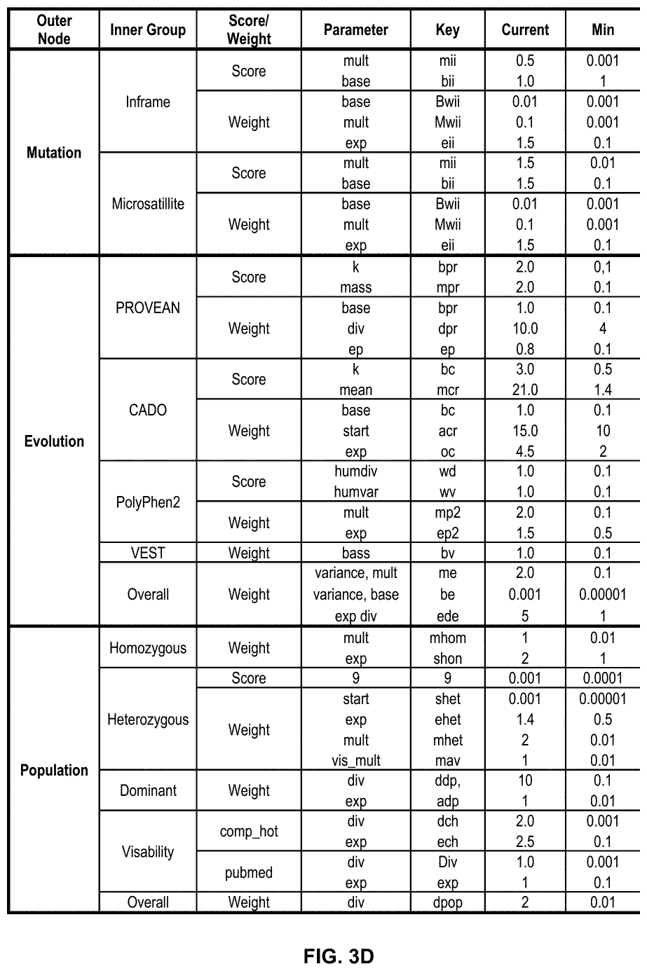

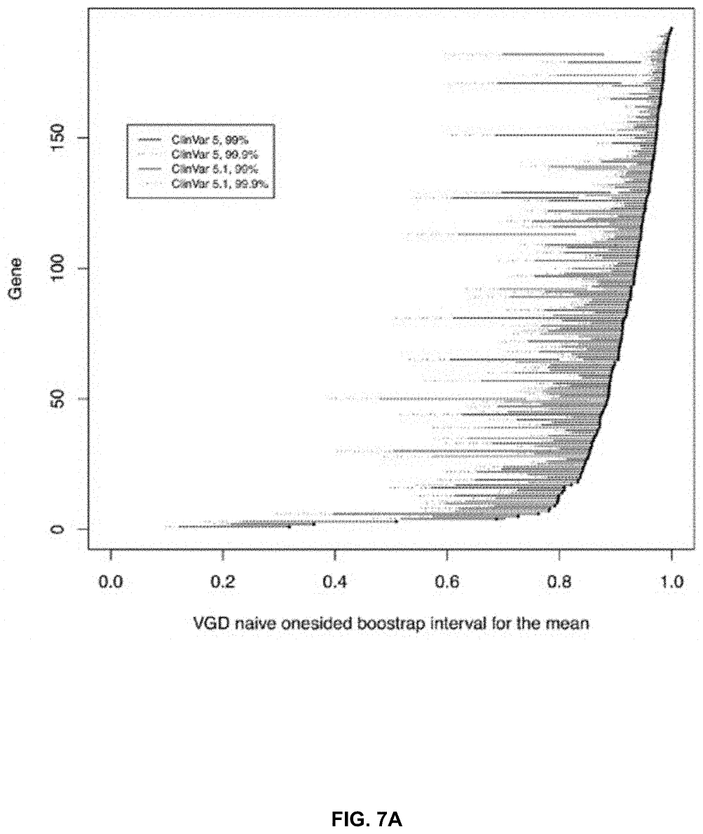

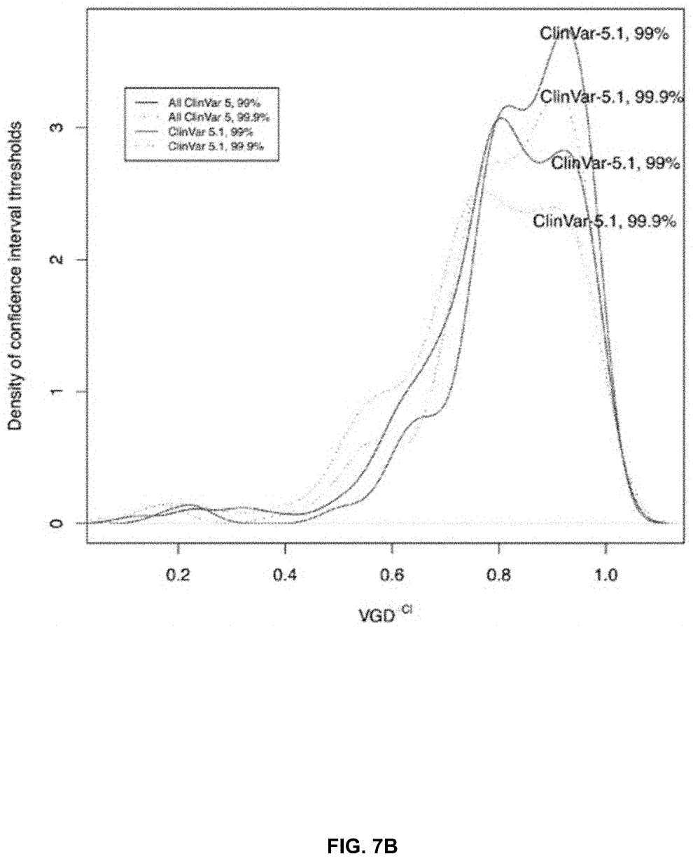

Embodiments of the invention replace the unrealistic conventional binary classification system of disease-risk with a continuous variant-weighted component-based dysfunction scoring system for each single gene copy. Embodiments of the invention may compute one or more likelihoods of variant-specific gene dysfunction by integrating multiple gene dysfunction categories. These multiple gene dysfunction categories may be weighted according to variant-defined levels of confidence and summed to generate a variant-specific gene dysfunction (VGD). The multiple gene dysfunction components may be combined using a neural network (e.g. FIGS. 3A-3D). Each node of the neural network may represent a different gene dysfunction component, for example, associated with one or more score(s) and weight(s). The neural network may be trained by machine-learning based on a training set of known variant dysfunction measures to optimize or tune network parameters to improve the network score(s) and weight(s) (e.g. see “Parameter” key in FIG. 3D). The neural network is trained, for example, using a cost or error factor (e.g. see “Optimization cost function” in FIG. 3A), for example, to optimize predictive agreement with one or more benchmark datasets of pathogenic and benign variants on a targeted set of multiple recessive disease genes. Clinical classifications of variants known to be “pathogenic” or “benign” may be used as a truth class to validate the model for each gene and set parameters (e.g., FIG. 3D) and sensitivity thresholds (e.g., FIGS. 7A-7B).

The multiple gene dysfunction categories integrated into the variant-specific gene dysfunction score may include, for example, clinical classification, mutation class, pathogenic predictors, evolutionary constraint, and population selection.

The population selection component measures a “homozygous effect,” a “heterozygous effect” and/or a “dominant effect” in each of a plurality of human populations (r=1, . . . R). In one example, populations include non-Finnish Europe, Finland, South Asia, East Asia, Africa, and the Americas, although other populations or groups may be used.

The homozygous effect may measure each population's natural selection against homozygote forms of a recessive genetic variant. The homozygous effect may compare the observed incidence or count of a homozygote of a variant genotype Qⱼ^{r,obs} (e.g. observed on both chromosomes) to a predicted or expected incidence or count of the homozygous variant genotype Qⱼ^{r,exp} (e.g. based on the observed allele frequency on either chromosome (fⱼ^{r}) and the population size (Nⱼ^{r}), such as, Qⱼ^{r.exp}=(fⱼ^{r})²Nⱼ^{r}), in each population. The homozygous effect is based on a “null hypothesis” that if a variant's effect is neutral (e.g. having substantially no negative or positive consequence on survival or reproductive ability), the observed incidence of the homozygous variant genotype would be approximately equal to the expected incidence of the homozygous variant genotype

$$( e . g . \frac{{Q}^{obs}}{{Q}^{exp}} \approx 1 ) .$$

If however, the variant genotype causes dysfunction, disease, or reduced likelihood of surviving or reproducing, there would be a selective force against that variant expressing in the population, thereby suppressing the observed incidence of the homozygous variant genotype relative to the heterozygote or total variant genotype

$$( e . g . \frac{{Q}^{obs}}{{Q}^{exp}} < 1 ) .$$

A relatively low observed incidence of the homozygous variant genotype compared to the expected incidence of the homozygous variant genotype (e.g. Q^{obs}<<Q^{exp}) results in a relatively high variant-specific gene dysfunction score, whereas a relatively neutral or high observed incidence of the homozygous variant compared to the expected incidence of the homozygous variant (e.g. Q_{obs}≥Qₑₓₚ) results in a relatively low variant-specific gene dysfunction score. At the limits of the homozygous effect, if there is no observed incidence of a homozygous variant genotype in a population (r) (e.g. Q^{r,obs}=0), the homozygous effect score reaches its maximum (e.g., T^{hom,r}=≈1) whereas if there are the same or more observed incidences of a homozygous variant genotype than expected in a population (r) (e.g. Q^{r,obs}≥Q^{r,exp}) the homozygous effect score reaches its minimum (e.g., Tᵣ^{hom}=0). The homozygous effect is a powerful and accurate measure of gene dysfunction caused by each variant (j) in a population (r), particularly in situations with a sufficiently large sampled population (e.g., Nⱼ^{r}>>1,000, such as >50,000 people) or a sufficiently high frequency of mutation (e.g., fⱼ^{r}>1%). However, in cases in which the population does not have many sequenced individuals (e.g. Nⱼ^{r}<1,000 people) or the mutation is at a sufficiently low allele frequency (e.g., fⱼ^{r}<0.1%), random fluctuations in mutations may skew the ratio of observed to expected homozygotes, and thus the homozygous effect becomes less powerful. To reflect the varied power in the score, the weight or confidence of the homozygous effect is proportional to the maximal number of observed or expected incidences of the homozygous variant genotype, and thus is also diminished in cases with low variant frequency and/or population size. In such cases when the power of the homozygous effect is diminished, the heterozygous effect may compensate.

The heterozygous effect may measure the impact of heterozygote forms of a recessive genetic variant. The heterozygous effect may measure the relationship between the count or frequency of a variant in each population (fⱼ^{r}) with the variant's clinical visibility. A variant (j)'s clinical visibility CVⱼ may be based on, for example a number of published articles that reference the variant (pm), a number of compound heterozygotes or combinations of the variant with other variants that is described in the clinical literature (ch), and/or a number of search results for the variant or an order or ranking of a search result for the variant in a database or web search. A rare variant or mutation is generally expected to only rarely (or never) appear in clinical studies. However, extensive documentation in clinical studies of a rare variant is shown to correlate with a higher likelihood that the variant causes gene dysfunction resulting in disease. The heterozygous effect increases when a variant has an unexpectedly or relatively high clinical visibility compared to its frequency in a population. The heterozygous effect is thereby of particular importance, for example, in cases where a variant has a high allele frequency (e.g. greater than 0.5%) or disproportionately high clinical visibility. In cases where a variant has a disproportionately high clinical visibility, the heterozygous effect may identify damaging or disease-causing variants even when they appear frequently in a population (e.g., with a sufficiently high frequency that would have otherwise indicated lack of damage). Although most disease variants are rare, for example, the variant primarily responsible for sickle-cell anemia (SCA) affects up to 10% of people in sub-Saharan countries with endemic malaria, which is a relatively high allele frequency for a damaging variant. However, the SCA variant has an extremely high clinical visibility, and thus both the score and weight for its heterozygous effect are relatively high. Allele frequencies from different populations may be treated unequally given that clinical studies are disproportionately prevalent in certain regions of the world. For example, for a variant with a null or 0 clinical visibility (e.g., not mentioned in the clinical literature), a 0.5% allele frequency in Europe would indicate a relatively low or minimal likelihood of that variant causing dysfunction, but a 0.5% allele frequency in Africa may still be aligned with the variant causing dysfunction, under the assumption that more clinical studies have been conducted in Europe than in Africa.

The dominant effect may measure each population's natural selection against dominant genetic variants. The dominant effect may measure the relationship between a variant's observed allele count (e.g., the number of people with either one or two copies of the variant) compared to an expected allele count for any pathogenic variant in the same gene, for example, based on a distribution of allele counts across a plurality of (or all) known pathogenic mutations in the gene. In a gene with a fully dominant disease, pathogenic variants are generally not expected to be observed in more than a small fraction of healthy individuals. If, for example, none or few of a gene's pathogenic variants have been observed in any individuals, and the number of pathogenic variants in the gene is sufficiently large (e.g., >30), then a variant that has been observed significantly more than would be expected of a pathogenic variant in that gene (e.g., allele count>2) would be given a relatively low dominant effect score. The weight or confidence of the dominant effect may be based on the variant's allele count, the number of pathogenic variants in the gene, and/or the distribution of allele counts across a plurality of pathogenic mutations in the gene.

Whereas the population selection component represents a variant's success or failure on a population-level, e.g., separately for each of a plurality of specific populations within the single human species (e.g., non-Finnish Europe, Finland, South Asia, East Asia, Africa, and the Americas), the evolutionary selection component represents a variant's success or failure on a species-level (in the “single-species” model) or across all species (in the “multi-species” model).

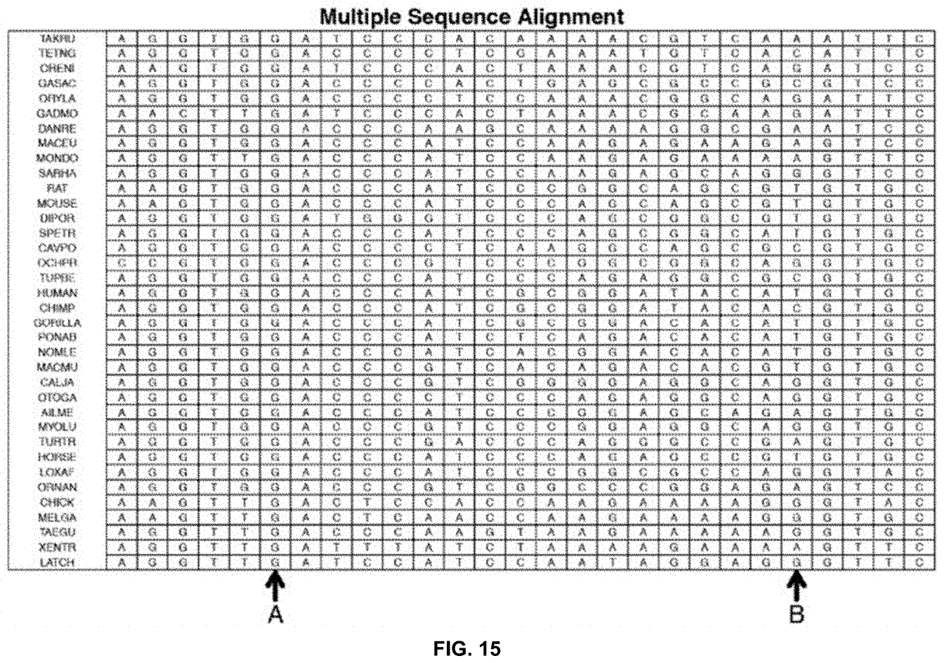

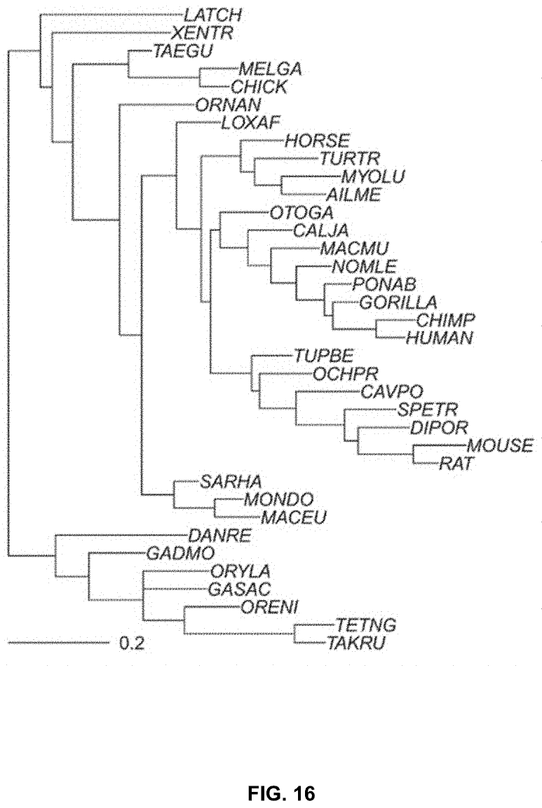

The evolutionary selection component predicts the likelihood that variants cause disease or dysfunction, for example, based on their frequency or rarity of occurrence, across multiple reference genetic sequences (e.g. see FIG. 15) sampled within one species (“single-species” model) or across multiple different species (“multi-species” model, see e.g. FIG. 16). The evolutionary selection component links a variant's success or failure to propagate throughout evolution by natural selection to the likelihood that the variant causes dysfunction or disease (rare variants) or that the variant is innocuous or positive (frequent variants). Accordingly, the evolutionary selection component for dysfunction may be inversely related to the frequency of the variant through evolution. Allele mutations or variations that have a relatively low frequency across the reference genetic sequences (e.g. negatively selected for evolutionarily) increase the variant-specific gene dysfunction score, whereas allele mutations or variations that are relatively more frequent across the reference genetic sequences (e.g. positively or neutrally selected for evolutionarily) decrease the variant-specific gene dysfunction score. At the limits of the evolutionary selection component, if there is no observed incidence of a variant in the specie(s) (e.g., fⱼ=0), the evolutionary selection component reaches its maximum (e.g., Sⱼ^{E}=1), whereas if the variant has a sufficiently high frequency (e.g. fⱼ>>0), the evolutionary selection component approaches its minimum (e.g., Sⱼ^{E}→0).

The evolutionary selection and population selection component may complement each other, providing improved prediction when used together than when used separately. In one instance, the evolutionary selection component may predict disease in variants that are suppressed throughout evolution, for example, the variant that eliminates the development of wisdom teeth. However, whereas wisdom teeth are typically essential to survival of many animal species, humans are an exception protected by the intelligence and altruism of our species and the adaptation of diet. This leads to an anomaly, whereas the evolutionary selection component alone would have caused a mischaracterization of the wisdom teeth variant as damaging, it combination with the population selection component neutralizes its effect.

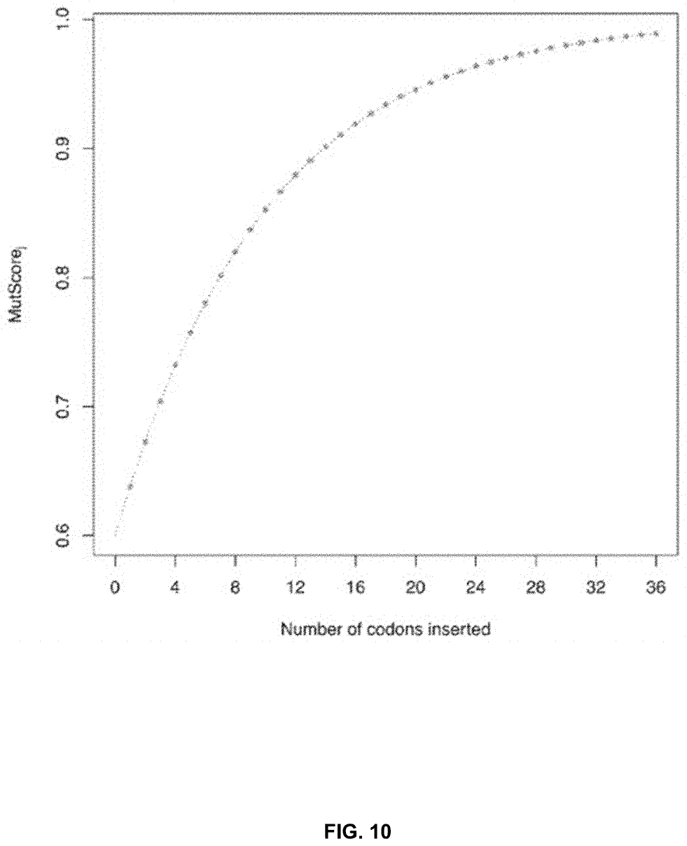

The mutation class component may measure the type or class of mutation of variant (j). Table 2 shows an example of the mutation class component for various mutation types. Example mutation classes include start-loss, stop-gain, stop-retained, frame-shift indel, essential splice site (associated with loss-of-function), splice region, untranslated region, microsatellite (e.g., a microsatellite or sequence of a repeating base type, such as, AAAAA . . . , of length STRj (short tandem repeats), synonymous (e.g., not affecting gene expression), intron, in-frame indel (e.g., an insertion or deletion of a multiple of three bases, or an integer number of amino acids AAj, so as not to shift the reading frame), missense (e.g., a non-synonymous variant that changes an amino acid), and stop-loss (e.g., loss of the normal stop codon by mutation to encode an amino acid). The loss-of-function (LoF) mutations, start-loss, stop-gain, frame-shift indel, essential splice site, typically cause complete dysfunction and may be assigned a relatively high or maximum dysfunction score (e.g., Sⱼ^{M}=1) with relatively high confidence (e.g., wⱼ^{M}>50). A missense mutation alters an amino acid and may be assigned an intermediate dysfunction score (e.g., Sⱼ^{M}=0.5), but because the amino acid may or may not damage a protein depending on which amino acid is damaged and other more complicated factors involved in the protein structure, it is associated with a relatively small weight (e.g., wⱼ^{M}=0.01). An in-frame indel mutation inserts or deletes an integer number (AAⱼ) of amino acids. Because the likelihood of dysfunction typically increases the greater the number of damaged amino acids, in-frame indel mutation dysfunction scores and weights increase as the number (AAⱼ) of damaged amino acids increases (e.g., as shown in FIG. 10). A micro-satellite mutation typically alters, adds, or deletes, one or more variants of a micro-satellite sequence. Because the likelihood of dysfunction typically decreases the longer the length of the micro-satellite (STRⱼ) the dysfunction score of a micro-satellite mutation is inversely proportional to the number of bases or length of the micro-satellite sequence (STRⱼ) of damaged amino acids (e.g., Sⱼ^{M}˜STRⱼ⁻¹) and the weight of the micro-satellite mutation is directly proportional to the length of the micro-satellite (e.g., wⱼ^{M}˜STRⱼ).

The clinical classification component may measure dysfunction for variants that were clinically validated, for example, as “pathogenic” (e.g., Sⱼ^{C}=1) or “benign” (e.g., Sⱼ^{C}=0). The weights of the score may be based on validated confidence levels (e.g., uncontested or certain classifications have a relatively high or maximal weight such as wⱼ^{C}=20, probable classifications have a relatively moderate weight such as wⱼ^{C}=10, and contested classifications have a relatively low or minimal weight of wⱼ^{C}=1). The clinical classification component may be null when there is no clinical classification for a variant, in which case, the remaining (e.g., non-zero) gene-dysfunction components compensate for the null clinical classification component to predict dysfunction for the variant in the absence of clinically validated data.

The pathogenic predictor component may measure a likelihood that a variant is pathogenic, inputting one or more of the following metrics: PROVEAN predicts whether a protein sequence variation affects protein function, Combined Annotation Dependent Depletion (CADD) predicts the effects of a single nucleotide variants as well as insertion/deletions variants, Variant Effect Scoring Tool (VEST) predicts the effects of missense mutations, and PolyPhen-2 (Polymorphism Phenotyping v2) predicts the effects of an amino acid substitution on the structure and function of a human protein. These metrics may be composed into the pathogenic predictor component as follows. The PROVEAN metric may be transformed to a linear scale subscore (e.g., the greater the PROVEAN metric, the more likely the variant damages a gene) and may be assigned a weight proportional to the metric (e.g., the greater the PROVEAN metric, the more certain its impact on gene function). The CADD metric may be transformed from a Phred scale to a linear scale subscore (e.g., the greater the CADD metric, the more likely the variant damages a gene) and may be assigned a weight that increases when the CADD metric is above a threshold parameter (sc) (e.g., above a threshold, the greater the CADD metric, the more certain its impact on gene function). PolyPhen-2 includes two metrics (HumDiv and HumVar) that may be averaged (e.g., the greater the combined metrics, the more likely the variant damages a gene) and assigned a weight inversely proportional to the difference between the metric (e.g., the more the two metrics disagree, the less reliable are the metrics). The VEST metric may be assigned as a subscore (e.g., the greater the VEST metric, the more likely the variant damages a gene) and may be assigned a constant weight. In general, all of these metrics have approximately 80% accuracy in assigning predicted non-pathogenic variants relatively low scores and predicted pathogenic variants relatively high scores. However, these four metrics are generally inconsistent in the scoring of a subset of partially functional pathogenic variants due to the overly simplistic pathogenic categorization in ClinVar. It is only by combining these components with other gene dysfunction components in the neural network that embodiments of the invention produce cumulative gene dysfunction scores with sufficient accuracy of for example greater than 90-95% as described below.

### Improvements

Embodiments of the invention may provide the following improvements and overcome the aforementioned longstanding issues inherent in the art:

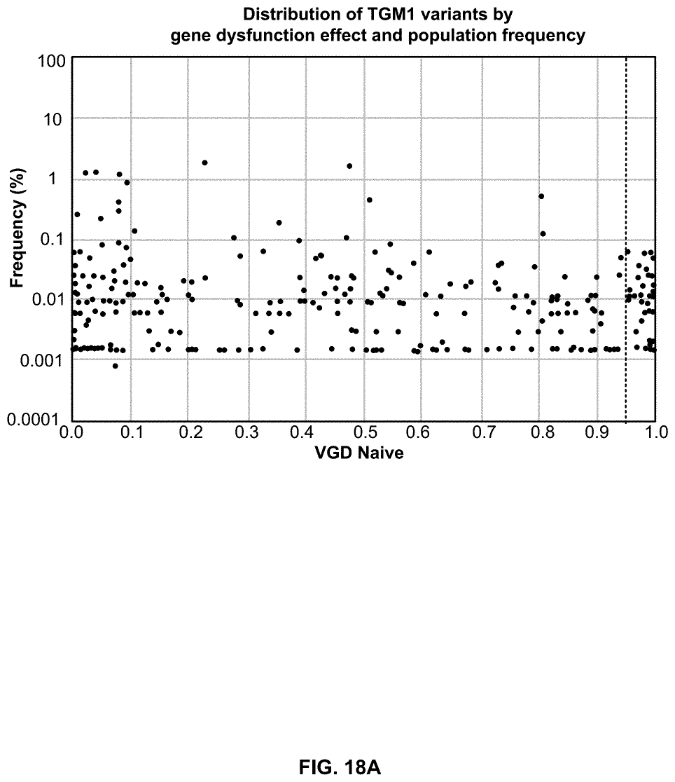

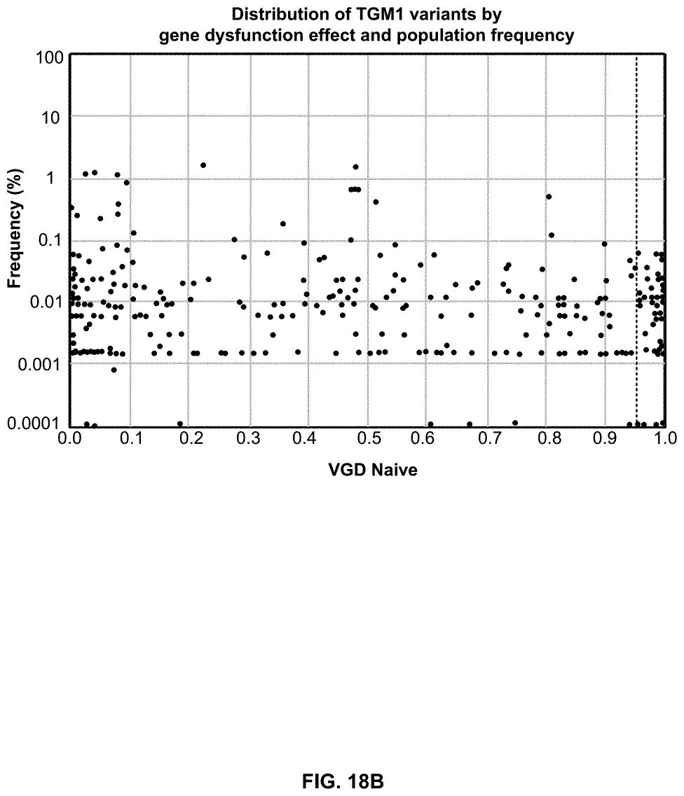

Improved results: Table 1 and FIGS. 18A-B (discussed below) both show examples of novel or de novo variants, missed by standard models, but identified by the VGD model as pathogenic or benign. The discovery of these novel variants is due to the introduction of several new metrics of gene-dysfunction—the population selection component, the homozygous effect, the heterozygous effect, the dominant effect and the evolutionary constraint component—as well as an optimization of their combination using a neural network.

Neural Network: The neural network composes multiple gene-dysfunction components representing different and complementary aspects of gene dysfunction in an optimized manner to produce a cumulative prediction that is greater than the sum of its parts. Whereas separately analyzing all of these gene dysfunction components one-at-a-time predicts for example up to at most only 80% of pathogenic variants, analyzing multiple gene dysfunction components optimized together in the neural network improves gene-dysfunction accuracy predicting for example greater than 90-95% of pathogenic variants (see e.g., discussion of FIGS. 18A-B below). For example, in one instance, the homozygous effect provides relatively high accuracy for predicting gene dysfunction of variants in cases with relatively high variant frequency or relatively large population size, but provides relatively low accuracy in cases with relatively low variant frequency or population size. In these latter cases, the homozygous effect weight is diminished and other components such as the heterozygous effect dominate to compensate for an inaccurate homozygous effect. For example, in cases where a population sample and/or variant frequency is small, a disease-gene may still have wide clinical visibility, bolstering its heterozygous effect, or may be identified as damaging by its clinical classification, pathogenic predictors or mutation class. In another case of dominant traits or disease, the homozygous effect (adapted to identify recessive traits) and the heterozygous effect (only significant if the trait has substantial clinical visibility) may be insufficient. For dominant traits, the dominant effect compensates for these other effects, accounting for dominant disease variants by diminishing scores of known gene mutation that have higher than expected incidence compared to an expected incidence for pathogenic mutations of the same dominant gene. In another example, relatively high frequency variants typically have a relatively low evolutionary constraint component. Such relatively high frequency variants would therefore be ignored if they were classified on the basis of the evolutionary constraint component alone. Because the variant-specific gene dysfunction integrates multiple gene dysfunction components, for diseases with relatively high frequencies in certain populations, such as sickle-cell anemia (SCA), this suppressed evolutionary component may be augmented by a relatively high population selection, pathogenic predictors, clinical classification and/or mutation class components. In one example, variants within a gene associated with deafness may have a very high-damage score for the evolutionary constraint component indicating death or low-likelihood of survival because the ability to hear is critical to survival for most species. However, human populations have adapted to deafness, so the ability to hear is no longer critical for survival in most human populations. Accordingly, the population selection component corrects for an otherwise inflated damage score for deafness due to the evolutionary constraint component. Thus, each of the variant-specific gene dysfunction components are adapted to identify gene dysfunction with more or less accuracy in different sets of circumstances, thereby filling in “blind spots” where other components may not fully capture the gene effect, so that the combination of the multiple variant-specific gene dysfunction components together more accurately predicts gene dysfunction than analyzing the individual components separately.

Continuous Classification: In contrast to conventional binary classification systems, embodiments of the invention provide a continuous classification system of scores distributed on a continuous (e.g., linear) scale. Because the causation between variants and gene-dysfunction is typically uncertain, in particular, when analyzing novel variants not yet clinically validated, or only validated to a contested or uncertain confidence, the continuous likelihood or variant-specific gene dysfunction described according to embodiments of the invention may more accurately represent pathology as compared to a conventional binary classification. Further, the continuous likelihood described according to embodiments of the invention may account for the varying degree of gene dysfunction, for example, to identify any continuum or intermediate effects (e.g., a degree of disease or partial functionality of a phenotype) or to differentiate between different degrees of gene-dysfunction caused by different allele or genotype combinations of the same gene.

Population Selection: In contrast to the conventional carrier testing which has no way to test for population-specific anomalies or differences in selective factors, such as, deafness or the elimination of wisdom teeth, embodiments of the invention provide a population selection component that measures the relative propensity or aversion for specific variants on a population-by-population basis.

Homozygous Recessive Predictive Screening: A recessive gene must typically be a homozygote (having two copies) to express a recessive disease or trait. Because many newly arising mutations in recessive disease genes have not yet expressed as homozygotes, or worse yet, have killed off all patients with those homozygotes, conventional carrier testing has no way to test for new recessive disease gene mutations. In contrast, embodiments of the invention provide a homozygous effect component that measures the observed incidence or frequency of homozygote variants compared to an expected homozygote incidence. The expected homozygote incidence may be generated by extrapolating from the heterozygote or total variant incidence to predict what the expected homozygote incidence would be if there was no selective factor against the homozygote form of the variant. A relatively low observed homozygote incidence compared to the expected homozygote incidence indicates a likely selective factor against the homozygote forms of the genotype, increasing the likelihood that the variant causes a recessive disease or dysfunctional trait.

Heterozygous Effect: In contrast to the conventional classification systems which have no way of testing the effect of heterozygous variants for recessive traits, embodiments of the invention propose a heterozygous effect component that is a relative measure between variant frequency and clinical visibility. The heterozygous effect component identifies variants that have a disproportionately high clinical visibility compared to their frequency in a population. This unexpectedly high clinical visibility may indicate that these variants are likely candidates for recessive disease or dysfunction. Conversely, variants that have a disproportionately low clinical visibility compared to their frequency in a population may indicate that these variants are unlikely to contribute to recessive disease or dysfunction.

Dominant Predictive Screening: Variants linked to dominant disease or gene-dysfunction are typically difficult to detect because organisms with those variants seldom survive, or only proliferate for a few generations. The VGD model however can predict dominant disease gene mutations based on the allele count of the variant compared to a distribution of allele counts across a plurality of pathogenic mutations in the same gene. If the allele count of that particular variant is relatively higher than expected based on the distribution of the allele counts of the pathogenic variants for that gene, the likelihood that the variant causes gene dysfunction as a dominant allele may be relatively decreased.

Evolutionary Selection: The evolutionary selection component may use evolution as a “four billion year experiment.” During the course of evolution, nearly all variants or mutations have likely been tested and their success or failure in propagating through one or more species by natural selection indicates which variants cause dysfunction or disease (rare variants) and which variants are innocuous or positive (frequent variants). The evolutionary selection component may thus relate the measure of evolutionary variation of alleles at a genetic locus to the likelihood that a variant at that locus will cause disease or dysfunction.

Pre-clinical screening: In contrast to the conventional carrier testing which only tests for clinically validated disease-causing variants, embodiments of the invention may use the population selection and evolutionary selection components to predict the effect or disease-risk of allele variations without clinicians having ever observed those allele variations in diseased patients.

Reference is made to FIGS. 18A and 18B, which are graphs of the VGD scores for 400 variants of an example disease gene, transglutaminase (TGM) 1, compared with clinically validated results in (A) 2012 and (B) 2016 according to an embodiment of the invention. Each dot in each graph represents one of the about 400 variants that were analyzed. The VGD score for each variant is plotted along the x-axis of both graphs. The vertical dashed line in the graphs (e.g., at VGD=0.953) delineates a pathogenic threshold, above which variants are predicted to be pathogenic or disease-causing. The key at the bottom of the graphs represents clinical data indicating which variants were clinically validated (observed to cause an effect in a real patient) to be “Benign”, “Contested Pathogenic”, “Probably Pathogenic”, or “Pathogenic” (observed to cause disease in a real diseased patient), and which variants were not yet clinically validated “No ClinVar Pathogenicity Classification” (not yet observed to cause an effect in a real patient). The two graphs show different years of clinical validation data-FIG. 18A shows clinical validation data for 2012, labeling the only 11 variants that were clinically validated as pathogenic for TGM1 in 2012, and FIG. 18B shows clinical validation data for 2016, labeling the increased number of 26 variants that were clinically validated as pathogenic for TGM1 by 2016.

The 2012 graph in FIG. 18A shows that the VGD model correctly identified 10 out of 11 pathogenic variants known for the TGM1 gene in 2012 (90.9% accuracy). The 2016 graph in FIG. 18B shows that the VGD model correctly identified 24 out of 26 pathogenic variants known for the TGM1 gene in 2016 (92.3% accuracy). Comparing the two graphs in FIGS. 18A and 18B, the VGD model predicted 14 out of the 15 variants (93.3%) of the TGM1 gene that were newly validated as Pathogenic between 2012 and 2016 (variants whose classification switched from a “No Clinical Validation” classification in the 2012 graph of FIG. 18A to a “Pathogenic” classification in the 2016 graph of FIG. 18B). Thus, 14 novel disease-causing variants were predicted based on the VGD scores (at the time of prediction, there was no clinical curation because these variants were not yet known).

The VGD scores use the population selection, mutation class, pathogenic predictors, and evolutionary constraint components to predict the effect or disease-risk of allele variations without clinicians having ever observed those allele variations in diseased patients. This allows geneticists to assess the disease-risk of novel or de novo mutations or variants that have never before been validated or studied in diseased patients. The graphs in FIGS. 18A and 18B show that the VGD score accurately predicted pre-clinical disease-risk with a confidence of greater than 90% and predicted 93.3% of newly validated variants as disease-causing in the example of the TGM1 gene. Carrier screening is thereby no longer restricted to testing for disease caused by the limited number of already-known clinically validated disease variants. These graphs show that, in 2012, carrier screening only screened for at most 11 pathogenic variants of TGM1, while the VGD model screened for 24 pathogenic variants of TGM1. This indicates that the VGD score accurately predicted 13 new pathogenic or disease-causing variants for the TGM1 gene (only later validated in 2016) before geneticists would have ever tested for them in carrier screening because those 13 variants had not yet been validated as disease-causing at the time of testing (non-validated in 2012). This improves genomics and carrier screening by discovering disease-correlated variants before the variants are clinically validated, thereby predicting disease risk in patients that would have otherwise been ignored, improving the accuracy of carrier screening and potentially mitigating disease and saving lives. (Table 1 also provides a list of example de novo variants, missed by standard models, but identified by the VGD model as likely to be disease-contributing or neutral.)

Pre-conception screening: Conventional carriers screening only tests for variants that have already been identified and validated in a living diseased child. Some embodiments of the invention compute the VGD score or likelihood of disease-risk of allele variations in “virtual progeny” (non-existing, pre-conception progeny) based on measures of population selection or evolutionary variation, instead of only based on clinically validating the genomes of real diseased children (existing, post-conception progeny). Embodiments of the invention can thereby predict the likelihood that an allele variation will cause disease without requiring that any child ever be conceived with that disease or disease-causing variant. As shown in FIGS. 18A and 18B, the VGD score can be used to identify novel, never before validated, variants as disease-causing with extremely high accuracy (93.3% of newly validated variants in the example of the TGM1 gene), without ever having to clinically observe such a variant in a real living organism. This improves genomics by discovering disease-causing variants at an earlier stage, before a child is ever conceived with the disease or disease-causing variant, thereby potentially reducing disease in children.

Other or different advantages may be realized according to embodiments of the invention.

### System Overview

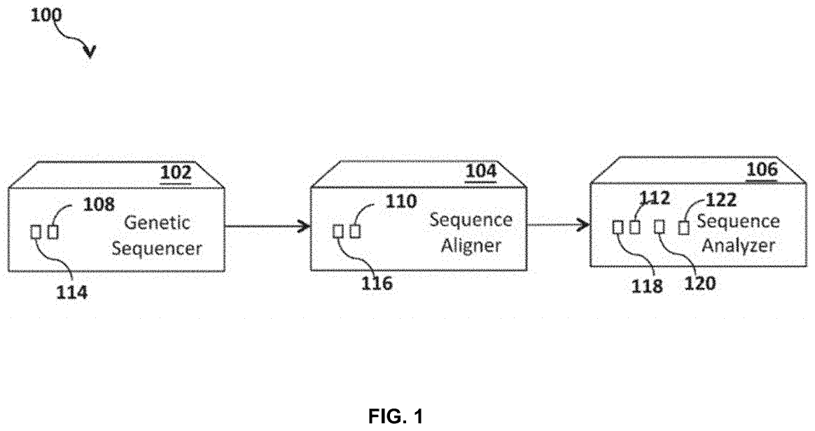

Reference is made to FIG. 1, which schematically illustrates a system 100 for assessing risk or likelihood of variant-specific gene dysfunction according to an embodiment of the invention.

System 100 may include a genetic sequencer 102, a sequence aligner 104 and/or a sequence analyzer 106. Units 102-106 may be implemented in one or more computerized devices as hardware and/or software units, for example, specifying instructions configured to be executed by a processor. One or more of units 102-106 may be implemented as separate devices or combined as an integrated device.

Genetic sequencer 102 may input DNA obtained from biological samples, such as, blood, tissue, or saliva, of one or more real living organisms and may output each organism's genetic sequence including the organism's genetic information at one or more genetic loci, for example, a human genome. A single organism's DNA sample may be sequenced for performing carrier testing on that individual, two potential parents' DNA samples may be sequenced for performing carrier testing on a virtual progeny generated by combining at least a portion of the two potential parents' genetic sequences, or a single potential parent's DNA sample may be sequenced for combining with each of a plurality of candidate donor sequences to generate a plurality of virtual progeny to determine an optimal and/or a least optimal subset of one or more donors.

Sequence analyzer 106 may generate virtual progeny by inputting two potential parents' genetic sequences to simulate a mating by combining at least a portion of genetic information derived therefrom and output a virtual progeny genetic sequence of a virtual gamete, for example, as described in reference to FIG. 17.

Sequence aligner 104 may align one or more loci of the organism or virtual progeny's genetic sequence with a plurality of reference genetic sequences of extant organisms from (a) one or more different populations for generating a population selection component and (b) one or more different species for generating an evolutionary constraint component. In some embodiments, a sequence aligner need not be used.

Sequence analyzer 106 may input the multiple sequence alignment and may compute measures of (a) population-specific variation of alleles and (b) species-wide evolutionary variation of alleles at one or more aligned genetic loci. Sequence analyzer 106 may generate (a) a population selection component and (b) an evolutionary constraint component based on these measures (e.g. as shown in FIG. 3A-B). Sequence analyzer 106 may use a neural network to combine these and other components (e.g., population selection component, evolutionary constraint component, clinical classification component, and/or mutation class components) to generate one or more variant-specific gene dysfunction likelihoods or scores measuring the relative propensity or risk that these alleles are damaging in a living organism or would be damaging if produced in a child. Sequence analyzer 106 may have a training phase and a runtime phase. In the training phase, sequence analyzer 106 may compare VGD predictions to known results to validate the model and set parameters (see e.g., “Parameter” in FIG. 3D), confidence weights, and sensitivity thresholds. In the runtime phase, sequence analyzer 106 may generate VGD scores to test the risk of gene dysfunction due to specific allele variants or mutations, including de novo variants (not yet clinically validated), in living organisms or in virtual progeny of potential parents (before a child of those potential parents is conceived). In some embodiments, sequence analyzer 106 may analyze a specific one or more targeted variants, or may progress locus-by-locus to analyze all (or a plurality) of variants throughout the human genome. Sequence analyzer 106 may analyze variants in one pass (a full analysis of all components), or multiple passes (e.g., using a coarse analysis of only one or a subset of components in a first pass to flag a subset of variants with an above-threshold likelihood to be more closely analyzed in a second pass). In the training and/or runtime phases, sequence analyzer 106 may compute each node in the neural network or variant, in series or in parallel.

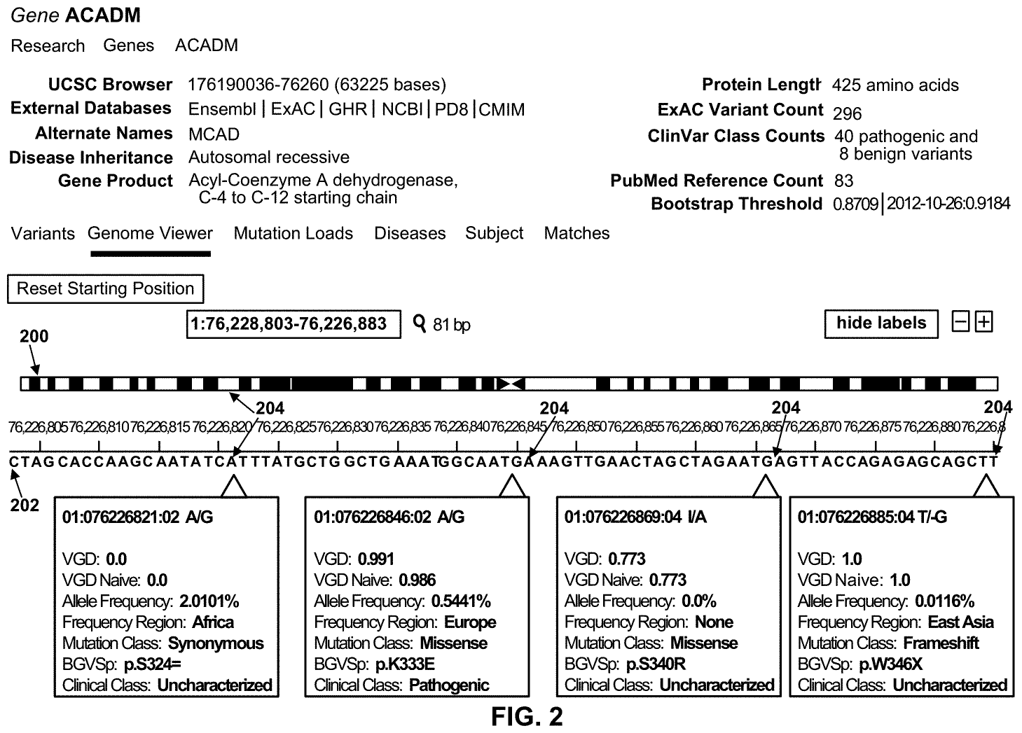

Genetic sequencer 102, sequence aligner 104, and sequence analyzer 106 may include one or more controller(s) or processor(s) 108, 110, and 112, respectively, configured for executing operations and one or more memory unit(s) 114, 116, and 118, respectively, configured for storing data such as genetic information or sequences and/or instructions (e.g., software) executable by a processor, for example for carrying out methods as disclosed herein. Processor(s) 108, 110, and 112 may include, for example, a central processing unit (CPU), a digital signal processor (DSP), a microprocessor, a controller, a chip, a microchip, an integrated circuit (IC), or any other suitable multi-purpose or specific processor or controller. Processor(s) 108, 110, and 112 may individually or collectively be configured to carry out embodiments of a method according to the present invention by for example executing software or code. Memory unit(s) 114, 116, and 118 may include, for example, a random access memory (RAM), a dynamic RAM (DRAM), a flash memory, a volatile memory, a non-volatile memory, a cache memory, a buffer, a short term memory unit, a long term memory unit, or other suitable memory units or storage units. Genetic sequencer 102, sequence aligner 104, and sequence analyzer 106 may include one or more input/output devices, such as output display 120 (e.g., such as a monitor or screen) for displaying to users results provided by sequence analyzer 106 (e.g., visualizing FIGS. 2, 4-18) and an input device 122 (e.g., such as a mouse, keyboard or touchscreen) for example to control the operations of system 100 and/or provide user input or feedback, such as, selecting one or more models or phylogenetic trees, selecting one or more genus or species to use for generating the models, selecting input genetic sequences, selecting two potential parents or multiple donor candidates from a pool of potential parents with which to simulate mating, selecting a number of iterations for simulating a mating with a different pair of virtual gametes in each iteration from each pair of potential parents, etc.

Reference is made to FIG. 2, which schematically illustrates a display visualizing a DNA image 200 and/or sequence 202 of an organism comprising one or more variants or mutations 204 labeled in the DNA together with detailed information 206 including a likelihood of variant-specific gene dysfunction according to an embodiment of the invention.

DNA image 200 may be an image of DNA extracted from biological samples, such as, blood, tissue, or saliva. The organism may be a living organism or a virtual organism. When screening a living organism, DNA image 200 may be an image of DNA of the living organism undergoing screening. When screening a virtual organism, DNA image 200 may be an image of DNA of one or more of the two living potential parents whose DNA is combined to generate the virtual organism undergoing screening. For example, when two potential parents undergo carrier screening to predict disease or dysfunction in their potential child, the two potential parents' DNA may both be imaged, whereas when one potential parent seeks screening with a pool of donor candidates, the image of the DNA of the one potential parent may be displayed alone (without DNA images of candidate donors, e.g., for privacy issues) or together in a sequence of displays with the DNA image of each respective candidate donor. DNA image 200 may display a portion of or the entire length of a human genome.

DNA sequence 202 may be a schematic representation of the DNA described above, such as a sequence of nucleotides, bases, amino acids or other genetic information representing the DNA (e.g., sequenced by genetic sequencer 102 of FIG. 1). In one embodiment, DNA sequence 202 may be a magnified view of a section or segment of DNA image 200, and DNA image 200 may be highlighted to label the DNA segment to which DNA sequence 202 corresponds. DNA image 200 and/or DNA sequence 202 may be dynamic displays. For example, a user may control DNA image 200 by translating or zooming in or out of the stream, or may control DNA sequence 202 by selecting a corresponding center-point, segment, or magnification range, of DNA image 200.

One or more genetic mutations 204 or their positions may be identified or labeled in DNA image 200 and/or DNA sequence 202 e.g., by color, tone, labeling, highlighting, etc. Identified genetic mutations 204 may be variants identified or flagged by a processor (e.g., 112 of FIG. 1) to have an above threshold likelihood of variant-specific gene dysfunction (e.g., flagged as likely “pathogenic”), or variants selected by user-input to undergo screening.

The display may provide detailed information 206 about the identified genetic mutations 204, for example, automatically where flagged as pathogenic or if selected or identified by a user (e.g., by hovering a cursor over the variant in DNA image 200 and/or DNA sequence 202). The detailed information 206 may include, for example, one or more likelihoods that the identified genetic mutation 204 causes gene-dysfunction in the organism (e.g., a VGD score and/or a naïve VGD score, VGD−Cl, omitting clinical classification data), a frequency of the genetic mutation (e.g., a total number of instances of the mutation in one or more populations, and population in formation), a mutation class, HGVsp (the standard notation for identifying an amino acid substitution), and/or clinical classification. Other information, combinations of information, or visualizations of information.

### Example Data Sources

Analytic targets-Analyses described according to embodiments of the invention targeted an example set of exons from 480 genes associated with autosomal recessive disease. The gene set was composed of all autosomal genes covered by Illumina's TruSight Inherited Disease Sequencing Panel (URL: http://support.illumina.com/downloads/trusight_inherited_disease_product_files.html accessed on Sep. 24, 2014) as well as all additional autosomal genes targeted by Counsyl's Family Prep platform. Illumina TruSight One Sequencing Panel's intervals were used to target the exon regions of each gene analyzed. Exon intervals were padded with a minimum of 10 by (base pairs) to a maximum of 50 by of intronic sequence to include variants listed as “pathogenic” in the ClinVar datasets.

ExAC dataset-Population-specific allele and genotype frequency data for variants in targeted intervals were obtained from an example of 60,706 sequenced exomes that were consolidated and processed by the Exome Aggregation Consortium (ExAC) and made publically available through the Consortium's website (Version 0.3, URL: http://exac.broadinstitute.org accessed on Jan. 13, 2015). The ExAC cohort is composed of unrelated individuals from six geographically defined, and in most cases, genetically distinguishable populations: non-Finnish Europe (NFE; population size, n=33,370), Finland (FIN; n=3,307), South Asia (SAS; n=8,256), East Asia (EAS; n=4,327), Africa (AFR; n=5,203), and the Americas (AMR; n=5,789). ExAC subjects represent individual participants in several large-scale disease-specific and population genetic studies. Persons diagnosed with targeted pediatric diseases were generally excluded from participation.

ClinVar annotation archive—The ClinVar archive of DNA variants with clinical annotations is maintained in two partially overlapping datasets indexed on the human reference genome (hg19) or the HGVS format for describing variation in transcribed genomic intervals. Clinical assertions were retrieved related to variants located in targeted regions by parsing the two files clinvar.vcf and variant_summary.txt (both accessed at URL: http://www.ncbi.nlm.nih.gov/clinvar/ on Mar. 6, 2015; file parsing details can be found in more detail below).

VariBench dataset-Data from the VariBench website (URL: http://structure.bmc.lu.se/VariBench/, accessed on Feb. 6, 2015) was downloaded and processed as a second variant benchmark set. The VariBench metric clusters experimentally verified variants into “pathogenic” and “neutral” (or synonymously benign) datasets. The VariBench variants were divided into subsets that overlap targeted intervals.

Online Mendelian Inheritance in Man (OMIM)—OMIM is a catalogue of human genes and diseases with separate English language narrations provided for major allelic variants of disease genes (accessed as the compressed file omim.txt.Z located at URL: ftp.omim.org/OMIM/ on Mar. 3, 2015). Among other information, allele-specific narrations contain references to other indexed alleles in the same gene that have been detected in compound heterozygous patients. This analysis used a “natural language” approach and cross-indexing among alleles to enumerate distinct compound heterozygous second alleles found in association with each primary allele.

All data sources and testing parameters are described as examples, and are optional and may be omitted, or replaced by equivalents.

### Variant-Specific Gene Dysfunction Modeling

Reference is made to FIGS. 3A-3D, which schematically illustrate an example of (A) a neural network combining multiple gene dysfunction components for determining the risk of variant-specific gene dysfunction in an organism; (B) an exploded view of the population selection component; (C) an exploded view of the clinical classification component, mutation class component and pathogenic predictors component; and (D) optimized parameters for the components in FIGS. 3A-3C, according to an embodiment of the invention.

In FIG. 3A, the neural network combines multiple gene-dysfunction components for determining the risk or likelihood that a specific variant or genetic mutation causes gene-dysfunction in organisms. For example, the multiple gene-dysfunction components include clinical classification, mutation class, pathogenic predictors, evolutionary constraint, and/or population selection. The neural network includes a plurality of nodes (b=1, 2, . . . , B), each node representing a different gene-dysfunction component, for example, associated with one or more sub-likelihoods or subscore(s) Sⱼ^{b} and associated weight(s) wⱼ^{b}. For each of one or more variants, j (j=1, 2, . . . J), the neural network may compute the B dysfunction components or subscores at the nodes. Each subscore Sj b may be computed on a continuous scale (e.g., [0.0, 1.0], or more generally [N, M] where N, M are rational numbers). Each subscore, Sⱼ^{b} (b=1, 2, . . . B), may be multiplied by a corresponding “final” component weight, wⱼ^{b}, which may represent the relative level of confidence ascribed to a particular scoring component b for a particular variant j.

Component weights may initially be “raw” weights, wⱼ^{0b} (initial or raw denoted by a “0” in the superscript) on a continuous raw scale (e.g., [0.0 to 99]), for example, derived according to component-defined sets of functions and rules. Variant components with missing or unused values may be designated as “unassigned” and/or may be given a null raw weight of, for example, 0.0. The final or scaled component weights, w, for each variant j may be obtained by recalibrating the raw weights, for example, to sum to a maximal value on the scale (e.g., 1.0 or N):

$${w}_{j}^{b 0} = \frac{{w}_{j}^{0 b}}{{\sum }_{= 1}^{B} {w}_{j}^{0 b}} \qquad ( 1 )$$

The likelihood of variant-specific gene dysfunction for variant j, denoted as VGDj, may be computed as a weighted sum of the B dysfunction components:

$${VGD}_{j} = {\sum }_{b = 1}^{B} {w}_{j}^{b} {S}_{j}^{b} . \qquad ( 2 )$$

The specific component subscores and weights are explained in more detail in their respective sections below. The neural network of FIGS. 3A-3D may incorporate an unlimited number B of component dysfunction subscores (sⱼ^{b}) at its nodes, each weighted according to an independent confidence or weight (wⱼ^{b}). In some embodiments of the invention, the neural network is not static, but dynamic. Dynamic embodiments may add new component nodes or update the values for the current nodes (e.g., adding new clinical classification or mutation type data as it is discovered), or may update the system parameters (see e.g., “Parameter” in FIG. 3D), confidence weights, and sensitivity thresholds by re-training the model using new input data in a training phase. In some examples, the VGD model may be re-trained periodically and/or upon a trigger such as the uploading or indication of new input or training data. Typically, the population size (Nⱼ^{r}), variant frequencies (fⱼ^{r}) and/or clinical visibility (CV) are revised with the expansion of available data. The training phase may optimize the neural network based on a cost factor, for example:

$${C}_{VGD} = \frac{1}{3 G} {\sum }_{g}^{G} [ ( 1 - {t}_{g}^{bs} ) + \frac{{u}_{g}^{> bs}}{{U}_{g}} + {\hat{d}}_{g}^{cv 2} ] , \qquad ( 3 )$$

Where t_{b}^{bs} is a pathogenic bootstrap threshold providing a lower bound for the VGD scores of a predetermined number or percentage (e.g., 99%) of known pathogenic mutations, u_{g}^{>bs} is a measure of how many uncharacterized variants with a minimum allele count (e.g. allele count>5) fall above that pathogenic bootstrap threshold, U_{g} is a total count of uncharacterized variants to scale as a ratio, {circumflex over \hat{d}_{g}^{cv2} is a measure of the mean dysfunction values for benign variants (e.g., classified as ClinVar-2 or ClinVar-3). The neural network is optimized in the training-phase by reducing the cost factor CV_{G}D. Minimizing or reducing CVGD may optimize the VGD model to better match three groups of variants: (1) known pathogenic variants are matched by shifting the center of VGD of known pathogenic mutations toward one or more maximal likelihoods (e.g., by minimizing (1−t_{g}^{bs}) in the cost factor when the maximal likelihood is 1); (2) known benign variants are matched by shifting the center of VGD of known benign mutations toward one or more minimal likelihoods (e.g., by minimizing \hat{d}_{g}^{cv2} in the cost factor); and (3) uncharacterized variants are matched by shifting the center of VGD of uncharacterized away from the one or more maximal and/or minimal likelihoods (e.g., by minimizing

$$\frac{{u}_{g}^{> bs}}{{U}_{g} \vert}$$

in the cost factor). The neural network is optimized in the training-phase on a gene-by-gene basis, after which the gene-specific optimization results are aggregated or summed across multiple genes, genome segments or an entire genome, for example, to obtain a combined genome-wide cost factor to be minimized.

Population Selection Component

The “population selection” component PopScoreⱼ(S^{P)} may provide a population-specific measure that each individual population suppresses or naturally selects against a particular allele variant or mutation. The population selection component may be inversely related to the observed frequency of the allele in the population. Because natural selection typically factors against damaging or disease-causing variants, the more frequent a variant is, the less likely the variant is to cause a trait that is considered dysfunctional or disease-causing in a particular population; whereas the less frequent a variant is, the more likely the variant is to cause such a trait. Because each population is unique, different populations will generally select differently against some traits (e.g., deafness may be considered acceptable for survival in some modern populations, but not in other populations such as hunting populations) and may select similarly against some common or universally dysfunctional traits (e.g., traits that threaten survival for all humans such as cancers). The population selection component balances a homozygous effect, a heterozygous effect and/or a dominant effect.

The homozygous effect may be used to identify variants that cause recessive disease or traits. The homozygous effect may measure the frequency or rarity of homozygote variants for each of multiple populations, for example, by comparing an observed homozygote incidence of a variant (e.g., measured on both chromosomes at the variant's genetic locus) relative to an expected homozygous incidence (e.g., based on a total observed incidence of the identified genetic mutation measured on either chromosome at the genetic locus and the population size) in each population. The likelihood of variant-specific gene dysfunction score may be inversely proportional to the ratio of the observed versus expected homozygote variants because a suppressed incidence of homozygotes in the population may indicate a population selection against those homozygotes (e.g., for causing a recessive trait or disease).

The heterozygous effect may measure the total frequency of variants (e.g. measured on either chromosome) in each population relative to its prevalence in the clinical literature. The likelihood of variant-specific gene dysfunction score may increase when the relative clinical visibility compared to its allele frequency in a population increases, indicating the variant is an anomaly or especially relevant to disease diagnostics.

The dominant effect may be used to identify variants that cause dominant diseases or traits. The dominant effect may measure a variant's observed allele count compared to an expected allele count for any pathogenic variant in the same gene, for example, based on a distribution (e.g., a Poisson or CFD distribution) of allele counts across a plurality of (or all) known pathogenic mutations in the gene. If the allele count of that particular variant is relatively higher than expected based on the distribution of the allele counts of the pathogenic variants for that gene, the likelihood that the variant causes gene dysfunction as a dominant allele is relatively decreased.

Pathogenic Predictors Component

The “pathogenic predictors” component PathScoreⱼ(Sⱼ^{PP})j may compose one or more metrics (Rⱼ^{PP}) that predict a predicted degree of pathology of a variant or variant class under analysis. For example, a PROVEAN metric (Rⱼ^{PR}) and a VEST metric (Rⱼ^{V}) predict whether a protein sequence variation affects protein function, a CADD metric (Rⱼ^{C}) predicts the effects of any type of variant, and PolyPhen-2 metric (Rⱼ^{P2}) predicts the effects of a missense amino acid substitution on the structure and function of a human protein. All four pathogenic predictor metrics are trained by machine-learning on sets of presumed pathogenic and presumed benign variants. The pathogenic predictors component (Sⱼ^{PP}) may be defined, for example, as:

Sⱼ^{PP}=Σₚₚ₋₁^{PP}uⱼ^{PP}Rⱼ^{PP}| (4),

where (Rⱼ^{PP}) is the predictive damage score generated from each of PP pathogenic predictor metrics (pp=1, 2, . . . , P), and (Uⱼ^{PP}) is the corresponding subcomponent weight. The choice of pathogenic predictor metrics (Rⱼ^{PP}) may also be dynamic; the pathogenic predictors component may add, recompose, and remove subcomponent metrics in equation (4), for example, eliminating nodes that have no data (e.g., “unassigned”) or when a confidence weight is substantially negligible (e.g., nodes with negligible weight may be considered equivalent to discounting the nodes completely, as long as other nodes have significant weight).

Raw data for the pathogenic predictors, PolyPhen-2, VEST, CADD, and PROVEAN, may be obtained, for example, by sending query batches to the respective websites (http://genetics.bwh.harvard.edu/pph2/bgi.shtml, http://www.cravat.us/, http://cadd.gs.washington.edu/score, and http://provean.jcvi.org/genome_submit_2.php).

Each of the raw pathogenic predictor data may be differently scaled and the pathogenic predictor metrics (Rⱼ^{PP}) may map or transform the raw pathogenic predictor data onto a uniform scale. The scale for this and all component scores may be, for example, a [0.0, 1.0]) scale, or any [M, N] scale, where M, N are rational numbers such as integers.

Raw VEST data is provided on a [0.0, 1.0] scale. The VEST metric (Rⱼ^{V}) may map the raw VEST values linearly by a constant factor. For example, if the pathogenic predictors component is provided on a [0.0, N.0] scale (where N is an integer), the raw VEST values may be multiplied by N. In the example where the pathogenic predictors components (Rⱼ^{PP}) use the same [0.0, 1.0] scale as the raw VEST values, the raw VEST values need not be scaled and may be set to equal the VEST metric (Rⱼ^{V}).

Raw PolyPhen-2 data provides two metrics, HumDiv and HumVar, which are trained using two different models. Each of the HumDiv and HumVar values are provided on a [0.0, 1.0] scale. The PolyPhen-2 metric (Rⱼ^{P2}) averages these two metrics (HumDiv and HumVar), thereby mapping each of them onto a half-scale (dividing each metric by a factor of 2). For example, if the pathogenic predictors component is provided on a [0.0, N.0] scale (where N is an integer), the raw PolyPhen-2 values HumDiv and HumVar may each be multiplied by N/2. In the example where the pathogenic predictors components (Rj pp) use the same [0.0, 1.0] scale as the raw PolyPhen-2 values, the raw PolyPhen-2 values HumDiv and HumVar are each scaled by ½. In other embodiments, the HumDiv and HumVar may be scaled differently (e.g., 1/n and (n−1)/n), for example, depending on the relative sample size or confidence level of the damaging vs. non-damaging training set. The raw PolyPhen-2 weight (wⱼ^{P2}) is a measure of the confidence of the PolyPhen-2 data, for example, based on the consistency or difference between its two values, (HumDiv-HumVar).

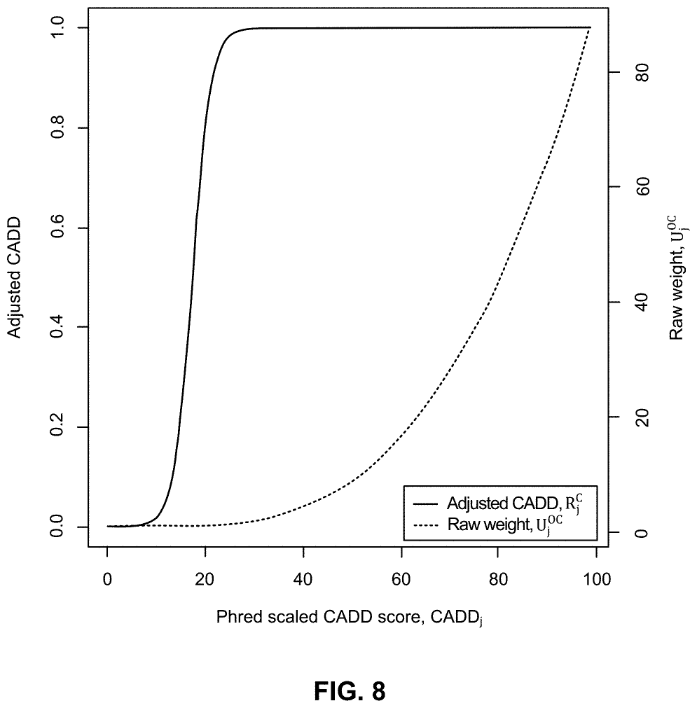

Raw CADD data may provide a “C-score” (CADDⱼ) for each variant representing its pathogenic or deleteriousness ranking, for example, relative to the approximately 8.6 billion (^{˜}109.9) single base changes that could occur in the human genome. The raw CADD C-score (CADDⱼ) is defined on a “Phred” scale, which is a base-10 logarithmic scale in which each 10 points corresponds to an order of magnitude. The raw CADD C-scores may be transformed, for example, based on an optimized logistic function into a sigmoidal distribution of the adjusted CADD scores (e.g., equation (6)). Reference is made to FIG. 8, which is a graph of an example logistic transformation from the raw Phred scaled CADD scores (CADDⱼ) to the adjusted CADD scores (Rⱼ^{C}) (solid line) according to an embodiment of the invention. The raw Phred scaled CADD scores (CADDⱼ) may be transformed to a linear scale such as [M, N], for example, [0.0, 1.0].

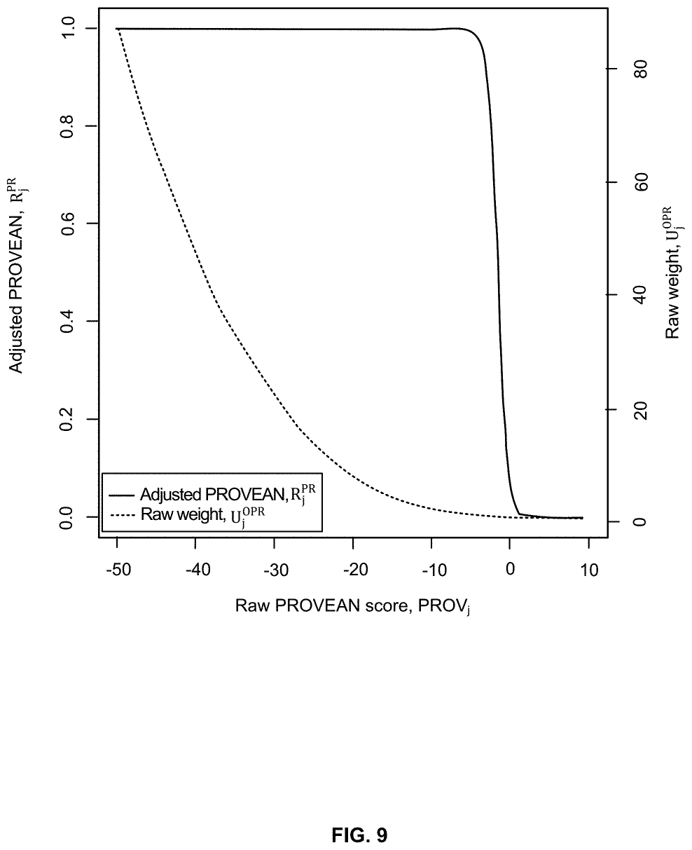

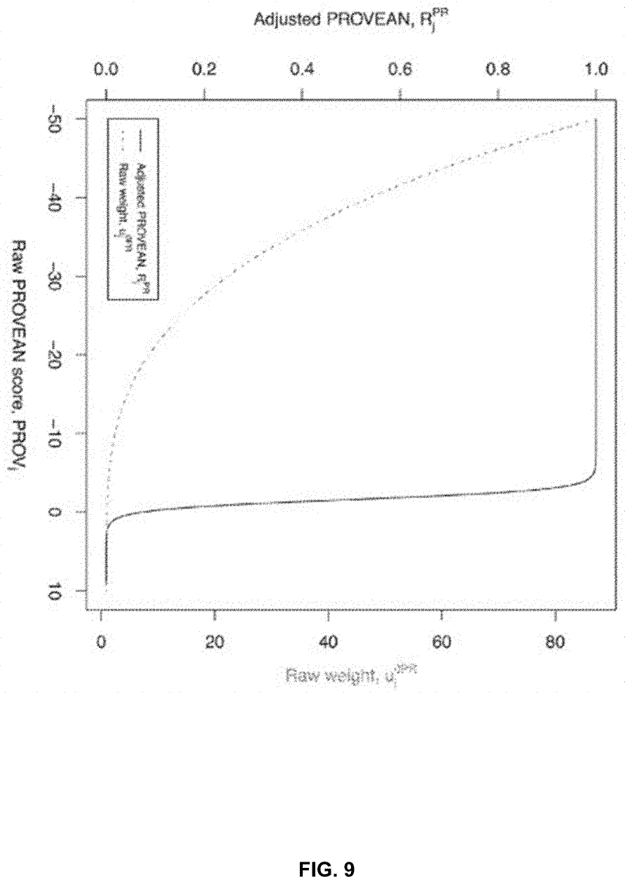

Raw PROVEAN scores (PROVⱼ) may be transformed to a linear scale using an optimized logistic transformation (e.g., equation (7)). Reference is made to FIG. 9, which is a graph of an example logistic transformation from the raw PROVEAN scores (PROVⱼ) to the adjusted PROVEAN scores (Rⱼ^{PR}) (solid line) according to an embodiment of the invention. The raw PROVEAN scores (PROVO may be transformed to a linear scale such as [M, N], for example, [0.0, 1.0].

The raw CADD and PROVEAN values may also indicate intrinsic levels of confidence implied by initially extreme raw CADD and PROVEAN scores. For example, a variant ranked by CADD as among the top 0.1% of variants (e.g., CADD>30) or among the top 0.0000001% of variants (e.g., CADD>90), in which all (or substantially all) of the contributing support vectors agree that the variant is damaging, may be transformed to the most damaging pathogenic predictor level (e.g., Rⱼ^{C}≈1.0). The confidence level of the CADD and PROVEAN values may be represented by the pathogenic predictor weights, (uⱼ^{C}) and (uⱼ^{PR}). The raw CADD and PROVEAN values are transformed to raw CADD and PROVEAN weights, (uⱼ^{0C}) and (uⱼ^{0PR}), for example, using a power function such as in equations (8) and (9), respectively. FIG. 8 shows the relationship between the raw CADD scores (CADDⱼ) and the raw CADD weights(uⱼ^{0C}) and FIG. 9 shows the relationship between the raw PROVEAN scores (PROVⱼ) and the raw PROVEAN weights (uⱼ^{0PR}) (dashed lines). These non-linear exponential weight relationships provide disproportionately greater weights to the extreme-valued raw metrics (extremely high CADD values and extremely small PROVEAN values, as compared to moderate or relatively small CADD values and relatively large PROVEAN values). Accordingly, relatively highly deleterious raw values of CADDⱼ and PROVⱼ result in relatively higher weights for (uⱼ^{C}) and (uⱼ^{PR}), respectively (e.g., as shown in FIGS. 8 and 9).

When there is no raw data for a particular variant, the respective subcomponent score may be designated as “not assigned,” and the raw weight, (uⱼ^{0PP}), may be set to a null or negligible value, for example, to 0.0. The final individual metric weights, (uⱼ^{PP}), may be recalibrated, for example, so that they sum to 1.0. The overall raw weight for the predictive damage component in equation (2), (wⱼ^{PP}), may be set equal or proportional to the sum of the final individual metric weights (e.g., (wⱼ^{0PP}˜Σₚₚ₌₁^{PP}uⱼ^{PP}), as shown in FIG. 3C).

Mutation Class Component

The mutation class component MutScoreⱼ(Sⱼ^{M}) may represent a mutation type, category or class, of a variant. Table 2 shows an example of mutation class subscores (Sⱼ^{M}) and raw weights (wⱼ^{0M}) for various mutation types. Mutation types may be a first order categorization of molecular impact on RNA splicing, mRNA translation, and protein function. Examples of mutation types include, start-loss, stop-gain, frame-shift indel, essential splice site, microsatellite, synonymous, in-frame indel, missense, and stop-loss, although other mutation types may be used.

Start-Loss, Stop-Gain, Frame-shift Indel, and Essential Splice Site Mutation Classes: These mutation classes are associated with severe gene dysfunction. Variants in these mutation classes may be assigned a relatively high mutation class subscore (e.g., Sⱼ^{M}=1.0) and a relatively high confidence raw weight (e.g., wⱼ^{0M}=99).

Synonymous Mutation Class: These mutation classes are associated with low incidences of gene dysfunction. Variants in the synonymous mutation class may be assigned a relatively low or null mutation class subscore (e.g., Sⱼ^{M}=0.0). The synonymous mutation class is given a neutral confidence raw weight (e.g., wⱼ^{0M}=1.0).

Microsatellite Mutation Class: For variants in the microsatellite mutation class, the longer the length of the repeating microsatellite sequence, the less likely the microsatellite impacts gene function and the less likely that a mutation of one of its alleles will cause dysfunction. Accordingly, the mutation class subscore for variants in a microsatellite class may be inversely proportional to the length of the repeating microsatellite sequence (e.g., Sⱼ^{M}=1/(1+mmsSTR^{ems}, where STR is equal to the number of short tandem repeats that exist in all of the microsatellites in ExAC at a given position, and ems and mms are tunable parameters). The microsatellite mutation class may be given a neutral confidence raw weight (e.g., wⱼ^{0M}=1.0).

Missense and Stop-loss Mutation Class: Variants in the missense and stop-loss mutation classes may be assigned a relatively moderate mutation class subscore (e.g., Sⱼ^{M}=0.5) because their impact on gene function is highly variable.

In-Frame Indel Mutation Class: An in-frame indel mutation typically inserts or deletes an integer number (AAⱼ) of amino acids or codons. Because the likelihood of dysfunction typically increases the greater the number of damaged amino acids, in-frame indel mutation dysfunction scores may increase with the number of codons added or subtracted, for example, asymptotically approaching a maximum score (e.g., 1.0), for example, as defined in equations (10a) or (10b). Reference is made to FIG. 10, which is a graph of an example relationship between in-frame indel mutation class scores (Sⱼ^{M})vs. the number of inserted codons according to an embodiment of the invention. Red asterisks identify codons.

In-frame indels, missense, stop-loss and other mutation classes may be assigned a relatively low raw weight (e.g., wⱼ^{0M}=0.01) because their impact on gene function may be more accurately assessed by other scoring components. The mutation class subscore for variants of these mutation classes will thus only significantly contribute to the aggregate VGDj score if the variant's remaining gene dysfunction components also have relatively small weights or are unassigned. All remaining mutation classes, including introns and un-translated region, may be assigned a relatively low or null mutation class score (e.g., Sⱼ^{M}=0.0).

Clinical Classification Component

The clinical classification component, ClinScoreⱼ(Sⱼ^{Cl}), may represent a clinical classification assigned to a variant j. The clinical classification component is typically only considered (e.g., non-zero) when a variant has been clinically validated as causing disease or dysfunction in a living patient with that disease or dysfunction. The clinical classification component may therefore not be considered (e.g., zero) for all new or de novo variants not yet linked to cause disease or dysfunction in a clinical setting (e.g., variants listed in Table 1 or labeled as “No ClinVar Pathogenicity Classification” in FIG. 18). Where a variant is clinically validated, for example as “pathogenic” or “benign”, that information should be weighed heavily against other predictive factors.

FIG. 3C shows one embodiment of clinical classification subscores (Sⱼ^{Cl}) and raw weights (wⱼ^{Cl}) for various types of clinical classification. The clinical component, ClinScoreⱼ (Sⱼ^{Cl}) may be assigned a maximal relatively high value (e.g., Sⱼ^{Cl}=1.0) for all variants that have a (e.g., ClinVar) classification of “pathogenic” and a minimal or relatively low value (e.g., Sⱼ^{Cl}=0.0) for all variants that have a most damaging (e.g., ClinVar) classification of “benign” (see e.g., Table 3). The raw clinical classification weight (wⱼ^{0Cl}) may represent the certainty of the classification. For example, variants classified as “uncontested” pathogenic or benign may be assigned a maximal or relatively high weight (e.g., wⱼ^{0Cl}=20), variants classified as “probably” pathogenic or benign may be assigned a moderate or relatively lower weight (e.g., wⱼ^{0Cl}=10), and variants classified as “contested” pathogenic or benign may be assigned a minimal or relatively lowest weight (e.g., wⱼ^{0Cl}=1) (see e.g., Table 3).

In some embodiments, to counterbalance low weights for variants with probable or contested pathogenic classifications, the weight for all non-benign variants may be increased based on a compound heterozygote count, hⱼ

$$( e . g . \frac{{h}_{j}^{2}}{3} ) ,$$

where hⱼ may be the number of alternative alleles reported in compound heterozygote patients, for example, by a disease database such as OMIM. The compound heterozygote count, hⱼ, may act as a proxy for independent clinical replication of pathogenic findings. Thus, the compound heterozygote count, hⱼ, may also be used to reduce the confidence assigned to a benign classification, e.g., with hⱼ≥3, for example to reduce its weight (e.g., to wⱼ^{0Cl}=0.0).

Bootstrap Pathogenicity Thresholds

Pathogenicity thresholds may be used to delineate a continuous range of VGD scores that are pathogenic or associated with gene dysfunction. Reference is made to FIG. 7A, which is a graph of an example of one-sided bootstrap confidence intervals per gene (t_{g}^{bs}) according to some embodiments of the invention. One-sided bootstrap confidence intervals may be generated, e.g., during a training-phase, as a lower bound for the VGD scores of a plurality or majority (e.g., 99%) of known pathogenic mutations. That is, a predetermined number or percentage (e.g., 99%) of a random sampling of uncontested pathogenic variant's VGD scores are computed above this threshold. In one example of a training-phase, the one-sided bootstrap confidence intervals may be generated to conform non-clinical VGD scores (e.g., average VGDⱼ^{-Cl} without the clinical classification (ClinVar) component among ClinVar-5 and 5.1 variants for each gene) with clinical classification data. In some embodiments, an optimization may be executed to shift the center of the VGD scores of known pathogenic variants above the threshold and shift the center of the VGD scores of uncharacterized variants below the threshold. In one example training-phase, 10,000 boot-strapped samples were used in the training set to calculate pathogenicity thresholds and genes were only considered with at least ten ClinVar-5 variants. Other training parameters, optimizations, and training sets may be used. Once bootstrap pathogenicity thresholds are computed, they may be applied in a run-time phase for example to detect novel or de novo (e.g., uncharacterized) variants as dysfunctional that have VGD scores above these thresholds (see e.g., Table 1).

Expected Discovery Rate of Novel Disease Variants in Heterozygotes or Homozygotes

An expected newborn frequency of deleterious variants in heterozygotes compared to homozygotes (HET:HOM) is derived from the Hardy-Weinberg equilibrium as a function of disease incidence:

$$\frac{HET}{HOM} = \frac{2}{\sqrt{1}} - 2. \qquad ( 5 )$$

For example, based on a disease incidence of 1/2,500, a cystic fibrosis-causing CFTR variant is 98-fold more likely to appear in an unaffected person compared to an affected newborn. Most serious recessive diseases have lower individual incidences and, thus, higher predicted HET:HOM ratios.

Clinical Classification Data Parsing Details

ClinVar has two sources of data: clinvar.vcf (CV in standard VCF format) and variant_summary.txt (VST) files. The CV and VST files label positions of deletions differently and their data are thus difficult to combine. The VST file right-aligns the position of deletions, while the CV file (like other VCF files) left-align the position of deletions. For example, in a situation where the first four positions of a reference sequence is ‘AAAA’, and there is a deletion of a single ‘A’. There is no way to determine which of the 4 ‘A’s is actually deleted; however the deletion must be assigned a single position. VCF format labels this deletion at position 0, while VST labels this same deletion at position 4; yet they refer to the exact same variant. A similar issue occurs when labeling a specific sequence change of any indel. There is a need in the art to provide a consistent way to label specific sequence changes that works with any initial format, for example, even identical indels with different reference alleles.

To properly recognize any indel, embodiments of the invention propose a new technique to transform all variants into a standard labeling format. In this format, the reference is a single base, and an indel may be indicated by a first code or symbol (e.g., “+”) preceding all inserted bases and a second code or symbol (e.g., “−”) preceding all deleted bases. This format properly labels the insertion(s) and deletion(s) made to mutate the reference base into a mutated variant or allele. Additional unique difficulties and exceptions may be handled on a case-by-case basis for the corresponding variants.

Population Details

In some embodiments, allele frequency in the Finnish population may be ignored because it provides skewed results due to a bottleneck effect that took place during the founding of Finland roughly 2,000 years ago. This bottleneck effect has contributed to “Finnish Disease Heritage,” in which deleterious mutations that are rare elsewhere may exist at disproportionately higher frequencies in Finland. Accordingly, in such embodiments, the Finnish data set may be ignored or only used as part of the global data (in other embodiments, the Finnish data set may be considered).

In some embodiments, low-quality samples (e.g., indicated with a low allele number such as AN<500) may be selectively removed or ignored in the training set on a variant-by-variant basis. In one example, frequencies for a variant were only considered based on super-populations with an above threshold AN (e.g., ANₛᵤₚₑᵣ₋ₚₒₚ≥500). If none of the super-populations surpassed this threshold, but a global AN exceeded a global threshold (e.g., AN_{global}≥1,000) for the variant, the global population frequency may be used instead. For a rare case in which the global AN is below the global threshold, or the variant was not observed in ExAC, the variant may be determined to be novel.

Transformation of CADD and PROVEAN Values

The raw CADD and PROVEAN values may be mapped or transformed onto a uniform linear VGD scale, for example, a [M, N] scale such as [0.0, 1.0]).

To transform the raw CADD values, the Phred scale CADD C-scores may be transformed to values on a continuous VGD scale (e.g., [0.0, 1.0]), for example, using the following optimized logistic equation with midpoint (m_{C}) and steepness (k_{C}) parameters:

$${R}_{j}^{c} = \frac{1}{1 + {10}^{kc ( mc - {CADD}_{j} ) \text{/} 10}} . \qquad ( 6 )$$

FIG. 8 shows a graph of this example logistic transformation from the raw Phred scaled CADD scores (CADDⱼ) to the adjusted CADD scores (Rⱼ^{C}) (solid line). In one example implementation, parameters were optimized to generate a midpoint (m_{C}) of 20.4 and a steepness (k_{C}) of 2.3 (although other parameters and values may be used).

Similarly, the raw PROVEAN values (PROVⱼ) for each variant (j) may be transformed to the continuous VGD scale (e.g., [M, N], [0.0, 1.0]), for example, using the following equation:

$${R}_{j}^{pr} = \frac{1}{1 + {e}^{kpr ( {PROV}_{j} + mpr )}} . \qquad ( 7 )$$

FIG. 9 shows a graph of this example logistic transformation from the raw PROVEAN scores (PROVⱼ) to the adjusted PROVEAN scores (Rⱼ^{PR}) (solid line). In one example implementation, the parameters were optimized to generate a midpoint (m_{PR}) of 1.6 and a steepness (k_{PR}) of 1.6 (although other parameters and values may be used).

The confidence of the raw CADD and PROVEAN values may increase disproportionately greatly for highly deleterious values of CADDⱼ and PROVⱼ(e.g., extremely high, such as top 0.1%, of CADDⱼ values and extremely low, such as bottom 0.1%, of PROVⱼ values, since PROVⱼ values are negative). Accordingly, the raw CADD and PROVEAN weights, uⱼ^{0C} and uⱼ^{0PR}, may be defined, for example, as:

$$\begin{matrix}{u}_{j}^{0 c} = bc + \frac{{max ( {CADD}_{J} - sc , 0.0 )}^{ec}}{10} . & ( 8 ) \\ {u}_{j}^{0 pr} = bpr + {( \frac{{PROV}_{J} + mpr}{dpr} )}^{2} ( {e}^{- sp + [ {PROV}_{J} + MPR ]} ) . & ( 9 )\end{matrix}$$

FIG. 8 shows the relationship between the raw CADD values (CADD) and the raw CADD weights (u_{J}^{0C}) and FIG. 9 shows the relationship between the raw PROVEAN values (PROVⱼ) and the raw PROVEAN weights (u_{J}^{PR}) (dashed lines). These relationships provide disproportionately greater weights to extreme-valued raw metrics (extremely high CADD values and extremely small PROVEAN values). For example, the raw CADD and PROVEAN weights, uⱼ^{0C} and uⱼ^{0PR}, scale variants with the most deleterious values (e.g., the top 0.1% of values) to have relatively high raw weights (e.g., uⱼ^{0C} and uⱼ^{0PR}≈99), while the remaining majority of variants have relatively negligible or neutral raw weights (e.g., uⱼ^{0C} and uⱼ^{0PR}≈1.0). The precise power functions and tuning metrics may be optimized in the training-phase.

Mutation Class Component MutScoreⱼ of In-Frame Indels

The mutation class component MutScoreⱼ(Sⱼ^{M}) of in-frame indels typically increases with the number of codons added or subtracted for coding amino acids, denoted (AAⱼ) (e.g., AAⱼ=⅓* number of bases added or subtracted, so that if 12 bases are inserted, AAⱼ=4). As AAⱼ increases, the likelihood of the mutation disrupting protein function increases, but at a diminishing rate (e.g., asymptotically approaching a maximum value, such as, 1.0).

In one embodiment, as shown in FIG. 10 and Table 2, the mutation class component MutScoreⱼ(Sⱼ^{M}) of in-frame indels may be calculated, for example, as:

Sⱼ^{M}=Iⱼ[0.6+0.4*(1−e^{-AAj/10})] (10a)

where Iⱼ is an indicator variable, for example, that is 1 if variant j is an in-frame indel, and 0 otherwise.

In another embodiment, shown in FIG. 3, the mutation class component MutScoreⱼ (Sⱼ^{M}) of in-frame indels may be calculated, for example, as:

$${S}_{j}^{M} = \frac{1}{1 + mii ( {bii}^{- AAj} )} . \qquad ( 10 b )$$

Evolutionary Selection Component

The “evolutionary selection” component EvoScoreⱼ (Sⱼ^{E})may provide a single or cross-species measure that natural selection suppresses a particular allele variant or mutation. An evolutionary model may predict likelihoods that allele mutations or variations would be deleterious based on their frequency or rarity of occurrence across multiple reference genetic sequences in a single species (“single-species” model) or multiple different species (“multi-species” model). For example, allele mutations or variations that are relatively more rare across the reference genetic sequences may be considered negatively selected for evolutionarily (e.g. associated with a deleterious trait for which an organism cannot or has a relatively lower likelihood of surviving or reproducing), while allele mutations or variations that are relatively more common across the reference genetic sequences may be considered positively or neutrally selected for evolutionarily (e.g. not associated with a deleterious trait, but traits for which an organism has a neutral or improved likelihood of surviving or reproducing). Embodiments of the invention may compute a measure of evolutionary variation of alleles (fⱼ) at each of one or more aligned genetic loci as a function of variation in alleles at corresponding aligned genetic loci in the multiple sequence alignment (MSA) (e.g., FIG. 15). The measure of evolutionary variation of alleles may be transformed into a likelihood or subscore (Sⱼ^{M}) associated with a relative propensity that this allele mutation is damaging in an organism or would be damaging if produced in a child. The evolutionary selection component (Sⱼ^{E}) may represent one or more likelihoods that an allele mutation at each of the one or more genetic loci will cause dysfunction or disease based on the measure of evolutionary variation (fⱼ) of alleles at the corresponding aligned genetic loci for one or multiple organisms.

Some embodiments may assign one or more likelihoods that an allele mutation in an organism is deleterious based on the evolutionary variation at the allele loci in real extant species or populations, for example, in order to diagnose living organisms, cells or tumors, or to analyze virtual progeny to filter out prospective pairings of gametes prior to conception.

In some embodiments, multiple reference genetic sequences from the one or more species may be aligned to link or associate one or more genetic loci in each of the multiple different sequences. Aligned loci of the different sequences may be derived from one or more common ancestral genetic loci and/or may relate to the same features, diseases or traits. A measure of evolutionary variation of alleles at one or more of the aligned genetic loci may be computed, for example, as a function of variation in alleles at corresponding aligned genetic loci in the multiple aligned reference genetic sequences. Aligned genetic loci associated with a relatively lower frequency of allele variation may indicate that the alleles are “functional” or relatively important to an organism's survival and their mutations may have a relatively higher likelihood of causing deleterious traits in an organism, whereas aligned genetic loci associated with a relatively higher frequency of allele variation in the reference genetic sequences may indicate that the alleles are less or non-functional and may be mutated with a relatively lower likelihood of impacting the survival or formation of deleterious traits in an organism. In some embodiments, the reference genetic sequences in the model may be weighted according to their evolutionary proximity of its population or species to the population or species of the organism and/or potential parent. For example, more weight may be assigned to reference genetic sequences of populations or species that are relatively more evolutionarily related (e.g., closer on a phylogenetic tree or having a relatively smaller Hamming distance).

Genetic sequences may be obtained from a living organism or two potential parents, such as, two individuals that plan on mating or between one individual seeking a genetic donor and each of a plurality of candidates from a pool of genetic donors. The potential parents' genetic sequences may be obtained from genetic DNA samples of biological material from the potential parents. A mating may be simulated between two potential parents, for example, by combining the genetic information from the two potential parents' genetic sequences to generate one or more genetic sequences of simulated virtual progeny.

The living or virtual organism's genetic sequence may be aligned with one or more of the reference genetic sequences to identify one or more alleles that evolved from the same ancestral genetic loci. The organism may be assigned one or more of the likelihoods of exhibiting deleterious traits associated with one or more alleles or mutations in the virtual progeny genetic sequences based on the measure of evolutionary variation of alleles at the corresponding aligned genetic loci in the reference genetic sequences.

Embodiments of the invention overcome the limitations of relying on specific information derived from human or cellular studies of the effect of mutation in order to score the propensity or probability that a particular mutation or allele will cause a deleterious phenotype, trait or disease.

An insight recognized according to embodiments of the invention is that extant genetic variation, that is existing or surviving genetic variation present amongst homologous or paralogous reference DNA sequences present in different organisms or members of a population, represents the outcome of an experiment that can be informative for predicting whether a given mutation or allele variation in an organism's genome is likely to be deleterious.

This experiment is the four billion year long process of evolution, which has governed the replication and diversification of life on Earth. Today, there are many species, and individuals within a species all contain copies of genetic material, which is derived from common ancestral versions. As species and individuals reproduce and copy their DNA, mutations appear which make these descendent copies distinct from the parental versions. The eventual fate of such new mutations, whether they will continue to be passed along to offspring or eventually die out, is determined by a stochastic process that is influenced by the mutation's effect on the reproductive fitness of the organism. Mutations that have no functional effect (neutral mutations) or are beneficial to an organism (positive mutations) are more likely to eventually increase in frequency and persist in the population, increasing diversity or replacing their parental version. In contrast, mutations which lower the reproductive fitness of an organism (negative or deleterious mutations) are unlikely to persist and contribute to future genetic variation.

Over the course of evolutionary time, a great many mutations have appeared and persisted, leading to the present diversity amongst DNA sequences derived from a common ancestor. However, this diversity is not equally distributed amongst all sequence positions in a genome. Although mutations are essentially introduced during the replication process independent of any functional effect they may have, the evolutionary filtering process is greatly influenced by such effects. As such, when comparing the genomes of several species or individuals today, some areas are conserved (such as having the same coding sequence and/or non-coding sequence), while others have much more greatly diverged (having very diverged sequences from each other or relative to the ancestral copy number).

Reference is made to FIG. 15, which shows an example of an alignment of multiple genetic sequences (SEQ ID Nos.: 1-36, proceeding from top to bottom, respectively) according to an embodiment of the invention. The sequences are submitted electronically and incorporated by reference herein.

The abbreviations for the species in FIG. 15 are as follows: LATCH=Latimeria chalumnae; XENTR=Xenopus tropicalis; TAEGU=Taeniopygia guttata; MELGA=Meleagris gallopavo; CHICK=Gallus gallus; ORNAN=Ornithorhynchus anatinus; LOXAF=Loxodonta africana; HORSE=Equus caballus; TURTR=Tursiops truncatus; MYOLU=Myotis lucifugus; AILME=Ailuropoda melanoleuca; OTOGA=Otolemur garnettii; CALJA=Callithrix jacchus; MACMU=Macaca mulatta; NOMLE=Nomascus leucogenys; PONAB=Pongo abelii; GORILLA=Gorilla gorilla; CHIMP=Pan troglodytes; HUMAN=Homo sapiens; TUPBE=Tupaia belangeri; OCHPR=Ochotona princeps; CAVPO=Cavia porcellus; SPETR=Spermophilus tridecemlineatus; DIPOR=Dipodomys ordii; MOUSE=Mus musculus; RAT=Rattus norvegicus; SARHA=Sarcophilus harrisii; MONDO=Monodelphis domestica; MACEU=Macropus eugenii; DANRE=Danio rerio; GADMO=Gadus morhua; ORYLA=Oryzias latipes; GASAC=Gasterosteus aculeatus; ORENI=Oreochromis niloticus; TETNG=Tetraodon nigroviridis; TAKRU=Takifugu rubripes.

In the example of FIG. 15, the multiple aligned reference genetic sequences represent a portion of the DNA sequence coding for the PEX10 proteins present in organisms from multiple vertebrate species. Item A in the figure shows a nucleic acid genetic locus which is completely conserved across all species in the alignment, as all species have a Guanine (symbolized by the letter G) at this locus position. Although many mutations that change the amino acid at this position have undoubtedly been introduced into this gene over the course of the 500 million years of vertebrate evolution, the fact that no such mutation persists today is a strong indication that such mutations are likely to be deleterious and reduce evolutionary fitness. In contrast, the position in the gene indicated by item B in FIG. 15 is much more variable, with different species having at that locus position one of the following DNA bases: Guanine (G), Adenine (A), Thymine (T), Cytosine (C). The diversity of DNA (or alternatively the amino acids encoded by the DNA) at this genetic locus provides an indication that it is relatively less likely that a mutation at this position in an organism's genotype will be deleterious, relative to a mutation at the genetic locus position indicated by item A.

A multiple sequence alignment of present day reference genetic sequences may be derived from common ancestral genetic loci of multiple species (e.g. different vertebrate sequenced genomes) or multiple individuals within a single species (e.g. a collection of human sequences). A substantially large sample size of organisms, populations or species (e.g., tens, hundreds, or more) may be used for statistically significant likelihoods, for example, to reduce bias error due to a skewed sample set.

Embodiments of the invention may compute a measure of evolutionary variation of alleles f at each of one or more aligned genetic loci as a function of variation in alleles F at corresponding aligned genetic loci in the multiple sequence alignment (MSA). The measure of evolutionary variation of alleles f may be transformed into a likelihood or sub score (Sⱼ^{E}) associated with a relative propensity that this allele mutation would be damaging in an organism. This likelihood or subscore (Sⱼ^{E}) may be derived, for example, using two functional transformations F and S, to convert columns of aligned genetic loci of a multiple sequence alignment (MSA) and a putative mutation or allele in an organism into a propensity score or likelihood (Sj E) relevant to assessing the effect of that particular allele or mutation on the organism, for example, as:

Multiple Sequence Aligment (MSA)→f=F(MSA)→Sⱼ^{E}=S(f) (11)

The first functional transformation shown in equation (11), f=F(MSA), may be used to compute a measure of evolutionary variation of alleles f at each of one or more genetic loci derived from one or more common ancestral genetic loci in the multiple organisms as a function of variation in alleles F at corresponding aligned genetic loci in the multiple aligned genetic sequences. The first functional transformation may create a raw score that quantifies the relative amount of sequence conservation at the one or more genetic loci. There are many possible instantiations of this function that may be used according to embodiments of the invention. For example, one such function may input information from the DNA or amino acid genetic sequences present in the alignment and output a Shannon entropy of the sequence characters at each of the one or more genetic loci. Denoting a frequency of a particular symbol (DNA base or amino acid) at a particular genetic locus or column position, (j), in a multiple sequence alignment as Pᵢ, i={A, C, G, T} (for DNA, or the set of amino acid symbols if considering a protein alignment), the Shannon entropy function may be calculated, for example, as shown in equation (12):

F(MSAⱼ)=Σᵢpᵢ log₂pᵢ (12)

Another example of the first functional transformation shown in equation (11), f=F(MSA), may take the average pairwise difference between different symbols (S) in an aligned sequence column of length N, for example, as in equation (13):

$$F ( {MSA}_{j} ) = {( \begin{matrix}N \\ 2\end{matrix} )}^{- 1} {\sum }_{i = 1}^{N} {\sum }_{k = ( i + 1 )}^{N} \{ \begin{matrix}1 & if {S}_{i} \neq {S}_{k} \\ 0 & if {S}_{i} = {S}_{k}\end{matrix} \} \qquad ( 13 )$$

Other possible functional forms of the first functional transformation, F(MSA), may calculate a distance metric from a particular species or sequence in the reference alignment. For example, the function may rank all the sequences in the alignment according to their Hamming distance from the reference (e.g., human) sequence, and then calculate the rank of the first sequence with a divergent symbol at the relevant position in the alignment, or if ranking a particular mutation, the rank of the first sequence matching that particular mutation. Additional functional forms such as not using the ordinal rankings of sequences by Hamming distance, but instead using the Hamming distance itself as the metric may be used.

Additionally, the function F(MSA) need not return a single value or be a function of a single column in the multiple sequence alignment. The function may be a composite function of one or more of the functions previously described in addition to others (e.g., F₁, F₂, F₃, . . . ), and may output a vector of values (e.g., (s₁, s₂, S₃, . . . )) rather than a single value. The function may also take as input multiple columns (j), or even the entire alignment, when calculating the value(s), f, and may also take as input a particular mutation under consideration, which may or may not affect the calculation of the values returned by the function.

The second functional transformation specified by equation (11), Sⱼ^{E}=S(f), is a function which converts the measure of evolutionary variation of alleles into a subscore or likelihood of being damaging. Many possible instantiations of this function are also possible. For example, a function Sⱼ^{E}=S(f) may score the value(s) of f according to its ranking in the empirical distribution of values for all mutations or alleles considered, or that could be considered, based on a collection of multiple reference sequence alignments. Other scoring methods may also be used. For example, a function trained to discriminate mutations in a database of known or suspected to be damaging alleles from neutral alleles may be used to assess the likelihood of damage (e.g., using any variety of statistical models or derived variants from experimental findings), or a function which is trained to assess the likelihood of a mutation reaching a certain frequency in the population. In all cases, this functional transformation, in combination with the first, allows particular genetic allele mutations to be ranked and assessed for likelihood of survival or damage if produced in a child.

Embodiments of the invention may create functional forms of the measure of evolutionary variation F(MSA) using a phylogenetic history for assessing likelihoods of deleterious effects of alleles. Because DNA replication is semi-conservative, the evolutionary history of a DNA molecule may be represented by a bifurcating tree, known as a “phylogenetic” tree, that represents the known or inferred historical or evolutionary relationships between present day extant reference genetic sequences. A large body of scientific literature has developed over the past 30 years that studies the problem of inferring this tree from present day sequences. Typically, such models envision the evolutionary process between nodes of the tree as being similar to a general time reversible (GTR) Markov chain. In these models, in an interval of time, t, there is a certain probability that a base in the sequence will mutate, or transition to another base (e.g., A→C). Such models may be described using a transition matrix that describes the relative probability of transition from one base to another, for example, as shown in equation (14):

$$\begin{matrix} T C A G \\ Q = \begin{matrix}T \\ C \\ A \\ G\end{matrix} ( \begin{matrix}\cdot & a {π}_{c} & b {π}_{A} & c {π}_{G} \\ a {π}_{T} & \cdot & d {π}_{A} & e {π}_{G} \\ b {π}_{T} & d {π}_{C} & \cdot & f {π}_{G} \\ c {π}_{T} & e {π}_{C} & f {π}_{A} & \cdot \end{matrix} )\end{matrix} \qquad ( 14 )$$

In equation (14) above, the elements of the transition matrix Q may define a probability that each base denoted by the row will transition to each base denoted by the column, for example, in an infinitely small evolutionary time interval Δt. Note that the diagonal terms in the transition matrix are not shown, as they are simply equal to one minus the other elements in the row (the probabilities of the elements in each row sum to 1). The πᵢ terms represent the equilibrium frequency of the nucleotide bases {i=A, C, G, T}, and the symbols a, b, c, d, e and f are parameters that further govern the substitution dynamics. This matrix represents a generalized time-reversible model, in which each rate below the diagonal equals a reciprocal rate above the diagonal multiplied by the equilibrium ratio of the two bases. Equation (14) is only one example of a substitution matrix used for phylogenetic inference.

Reference is made to FIG. 16, which schematically illustrates an example of a phylogenetic tree inferred from a multiple sequence alignment, for example, as shown in FIG. 15, according to an embodiment of the invention. The length of the branches shown in the tree may represent or be a function of an inferred number of substitutions or allele mutations per genetic locus that have occurred from its direct ancestral sequence. In the example shown in FIG. 16, a scale bar for the branch lengths is shown for a 0.2 branch length. The phylogenetic tree may be directly inferred using the Markov model described above. In inferring the tree, the likelihood of a tree and its branch length (e.g., given in units of expected substitutions) are maximized by varying the tree or branch lengths, until a tree with the highest likelihood is found. Alternatively, the neural network described herein may be used to compute all parameters in the phylogenetic model. In another embodiment, a prior probability distribution, as used in Bayesian statistics, may be specified for all parameters in the phylogenetic model, and several trees may be sampled according to their posterior probability distribution, as used in Bayesian statistics.

After fitting the Markov model that accounts for the phylogenetic history of the sequence alignment, whether using a neural network, Bayesian approach or maximum likelihood approach, embodiments of the invention may provide not only an inferred phylogenetic tree, but also a model for the evolutionary process of the multiple reference sequences in that tree.

Embodiments of the invention may use this evolutionary model to directly assess the likelihood that a mutation is damaging by examining the probability that a mutation found in an organism's genome persists into the future. That is, rather than using the model to infer the past history of evolution, the model is trained using the outcomes of the past and then used to calculate the likelihood of an allele persisting into the future. Because this likelihood is directly related to the probability that the allele is damaging (less likely mutations being more likely to be damaging), this phylogenetic approach, which directly accounts for the past history of the sequence in a parametric model, is a uniquely valuable functional form for the F(MSA) specified by equation (11).

Important extensions to the phylogenetic model are those which either change the model to account for sequence context (e.g., information about sequence location or what a sequence encodes, such as, methylation or homopolymer status) and functional effect (e.g., synonymous vs. non-synonymous, or affecting or not affecting expression), or that partition the sequence in some way to account for varying rates of substitutions, for example, based on the location of loci in the genome.

To account for varying rates of substitutions, for example, instead of the direct application of the substation rate matrix in equation (14) to all sequence substitutions, an alternate model may specify that although the relative rates of different types of substitutions at all alignment positions was governed by (14), the global rate at each site (that is the total mutation rate, denoted μ), may vary across sites or loci in the genome. A multitude of such models are possible. For example, the rates at different genetic loci may be drawn from a parametric distribution, such as a F-distribution that is also fit during the modeling procedure, or the distribution of rates may be derived from several categories of possible distributions. In one embodiment, this model may specify two different distribution categories (such as, conserved or rapidly evolving categories) and then train the model to identify to which distribution category the observed sequence belongs, for example, using a hidden Markov procedure. In some embodiments of the invention, the inferred or posterior probability that a genetic locus or mutation belongs to a category in the phylogenetic model may be returned by the F(MSA) function instead of the likelihood itself.

To account for functional effects, the phylogenetic model used to form the likelihood for the F(MSA) function may directly account for the functional consequence of a mutation. For example, the coding sequence of a protein is determined by triplets of neighboring DNA nucleotides that form a functional unit referred to as a “codon.” A mutation in a nucleotide within a codon may either have a functional effect of changing the amino acid sequence encoded by that codon (in which case it is referred to as “non-synonymous” since the mutation encodes for a different amino acid sequence) or may be a substitution with no functional effect on the amino acid encoded due to the redundancy of the genetic code (in which case it is referred to as “synonymous” since the mutation encodes for the same amino acid sequence). The Markov model that may be used in predicting the likely damaging effect of a mutation may directly account for such functional effects. For example, the transition probability of mutating from nucleotide i to j specified by the ijth matrix Q element qᵢⱼ in equation (14), may be replaced by an instantaneous transition probability tᵢⱼ, for example, defined by equation (15).

$${I}_{ij} = \{ \begin{matrix}{wq}_{ij} & if i \to j is non \text{-} synonymous \\ {q}_{ij} & if i \to j is synonymous \end{matrix} \qquad ( 15 )$$

This would allow a new instantaneous transition matrix, T, to be used in the model, and a new parameter, ω, which is equal to the non-synonymous to synonymous substitution rate to be used in predicting the likelihood that an allele is damaging or that it persists into the future. In practice, the ω parameter may be constant for an entire multiple sequence alignment, may be assigned to each codon position in the alignment by assuming they are drawn from some hierarchical distribution, or may be uniquely assigned to each codon position. The substitution model specified by equation (15) may also be altered to account for each combination of the 64×64 possible elements of a transition matrix representing the rate in which each of the 64 possible codons (e.g., the 4³=64 different combinations of four nucleotide states (A, T, C, G) at three nucleotide positions in each codon) transition to each of the 64 possible codons. In all instantiations of the evolutionary model, the functional effect of a sequence change, whether on the amino acid, regulatory context or other biological context may be directly accounted for, and used to predict the likelihood that the allele was damaging.

### Results and Discussion

The particular values, scores, subscores, parameters, graphs, functions, thresholds, relationships, slopes, or rates depicted herein may vary for example based on the input data, validation datasets, updated disease classifications, analyzed genome sequence, reference genome sequences, or other data; however, the trends and relationships such as increasing values, decreasing values, direct and indirect proportionality, continuity, stair-step relationships, etc. may be generalized in some embodiments of the invention to any or a broad range of datasets. These values were obtained using datasets that may have been limited and may be revised with more current datasets to provide more accurate results.

Example Data Acquisition and Exome Sequencing Interval Processing

The VGD model generated according to some embodiments of the invention was trained in one example using variant calling metadata from 60,706 ExAC samples. This dataset was filtered into non-overlapping intervals covering coding regions of 480 autosomal genes associated with severe recessive pediatric disease. These regions were selected because they are known to contribute to genetic disease and collectively constitute a commonly used genetic testing panel.

Filtered intervals covered a total of 1,343,919 base pairs of the human genome. E.g., three overlapping subsets of variants were considered located in targeted regions: 238,461 variants were observed in at least one subject in these regions in the ExAC dataset, 17,748 ClinVar variants, and 6,274 VariBench variants. Only 8,341 ClinVar and 2,345 VariBench variants in our intervals were present in the ExAC samples. Curated variants not found in the ExAC samples (e.g., having a frequency of <1/124,000) may be assigned an allele frequency of zero. In total, individual variant-specific gene dysfunction (VGD) contributions were determined for 250,419 unique variants.

Novel Variant Scoring in a Genotype-Based Disease Liability Analysis

Embodiments of the invention provide a novel and dynamic neural network that incorporates multiple sources of information to compute one or more likelihoods of variant-specific gene dysfunction, VGDⱼ, for each variant j in a variant dataset (j=1, 2, . . . , J). VGDⱼ may be computed as the weighted sum of B component subscores (e.g., equation (2)). Each component subscore may be computed on a continuous uniform scale (e.g., a linear scale defined by [M,N], where M, N are rational numbers, such as a [0.0, 1.0] scale). In one embodiment, the VGD score may be directly proportional to gene dysfunction, for example, where substantially low or below threshold values correlate with a fully functional gene and substantially high or above threshold values correlate with complete gene dysfunction. (Alternatively, the VGD score may be inversely proportional to gene dysfunction, with opposite trends). In one embodiment, the VGD score may be computed as a combination of any two or more of the following components:

VGDⱼ=wⱼ^{Cl}Sⱼ^{Cl}+wⱼ^{M}Sⱼ^{M}+wⱼ^{PP}Sⱼ^{PP}+wⱼ^{E}Sⱼ^{E}+wⱼ^{P}Sⱼ^{P} (16)

where Sⱼ^{Cl} (or ClinScorej) is the clinical assessment of “pathogenicity,” S_{jP} (or PathScorej), Sⱼ^{M} (or MutScoreⱼ) is the expected impact of the mutation class e.g., on RNA processing and translation, S^{PP} (or PathScorej) is a combination of predictive damage values obtained from established tools, Sⱼ^{E} (or EvoScoreⱼ) is the evolutionary constraint factor measuring the natural selection against a variant across one or more species, and Sⱼ^{P} (or PopScoreⱼ) is the population selection factor measuring the natural selection against a variant within each of multiple individual human populations. Each subscore Sⱼ^{b} is associated with a weight, wⱼ^{b}, for example, directly determined by the attributes of the corresponding variant and representing a level of confidence in the corresponding scoring component. A general overview of example equations, parameters and values used to calculate these components is outlined in FIGS. 3A-3D, though other equations, parameters and values may be used.

Embodiments of the invention may thereby provide a solution that accounts for many distinct types of variants and incorporates many genetic properties and components to increase detection sensitivity. Rather than indiscriminately accepting any single metric (or a fixed combination of metrics) as being the most appropriate tool for all variants, embodiments of the invention may evaluate the pertinence or weight of the available data for each unique variant. None of the utilized public metrics is the “best” for all variants and each misses diagnosing variants of a sub-optimal type; the tool with the largest numerical contribution to the final VGDⱼ score is directly determined by the corresponding variant. Further, the final VGDⱼ score may be dynamically updated at any time with the advent of additional data or variant evaluation tools.

Impact of Allele Frequency and Clinical Annotations on VGD Score

The VGD model described according to embodiments of the invention uniquely incorporates observed population frequencies and species frequencies from a large ethnically diverse cohort in order to generate a summary disease contribution score VGDj for each variant j. In this way, the VGD model is able to recognize mislabeled variants that others may miss (see e.g., Table 1 and description of FIGS. 18A-18B). Embodiments of the invention generally assume that truly deleterious mutations typically cannot be maintained in a population or species at substantial frequencies due to negative selection. Accordingly, the VGD model generates a population selection component based on each variant's frequency on a population level in each of a plurality of distinct populations or super-populations. The VGD model also generates an evolutionary selection component based on each variant's frequency on a species-level using an allele frequency in each of a plurality of reference genomes within a single or across multiple species. Embodiments of the invention generally assume that if a variant is of high enough frequency in a single super-population to not be deleterious in that population, it is highly unlikely for that variant to be truly deleterious in any population. The same is generally assumed across multiple different species, though the correlation between human survival and survival in other species may be weighed based on the evolutionary proximity of the other species to humans.

Failure to find literature-based evidence for disease liability becomes less and less tenable as the frequency of disease variants increases. Embodiments of the invention generally assume that if a variant were both truly damaging and relatively common, the variant would have a sufficiently high clinical visibility (bolstering the heterozygous effect in the population selection component) and there would be a sufficient number of affected individuals to lead to this variant's clinical classification (e.g., in the ClinVar database) (bolstering the clinical classification component). Therefore, variants with a high frequency in a population or ExAC dataset, in the absence of annotation in clinical databases, are very unlikely to actually be damaging, regardless of the other scoring factors.

Among the variants with existing clinical annotation, in an example ClinVar dataset, 6,651 variants were unambiguously labeled as “pathogenic.” These variants were classified as “ClinVar-5.1” and have a VGD score approximately equal to Aj. Because damaging variants are unlikely to exist at high frequencies in the population, the vast majority of ClinVar 5.1 variants will have scores close to 1.0 (see below).

An additional 140 ClinVar-5 variants with replicated homozygosity for the presumed disruptive allele in the ExAC cohort, and/or also assigned to one of the “non-pathogenic” ClinVar (ClinVar-2 or ClinVar-3) categories were labeled as “ClinVar-5.2” or “ambiguously assigned ClinVar 5's” (Table 4).

Though the vast majority of ClinVar-5s do not exhibit replicated homozygosity, the few that do are disproportionately represented in the population. To estimate each set of variants' influence on the population, the number of chromosomes in the ExAC cohort were totaled for all variants in each category. For example, the 6,651 ClinVar-5.1 variants were detected in 31,948 chromosomes, while the 140 ClinVar-5.2 variants were detected in 425,139 chromosomes. Because one individual can have multiple different ClinVar-5 variants, some chromosomes may be counted more than once, and the total number of chromosomes for a set of variants may exceed the actual number of chromosomes in the ExAC data.

An alternative cause of inconsistency between a variant's raw VGD score and frequency may occur when there is a true disease liability in homozygotes, but the allele frequency is lifted for example by a heterozygous advantage. A well-known example of such a variant is the HBB sickle cell variant G6V. Under the assumption that all such instances would have been identified and recorded by now, the corresponding allele database (e.g., OMIM) is expected to include compound heterozygote descriptions for the variant. This incidence of compound heterozygote is accounted for in the VGD score as the heterozygote effect parameter (ch) defining a number of compound heterozygotes or combinations of the variant with other variants that is described in the clinical literature. The more compound heterozygote instances a variant has, the more likely the variant has a disease contributing effect, boosting the final VGD damage score. Of the 17,748 ClinVar variants tested, 2,008 were found to be compound heterozygotes, only 163 of which have more than 2 compound heterozygotes instances. For reference, the well-known CFTR variant F508del was measured to have 19 compound heterozygotes, more than any other variant examined in these tests.

Example Distribution of VGD Scores

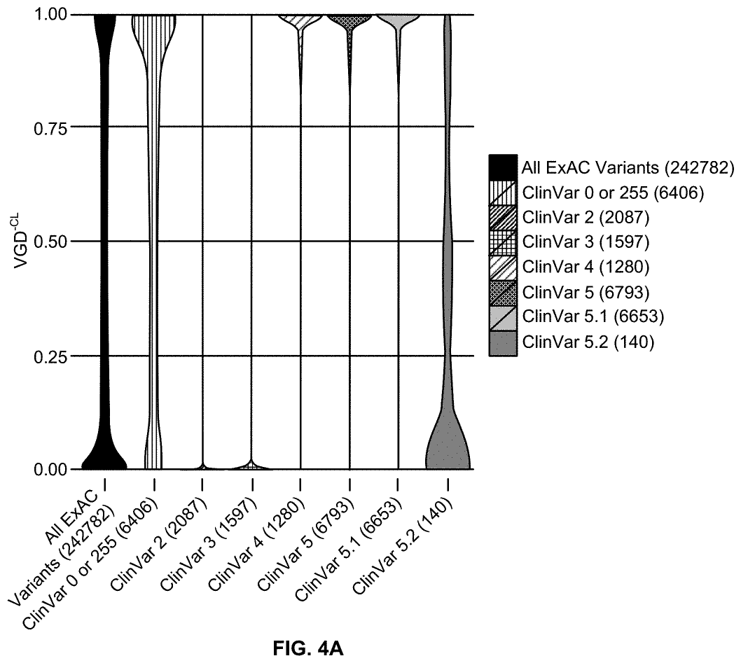

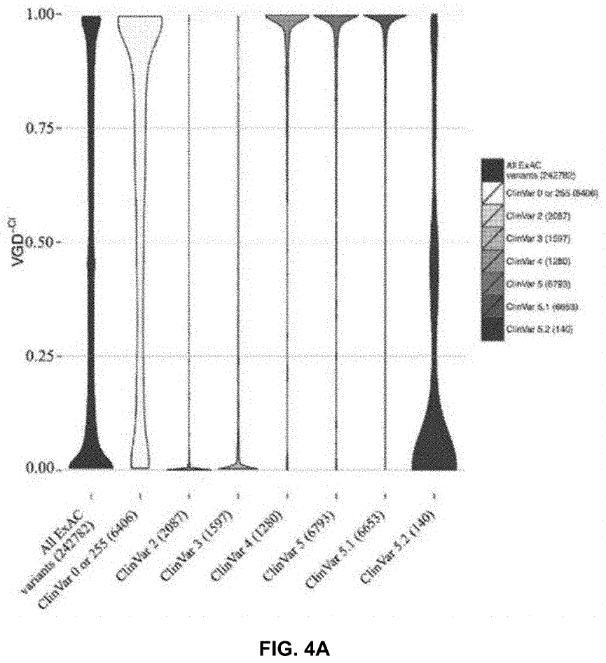

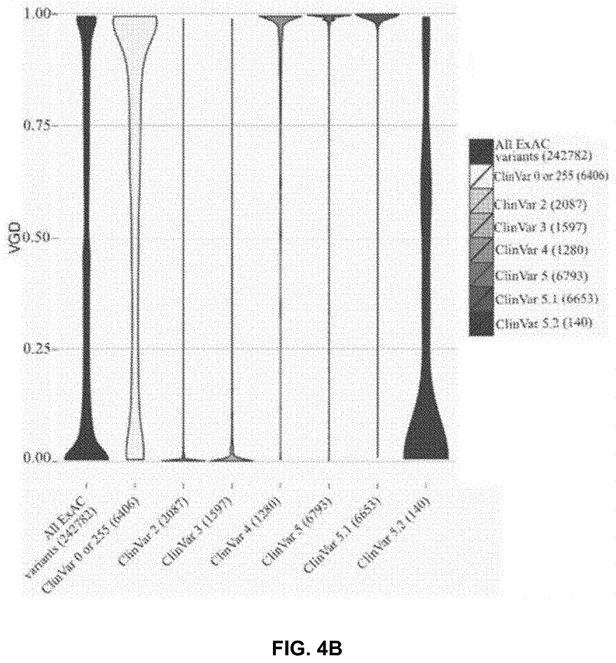

Not surprisingly, in one example test, the VGD model identified a majority (e.g., >50%) of the 238,461 ExAC variants examined as not causing disease of gene dysfunction. The median damage score was 0.32 with a large interquartile range (IQR) of (0.03, 0.77) (Table 5). The distribution of the final VGD score may be bimodal, with a large peak e.g., near 0.0 and a more moderate peak near e.g., 1.0; however, there are many variants with scores in between these extremes (see e.g., FIGS. 4A-B). Only 68,406 (28.2%) ExAC variants were observed to have a damage score less than 0.05 and only 29,972 (12.3%) variants to have a VGD greater than 0.95. Fewer than 25% of ExAC variants were ever observed in the homozygous state, and only half have a maximum population frequency greater than 8.66×10-5 (Table 5).

VariBench Categorization Results

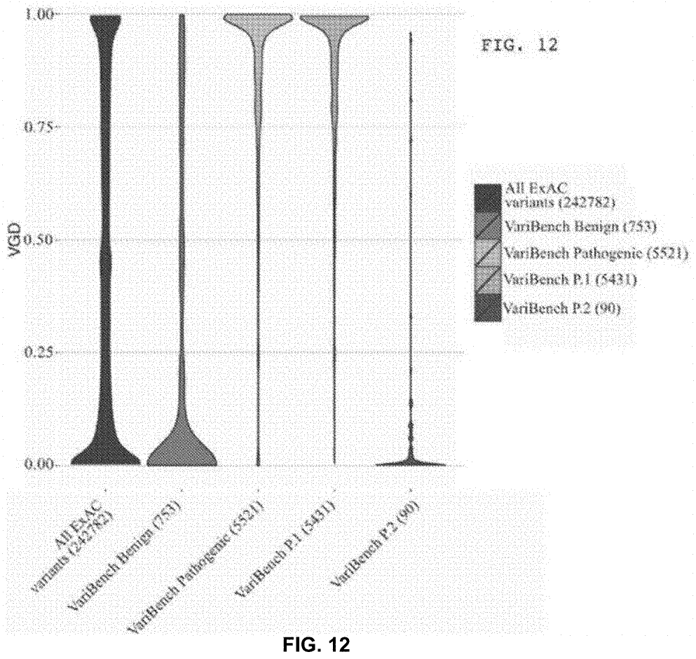

VariBench data was also analyzed as an alternate to the ClinVar data described herein. Reference is made to FIG. 12, which shows a violin plot of an example of variant-specific gene dysfunction for each variant stratified by VariBench category according to an embodiment of the invention. “Pathogenic” VariBench variants have generally very high disruption scores, while “benign” variants have generally very low scores. VariBench P.2 variants, which are homozygous in at least two ExAC subjects have generally lower scores than the other pathogenic VariBench variants.

The VGD median score of benign VariBench variants is e.g., 0.00 (IQR: 0.00, 0.35) compared to e.g., 0.98 (IQR: 0.83, 1.00) for pathogenic VariBench variants (Table 5). The 86 “pathogenic” VariBench variants were classified with replicated homozygosity in the ExAC data as “VariBench P.2” variants. These VariBench P.2 variants were assigned a much lower score than their VariBench P.1 counterparts, which lacked replicated homozygosity in the ExAC data (e.g., having a median VGD score of 0.00 and 0.98, respectively) (Table 5).

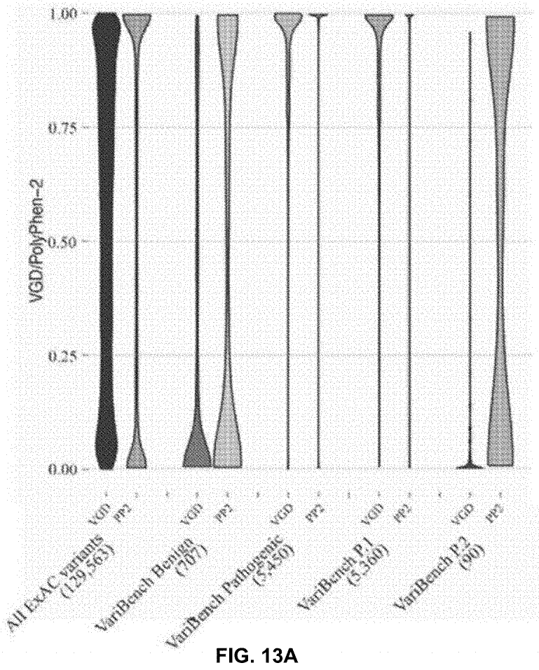

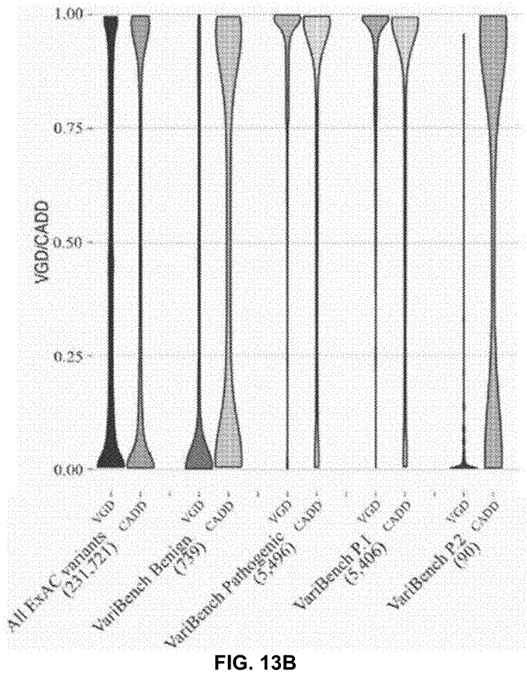

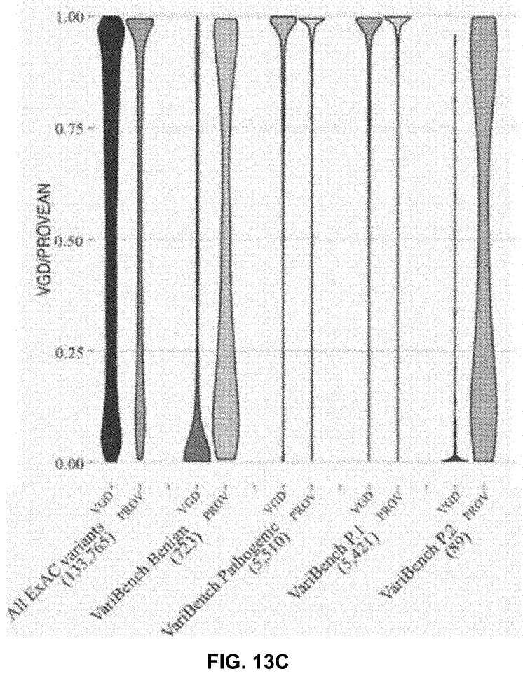

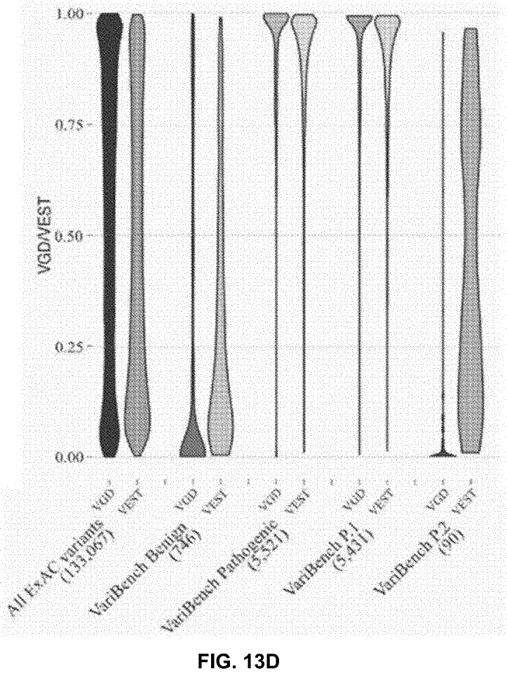

The pathogenic predictors, PolyPhen-2, VEST, CADD, and PROVEAN, were computed for the VariBench P.2 variants, which produced less deleterious damage scores on average than the VariBench P.1 variants (see e.g., FIG. 13A-13D, Table 5). Unlike the final VGD model, however, these raw pathogenic predictors, PolyPhen-2, VEST, CADD, and PROVEAN, fail to calculate a low probability of damage to the large majority of “benign” VariBench variants, and instead assigned these 752 variants a large spread of damaging scores (see e.g., FIGS. 13A-13D).

Comparison of ClinVar and VariBench Categorizations

Demonstrating their ascertainment biases, the ClinVar and VariBench datasets both have a relatively high median damage score of at least 0.96 for all analyzed variants (Table 5). The ClinVar variants were slightly more common than those in the VariBench database. Both types of variants had a median maximum population frequency of 0.0, but the upper bound of the frequency IQR was 4.05×10⁻⁴ for ClinVar and only 8.77×10⁻⁵ for VariBench (Table 5). In addition, 25% of all ClinVar variants were homozygous in at least three ExAC individuals (Table 5).

To determine the accuracy of the VGD model compared to clinical models, all ClinVar variants were analyzed using the VGD model to generate a naive VGD score, VGD^{-Cl}, calculated without any clinical classification (e.g., ClinVar) information. Reference is made to FIGS. 4A and 4B, which show violin plots of an example of VGD scores computed (a) without clinical classification data, VGD^{-Cl}, and (b) with clinical classification data, VGD, respectively, according to an embodiment of the invention.

Even without their clinical classification data, the VGD model assigned “likely pathogenic” (ClinVar-4) and “pathogenic” (ClinVar-5) variants naive dysfunction scores, VGD^{-Cl}, significantly greater than their benign counterparts (see e.g., FIG. 4A). Upon adding the clinical classification data, as expected, the variants' final VGD scores were further elevated (e.g., medians of 0.99 for ClinVar-4 and 1.00 for ClinVar-5) and low frequencies and homozygous incidences (e.g., all with medians of 0) (see e.g., FIG. 4B, Table 5). The 6,651 ClinVar-5.1 variants for which there was no conflicting classification (either in ExAC or the literature) have a median final VGD score of 1.0 (see e.g., FIG. 4B).

ClinVar-5.2 variants have significantly lower VGD scores than their ClinVar-5.1 counterparts (e.g., median score of 0.02 compared to 1.0). The VGD model differentiates these two types of variants and scores them accordingly.

Variants categorized as “non-pathogenic” (ClinVar-2) or “likely non-pathogenic” (ClinVar-3) generally have very low VGD scores; for example, both variant types have median VGD scores of for example 0.00 and average values for example less than 0.07 (Table 5, FIG. 4B). Consistent with their benign effect, these variants also have relatively high median maximum population frequencies (0.021 and 0.015, respectively) and numbers of homozygotes in the ExAC dataset (8 and 5, respectively) (Table 5).

Embodiments of the invention may similarly identify both benign and pathogenic variants characterized by the VariBench data source (see e.g., FIG. 12, Table 5). The VGD model classification was therefore shown to concur with clinically validated results, even in a blind test without considering clinical classification data: variants that are known to be non-pathogenic were assigned a relatively low probability of disease contribution according to their VGD scores and variants that are known to be pathogenic were assigned a relatively high probability of disease contribution according to their VGD scores.

VGD Score for Each Mutation Type

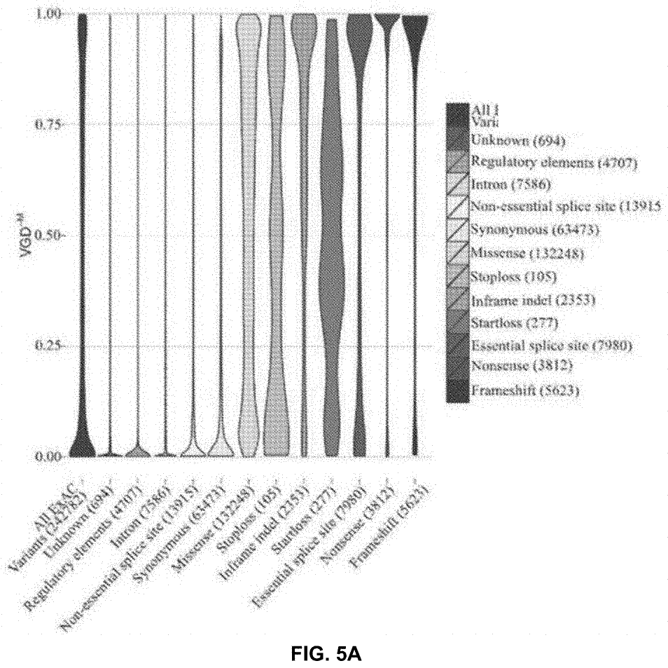

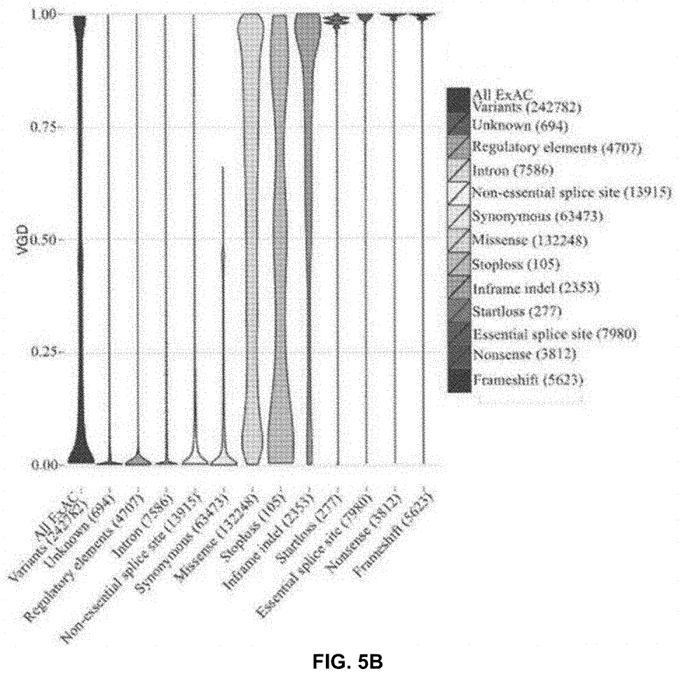

To determine the accuracy of the VGD model as compared to mutation type data, all variants were analyzed using the VGD model to generate the naive VGD score, VGD−M, calculated without any mutation type data (see e.g., FIG. 5A). Reference is made to FIGS. 5A and 5B, which show violin plots of an example of VGD scores computed (a) without mutation type data, VGD^{-M}, and (b) with mutation type data, VGD, respectively, stratified by mutation type for all variants, according to an embodiment of the invention.

Start-loss, essential splice site, nonsense, and frame-shift variants have the largest impact on the resulting protein, and by design, also have the largest scores in the VGD model (Table 2, FIGS. 5A and 5B). The score distribution of variants of these types is highly skewed toward a maximum value (e.g., 1.0), especially when comparing dysfunction scores without VGD^{-M} and with VGD the mutation type contribution (FIGS. 5A and 5B). The median VGD score for each of these variants is at least 0.98 (Table 6). In-frame indels also have relatively high damage scores (e.g., median VGD of 0.82).

Missense variants have a large spread of damage scores, with slightly more deleterious than non-deleterious variants (e.g., median VGD=0.57). Stop-loss variants have an intermediate level of damage (e.g., median VGD=0.36). All other mutation types are associated with lower damage scores (e.g., medians less than 0.02) (Table 6, FIG. 5B).

The VGD model classification was therefore shown to generally concur with the variant's mutation type data, even in a blind test without considering the mutation type data: variants that are associated with non-damaging mutation types e.g. synonymous mutations, non-essential splice sites, etc., were assigned a relatively low probability of disease contribution according to their VGD scores and variants that are associated with damaging mutation types e.g. start-loss, essential splice site, nonsense, and frame-shift variants, were assigned a relatively high probability of disease contribution according to their VGD scores.

Comparison of Results to Other Variant Scoring Techniques

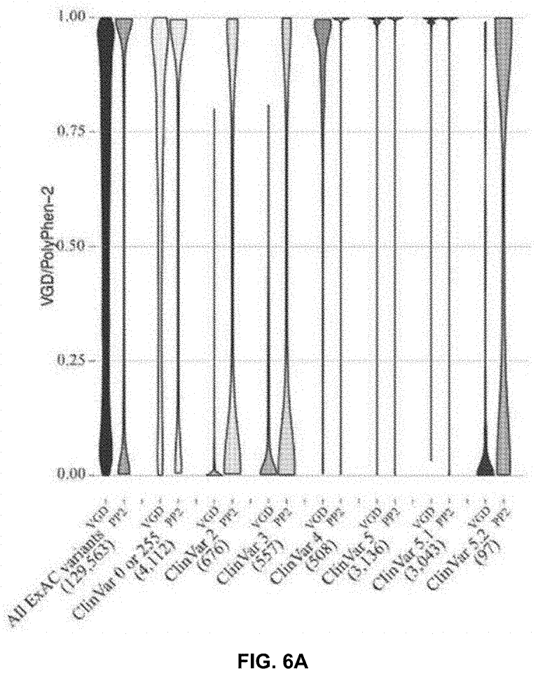

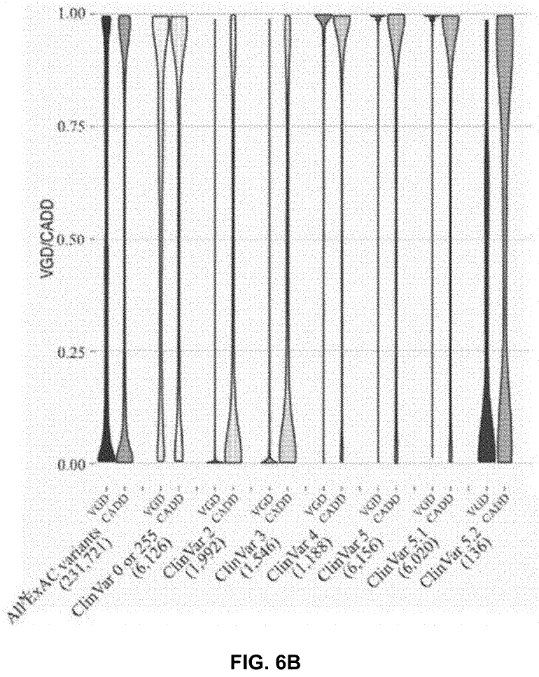

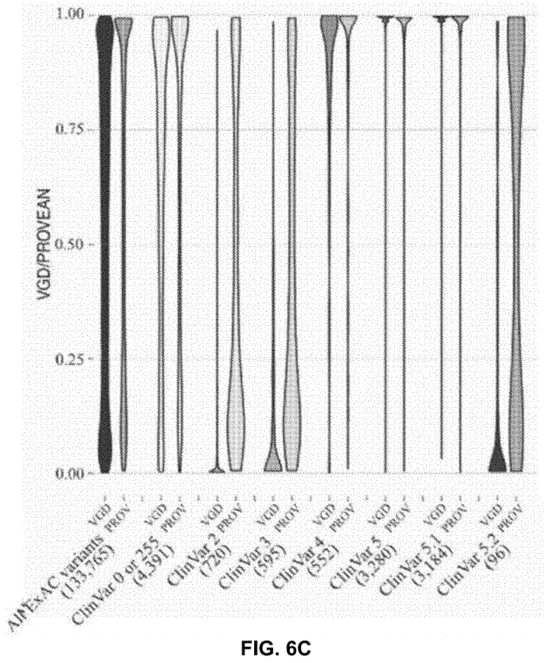

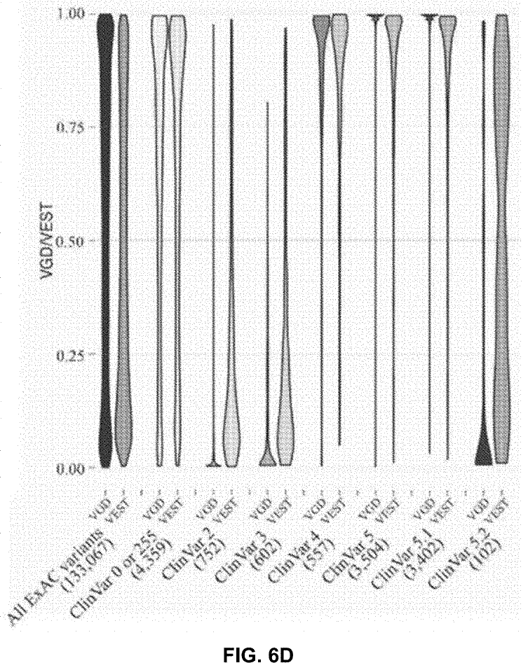

To determine the accuracy of the VGD model as compared to pathogenic predictor metrics, a side-by-side comparison of VGD scores and PolyPhen-2, PROVEAN, CADD, and VEST scores is provided. Reference is made to FIGS. 6A-6D and FIGS. 13A-13D, which show violin plots of an example side-by-side comparison of VGD scores and (A) PolyPhen-2, (B) adjusted CADD, (C) adjusted PROVEAN, and (D) VEST scores, stratified by clinical classification category, according to an embodiment of the invention. FIGS. 6A-6D are stratified by ClinVar category and FIGS. 13A-13D are stratified by VariBench category. FIGS. 6A-6D use the same colors for each ClinVar category as used in FIGS. 4A-4B and FIGS. 13A-13D use the same colors for each VariBench category as used in FIG. 12. Only the variants with available pathogenic predictor metrics are included in these figures.

In general, for many of the analyzed variants, each of the four pathogenic predictor metrics correctly assigns a deleterious score to “pathogenic” and a non-deleterious score to “benign” ClinVar and VariBench variants (see e.g., FIGS. 6A-6D, FIGS. 13A-13D, Table 5). However, these four pathogenic predictor metrics are not sensitive enough to handle variants with conflicting evidence such as our ClinVar-5.2's. All of the four pathogenic predictor metrics inaccurately assign variants of these types relatively high probabilities of damage, whereas the VGD model more accurately assigns variants of these types relatively low probabilities of damage (see e.g., FIGS. 6A-6D, Table 5).

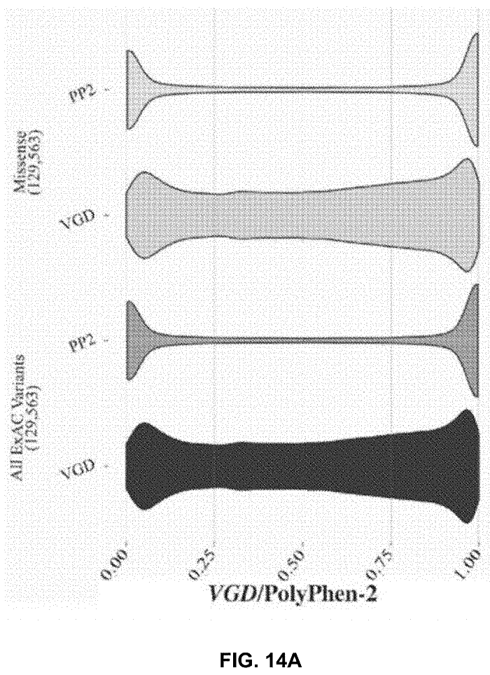

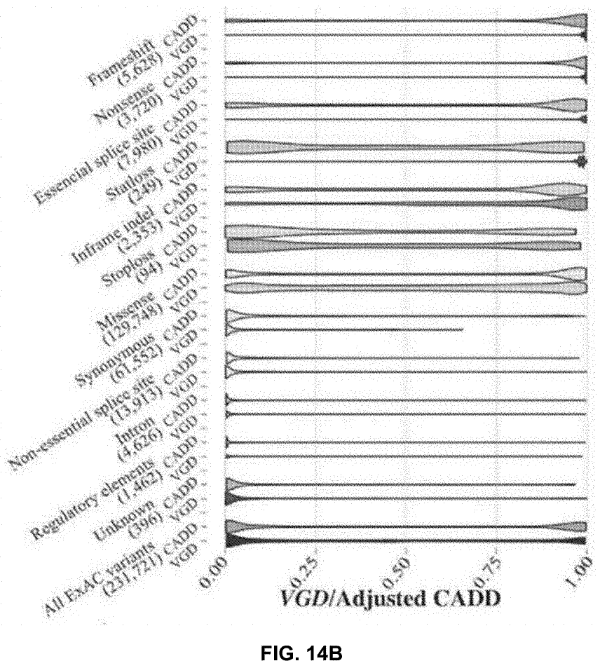

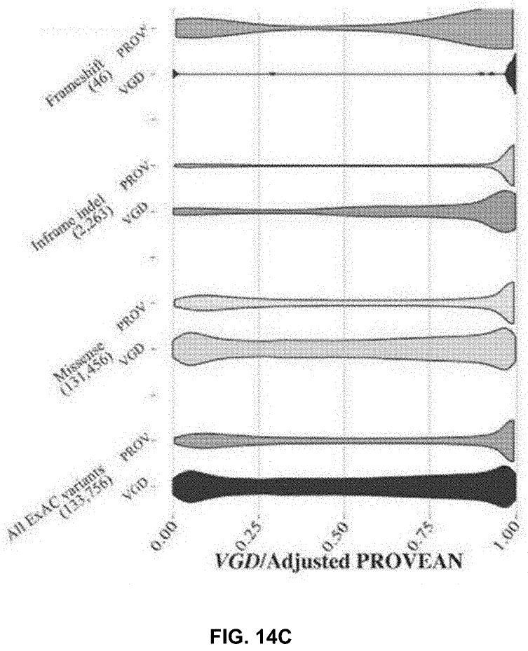

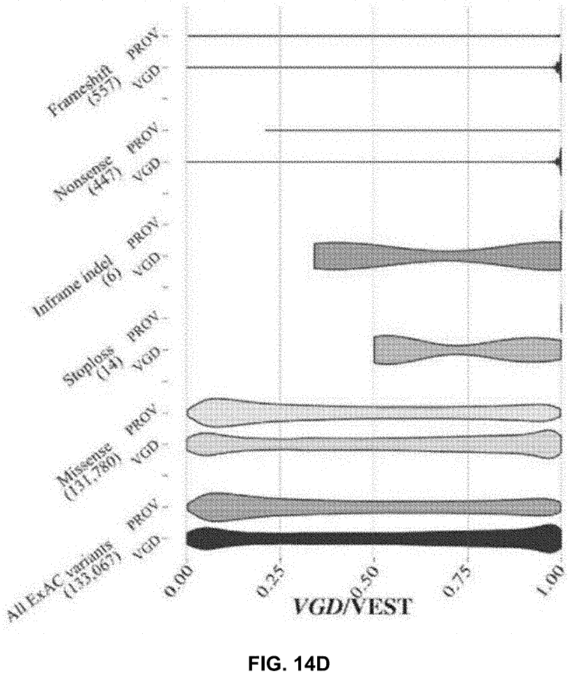

Further limitations of the four pathogenic predictor metrics are shown in FIGS. 14A-14D, which show violin plots of an example comparison of variant-specific gene dysfunction and (A) PolyPhen-2, (B) adjusted CADD, (C) adjusted PROVEAN, and (D) VEST scores, stratified by mutation type for all variants, according to an embodiment of the invention. FIG. 14A shows that PolyPhen-2 is inherently binary (e.g., few of these variants have intermediate PolyPhen-2 values), and assigns very high or low damage scores to all missense variants. FIG. 14B shows that CADD also assigns a lower damage score to stop-gain and stop-loss variants and is inherently binary for all missense variants (see e.g., FIG. 14B, Table 6).

VGD Outperforms Existing Variant Classification Databases

Embodiments of the invention may assess the disease-risk of novel or de novo mutations or variants that have never before been validated or studied in diseased patients. The VGD model identified 29,317 novel variants as pathogenic (e.g., with VGD scores of at least 0.95) that were not yet clinically classified (e.g., by ClinVar or VariBench) as well as 68,594 novel variants as benign (e.g., with VGD scores less than or equal to 0.05) (Table 1). Accordingly, embodiments of the invention may be used to discover disease-correlated variants before the variants are clinically validated, thereby predicting disease risk in patients that would have otherwise been ignored. The majority of these novel variants have relatively low maximum population frequencies (e.g., median of 6.06×10-5 for the likely damaging variants and 9.63×10-5 for the likely benign variants), which may explain why they have yet to be observed in enough diseased individuals to be included in clinical (e.g., ClinVar and VariBench) curations.

For the 192 genes with at least ten ClinVar 5 and 5.1 variants, the 99% and 99.9% one-sided bootstrap confidence intervals were calculated for the average VGDⱼ^{-Cl} (VGDⱼ without including the ClinVar information) among all ClinVar 5 and just ClinVar 5.1 variants (see e.g., Table 7, FIG. 7A). FIG. 7B shows a distribution of the density of confidence interval thresholds versus variant-specific gene dysfunction (VGD^{-Cl}) among all ClinVar 5 variants and ClinVar 5.1 variants for each gene at two confidence levels (99% and 99.9%). In FIG. 7B, the distribution of the lower confidence interval bounds is highly skewed, with most genes having values above 0.80. There is a 99% (or 99.9%) confidence that the average per-gene naive VGD score for ClinVar 5.1 (and 5) variants is above the corresponding threshold. These intervals may thus be utilized as per-gene lower bound proxies for “likely pathogenic” naive VGD scores for variants not covered by ClinVar.

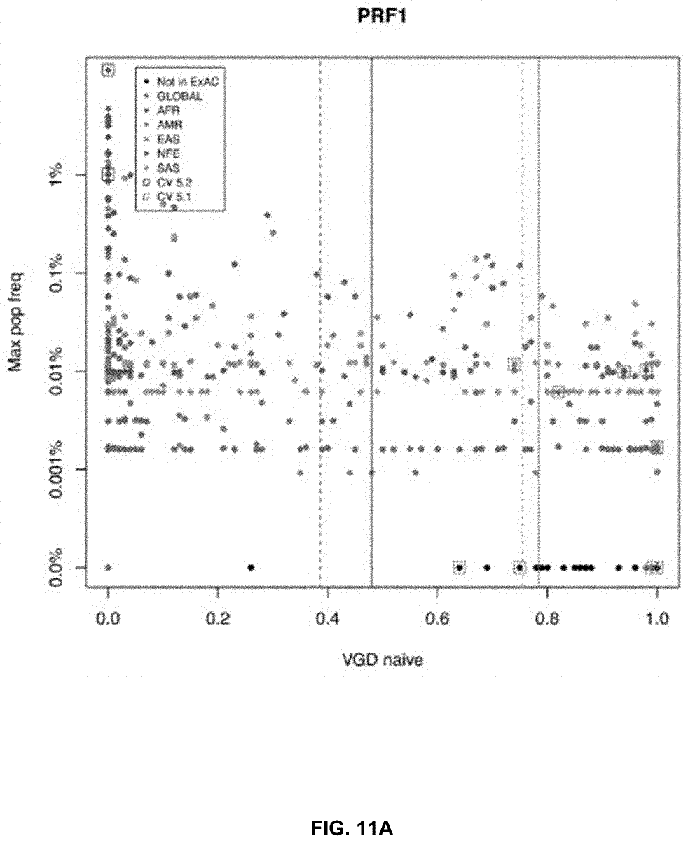

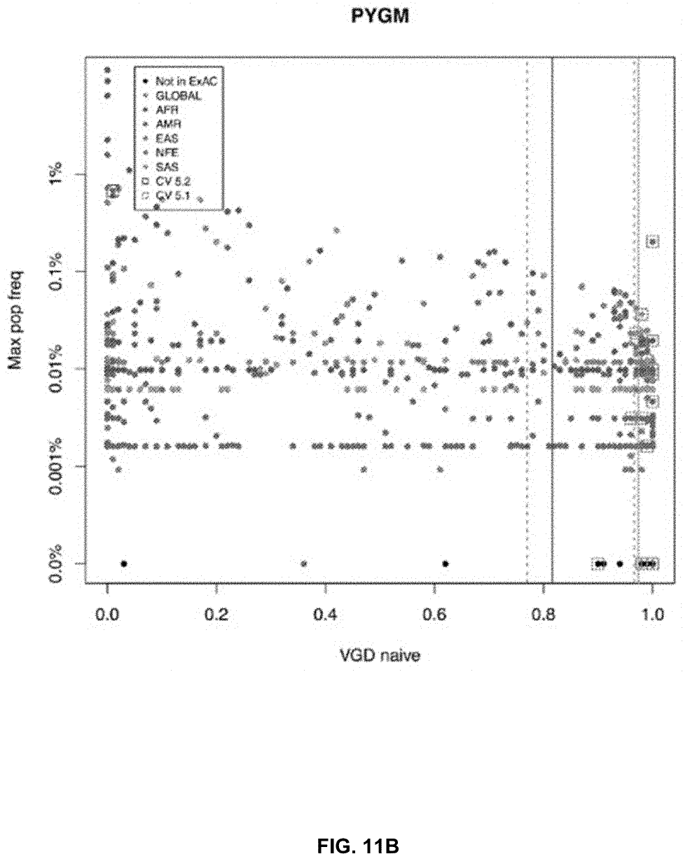

Several genes were found that have a relatively large difference between their interval bounds for their ClinVar 5 and 5.1 variants. Such relatively large intervals are shown, for example, in FIG. 11A, which shows all variants in Pore Forming Protein 1 (PRF1), and in FIG. 11B, which shows all variants in Phosphorylase, Glycogen, Muscle (PYGM), according to some embodiments of the invention. The median maximum population frequency of the 20,055 non-ClinVar “likely pathogenic” variants (with naive VGD scores greater than their per-gene thresholds) is 6.06×10⁻⁵ (IQR: 6.06×10⁻⁵ to 0.00012), which is higher than their ClinVar 5.1 counterparts (0; IQR: 0 to 3.02×10⁻⁵) (Table 5).

### Living Organisms

An organism that is genetically screened according to embodiments of the invention may be a living organism whose DNA is obtained from the organism's biological sample and sequenced to identify a genetic mutation and assess one or more likelihoods that the genetic mutation causes gene-dysfunction in organisms. The living organism may be one or more of a pre-natal organism, a fetus, a newborn, a post-natal organism, a child, an adult, blood, tissue, saliva, a stem-cell, and a tumor, to perform genotype screening, such as, pre-natal genotype screening, post-natal genotype screening, newborn genotype screening, stem-cell screening, and tumor screening. Other types of living organisms and genotype screenings may be used.

### Virtual Organisms

An organism that is genetically screened according to embodiments of the invention may be a virtual or simulated organism. Although the organism is virtual, the organism's genetic information is real, for example, derived by combining at least a portion of real genetic information sequenced from DNA obtained from biological samples of two living potential parents. Accordingly, a virtual organism's genetic information represents transformed, permuted, or intertwined biological DNA samples of two living human organisms.

Reference is made to FIG. 17, which schematically illustrates an example of simulating a hypothetical mating of two (i.e. a first and a second) potential parents for generating a virtual progeny according to an embodiment of the invention.

For each of the two potential parents, a processor (e.g., sequence analyzer processor 112 of FIG. 1) may receive a potential parent's diploid genetic sequence 402, 404. A “diploid” genetic sequence includes two alleles from the two sets of chromosomes respectively labeled “A” and “B” at each genetic locus of a diploid cell of the potential parent, whereas a “haploid” genetic sequence includes one allele from one chromosome at each genetic locus of a haploid cell of the potential parent. For each of the two potential parents' diploid genetic sequences 402 and 404, the processor may simulate genetic recombination of the two sets of chromosomes A and B from the parent's diploid genetic sequence 402, 404 (having two alleles at each genetic locus) to generate a virtual gamete haploid genetic sequence 406, 408 (having one allele per genetic locus). The processor may simulate recombination by progressing locus-by-locus along a “haplopath” through each parent's diploid genetic sequence 402, 404 and selecting one of the two alleles at each genetic locus (either the allele in chromosome A or the allele in chromosome B). The selection of alleles may be at least partially random and/or at least partially non-random, for example, based on defined correlations between alleles at different loci referred to as “linkage disequilibrium”. The haploid genetic sequence may mimic or simulate recombination of the genetic material in the two chromosomes A and B to form a discrete haploid genetic sequence of a virtual gamete 406, 408, e.g., a virtual sperm or virtual egg.

The two virtual gamete haploid genetic sequences 406 and 408 for the two respective potential parents may be combined to simulate a mating between the first and second potential parents resulting in a virtual progeny diploid genetic sequence 410 (a discrete genome of a child potentially to be conceived).

Since the selection of alleles is at least partially random, this mating is just one of the many possible genetic combinations for the first and second potential parents. This process may be repeated multiple times (e.g., hundreds or thousands of times), each time following a different recombination path (e.g., a different sequence of alleles selected) for one or both of the potential parents, to generate multiple genetic permutations that are possible for mating the first and second potential parents. The virtual progeny diploid genetic sequence 410 may include a single (e.g., most probable) genetic sequence or a probability distribution of multiple possible sequences, for example, to indicate, for many possible matings, the overall likelihood of each of multiple alleles at each of one or more loci in a virtual or hypothetical progeny.

Embodiments of the invention may use methods for simulating a mating between two potential parents and generating a virtual progeny genetic sequence described in U.S. Pat. No. 8,805,620, which is incorporated herein by reference in its entirety. Other methods may also be used.

Once the virtual progeny genetic sequence 410 is generated, it may be assigned one or more of the likelihoods that one or more alleles or mutations in the virtual genetic sequence 410 would be deleterious, for example, if replicated in a real living progeny.

### Workflows

Operations described herein may be executed by one or more one or more processor(s) (e.g., controller(s) or processor(s) 108, 110, and/or 112 of FIG. 1), data or data structures described herein may be stored in one or more memor(ies) (e.g., memory unit(s) 114, 116, and/or 118 of FIG. 1), and any visualizations, or data may be displayed on one or more display(s) (e.g., output display 120 or any use display).

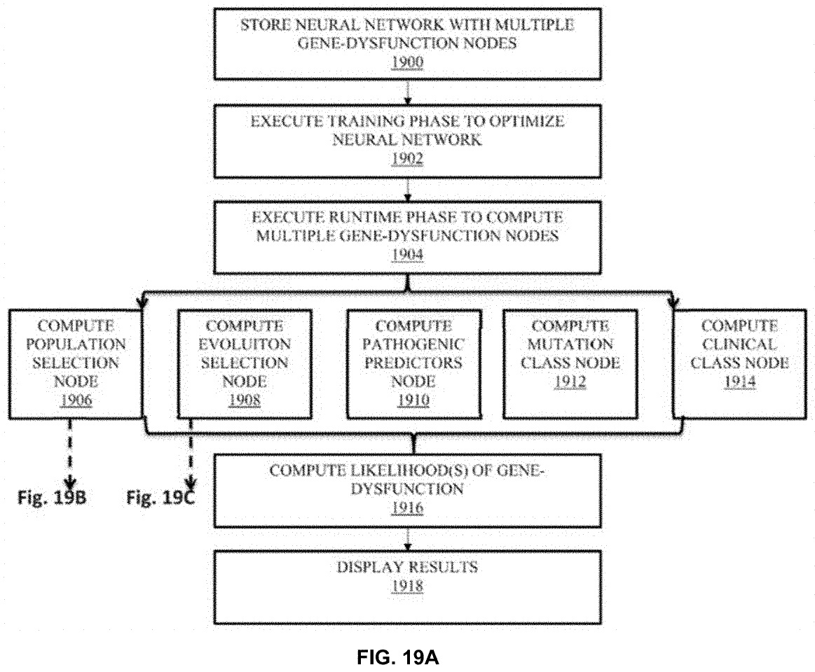

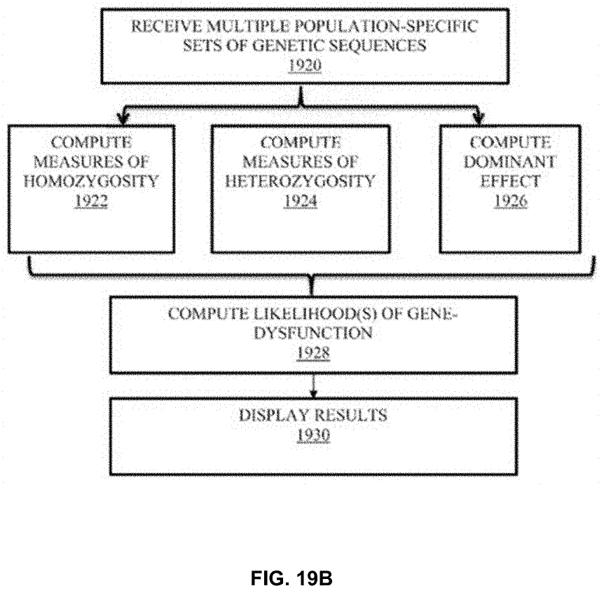

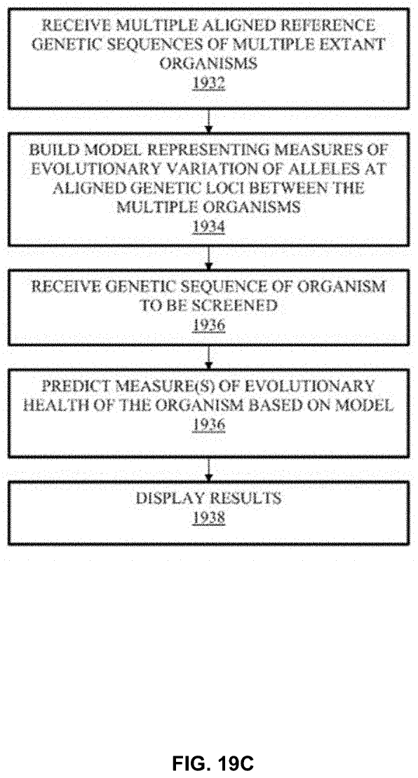

Reference is made to FIG. 19A, which is a flowchart of a method for assessing risk of variant-specific gene dysfunction according to an embodiment of the invention.

In operation 1900, a memory may store and a processor may access a neural network including multiple nodes respectively associated with multiple different gene-dysfunction metrics and multiple different confidence weights. The neural network may combine, aggregate or compose the multiple gene-dysfunction metrics according to the respective associated confidence weights to generate one or more likelihoods that a genetic mutation causes gene-dysfunction in organisms.

In operation 1902, a processor may execute a training-phase. In the training-phase, the processor may train the neural network using an input data set including one or more genetic mutations to generate new gene-dysfunction metrics and new associated confidence weights that optimize the neural network based on a cost factor. In some embodiments, the processor may optimize the neural network in the training-phase by shifting a center of the one or more likelihoods of known pathogenic mutations toward one or more maximal likelihoods, shifting a center of the one or more likelihoods of known benign mutations toward one or more minimal likelihoods, and/or shifting a center of the one or more likelihoods of uncharacterized mutations away from the one or more maximal or minimal likelihoods. In some embodiments, the processor may optimize the neural network in the training-phase by reducing the cost factor associated with the known pathogenic mutations by generating one or more pathogenic thresholds providing a lower bound for the one or more likelihoods of a plurality of the known pathogenic mutations and minimizing the difference between the one or more pathogenic thresholds and respective one or more maximal likelihoods. In some embodiments, the processor may optimize the neural network in the training-phase by reducing the cost factor associated with the uncharacterized mutations by generating one or more pathogenic thresholds providing a lower bound for the one or more likelihoods of a plurality of the known pathogenic mutations and minimizing the number of the uncharacterized mutations having one or more likelihoods above the one or more pathogenic thresholds. In some embodiments, the processor may optimize the neural network in the training-phase by reducing the cost factor associated with the known benign mutations by minimizing mean distribution values of the one or more likelihoods of the known benign mutations. In some embodiments, the processor may optimize the neural network in the training-phase on a gene-by-gene basis and may aggregate gene-specific optimization results, for example, across a genome to obtain a combined genome-wide cost factor. Training phase operation 1902 may be repeated, for example, each time a new input data set is received, new nodes are added to the neural network, optimization parameters are changed, or periodically.

In operation 1904, a processor may execute a run-time phase. In the run-time phase, the processor may identify a genetic mutation and compute the multiple gene-dysfunction metrics for the identified genetic mutation based on the new gene-dysfunction metrics and the associated new confidence weights of the neural network. The run-time phase may include one or more of operations 1904-1918.

In operation 1906, a processor may compute one or more population selection nodes in the neural network associated with multiple population-specific measures of homozygosity for each of multiple populations, which is further described in reference to FIG. 19B. The processor may generate each of the multiple population-specific measures of homozygosity by comparing the count of observed homozygotes of the identified genetic mutation measured on both chromosomes at a genetic locus in a population-specific set of genetic sequences and an expected homozygote count based on a total observed count of the identified genetic mutation measured on either chromosome at the genetic locus in the population-specific set. The processor may weigh the measures of homozygosity with the one or more population-specific confidence weights defined based on a magnitude of the observed or expected homozygote counts in each population-specific set. The processor may compute one or more population selection nodes in the neural network associated with multiple population-specific measures of heterozygosity for each of multiple populations. The processor may generate each of the multiple population-specific measures of heterozygosity based on the total observed count of the identified genetic mutation and the clinical visibility of the identified genetic mutation. The processor may weigh the measures of heterozygosity with one or more population-specific confidence weights defined based on a total count of the identified genetic mutation in the corresponding population. The processor may compute one or more population selection nodes in the neural network associated with multiple population-specific measures of a dominant effect based on an allele count of the identified genetic mutation compared to a distribution of allele counts across a plurality of pathogenic mutations in an identified gene. The processor may weigh the measures of the dominant effect with one or more confidence weights defined based on a number of the plurality of pathogenic mutations in an identified gene. The population selection node is described in further detail in reference to FIG. 19B.

In operation 1908, a processor may compute one or more evolutionary constraint or selection nodes in the neural network associated with a measure of evolutionary variation of alleles at each of one or more common ancestral genetic loci in multiple organisms corresponding to one or more loci of the identified genetic mutation. In one embodiment, the one or more likelihoods that the identified genetic mutation causes gene-dysfunction in organisms may be indirectly proportional to the measure of evolutionary variation in alleles. In one embodiment, the processor may compute the measure of evolutionary variation corresponding to the identified genetic mutation based on a frequency with which the genetic mutation has occurred and persisted in the multiple organisms over evolutionary history. In another embodiment, the processor may compute the measure of evolutionary variation corresponding to the identified genetic mutation based on a proximity in a phylogenetic tree representing an evolutionary timescale between a reference genetic sequence of the same species as the organism and one or more other species in which the genetic mutation has occurred. In another embodiment, the processor may compute the measure of evolutionary variation corresponding to the identified genetic mutation based on an average pairwise difference between different alleles at the corresponding one or more loci of the identified genetic mutation. In another embodiment, the processor may compute the measure of evolutionary variation corresponding to the identified genetic mutation based on a distance metric between a reference genetic sequence and a genetic sequence of the organism. In another embodiment, the processor may compute the measure of evolutionary variation corresponding to the identified genetic mutation based on a probability of transitioning from a reference allele to the genetic mutation. In another embodiment, the processor may compute the measure of evolutionary variation corresponding to the identified genetic mutation based on ratio w of a non-synonymous substitution rate to a synonymous substitution rate, wherein a non-synonymous substitution is an allele substitution in a codon that does not change an amino acid encoded by the codon and a synonymous substitution is an allele substitution in the codon that does change the amino acid. The processor may weigh the evolutionary constraint nodes with one or more evolutionary constraint confidence weights based on a distribution of mutation rates at different genetic loci in the multiple aligned genetic sequences. In some embodiments, the multiple organisms may be from multiple different species, whereas in other embodiments, the multiple organisms are from a single species. The evolutionary selection node is described in further detail in reference to FIG. 19C.

In operation 1910, a processor may compute one or more mutation class nodes in the neural network that measure a mutation type metric associated with a mutation type of the identified genetic mutation. In various embodiments, the mutation type of the identified genetic mutation may include one or more of: start-loss, stop-loss, stop-gain, stop-retained, frame-shift indel, in-frame indel, essential splice site, splice region, microsatellite, synonymous, missense, intron, and untranslated region. In one embodiment, the processor may compute the mutation class metric for the identified genetic mutation of a microsatellite mutation type inversely proportionally to a length of a repeating microsatellite sequence in the identified genetic mutation. The processor may weigh the microsatellite mutation class metric with a microsatellite mutation class weight that is directly proportional to the length of the repeating microsatellite sequence in the identified genetic mutation. In one embodiment, the processor may compute the mutation class metric for the identified genetic mutation of an in-frame indel mutation type directly proportionally to a number of codons inserted or deleted by the identified genetic mutation. The processor may weigh the in-frame indel mutation class metric with an in-frame indel mutation class weight that is directly proportional to a number of codons inserted or deleted by the identified genetic mutation.

In operation 1912, a processor may compute one or more pathogenic predictor nodes in the neural network that measure one or more pathogenic predictor metrics predicting a degree of pathology of the identified genetic mutation. In one embodiment, the processor may compute the one or more pathogenic predictor metrics by transforming a PROVEAN value of the identified genetic mutation to a linear scale and an associated confidence weight proportional to the PROVEAN value. In one embodiment, the processor may compute the one or more pathogenic predictor metrics by transforming a CADD value of the identified genetic mutation from a Phred scale to a linear scale and an associated confidence weight proportional to the CADD value. In one embodiment, the processor may compute the one or more pathogenic predictor metrics by transforming two PolyPhen-2 values of the identified genetic mutation, HumDiv and HumVar, into an average value and an associated confidence weight that is inversely proportional to the difference between the two PolyPhen-2 values. In one embodiment, the processor may compute the one or more pathogenic predictor metrics from a VEST value of the identified genetic mutation.

In operation 1914, a processor may compute one or more clinical classification nodes in the neural network that measure one or more clinical classification metrics defining available clinical classification data for the identified genetic mutation. In one embodiment, the processor may compute the clinical classification metrics to be substantially maximal when the identified genetic mutation is clinically classified as pathogenic and substantially minimal when the identified genetic mutation is clinically classified as benign. In one embodiment, the processor may compute a clinical classification confidence weight associated with the clinical classification metrics to be substantially maximal when the clinical classification is uncontested and relatively lower than maximal when the clinical classification is contested.

In operation 1916, a processor may combine or aggregate the values or outputs form the multiple gene-dysfunction metrics to compute one or more likelihoods that the identified genetic mutation causes gene-dysfunction in the organism. In one embodiment, in the run-time phase, a processor may compare the one or more likelihoods to one or more pathogenic threshold ranges to predict if the genetic mutation will cause gene-dysfunction in the organism.

In operation 1918, a display may display results, input data, or intermediate data from operations 1900-1916 or data represented as FIGS. 2, 4-18. In some embodiments, the display may display a visualization of the genetic mutation predicted to cause gene-dysfunction in an image or sequence of the organism's DNA together with the one or more likelihoods that the genetic mutation causes gene-dysfunction.

Reference is made to FIG. 19B, which is a flowchart of a method of predicting gene-dysfunction associated with a genetic mutation in an organism based on population-specific selection factors or nodes according to an embodiment of the invention.

In operation 1920, a processor may receive multiple population-specific sets of genetic sequences each including multiple genetic sequences obtained from genetic samples of organisms from a different respective one of multiple populations.

In operation 1922, a processor may generate each of multiple population-specific measures of homozygosity of the genetic mutation for each of the respective multiple populations. The measures of homozygosity in each population may be computed by comparing the count of observed homozygotes of the genetic mutation measured on both chromosomes at a genetic locus in the population-specific set and an expected homozygote count based on a total observed count of the genetic mutation measured on either chromosome at the genetic locus in the population-specific set.

In operation 1924, a processor may generate each of multiple population-specific measures of heterozygosity associated with the genetic mutation for each of the respective multiple populations based on the total observed count of the genetic mutation and the clinical visibility of the genetic mutation. In one embodiment, each of the multiple population-specific measures of heterozygosity is directly proportional to the clinical visibility of the genetic mutation and indirectly proportional to the total count of the genetic mutation in the corresponding population. In one embodiment, each of the multiple population-specific measures of heterozygosity increases when the clinical visibility of the genetic mutation is relatively greater than the total count of the genetic mutation in the corresponding population. In one embodiment, the processor may weigh the multiple population-specific measures of heterozygosity based on the total frequency of the genetic mutation in the corresponding population. In one embodiment, the processor may compute the clinical visibility of the genetic mutation based on one or more measures of: a frequency with which the genetic mutation is cited in clinical studies or literature, a number of published articles referencing the genetic mutation, a number of compound heterozygotes of the genetic mutation with other variants described in clinical studies or literature, and a number of search results or an order or ranking in a search result for the genetic mutation.

In operation 1926, a processor may generate one or more measures of a dominant effect associated with the genetic mutation based on an allele count of the identified genetic mutation compared to a distribution of allele counts across a plurality of pathogenic mutations in an identified gene. In one embodiment, the processor may weigh the measures of the dominant effect with one or more confidence weights defined based on the allele count, a number of the pathogenic mutations in the identified gene, and/or a distribution of allele counts across the plurality of pathogenic mutations in the identified gene.

In operation 1928, a processor may compute one or more likelihoods that the genetic mutation causes gene-dysfunction in the organism based on one or more of the multiple population-specific measures of homozygosity. In one embodiment, the processor may compute the one or more likelihoods that the genetic mutation causes gene-dysfunction to be greater when the observed homozygote count is less than the expected homozygote count and to be smaller when the observed homozygote count is greater than the expected homozygote count. In one embodiment, the processor may compute the one or more likelihoods that the genetic mutation causes gene-dysfunction to increase when the observed homozygote count is less than the expected homozygote count. In one embodiment, the processor may compare the one or more likelihoods to one or more pathogenic threshold ranges to predict if the genetic mutation will cause gene-dysfunction in the organism. In one embodiment, the processor may compare the one or more likelihoods to one or more benign threshold ranges to predict if the genetic mutation is benign in the organism. In some embodiments, the processor may compute the one or more likelihoods based on a combination of the multiple population-specific measures of homozygosity corresponding to the multiple populations, each weighted according to an independent population-specific weight. In one embodiment, each of the population-specific weights is defined based on a magnitude of the observed or expected homozygote count in each population-specific set. In one embodiment, the population-specific weights are defined based on degrees from which the organism descended from each population. In some embodiments, the processor may compute the one or more likelihoods based on a single population-specific measure of homozygosity corresponding to a single primary population from which the organism descended. In some embodiments, the processor may compute the one or more likelihoods and weights by training a model to discriminate between genetic mutations known to cause pathology and genetic mutations known to be benign. The one or more likelihoods may be computed on a continuous scale.

In operation 1930, a display may display results, input data, or intermediate data from operations 1920-1928 or data represented as FIGS. 2, 4-18. In some embodiments, the display may display a visualization of the genetic mutation predicted to cause gene-dysfunction in an image or sequence of the organism's DNA together with the one or more likelihoods that the genetic mutation causes gene-dysfunction.

Reference is made to FIG. 19C, which is a flowchart of a method of predicting gene-dysfunction associated with a genetic mutation in an organism based on evolutionary selection factors or nodes according to an embodiment of the invention.

In operation 1932, a processor may receive multiple aligned reference genetic sequences of multiple extant organisms representative of one or more species or populations (e.g., as shown in FIG. 15). The reference genetic sequences may be sequenced by a genetic sequencer (e.g., sequencer 102 of FIG. 1) or pre-stored and retrieved from a memory or database (e.g., memory unit(s) 114, 116, and/or 118 of FIG. 1) and may be aligned by a sequence aligner (e.g., sequence aligner 104 of FIG. 1) or pre-aligned in the memory or database.

In operation 1934, the processor may build or obtain a model representing measures of evolutionary variation of alleles or nucleotides at one or more aligned genetic loci between the multiple organisms. The model may be a single-species model (e.g., the multiple organisms are from the same single species) or a multi-species model (e.g., the multiple organisms are from different multiple species). The model may include, for example, a phylogenetic tree (e.g., as shown in FIG. 16), or another data structure.

In operation 1936, the processor may receive a genetic sequence of an organism to be genetically screened.

In operation 1938, the processor may use the model of operation 1934 defining the evolutionary past variation among multiple extant organisms of different populations or species to predict or interpolate a likelihood or probability of evolutionary health of the organism in operation 1936. The processor may determine the differences between the organism's genetic sequence and one or more aligned reference genetic sequences and may assign each allele (or only different or mutated alleles) a measure of evolutionary variation that is a function of variations in alleles at corresponding aligned genetic loci in the multiple aligned genetic sequences (e.g., loci derived from one or more common ancestral genetic loci in the multiple organisms). The processor may compute one or more likelihoods that an allele mutation at each of the one or more genetic loci in the organism is deleterious based on the measure of evolutionary variation of alleles at the corresponding aligned genetic loci for the multiple organisms. The likelihoods may include one or more likelihoods or likelihood distributions for one or more alleles, one or more allele mutations, one or more genes, one or more codons, one or more genetic loci or loci segments, for one or more living organisms or virtual progeny of two potential parents (e.g., generated by repeatedly simulating a mating using different virtual gamete(s) in each iteration) and/or for one or more pairs of potential parents (e.g., generated by repeatedly simulating a mating step, in each iteration using the genetic information obtained in operation 1936. The one or more likelihoods may be compared to one or more thresholds or other statistical models to predict if (or a likelihood or degree in which) an allele mutation is deleterious in the organism. For example, mutations at genetic loci with relatively constant or fixed alleles and relatively lower measures of evolutionary variation may be associated with relatively higher likelihoods of deleterious traits, whereas mutations at genetic loci with relatively volatile or changing alleles and relatively higher measures of evolutionary variation may be associated with relatively lower likelihoods of resulting in deleterious traits.

In operation 1940, an output device (e.g., output device 320 of FIG. 3) may output or display, e.g., to a user, the one or more likelihoods or likelihood distributions that the organism would have deleterious traits, or other results, input data, or intermediate data from operations 1932-1938. For example, the output device may output one or more likelihoods that an allele mutation at each of the one or more genetic loci in the simulated virtual progeny will be deleterious based on the measure of evolutionary variation of alleles at the corresponding aligned genetic loci for the multiple organisms.

The organism screened for gene-dysfunction in FIGS. 17A-17C may be a living organism or a virtual organism. When the organism is a living organism, a processor may sequence the organism's DNA obtained from a biological sample to identify the genetic mutation.

When the organism is a virtual organism, a processor may generate the virtual progeny by combining at least a portion of genetic information representing DNA obtained from biological samples of two living potential parents. In one embodiment, the processor may simulate a mating between the two potential parents by combining their genetic sequences to generate one or more virtual progeny genetic sequences (e.g., sequence 410 of FIG. 17). The processor may generate a virtual gamete (haploid genetic sequence) for each potential parent by at least partially randomly selecting one of two allele copies in the parent's two chromosomes (diploid genetic sequence) to simulate recombination at each of a sequence of genetic loci. A virtual gamete for each of the two potential parents (e.g., one virtual sperm and one virtual egg) may be combined to generate the genetic sequence of the virtual progeny. Multiple virtual gametes may be generated for each potential parent by repeating the recombination process each time selecting a different at least partially random sequence of alleles. Multiple virtual progeny genetic sequences may be generated for multiple pairs of potential parents by repeating the step of combining two virtual gametes for each of a plurality of different combinations of two virtual gametes. In one embodiment, the independent carrier status of an individual may be determined by simulating a mating combining the individual's genetic sequence information with that of a sample, averaged, or reference genetic sequence of the same species.

### Virtual Progeny Analytics (VPA)

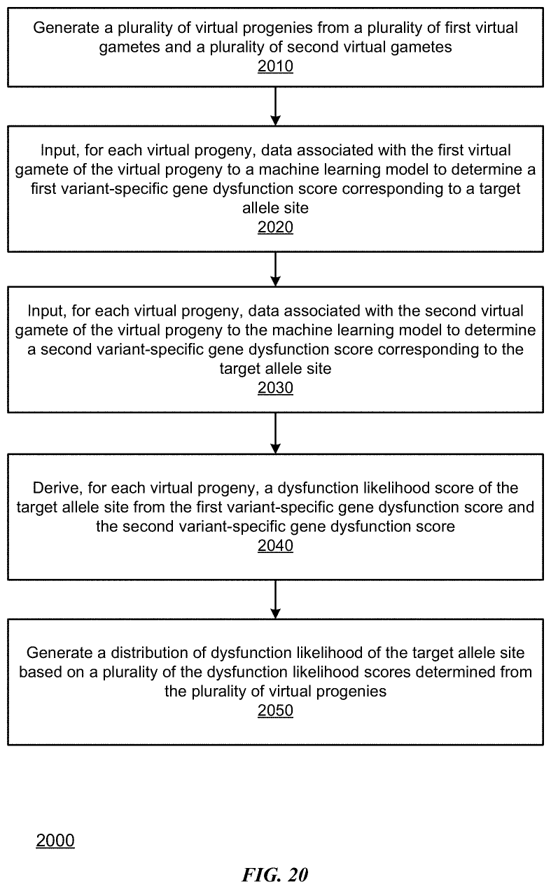

FIG. 20 is a flowchart depicting an example process 2000 of determining an allele dysfunction likelihood that may be used to predict the expression of a phenotype, in accordance with an embodiment. In operation 2010, a processor may generate a plurality of virtual progenies from a plurality of first virtual gametes and a plurality of second virtual gametes. To generate a virtual progeny, a processor may select one of the first virtual gametes from a first pool and select one of the second virtual gametes from a second pool.

The first and second virtual gametes may be generated from genetic datasets of two potential parents. In one embodiment, each of the first virtual gametes may be a sequence of haploid alleles of a first potential parent. Each of the haploid alleles in the sequence may be selected from one of two alleles of the first potential parent. For example, a processor may retrieve a genetic dataset of the first potential parent that may include diploid genotype sequences of the first potential parent. The processor may generate the virtual gamete's sequence, which may also be referred to as a haplopath, by dividing the parent's diploid genotype sequence into a plurality of genetic loci and selecting one of the two alleles of the parent at each locus. The genetic loci may be any intervals of the parent's genotype sequence and can be in any lengths. Different genetic locus can each be in a different length.

In selecting the alleles to form the virtual gamete's sequence, a processor may choose one of two alleles in a semi-random fashion. The processor may rely on a random number generator to choose a random allele at a random initializing locus in the set of N such loci. Each prior or subsequent allele sites along the sequence can be generated according to a normalized likelihood derived from locus-specific association matrices. The likelihood in choosing one of the two alleles at prior or subsequent allele sites may further be based on linkage disequilibrium. For example, the selection may be at least based on rules of genetics such as allele segregation, independent assortment, the linkage between genetic loci, recombination suppression, Hardy-Weinberg equilibria, and other probabilistic genetic processes. Formal rules may be used to estimate the likelihood of transmission of particular alleles and combinations of alleles at multiple loci, from the first potential parent to a virtual gamete.

After a first virtual gamete's sequence is generated, a processor may repeat the selection process for a plurality of first virtual gametes to generate a first pool of first virtual gametes. Similarly, a processor may generate a second pool of second virtual gametes based on the genetic dataset of a second potential parent.

A processor may select one of the first virtual gametes from the first pool and one of the second virtual gametes from the second pool to form a virtual progeny. Male and female virtual gametes are combined pairwise to produce a pool of diploid virtual progenies. For example, the processor may repeat the selection process to generate a plurality of virtual progenies.

In operation 2020, for each virtual progeny, a processor may input data associated with the first virtual gamete of the virtual progeny to a machine learning model to determine a first variant-specific gene dysfunction score (VGDⱼ) corresponding to a target allele site j. Variant-specific gene dysfunction score (VGD) may elucidate the degree of dysfunction that a variant imposes on the haploid activity of a single allele within a diploid cell. Dysfunction values, e.g., on a 0.0 to 1.0 range, may be computed through the application of a multi-layer machine learning model that include variant-specific weighted nodes representing biological, clinical, literature-visibility, and population attributes.

The machine learning model may be a neural network described in association with FIGS. 3A, 3B, and 3C or may be another suitable machine learning model. For example, an example neural network may include multiple nodes, multiple gene-dysfunction metrics, and multiple confidence weights. Each node may be associated with one or more of the multiple gene-dysfunction metrics. Each of the gene-dysfunction metrics may be associated with one of the confidence weights. The neural network may combine one or more of the gene-dysfunction metrics according to the confidence weights to generate one or more variant-specific gene dysfunction scores.

In addition to the genetic sequence input, the machine learning model may also be trained to take into account of other data sources, such as Population-specific alleles and genotype frequencies that are derived from the Exome Aggregation Consortium (ExAC) and the Genome Aggregation Database (gnomAD) maintained by the Broad Institute. Additionally, or alternatively, the nodes in the machine learning model may further include nodes that take into account of general and gene-specific effects of different mutation types, and quantitative output from different models of evolutionary constraint and protein damage as discussed above including PolyPhen-2, VEST, CADD, PROVEAN, and GERP++. The machine learning model may also include nodes that evaluate clinical data, which may be referred to as clinical nodes. The clinical nodes evaluate ClinVar assertions and other locus-specific database information. In one or more of the clinical nodes, literature-visibility may be ascertained through a ClinVar database list of PubMed citations and natural language processing of the OMIM database to count distinct variant associated compound heterozygotes.

In operation 2030, for each virtual progeny, a processor may also input data associated with the second virtual gamete of the virtual progeny to the machine learning model to determine a second variant-specific gene dysfunction score corresponding to the target allele site. Hence, each virtual progeny may be associated with two variant-specific gene dysfunction scores, one derived from the first gamete and another derived from the second gamete.

In operation 2040, for each virtual progeny, a processor may derive a dysfunction likelihood score of the target allele site from the first variant-specific gene dysfunction score and the second variant-specific gene dysfunction score. Various suitable ways may be used to generate the dysfunction likelihood score. In one embodiment, the dysfunction likelihood score is non-binary (e.g., having more than two possible values). For example, a continuous trait model (CTM) may be used. The CTM model may be normalized to a range (e.g., between 1 and 0) with a first end of the range representing that the allele dysfunction is certain and a second end of the range representing no likelihood of allele dysfunction. The CTM may use the geometric mean of allele dysfunction as an estimate of phenotypic response to a genotype with alleles dⱼ and d_{j′}. The genotype may be the two alleles at the target allele site of the virtual progeny. In this CTM model, the dysfunction likelihood score of the target allele site may be determined by the following:

φ_{jj′}=\sqrt{dⱼd_{j′}}

In one case, this CTM model may be based on the recessive disease assumption that a single normally functioning allele (dⱼ=0) is sufficient to prevent any phenotypic change. In other words, φ_{jj′} equals to zero because one of the term dⱼ or d_{j′} is zero. At the opposite extreme, when both alleles are completely dysfunctional, the phenotypic response is at the maximum (e.g., certainty) for the gene under the CTM model because φ_{jj′} equals to one if both dⱼ and d_{j′} equal to one.

In another embodiment, another CTM model may be used that assigns partial functionality to variant. This CTM may be particularly helpful for evaluation of the phenotype expression of autosomal recessive diseases. For example, the CTM model may combine features of genetics-framed models and features of cooperative binding biochemistry-framed models. The dysfunction likelihood score, which may also be referred to as a phenotypic response, may take the following form:

$${ϕ}_{g} ( D ) = 1 - [ \frac{( 1 + β ) \times {( 1 - D )}^{{η}_{g}}}{β + {( 1 - D )}^{{η}_{g}}} ]$$

In the equation above, D may represent a diploid gene dysfunction score derived from the first variant-specific gene dysfunction score and the second variant-specific gene dysfunction score, β may represent a system-wide constant, and η_{g} may represent a gene-specific parameter that is specific to a gene to which a target allele site belongs. For example, η_{g} may be determined based on existing allele and genotype dysfunction data and existing genotype-phenotype correlation data in the literature. For example, for the gene BTD associated with the enzyme biotinidase, η_{g} may be equal to 4.

In some embodiments, before the dysfunction likelihood score is determined, at least one of the first variant-specific gene dysfunction score or the second variant-specific gene dysfunction score may be adjusted with a confliction coefficient. A confliction coefficient may represent the degree of discordance among the dysfunction predictions in some of the subsets of the nodes in the machine learning model, which may be referred to as penultimate network nodes. A confliction coefficient of 0.2 or greater may be associated with a significantly increased likelihood (>95%) that a variant is clinically invisible in homozygotes while the variant likely contributes a clinical phenotype in compound heterozygotes with a more dysfunctional second allele. Hence, based on clinical data and in response to that the target allele site expressing gene dysfunction for heterozygotes exceeds a first threshold portion and that the target allele site expressing no gene dysfunction for homozygotes exceeds a second threshold portion, the processor may adjust one or more of the variant-specific gene dysfunction scores.

In operation 2050, a processor may generate a distribution of dysfunction likelihood of the target allele site based on a plurality of the dysfunction likelihood scores determined from the plurality of virtual progenies. For example, each virtual progeny may have a different dysfunction likelihood score based on the CTM model. A distribution may be generated as a result. The distribution may be used to predict the expression of a target phenotype. For example, the prediction of a target phenotype may be based on a single allele site or multiple allele sites. Hence, the prediction may be based at least on the distribution of dysfunction likelihood of the target allele site, or may further be based on one or more other allele sites that are different from the target allele site. In one embodiment, the target phenotype may be an autosomal recessive disease.

The process 2000 provides a way to perform preconception screening for disease risk that is improved over other methods of screenings. The process enables increased accuracy of clinical decision-making through an assignment of partial functionality to variants and a continuous trait model to diseases such as recessive diseases. In one embodiment, this genetic testing process redirects both the target of analysis (from prospective parents to simulated offspring) and the measured attribute (from carrier status to disease status). The process is more accurate than carrier testing. This virtual progeny analysis enables the assignment of partial functionality to variants and a continuous trait model to recessive disease. It also highlights the prevalence of reported-pathogenic variants that cause disease in compound heterozygotes but not in homozygotes, with implications for clinical decision making.

The process 2000 may be compared to an individual carrier test (ICT). One purpose of a recessive disease carrier test is to identify prospective parents who may be at risk of having a child with disease. The positive predictive value (PPV) of a carrier screen diagnosis is computed as the proportion of potential reproductive couples with true preconception risk, given that one of the individuals is identified as a carrier. From Hardy-Weinberg equilibrium HWE, the denominator of this proportion (representing cases that could be identified as at-risk by a single-individual tested with a maximally sensitive carrier screen) may be computed to be 2pQ(1−Q²) where Q is the square-root of disease incidence, and p is 1−Q. The numerator (representing all couples with true risk) may be computed to be (2pQ)² or 4p²Q². Thus, PPV may be computed to be 4p²Q²/2pQ(1−Q²) or 2(1−Q)Q/(1−Q²). As an example, in northern Europe, the incidence of cystic fibrosis is one in 2,500, yielding a value for Q of 0.02, which indicates a PPV of 3.92%.

### Carrier Testing Estimate

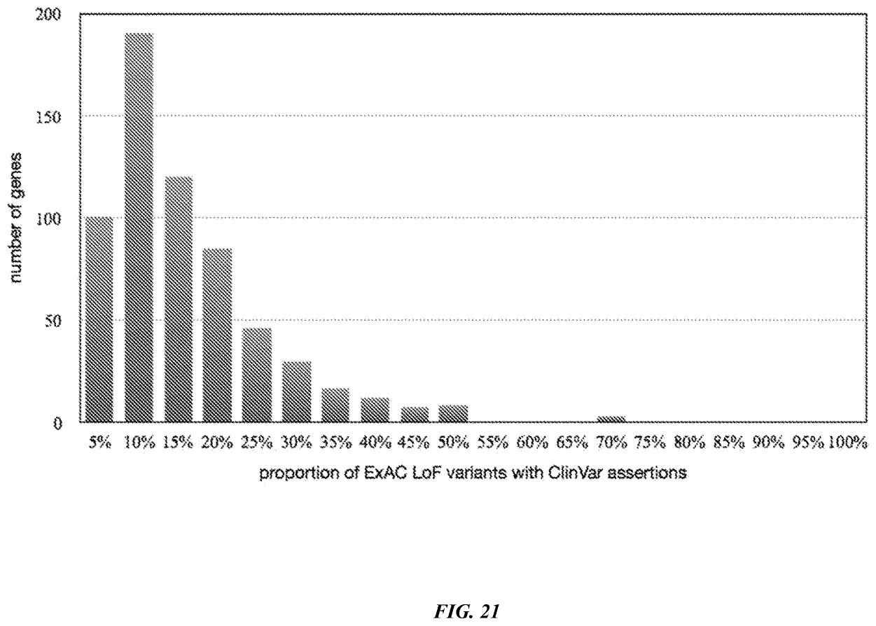

In some embodiments, an estimate of the sensitivity of carrier testing to the detection of recessive disease-associated variants may be obtained by determining the proportion of ExAC (Exome Aggregation Consortium) loss-of-function (LoF) variants that are recorded in the ClinVar database on a per-gene basis. For example, a set of 621 recessive disease genes may be curated for which at least 1% of total ExAC LoF variants are also recorded in ClinVar as a demonstration that LoF is a mechanism of disease. In one case, on a per-gene basis, the average proportion of LoF variants covered by ClinVar is about 14.2%. Since the criteria for asserting disease-association of missense variants are more stringent, it is reasonable to assume that an even lower proportion of disease-associated variants in this class may be reported. FIG. 21 shows a ranking of genes by a proportion of ExAC LoF variants in ClinVar.

In one embodiment, the appearance ratio of a rare recessive disease variant i may be defined as the proportion of newborns in a population who are heterozygous for the variant divided by the proportion of affected newborns who carry the variant in genotypic combination with any disease-associated variant. Given a specific variant i with an allele frequency qᵢ, and a total gene-specific disease mutation load of Q=Σqᵢ, where Q² is the disease incidence at birth, the appearance ratio may be calculated from HWE. For example, the heterozygote proportion is 2qᵢ(1−Q), the affected proportion is qᵢQ, and the appearance ratio may be computed to be 2qᵢ(l−qᵢ)/qᵢQ. Approximating (l−qᵢ)˜1 and canceling out qᵢ yields a variant frequency-independent ratio of 2/Q. An expository example is MCAD (Medium-chain acyl-CoA dehydrogenase) deficiency with an incidence of one in about 10,000 which implies a total mutation load of Q=0.01. This yields an affected-to-carrier appearance ratio of 200:1. Continuing from this example, a rare MCAD mutation present once in every 10,000 people would be observed in an affected child once in every two million births. The carrier testing estimate may be integrated into one of the nodes in a machine learning model to determine variant-specific gene dysfunction score (VGD).

### Experimental Setup

In one experiment in accordance with an embodiment, a set of participants completed a carrier testing process. A core set of at least 93 autosomal genes were included in the carrier tests performed on these participants. Two participants were tested on a limited panel, which included common diseases in the Ashkenazi Jewish population in addition to SMNI, CFTR, and DHCR7. Virtual progeny analytics (VPA) such as process 2000 did not expose a disease-contributing variant in these subjects that the carrier testing process did not already identify. This limited carrier test illustrates the variability in carrier testing coverage over time, but does not affect the results of the study. Participants who did not carry a reported pathogenic variant in any one of the 93 genes were considered individual carrier testing (ICT) negative. Otherwise, participants received an ICT positive report with a list of genes in which reported-pathogenic variants were detected.

Participant specimens were anonymized and processed for gene-targeted exome sequencing with Illumina's Next Generation Sequence TruSightOne enrichment system. Indexed FASTQ sequence reads were aligned to the GRCh37/hg19 human reference genome with BWA to produce individual BAM files. GATK version 3.5 licensed from the Broad Institute (Cambridge, Mass.) was used to obtain diploid sequence information for the complete set of coding regions and 20 bp of each splice site-adjacent intronic region of the core set of 93 autosomal genes.

By way of example, among the participants, fourteen couples with a fertile male who were seeking an egg donor to conceive by IVF were recruited by Reproductive Medicine Associates of New York (RMANY) together with 22 RMANY-recruited egg donors under an IRB-approved protocol and consent (WIRB study number: 1157948). Individual carrier testing (ICT) was performed by one of two independent laboratories. Female and male test reports were matched pairwise for a total of 308 (22×14) paired carrier test (PCT) ascertainments. A match was called “PCT-positive” if the two participants were carrier-positive on the same gene. Each individual participant also provided a saliva specimen for gene-targeted exome next-generation sequencing (NGS).

### Experimental Results

Results of the experiments show that virtual progeny analysis increase detection of preconception disease risk. Twelve (33.3%) of the 36 study participants were ICT-positive for reported-pathogenic variants in one or more genes. Of the 308 matches subjected to PCT, two were same-gene carriers and thus, PCT-positive.

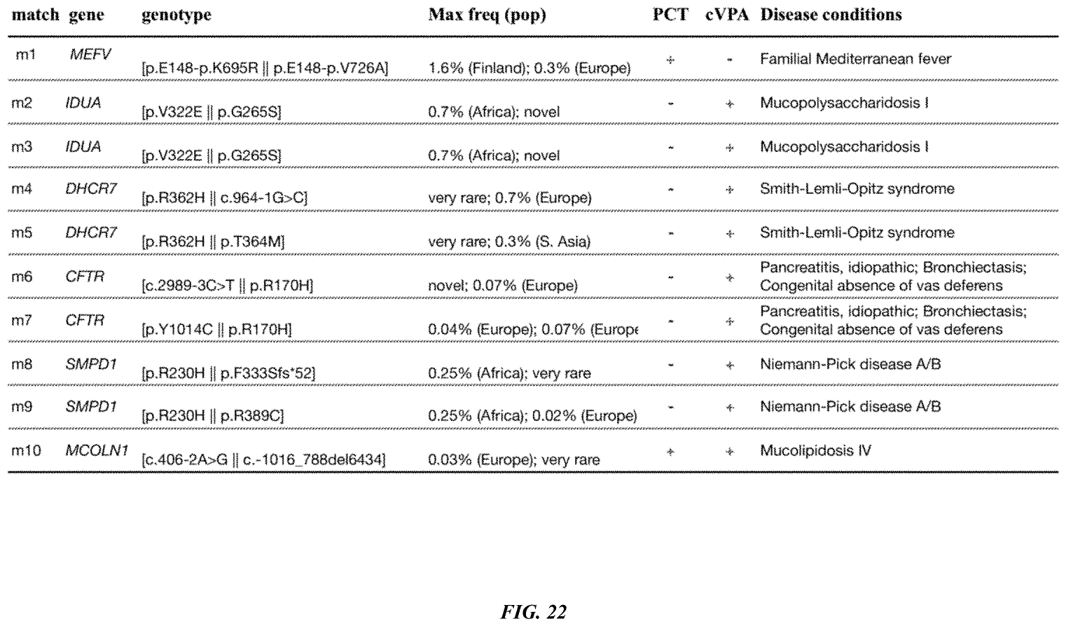

FIG. 22 shows the results of some of the matches and genotypes predicted to be at preconception risk by the PCT test and VPA test. Variants are indicated with standard human genome variation society (HGVS) notation for a coding region or protein variation. Haplotypes with one or more variant loci are notated as “[maternal allele|” or “|paternal allele]”; a “−” is used to separate two loci in the same haplotype. Genotypes are formed by joining haplotypes: [maternal allele∥paternal allele]. “Max freq” is the highest population-specific allele frequency; “pop” is the regional population with the highest frequency (ExAC database). Alleles with a maximum allele frequency of less than 0.01% may be considered “very rare,” and alleles not found in ExAC or dbSNP version 144 may be considered “novel.”

Among the variants identified by PCT, however, only one of these entails significant genetic risk. Thus, in the context of this study, the individual-level positive predictive value (PPV) of ICT is less than 17% (ten of 12 carriers were not at risk of transmitting disease). The same sets of matches and genes were subjected to the automated VPA process yielding a total output of 28,644 couple- and gene-specific test reports. Hundreds of damaging, potentially disease-causing variants are present in each participant genome. But the testing of virtual progeny directly for recessive disease status provides a highly efficient filter for eliminating a majority of these variants as posing no risk to a couple. According to some embodiments, automated computational analysis flagged 14 genotypes (a rate of one in 2,046) derived from twelve (4% of) matches for clinical review. The review process dismissed five genotypes from further consideration, including three that contained CFTR variants incorrectly asserted to be ClinVar-pathogenic, and two with non-disease-associated MEFV genotypes elucidated below. Nine (2.9%) of the 308 matches remained cVPA-positive, including eight with dysfunctional genotypes not detected by PCT (FIG. 22). In total, thirteen different variants in five genes contributed to disease risk. Nine of the 13 were not classified as pathogenic in ClinVar and were not reported by ICT. All clinically at-risk virtual genotypes are compound heterozygous with dysfunctional variants that display diverse regional distributions and population frequencies: three distinct variants are observed predominantly in non-European populations and four are novel or very rare (allele frequency of less than 0.01%); at the opposite end of the frequency spectrum, two variants are present at maximum population carrier frequencies greater than 1%.

### Gene Case Studies of Preconception Disease Risk

In this disclosure, several case studies have been chosen to illustrate specific features of the clinically-reviewed VPA computational process (e.g., process 2000) that provide the basis for a significant enhancement inaccurate reporting of preconception disease risk relative to carrier testing. The first case highlights two separate ICT-positive results which were considered in combination as a hypothetical pairing. The others describe virtual genotypes generated from paired matches of a potential egg donor with a male recipient.

### Case Study: BTD (Biotinidase) Deficiency

The limitations of the binary endpoint variant classification system in ICT are illustrated by the ICT positive results reported to two female participants for the same pathogenic variant (p.D444H) in the BTD gene associated with biotinidase deficiency. If they had been paired reproductively, these two participants would have been PCT-positive. In conflict with this assessment, VPA determines that virtual progeny with a [p.D444H∥p.D444H] genotype were not at risk for BTD associated clinical disease. Conflicting interpretations of this variant were also elicited in a recently published study examining the concordance of variant interpretation results by multiple clinical diagnostic laboratories using the American College of Medical Genetics (ACMG) guidelines. After an attempt at reaching consensus, different laboratories maintained opposite p.D444H assertions of “benign” and “pathogenic.”

The VPA process finds support for a p.D444H-caused diminution of gene activity based on high visibility in the clinical literature together with a moderately high, integrated evolutionary constraint and protein damage score (in the top 38th percentile of missense variants found in the ETD gene). However, application of the Hardy-Weinberg Equilibrium (HWE) to a population cohort selected not to have pediatric disease yields a high likelihood for the absence of any clinical effect on homozygotes.

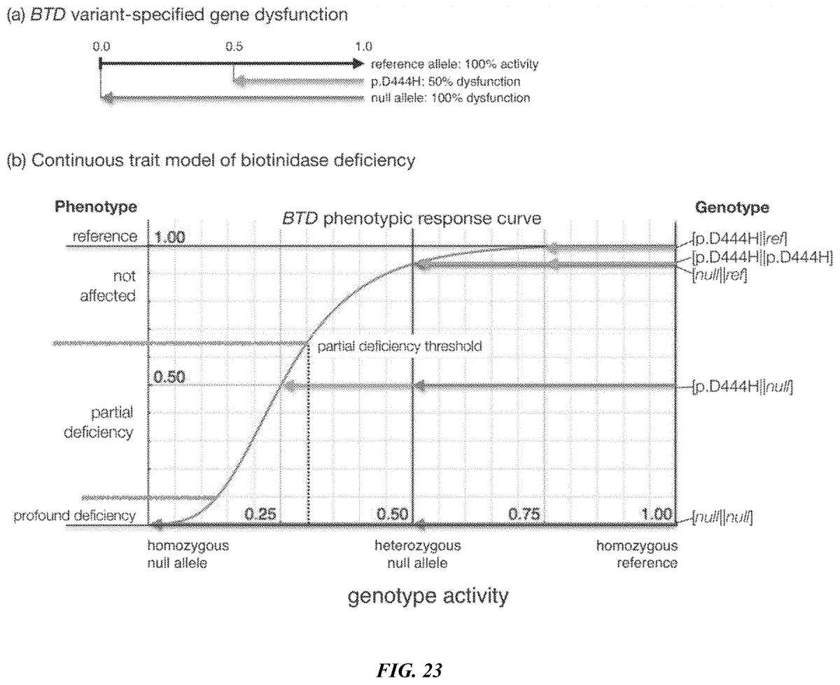

Reconciliation of these seemingly contradictory findings can be achieved with a continuous trait model (CTM) of the ETD genotype-phenotype system, as illustrated in FIG. 23. Process 2000 using geometric mean to determine dysfunction likelihood was used to generate the phenotypic response curve in FIG. 23 to model both the MEFV and ETD gene-disease systems. Variant-specified gene dysfunction is measured as a subtracted percentage of reference allele activity and indicated with a negative vector. Also, an ETD phenotypic response curve is generated as a function of genotype activity based on process 2000. In the diploid gene system, a reference allele has a normalized activity of 0.5. Genotype activity on the x-axis, and phenotypic expression on the y-axis are both calibrated on a 0 to 1 scale where (1,1) is a reference genotype and phenotype and (0,0) is a double-null genotype with a corresponding extreme phenotype. Three ranges of biotinidase deficiency have been established. Below 10% gene activity, a profound deficiency causes serious disease expression; between 10% and 30% activity, a mild clinical phenotype is observed; over 30% activity, biotinidase deficiency is observed through testing, but no clinical phenotype ensues.

One premise of CTM may be that Mendelian deficiency diseases are often recessive because reference alleles have evolved to express much higher activity (in terms of functional gene product) than required in order to maintain the robustness of complex biological pathways in the presence of random mutations. A robust gene allows a diploid organism to tolerate heterozygosity for a null allele that reduces total activity by 50% without any detectable effect on health. Functional studies may provide support for the VPA prediction with the demonstration of a 50% reduction in the activity of the p.D444H allele. Thus, the ETD activity of a diploid [p.D444H∥p.D444H] genotype is equivalent to that of a heterozygous null allele carrier [null∥ref].

As illustrated in FIG. 23, neither genotype causes clinical disease. In contrast, when p.D444H is present in a compound heterozygous genotype with a null allele [p.D444Hllnull], total ETD activity is reduced to 25% of reference levels, which is in the range of clinical detectability. The power provided by VPA for accurately predicting disease risk prior to conception is demonstrated by population modeling based on ETD allele frequency data. The predicted frequency of the major disease-causing genotype [p.D444H∥null] may be calculated as two times the product of each variant frequency according to Hardy-Weinberg statistics. With a global p.D444H frequency of 3.2% and a summed loss-of-function (null) frequency of 0.02%, the disease is predicted to occur in one out of every 78,000 persons, which matches the observed incidence rates of 1/50,000 to 1/100,000. Irrespective of the classification of p.D444H as benign, VUS, or pathogenic, ETD, ICT fails to distinguish between a genuine, but rare, preconception disease risk of 25% or a much more common risk of 0%. In one embodiment, a machine learning model, such as the one used in process 2000, may include one or more nodes that calculate population modeling based on ETD allele frequency data and the variant frequency may be determined based on HWE.

### Case Study: Familial Mediterranean Fever, Intragenic Epistasis Determines True Disease Risk

One of the two PCT-positive matches (m1 in FIG. 22) occurred between participants who are ICT-positive for different reported-pathogenic variants in the MEFV gene associated with familial Mediterranean fever. In conflict with this assessment, VPA predicts a negligible disease risk for the corresponding virtual progeny genotype [p.K695R∥p.V726A] based on a computational process similar to the one described above for ETD. Both p.K695R and p.V726A have uncontested ClinVar-pathogenic assertions and high visibility in the clinical literature. However, at the same time, each allele is found in Hardy-Weinberg equilibrium. Thus, p.K695R and p.V726A are each likely to reduce gene activity by no more than 50% in similarity to ETD: p.D444H. In compound heterozygosity, the two alleles together are predicted to have sufficient activity to avoid clinical symptoms. However, if either allele were inherited together with a fully dysfunctional MEFV allele, a high risk of disease is predicted in agreement with pathogenic variant assertions.

Allele frequency data from the Ashkenazi Jewish (AJ) population provide empirical support for the predictions of VPA Essentially all of this population's MEFV mutation loads reside in the two variants detected in match m1 along with a third variant, p.A744S, that is functionally indistinguishable. These three variants have a combined MEFV heterozygous carrier frequency of 12%, and give rise to genotypes predicted to be at risk for FMF at a rate of one in 266 individuals. However, FMF is a relatively rare condition in the AJ population with a prevalence estimated at one in 73,000. The 200-fold difference between expected and observed prevalence implies a PPV of less than 1% for the [p.K695R∥p.V726A] genotype.

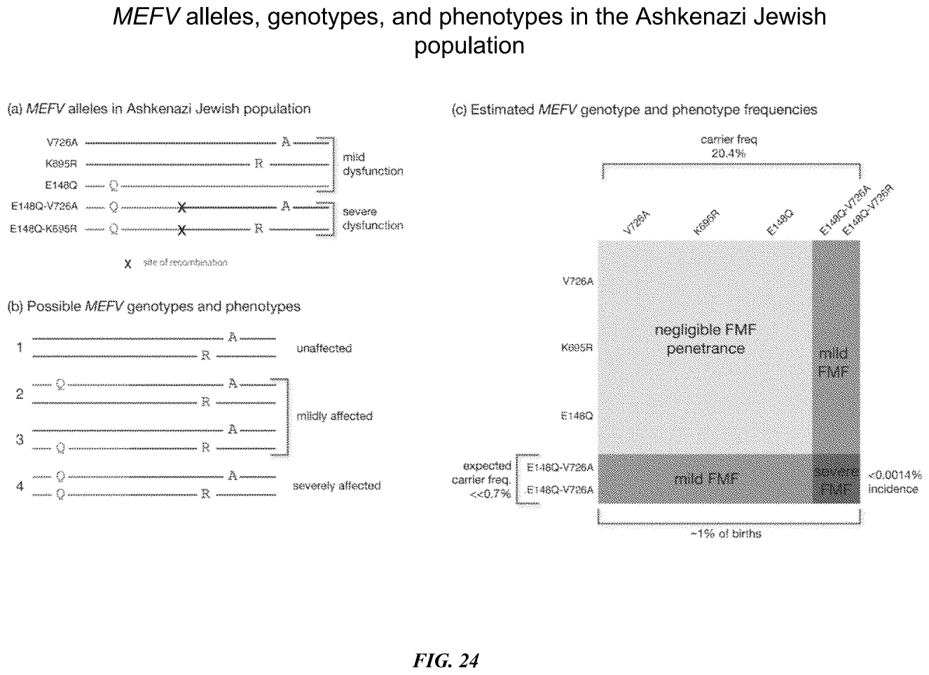

In FIG. 24, data illustrating MEFV alleles, genotypes, and phenotypes in the Ashkenazi Jewish population are shown. Results shown are based on data retrieved from the IBD Exomes Portal database. (a) Chromosome stick diagrams are shown for the three most common uncontested pathogenic MEFV variants in the AJ population and the two complex alleles that can be generated by intragenic recombination between the p.E148Q allele and the two downstream variants. Variant residues are indicated by their position in the MEFV polypeptide product. (b) Possible MEFV genotypes illustrate the necessity of the E148Q variant in a [p.K695R∥p.V726A] genotype to elicit disease. (c) Estimated MEFV genotype and phenotype frequencies expose the disconnect that exists between an ICT for any simple allele and the actual risk of disease, which can be derived from the full virtual progeny genotype. Genotype combinations expected to result in mild or severe FMF account for <8% of all genotypes derived from the simple and complex alleles.

If low PPV was the result of incomplete penetrance sensu stricto, the match m1 genotype would be clinically uninformative. However, a more likely explanation for the exceedingly low but non-zero disease association is negative epistasis between variants at two different codons in the same allele. High risk of disease is predicted to occur if either MEFV allele in the match m1 genotype contains a second MEFV variant, p.E148Q (FIG. 24). Although p.K695R, p.V726A and p.E148Q each appear most often on isolated alleles, a recombination hotspot can operate in compound heterozygous [p.E148Q∥V726A] or [p.E148Q∥K695R] parents to generate the complex allele haplotypes [p.E148Q-K695R] or [p.E148Q-V726A] which are both associated with severe gene dysfunction.

Incorporating the knowledge of epistatic interactions between p.E148Q and downstream variants into the automated VPA analysis provide the ability to distinguish matches at risk of FMF from those that have no substantial risk. In the case of the PCT-positive m1 genotype, neither MEFV allele contains the p.E148Q variant. Therefore, this match is not at risk for FMF. In one embodiment, a machine learning model, such as the one used in process 2000, may include one or more nodes that calculate epistatic interactions between a target allele site and downstream variants.

### Case Study: Mucopolysaccharidosis: A ClinVar-Asserted Benign Variant is Likely to be Disease-Contributing.

Two matches, m2 and m3 as shown in FIG. 22, produced virtual progeny with the same compound heterozygous IDUA genotype [p.V322E∥p.G265S] which VPA predicts to be disease-associated. The p.G265S variant contributed by the same male participant to both matches is a novel splice site mutation that is likely to be a null allele. The second variant, p.V322E, is present at a frequency of 0.7% in Africa, but is rare in Europe. It is described as a “pseudodeficiency” allele in a single abstract which led to a “benign” ClinVar assertion. In the VPA analysis, the highly concordant scoring of p.V322E by all evolutionary constraint and protein damage modeling tools as more damaging than 95% of observed IDUA missense variants outweighed the ClinVar assertion and single citation.

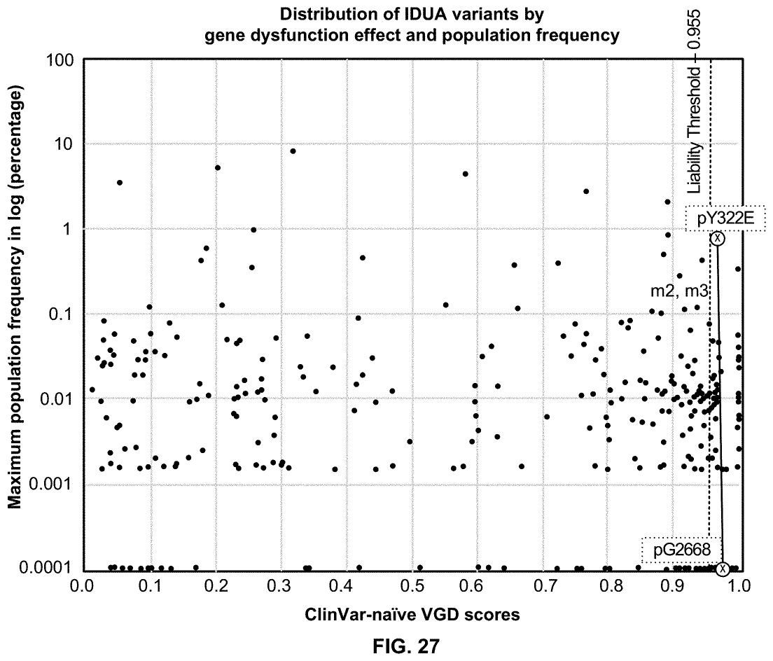

In clinical reviews of the VPA-flagged genotype, a determination was made that in the context of the cited abstract, pseudodeficiency was used as a synonym for a partial function which is consistent with the VPA outcome. The CTM model presented earlier with the established continuous spectrum of IDUA-associated disease indicates the likelihood that insufficient IDUA gene activity will lead to clinical disease in virtual progeny with the [p.V322E∥p.G265S] genotype generated by matches m2 and m3 in FIG. 22. A VGD score plot highlighting likely disease-causing genotypes for IDUA gene is shown in FIG. 27. In one embodiment, a machine learning model, such as the one used in process 2000, may include one or more nodes that calculate the likelihood of benign variant indicated by literature such as from ClinVar that might be disease contributing.

### Case Study: Smith-Lemli-Opitz Syndrome, a Missense Mutation Increases Likelihood of Disease.

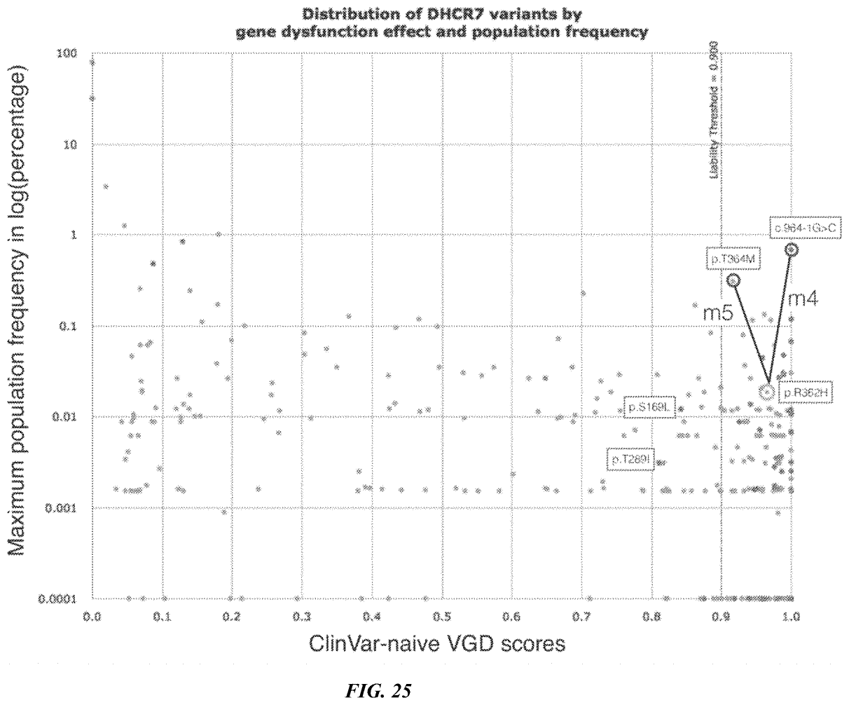

VPA predicts the likelihood of a pediatric disease recognized as Smith-Lemli-Opitz syndrome (SLOS) for virtual progeny genotypes generated by two matches, m4 and m5 (FIG. 22). Only one research participant, the male member of match m4, tested ICT-positive. This participant is heterozygous for the well-studied splice acceptor variant c. 964-1 G>C which typically abolishes gene function. In his case, the maintenance of a 50% level of DHCR7 activity due to the reference allele is sufficient to prevent any clinical symptoms or physiological deficiencies. However, if he had partnered with another carrier of the same variant, the couple would have been at risk of conceiving a homozygous embryo with a [c.964-IG>C∥c.964-IG>C] genotype that is associated with a complete absence of DHCR7 activity. Empirical findings indicate that, most often, such embryos spontaneously abort at a point prior to the 18th week of gestation. As a result, most SLOS-affected children carry at least one missense allele that partially reduces DHCR7 enzyme activity while still leaving enough to allow fetal development and survival during the second half of gestation to a live-born child. A genotype of this form [p.R362H∥c.964-IG>C] is generated from match m4. The result of the distribution of DHCR7 variants by gene dysfunction effect and population frequency is shown in FIG. 25.

In FIG. 25, DHCR7 variants identified in the ExAC or ClinVar databases are individually plotted with ClinVar-naive variant-specific gene dysfunction values on the X-axis and the logarithm of the maximum ExAC population allele frequency on the Y-axis. A variant may include uncontested pathogenic/likely pathogenic (appearing in a range greater than the 0.8 score), benign/likely benign (appearing in a range less than the 0.2 score), and contested pathogenic variants (appearing in a range between the 0.6 and 0.7 score). All uncharacterized variants and those without ClinVar-benign or pathogenic assertions are gray. Variants found in ClinVar, but not represented in ExAC are plotted along the X-axis line. One variant circled is the female contribution to both matches m4 and m5 in FIG. 22. Other two variants circled represent the two male contributions. Pathogenic variants used for comparison with p.R362H are labeled to the left of each plotted variant. The graph displays modified ClinVar-naive VGD scores that were optimized without the benefit of ClinVar assertions in order to evaluate the capacity of VGD to identify uncharacterized variants with the same degree of dysfunction as those that are clinically confirmed pathogenic.

The variant p.R362H is not found in the clinical literature. However, with a maximum population frequency of 0.02% in South Asia and 0.005% in Europe, the lack of clinical visibility is not informative to the VPA process, which relies primarily on the integrated evolutionary constraint and protein damage modeling tools. All five tools in the network analysis individually rank p.R362H above the clinically-asserted pathogenic variants p.S169L and p.T289I (FIG. 25) and the integrated p.R362H dysfunction score falls directly in the range of ClinVar-asserted DHCR7 pathogenic variants. Accordingly, the p.R362H variant is likely to express very low levels of enzyme activity, which puts a child who inherits the [p.R362H∥c.964-IG>C] genotype at high risk of severe disease. In one embodiment, a machine learning model, such as the one used in process 2000, may include one or more nodes that take account of any possibility of missense mutation.

### Case Study: CF-Related Disease, Evolutionary Constraint at an Intronic Base

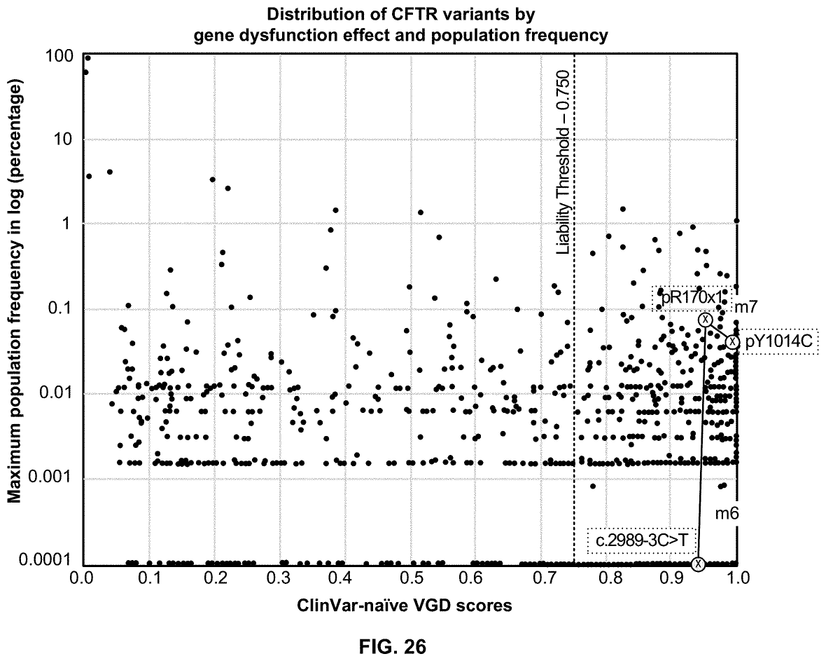

Match m6 generates the CFTR genotype [c.2989-3C>T∥p.R170H] which VPA flags as likely disease-causing (FIG. 22). The female-contributed variant (c.2989-3C>T) is a novel single base intronic substitution that is three bases from a splice site. VGD flagged this variant as likely to be strongly deleterious based on an extreme level of evolutionary constraint at the level of DNA sequence. This computational flag led to a literature search that uncovered another variant at the same position, substituting the C with a G instead of a T. The c.2989-3C>G variant was observed in compound heterozygosity with p.F508* in a six-month-old male patient with classic cystic fibrosis, which provides confirmatory evidence that the reference base “C” is likely to play an essential role in the splicing of CFTR transcripts. FIG. 26 is a CFTR gene plot highlighting likely disease-causing genotypes.

The VPA process may assign high, but not total, dysfunction to the male-contributed variant p.R170H based on high visibility in the clinical literature together with an integrated evolutionary constraint and protein damage score in the top 20% of all ExAC CFTR missense mutations. A literature review indicated the presence of p.R170H in multiple individuals diagnosed with congenital absence of the vas deferens (CAVD) or CFTR-related pancreatitis, always in combination with a strongly dysfunctional CFTR allele. Since the c.2989-3C>G variant detected in the maternal allele, in this case, is strongly dysfunctional, the VPA flagged genotype [c.2989-3C>T∥p.R170H] is clinically confirmed to be likely disease associated. In one embodiment, a machine learning model, such as the one used in process 2000, may include one or more nodes that calculate evolutionary constraint at an intronic base.

### Case Study: Niemann-Pick Disease: Population Bias in Variant Selection for Carrier Panels

VPA predicts a likely disease outcome for a compound heterozygous SMPD1 genotype in simulated offspring from an African-American egg donor with an AJ male recipient (match m9, FIG. 22). The variant contributed by the male (p.F333Sfs*52) is a clinically well-characterized frameshift variant detected by ICT. The variant contributed by the female (p.R230H) is a missense mutation that was not present in the ClinVar database at the time of this analysis; it has since been entered into ClinVar with a VUS (a variant of uncertain significance) assertion. For example, p.R230 is associated with highly concordant evolutionary constraint and protein damage modeling scores. The VPA flag of likely disease risk led to a literature search that revealed two reports of p.R230H in compound heterozygous patients with a missense variant ([p.R230H∥p.H427R]) or a frameshift variant ([p.R230H∥p.A597Pfs*6]). Both cases were associated with a milder form of Niemann-Pick disease. The [p.R230H∥p.F333Sfs*52] genotype of match m9 is functionally equivalent to the latter case and thus is expected to result in an affected child.

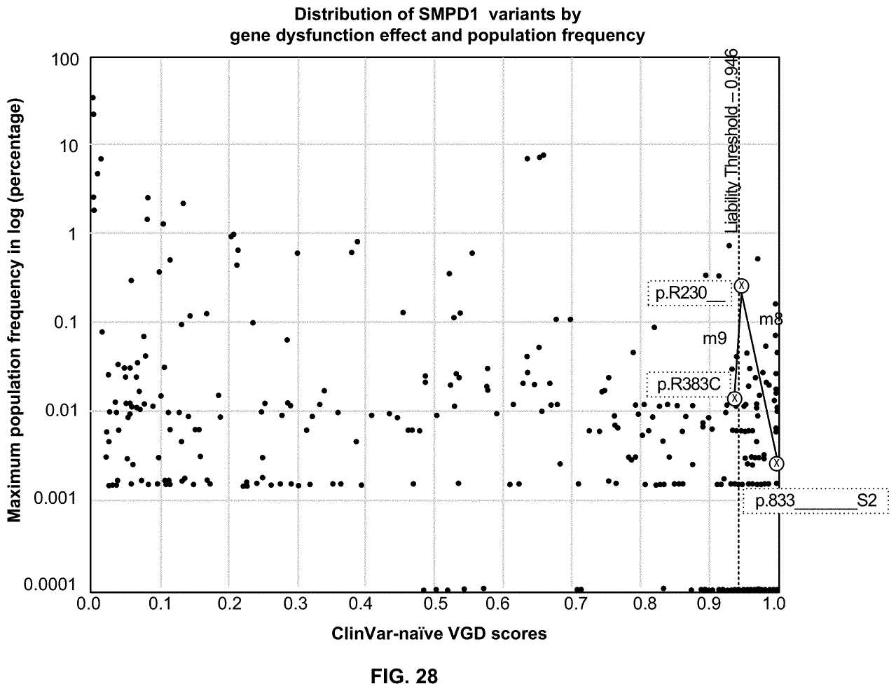

Carriers of p.F333Sfs*52 are very rare in a broadly sampled European population (0.003% allele frequency) and are not detected in other population samples. In contrast, the p.R230H variant appears in African populations at a 100-fold greater frequency of 0.2517%. A recessive disease-associated variant present at the same frequency in northern Europe would now typically be included in ClinVar and on expanded carrier tests. Its absence from the two carrier tests used in this study is an illustration of the continued impact of systematic ancestral population bias in the choice of subjects for biomedical research and variants for testing panels. A VGD score plot highlighting likely disease-causing genotypes and population frequency for SMPD1 gene is shown in FIG. 28. In one embodiment, a machine learning model, such as the one used in process 2000, may include one or more nodes that calculate population bias in variant selection.

### Implications for Clinical Care

These results show that most recessive disease-contributing variants are individually rare, not clinically defined, and yet collectively common in the general population. Variants that are novel or very rare contributed to seven out of the eight likely disease-causing genotypes, shown in FIG. 22, detected uniquely by the automated cVPA process 2000. The eighth uniquely cVPA genotype is composed of two CFTR variants described multiple times in the clinical literature, but with disease phenotypes other than “classic” cystic fibrosis.

The results also demonstrate the importance of evaluating variants by their molecular impact on gene activity instead of intrinsic pathogenicity, and of deriving a predicted phenotype by integrating the fractional activities of a genotype's alleles. In all cases, variant gene activity was estimated with computational tools used by clinical geneticists in the diagnosis of patients with a rare disease. Understanding the fractional functionality of variant alleles provides the framework for more accurately determining disease risk, which can range from asymptomatic or subclinical to a severe pediatric condition, or even prenatal lethality in the case of the most common DHCR7 disease genotype. Carrier testing, with its binary output of positive or negative results, does not produce the clinical nuance required to accurately predict preconception risk in a majority of the cases presented here.

Notably, current testing paradigms do not sufficiently reduce the risk of recessive disease transmission. Aspects that are missing include the pervasiveness of rare and novel high-damage mutations, wide population variance in both disease risk and allele spectra, and the high incidence of partially damaging variants that are disease-causing in some specific genotypes but defy simple classification as pathogenic or benign. The absence of these aspects may be unnecessarily limiting for the evaluation of non-existent genomes of intended children with hypothetical disease risk. A conservative approach that maximizes sensitivity is highly appropriate to preconception testing which can be used to inform patients and clinical decisions including the choice of a donor or pre-implantation genetic diagnosis (PGD).

Other or different operations or orders of operations may be used and operations may be repeated, e.g., until the likelihoods or optimization cost factor converge or asymptotically approach a statistically stable result.

Current literature and variant classification databases distribute recessive-disease associated variants into binary “pathogenic” and “benign” groupings. These generalizations are based on outdated and limited genetic practices.

There is a need in the art to incorporate multiple complementary resources to generate a more accurate computational score. With the advent of extensive disease databases, such as, the ExAC database, and with additional exome sequencing data of healthy and diseased individuals, the VGD model provides a comprehensive score that incorporates in silico models and actually observed variant frequencies. The VGD model is flexible and dynamic, for example, using a neural network to incorporate a growing combination of model components or nodes and to re-train the model based on growing clinical resources and genetic datasets. The VGD model may be used to assess gene-dysfunction for any variant and dynamic enough to handle unique variants. The VGD model combined with the reduction in cost of next-generation sequencing provides the capability to efficiently and accurately estimate disease contribution on a continuous scale for any variant.

There is an imperative need for a disease classification system that moves beyond the binary “pathogenic” and “benign” categorizations for recessive-disease associated variants. The VGD model hereby provides estimates of the functional impact that variants have on the expression of single-locus recessive diseases on a continuous scale. The VGD model further combines several metrics into one or more likelihoods, increasing the accuracy and sensitivity for assessing the probability that a variant is disease contributing, and identifying new variants that were previously ignored as well as mistaken variants that were previously erroneously classified as pathogenic due to more rudimentary methods, exposing their limitations and restrictions. The VGD model can be used to quantify the level of risk associated with any variant and represents a more accurate metric for describing potential disease contributing variants.

### Definitions

As used herein, a “chromosome” may refer to a molecule of DNA with a sequence of basepairs that corresponds closely to a defined chromosome reference sequence of the organism in question.

As used herein, a “gene” may refer to a DNA sequence in a chromosome that codes for a product (either RNA or its translation product, a polypeptide) or otherwise plays a role in the expression of said product. A gene contains a DNA sequence with biological function. The biological function may be contained within the structure of the RNA product or a coding region for a polypeptide. The coding region includes a plurality of coding segments (“exons”) and intervening non-coding sequences (“introns”) between individual coding segments and non-coding regions preceding and following the first and last coding regions respectively.

As used herein, a “locus” may refer to any segment of DNA sequence defined by chromosomal coordinates in a reference genome known to the art, irrespective of biological function. A DNA locus can contain multiple genes or no genes; it can be a single base pair or millions of base pairs.

As used herein, a “polymorphic locus” may refer to a genomic locus at which two or more alleles have been identified.

As used herein, an “allele” may refer to one of two or more existing genetic variants of a specific polymorphic genomic locus.

As used herein a “variant” or “genetic mutation” may be any one or more bases, nucleotides, or alleles, which may or may not differ compared to reference, common or expected bases, nucleotides, or alleles, for example, of one or more reference genetic sequences.

As used herein, “genotype” may refer to the diploid combination of alleles at a given genetic locus, or set of related loci, in a given cell or organism. A homozygote includes two copies of the same allele and a heterozygote includes two distinct alleles. In the simplest case of a locus with two alleles “A” and “a”, three genotypes can be formed: A/A, A/a, and a/a, of which A/A and a/a are homozygotes and A/a are heterozygotes.

As used herein, “genotyping” may refer to any experimental, computational, or observational protocol for distinguishing an individual's genotype at one or more well-defined loci.

As used herein, a “haplotype” may refer to a unique set of alleles at separate loci that are normally grouped closely together on the same DNA molecule, and are observed to be inherited as a group. A haplotype can be defined by a set of specific alleles at each defined polymorphic locus within a haploblock.

As used herein, a “haploblock” may refer to a genomic region that maintains genetic integrity over multiple generations and is recognized by linkage disequilibrium within a population. Haploblocks are defined empirically for a given population of individuals.

As used herein, “linkage disequilibrium” may refer to the non-random association of alleles at two or more loci within a particular population. Linkage disequilibrium is measured as a departure from the null hypothesis of linkage equilibrium, where each allele at one locus associates randomly with each allele at a second locus in a population of individual genomes.

As used herein, a “genome” may refer to the total genetic information carried by an individual organism or cell, represented by the complete DNA sequences of its chromosomes.

As used herein, a “genome profile” may refer to a representative subset of the total information contained within a genome. A genome profile contains genotypes at a particular set of polymorphic loci.

As used herein, a “personal genome profile”, abbreviated PGP, may refer to the genome profile of a particular individual person.

As used herein, a genetic “trait” may refer to a distinguishing attribute of an individual, whose expression is fully or partially influenced by an individual's genetic constitution.

As used herein, a “phenotype” may refer to a class of alternative traits which may be discrete or continuous.

As used herein, “haploid cell” may refer to a cell with a haploid number (n) of chromosomes.

As used herein, “gametes”, may refer to specialized haploid cells (e.g., spermatozoa and oocytes) produced through the process of meiosis and involved in sexual reproduction.

As used herein, “gametotype” may refer to single copies with one allele of each of one or more loci in the haploid genome of a single gamete.

As used herein, an “autosome” may refer to any chromosome exclusive of the X and Y sex chromosomes.

As used herein, “diploid cell” may have a homologous pair of each of its autosomal chromosomes, and has two copies (2n) of each autosomal genetic locus.

As used herein, a “haplopath” may refer to a haploid path laid out along a defined region of a diploid genome by a single iteration of a Monte Carlo simulation or a single chain generated through a Markov process. A haplopath can be formed by starting at one end of a personal chromosome or genome and walking from locus to locus, choosing a single allele at each step based on available linkage disequilibrium information, inter-locus allele association coefficients, and formal rules of genetics that describe the natural process of gamete production in a sexually reproducing organism. A “haplopath” is generated through the application of formal rules of genetics that describe the reduction of the diploid genome into haploid genomes through the natural process of meiosis.

As used herein, a “Virtual Gamete” may refer to a single haplopath that extends across an entire genome.

As used herein, a “Virtual Progeny” or “Virtual Progeny genome sampling” may refer to the discrete genetic product of two Virtual Gametes. Virtual Progeny may be generated as disclosed in U.S. Pat. No. 8,805,620, incorporated herein by reference in its entirety.

As used herein, a “Virtual Progeny genome” may refer to a collection of discrete Virtual Progeny genome samplings, each generated by combining two uniquely-derived random Virtual Gametes. In some instances, a Virtual Progeny genome is represented as a probability mass function over a sample space of all discrete genome states. In some instances, a Virtual Progeny is an informed simulation of a child or children that might result as a consequence of sexual reproduction between two individuals.

As used herein, a “Virtual Progeny phenome” may refer to a multi-dimensional likelihood function representing the likelihood and/or likely degree of expression of a set of one or more traits from a complete Virtual Progeny genome. In some instances, a Virtual Progeny phenome is represented as a probability mass function over a sample space of discrete or continuous phenotypic states. In some instances, a Virtual Progeny phenome is an informed simulation of a child or children that might result as a consequence of sexual reproduction between two individuals.

As used herein, “potential parent” may refer to an individual who genetic information is combined with another's genetic information to simulate a mating before a child is conceived. The mating may be simulated for two potential parents both interested in their combined genetic code, or a single individual iterating the mating over a plurality of candidate donors, for example, to select an optimal donor from a sperm or egg donor bank. A “partner” may refer to a marriage partner, sexual or reproductive partner, domestic partner, opposite-sex partner, and same-sex partner.

As used herein, a “living” organism may refer to a real, extant, surviving, currently living, or previously living (now deceased), organism. A “virtual” organism may refer to a never or non-living organism, for example, simulated by computer models, where all its genetic information is derived by combining data representing real biological DNA obtained from living organisms such as two potential parents by simulated a mating or conception process.

As used herein, a “DNA image” may refer to a magnified picture or image or real biological DNA, or may refer to a simplified schematic representation thereof such as a nucleotide or DNA sequence. In one example, a DNA image may be zoomed out view of a DNA sequence.

As used herein, “reference genetic sequence” may refer to a genetic sequence used to generate an evolutionary model, such as, a phylogenetic tree. Reference genetic sequences may include standardized genetic sequences from organisms representative of one or more evolutionarily extant (currently or previously living) populations or species, such as those released by genome consortia (e.g., human reference genome, such as, Genome Reference Consortium Human Build 37 (GRCh37) provided by the Genome Reference Consortium). Reference genetic sequences may additionally or alternatively include non-standardized sequences of organisms, such as, any member of a population or species. A single-species model may be generated using reference genetic sequences from multiple organisms of the same single species, e.g., 1,000 chimpanzee or humans. A multi-species model may be generated using reference genetic sequences from multiple organisms of multiple different species, e.g., one model using 1,000 humans, 10 chimpanzee and one gorilla, or another model using a single different organism from each different species as shown in FIG. 15. Reference genetic sequences may be used to analyze the evolution of successful (positive) or neutral (non-deleterious) allele mutations or variations across one or more extant species. An evolutionary model may predict likelihoods that allele mutations or variations would be deleterious based on their frequency or rarity of occurrence across the multiple reference genetic sequences. For example, allele mutations or variations that are relatively more rare across the reference genetic sequences may be considered negatively selected for evolutionarily (e.g. associated with a deleterious trait for which an organism cannot or has a relatively lower likelihood of surviving or reproducing), while allele mutations or variations that are relatively more common across the reference genetic sequences may be considered positively or neutrally selected for evolutionarily (e.g. not associated with a deleterious trait, but traits for which an organism has a neutral or improved likelihood of surviving or reproducing).

As used herein, “potential parent genetic sequences” may refer to genetic sequences of real (currently or previously living) potential parents, for example, from which genetic information is combined to simulate a virtual mating generating one or more virtual children or progeny, to predict before they conceive a child, a likelihood that such a child would have a deleterious trait. The potential parent genetic sequences may be obtained from genetic samples of two potential parents seeking to mate, or from a first potential parent seeking a genetic donor and a second potential parent from a pool of candidate donors.

As used herein, “virtual progeny genetic sequences” may refer to genetic sequences of simulated (never living) virtual progeny generated by simulating a mating or combining genetic information from two potential parent genetic sequences. Each virtual progeny genetic sequence may be a prediction or simulation of one possible genetic sequence of a child of the two potential parents, before that child is conceived. To achieve more robust results, the simulated mating may be repeated to generate multiple virtual progeny genetic sequences for each pair of potential parents. The virtual progeny genetic sequences may be compared to the reference genetic sequences, for example, to identify evolutionarily rare, and therefore, likely deleterious traits.

In some embodiments, genetic information may be used interchangeably for potential parent genetic sequences and reference genetic sequences. In one example, genetic information from a potential parent or donor may be used instead of, or in combination with reference consortium genetic sequences, to generate an evolutionary model or phylogenetic tree. In another example, reference consortium genetic sequences may be used instead of, or in combination with potential parent or donor genetic sequences, to simulate matings or predict likelihoods of deleterious traits in offspring.

As used herein, a “genetic sequence” may include genetic information representing one or more bases, nucleotides or alleles (sequences of nucleotides defining different forms of a gene) for any number of sequential or non-sequential genetic loci. For example, a “genetic sequence” may refer to allele information at a single genetic locus, or multiple genetic loci, such as, one or more gene segments or an entire genome. A genetic sequence is a data structure representing genetic information at one or more loci of a real or virtual genome. Genetic sequence data structures may include, for example, one or more vectors, scalar values, functions, sequences, sets, matrices, tables, lists, arrays, and/or other data structures, representing one or more bases, nucleotides, genes, alleles, codons or other generic material. The data structures representing each single chromosome sequence may be one dimensional (e.g., representing a single base or allele per locus) or multi-dimensional (e.g., representing multiple or all bases A, T, C, G or alleles at each locus and a probability associated with the likelihood of each existing in a potential progeny). The same (or different) data structures may be used for real and virtual genome sequences, though real genome sequences generally represent real genetic material (e.g., DNA extracted from a currently or previously existing genetic sample), while virtual genome sequences are generated by combining at least a portion of genetic sequences from biological DNA samples of two living potential parents.

As used herein, “a˜b” may represent a proportional “˜” relationship between a and b.

As used herein, “a≈b” may represent an approximate equivalence “≈” between a and b, for example, within 10% of either value.

### CONCLUSION

In the foregoing description, various aspects of the present invention are described. For purposes of explanation, specific configurations and details are set forth in order to provide a thorough understanding of the present invention. However, it will also be apparent to persons of ordinary skill in the art that the present invention may be practiced without the specific details presented herein. Furthermore, well known features may be omitted or simplified in order not to obscure the present invention.

Unless specifically stated otherwise, it is appreciated that throughout the specification discussions utilizing terms such as “processing,” “computing,” “calculating,” “determining,” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

Embodiments of the invention may include an article such as a computer or processor readable non-transitory storage medium, such as for example a memory, a disk drive, or a USB flash memory device (e.g., memory unit(s) 114, 116, and/or 118 of FIG. 1) encoding, including or storing instructions, e.g., computer-executable instructions, which when executed by a processor or controller (e.g., controller(s) or processor(s) 108, 110, and/or 112 of FIG. 1), cause the processor or controller to carry out methods disclosed herein.

Different embodiments are disclosed herein. Features of certain embodiments may be combined with features of other embodiments; thus certain embodiments may be combinations of features of multiple embodiments.

The foregoing description of the embodiments of the invention has been presented for the purposes of illustration and description. It is not intended to be exhaustive or to limit the invention to the precise form disclosed. It should be appreciated by persons of ordinary skill in the art that many modifications, variations, substitutions, changes, and equivalents are possible in light of the above teaching. For example, it should be appreciated that sign conventions are equivalent and that embodiments of the invention in which values are above a lower bound or threshold are equivalent to embodiments of the invention in which values are below an upper bound or threshold, since the difference is a mere convention of sign. It is, therefore, to be understood that the appended claims are intended to cover all such modifications and changes as fall within the true spirit of the invention.

TABLE 1
Summary of Variants Detected by VGD Model and Missed by ClinVar or VariBench
Table 1 lists de novo variants (not identified by the ClinVar or VariBench models) identified
according to embodiments of the invention to cause gene dysfunction, for example, with VGD
scores of above 0.95 or identified according to embodiments of the invention to be benign, for
example, with VGD scores below 0.05.

| | | | Median | | | | | |
|---|---|---|---|---|---|---|---|---|
| | VGD | VGD | # of | Median | PP2 | CADD | PROVEAN | VEST |
| | Median | mean | homozygotes | max pop. | median | median | median | median |
| | (IQR) | (sd) | (IQR) | freq (IQR) | (IQR) | (IQR) | (IQR) | (IQR) |
| ExAC variants | 0 | 0.0109 | 0 (0, 0) | 9.64e−05 | 0.001 | 7.5 | −0.01 | 0.066 |
| not in ClinVar | (0, 0.02) | (0.0156) | | (3e−05, | (0, | (2.42, | (−0.34, | (0.039, |
| with VGD <= 0.05 | | | | 0.000262) | 0.006) | 10.6) | 0.41) | 0.107) |
| ExAC variants | 0.99 | 0.985 | 0 (0, 0) | 6.06e−05 | 1 | 25.1 | −5.7 | 0.924 |
| not in ClinVar | (0.97, 1) | (0.0162) | | (1.51e−05, | (0.999, | (21.6, | (−7.31, −4.36) | (0.868, |
| with VGD >= 0.95 | | | | 9.92e−05) | 1) | 32) | | 0.965) |
| All ExAC | 0.32 | 0.402 | 0 (0, 0) | 8.64e−05 | 0.759 | 16.2 | −1.89 | 0.403 |
| variants not in | (0.03, | (0.369) | | (1.61e−05, | (0.022, | (9.81, | (−3.56, −0.75) | (0.165, |
| ClinVar | 0.77) | | | 0.000169) | 0.998) | 22.6) | | 0.727) |
| ExAC variants | 0 | 0.0107 | 0 (0, 0) | 9.82e−05 | 0.001 | 7.62 | −0.04 | 0.068 |
| not in VariBench | (0, 0.02) | (0.0155) | | (3.01e−05, | (0, | (2.5, | (−0.39, | (0.04, |
| with VGD <= 0.05 | | | | 0.000347) | 0.008) | 10.8) | 0.39) | 0.114) |
| ExAC variants | 0.99 | 0.985 | 0 (0, 0) | 6.06e−05 | 1 | 25 | −5.67 | 0.924 |
| not in VariBench | (0.97, 1) | (0.0161) | | (1.51e−05, | (0.999, | (21.5, | (−7.29, −4.31) | (0.867, |
| with VGD >= 0.95 | | | | 0.000102) | 1) | 32) | | 0.966) |
| All ExAC | 0.31 | 0.401 | 0 (0, 0) | 8.66e−05 | 0.76 | 16.2 | −1.89 | 0.403 |
| variants not in | (0.03, | (0.37) | | (1.65e−05, | (0.022, | (9.81, | (−3.55, −0.75) | (0.165, |
| VariBench | 0.77) | | | 0.000174) | 0.998) | 22.7) | | 0.729) |
| ExAC variants | 0 | 0.0109 | 0 (0, 0) | 9.63e−05 | 0.001 | 7.49 | 0.01 | 0.065 |
| not in ClinVar or | (0, 0.02) | (0.0156) | | (3e−05, | (0, | (2.42, | (−0.32, | (0.039, |
| VariBench with | | | | 0.00026) | 0.006) | 10.6) | 0.42) | 0.105) |
| VGD <= 0.05 | | | | | | | | |
| ExAC variants | 0.99 | 0.985 | 0 (0, 0) | 6.06e−05 | 1 | 25.1 | −5.7 | 0.923 |
| not in ClinVar or | (0.97, 1) | (0.0162) | | (1.51e−05, | (0.999, | (21.6, | (−7.31, −4.36) | (0.867, |
| VariBench with | | | | 9.9e−05) | 1) | 32) | | 0.964) |
| VGD >= 0.95 | | | | | | | | |
| ExAC variants | 0.98 | 0.935 | 0 (0, 0) | 6.06e−05 | 1 | 24.5 | −4.54 | 0.852 |
| not in ClinVar or | (0.92, 1) | (0.105) | | (1.51e−05, | (0.993, | (21.3, | (−6.31, −3.24) | (0.693, |
| VariBench with | | | | 0.000116) | 1) | 29.9) | | 0.936) |
| VGD naive >= | | | | | | | | |
| per gene | | | | | | | | |
| thresholds | | | | | | | | |
| All ExAC | 0.32 | 0.401 | 0 (0, 0) | 8.64e−05 | 0.753 | 16.1 | −1.89 | 0.401 |
| variants not in | (0.03, | (0.368) | | (1.61e−05, | (0.022, | (9.8, | (−3.55, −0.75) | (0.165, |
| ClinVar or | 0.76) | | | 0.000166) | 0.998) | 22.6) | | 0.724) |
| VariBench | | | | | | | | |

TABLE 2
Example Mutation Class Subscores and
Weights for Various Mutation Types

| Mutation Class | MutScore | Raw Mut Weight |
|---|---|---|
| Start-loss, stop-gain, | 1.0 | 99 |
| frame-shift, essential | | |
| splice site | | |
| Micro satellite | 1/STR\hat{ }2 | 1.0 |
| Synonymous | 0.0 | 1.0 |
| In-frame indel | 0.6 + 0.4*(1 − exp(−y/10)) | 0.01 |
| Missense, stop-loss | 0.5 | 0.01 |
| Other annotation | 0.0 | 0.01 |

TABLE 3
Example Clinical Classification Subscores
and Weights for Various Mutation Types

| ClinVar Class | | Raw ClinVar | |
|---|---|---|---|
| (numeric) | Clinical Class (description) | Weight | ClinScore |
| 5.1 | Uncontested “Pathogenic” | 20 | 1.0 |
| 4 | “Likely Pathogenic” | 10 | 1.0 |
| 5.2 | Contested “Pathogenic” | 1 | 1.0 |
| 3 | “Likely Benign” | 10 | 0.0 |
| 2 | “Benign” | 20 | 0.0 |
| 0, 1, 255 | Uncharacterized | 0.0 | 0.0 |
| not in ClinVar | | | |

TABLE 4
Example Scores for ClinVar 5.2 variants

| Chr:bp | Gene | VGD | Strand | Genotype | SNP | HGVSp |
|---|---|---|---|---|---|---|
| 01:043212925 | P3H1 | 0.36 | - | C/T | rs137853890 | p.A691=\|p.ARA689-691= |
| 01:046655645 | POMGNT1 | 0 | - | C/T | rs74374973 | p.D534N\|p.D556N |
| 01:063872032 | ALG6 | 0 | + | T/C | rs35383149 | p.Y131H |
| 01:076198337 | ACADM | 0.29 | + | G/A | rs147559466 | p.E43K\|p.E47K\|p.E7K\|p.P13= |
| 01:097915614 | DPYD | 0.16 | - | C/T | rs3918290 | . |
| 01:098348885 | DPYD | 0 | - | G/A | rs1801265 | p.C29=\|p.C29R\|p.R29C |
| 01:100672060 | DBT | 0.72 | - | T/G | rs12021720 | p.G323S\|p.G384= |
| 01:100672060 | DBT | 0 | - | T/C | rs12021720 | p.G323S\|p.G384=\|p.G384S\|p.S |
| | | | | | | 384G |
| 01:155204994 | GBA | 0.55 | - | C/G | rs1135675\|rs606231143 | p.A456P\|p.L444P\|p.V386=\|p.V |
| | | | | | | 412=\|p.V450=\|p.V460V\|p.V49 |
| | | | | | | 9= |
| 01:155205008 | GBA | 0.57 | - | C/G | rs368060 | p.A382P\|p.A408P\|p.A446P\|p.A |
| | | | | | | 456P\|p.A495P\|p.L444P\|p.V460 |
| | | | | | | V |
| 01:155206167 | GBA | 0.02 | - | C/T | rs2230288 | p.D140H\|p.E252K\|p.E278K\|p.E |
| | | | | | | 316K\|p.E326K\|p.E365K |
| 01:156108401 | LMNA | 0.46 | + | G/A | rs59886214 | p.V607=\|p.V607V |
| 01:156108404 | LMNA | 0.61 | + | C/T | rs58596362 | p.G608=\|p.G608G |
| 01:156848918 | NTRK1 | 0.01 | + | C/T | rs6336 | p.G607V\|p.H568Y\|p.H598Y\|p. |
| | | | | | | H601Y\|p.H604Y\|p.Q9X\|p.Y60 |
| | | | | | | 4H |
| 01:156848946 | NTRK1 | 0.01 | + | G/T | rs6339 | p.G577V\|p.G607V\|p.G610V\|p. |
| | | | | | | G613V\|p.H598Y\|p.Q9X\|p.V61 |
| | | | | | | 3G |
| 01:216498841 | USH2A | 0.34 | - | G/T | rs111033272 | p.R317= |
| 01:227170648 | ADCK3 | 0 | + | C/T | rs41303129 | p.F279=\|p.F331=\|p.F52= |
| 01:241665852 | FH | 0.99 | - | T/G | rs200796606 | p.Q376P |
| 01:241675301 | FH | 0.99 | - | G/C | rs199822819 | p.P174R |
| 02:026461825 | HADHA | 0.65 | - | G/T | rs147103714 | p.F10L\|p.R53= |
| 02:145156798 | ZEB2 | 0.33 | - | G/A | rs587784563 | p.Y652= |
| 02:215813331 | ABCA12 | 0 | - | C/T | rs726070 | p.D2047N\|p.D2363N\|p.D2365 |
| | | | | | | N |
| 02:219674479 | CYP27A1 | 0.31 | + | G/T | rs587778796 | p.G145=\|p.G51= |
| 02:219678877 | CYP27A1 | 0 | + | C/T | rs41272687 | p.P384L |
| 02:219754966 | WNT10A | 0 | + | G/A | rs147680216 | p.G213S |
| 02:219755011 | WNT10A | 0.2 | + | T/A | rs121908120 | p.F228I |
| 02:241808314 | AGXT | 0.01 | + | C/T | rs34116584 | p.P11L |
| 02:241817516 | AGXT | 0 | + | A/G | rs4426527 | p.I340M |
| 03:014200382 | XPC | 0 | - | G/T | rs74737358 | p.P218H\|p.P297H\|p.P334H |
| 03:015677019 | BTD | 0 | + | G/A | rs34885143 | p.G25R\|p.G45R\|p.G47R |
| 03:015677098 | BTD | 0 | + | T/C | rs397514333 | p.L51P\|p.L71P\|p.L73P |
| 03:015686243 | BTD | 0 | + | A/G | rs35976361 | p.I274V\|p.I294V\|p.I296V |
| 03:015686331 | BTD | 0 | + | A/G | rs397507176 | p.H303R\|p.H323R\|p.H325R |
| 03:015686534 | BTD | 0 | + | C/T | rs35034250 | p.P371S\|p.P391S\|p.P393S |
| 03:015686693 | BTD | 0.06 | + | G/C | rs13078881 | p.A171T\|p.D424H\|p.D444H\|p. |
| | | | | | | D446H\|p.F403V |
| 03:128598490 | ACAD9 | 0 | + | C/+TAAG | rs28384402\|rs369565142\|rs | . |
| | | | | | 387906242 | |
| 03:142275281 | ATR | 0.37 | - | T/C | rs587776690 | p.G674= |
| 03:150645894 | CLRN1 | 0.99 | - | A/C | rs121908140 | p.Y100X\|p.Y176X\|p.Y189X |
| 03:165491280 | BCHE | 0 | - | C/T | rs1803274 | p.A29T\|p.A539T\|p.A567T\|p.A9 |
| | | | | | | 7T |
| 03:165547569 | BCHE | 0.09 | - | C/A | rs28933390 | p.G390V\|p.G418V |
| 03:165548529 | BCHE | 0.09 | - | T/C | rs1799807 | p.D70G\|p.D98G |
| 03:183966564 | ALG3 | 0.64 | - | G/A | rs387906273 | p.G53=\|p.G55=\|p.H33Y |
| 04:005755524 | EVC | 0 | + | G/A | rs35953626 | p.R443Q |
| 04:187004074 | TLR3 | 0 | + | C/T | rs3775291 | p.L135F\|p.L348F\|p.L412F |
| 05:073981270 | HEXB | 0.31 | + | T/G | rs820878 | p.S62=\|p.S62Lc.185C= |
| 05:073981270 | HEXB | 0 | + | T/C | rs820878 | p.L62S\|p.S62=\|p.S62L |
| 05:118792052 | HSD17B4 | 0.66 | + | C/T | rs587777442 | p.A34V\|p.S37= |
| 05:131729380 | SLC22A5 | 0.01 | + | G/A | rs28383481 | p.R488H\|p.R512H |
| 06:007542236 | DSP | 0.06 | + | G/A | rs121912998 | p.V30M |
| 06:032006858 | CYP21A2 | 0.97 | + | C/G | rs6467 | p.P105A\|p.T100S\|p.T70S |
| 06:161159625 | PLG | 0.04 | + | G/A | rs121918027 | p.A601T\|p.A620T |
| 07:065432754 | GUSB | 0.37 | - | G/A | rs377519272 | p.S393=\|p.S488=\|p.S539= |
| 07:087060844 | ABCB4 | 0.1 | - | C/T | rs45575636 | p.R590Q |
| 07:087082273 | ABCB4 | 0 | - | T/C | rs58238559 | p.T175A\|p.T175V |
| 07:117149147 | CFTR | 0 | + | G/A | rs1800076 | p.R75Q |
| 07:117175372 | CFTR | 0 | + | A/G | rs121909046 | p.E187G\|p.E217G |
| 07:117188682 | CFTR | 0.04 | + | G/-TT | rs200454589\|rs727504486 | . |
| 07:117227874 | CFTR | 0 | + | A/G | rs75789129 | p.I495V\|p.I526\|p.I556V |
| 07:117243663 | CFTR | 0.48 | + | C/T | rs121909034 | p.G1244V\|p.S851L\|p.S882L\|p. |
| | | | | | | S912L |
| 07:117251704 | CFTR | 0.09 | + | G/A | rs78769542 | p.R1009Q\|p.R1040Q\|p.R1070Q |
| | | | | | | \|p.R12Q |
| 07:117267556 | CFTR | 0 | + | T/C | rs373002889 | . |
| 07:117304834 | CFTR | 0 | + | G/C | rs113857788 | p.Q1291H\|p.Q1322H\|p.Q1352 |
| | | | | | | H\|p.Q61H |
| 08:022021460 | SFTPC | 0 | + | G/A | rs34957318 | p.R108Q\|p.R114Q\|p.R161Q\|p. |
| | | | | | | R167Q |
| 08:086392989 | CA2 | 0 | + | A/G | rs2228063 | p.N252D |
| 08:090983460 | NBN | 0.4 | - | G/A | rs34767364 | p.R127W\|p.R133W\|p.R215W |
| 08:100832259 | VPS13B | 0.18 | + | A/G | rs28940272 | p.N2968S\|p.N2993S |
| 09:034647661 | GALT | 0.62 | + | T/C | rs367543254 | p.S112=\|p.V96A |
| 09:034648848 | GALT | 0.5 | + | G/A | rs111033761 | p.R259= |
| 09:034649442 | GALT | 0 | + | A/G | rs2070074 | p.L218L\|p.N205D\|p.N314D |
| 09:036217445 | GNE | 0.06 | - | C/T | rs121908627 | p.V586M\|p.V622M\|p.V691M\|p |
| | | | | | | .V696M\|p.V727M |
| 09:136301982 | ADAMTS13 | 0 | + | C/G | rs2301612 | p.C508Y\|p.Q120E\|p.Q417E\|p. |
| | | | | | | Q448E |
| 09:136302063 | ADAMTS13 | 0 | + | C/T | rs11575933 | p.P147S\|p.P444S\|p.P475S |
| 10:050678722 | ERCC6 | 0 | - | G/C | rs4253208 | p.P1095R\|p.P465R |
| 10:072358722 | PRF1 | 0 | - | T/C | rs28933375 | p.N252S |
| 10:072360648 | PRF1 | 0 | - | C/T | rs35418374 | p.R4H |
| 10:102749444 | C10orf2 | 0.58 | + | C/T | rs80356541 | p.A429= |
| 11:005247873 | HBB | 0.82 | - | C/T | rs33991993 | p.E6V\|p.K82B\|p.K83N |
| 11:005248029 | HBB | 0.98 | - | C/A | rs1135071 | p.R30S\|p.R31S |
| 11:005248177 | HBB | 0.64 | - | A/T | rs33951465\|rs75680770 | p.G24G\|p.G25=\|p.G25G |
| 11:017531093 | USH1C | 0.77 | - | G/C | rs41282932 | p.P608R\|p.R608P |
| 11:017548622 | USH1C | 1 | - | C/indelrs38 | . | c.496+59_496+103[9]\|c.VNTR |
| | | | | 7906330 | | |
| 11:017548878 | USH1C | 0 | - | C/T | rs55843567 | p.V130I\|p.V141I\|p.V99I |
| 11:017552978 | USH1C | 0.64 | - | C/T | rs151045328 | p.V41=\|p.V72=\|p.V72fs\|p.V83 |
| | | | | | | = |
| 11:036615075 | RAG2 | 0 | - | G/A | rs35691292 | p.T215I\|p.W215I |
| 11:064525266 | PYGM | 0.01 | - | C/T | rs116315896 | p.K127=\|p.K215= |
| 11:066287196 | BBS1 | 0 | + | G/A | rs35520756 | p.E122K\|p.E143K\|p.E234K\|p.E |
| | | | | | | 271K |
| 11:068562328 | CPT1A | 0 | - | C/T | rs2229738 | p.A275T\|p.A27T |
| 11:071906793 | FOLR1 | 0 | + | T/C | rs144637717 | . |
| 11:076869378 | MYO7A | 0.6 | + | G/A | rs41298135 | p.R291H\|p.R302H |
| 11:108143299 | ATM | 0 | + | A/G | rs3092857 | p.M1040V |
| 11:128709126 | KCNJ1 | 0 | - | A/G | rs59172778 | p.M338T\|p.M357T |
| 12:006458350 | SCNN1A | 0.05 | - | A/G | rs5742912 | p.W193R\|p.W493R\|p.W515R\|p |
| | | | | | | .W516R\|p.W552R |
| 12:102164255 | GNPTAB | 0 | - | T/G | rs7958709 | p.I348L |
| 12:121175678 | ACADS | 0 | + | C/T | rs1800556 | p.R147W\|p.R171W |
| 12:121176083 | ACADS | 0 | + | G/A | rs1799958 | p.G185S\|p.G205S\|p.G209S |
| 12:122277904 | HPD | 0.44 | - | G/C | rs137852868 | p.I296M\|p.I335M |
| 12:122295335 | HPD | 0 | - | T/C | rs1154510 | p.A33T\|p.T33A |
| 13:020763234 | GJB2 | 0.78 | - | T/C | rs80338949 | p.M163V |
| 13:020763553 | GJB2 | 0.98 | - | C/-A | rs80338942 | p.L56RfsX26 |
| 13:020763612 | GJB2 | 0 | - | C/T | rs72474224 | p.V37Ic.109G > A |
| 13:020763620 | GJB2 | 0.53 | - | A/G | rs35887622 | p.M34T |
| 13:020763685 | GJB2 | 0.94 | - | A/-C | rs1801002\|rs398123814\|rs8 | p.G12VfsX2 |
| | | | | | 0338939 | |
| 13:020763710 | GJB2 | 0.11 | - | C/T | rs111033222 | p.G4D |
| 13:052513266 | ATP7B | 0 | - | T/C | rs7334118 | p.H1000R\|p.H1096R\|p.H1129R |
| | | | | | | \|p.H1142R\|p.H1207R\|p.H418R\| |
| | | | | | | p.H777R |
| 13:052532497 | ATP7B | 0.96 | - | T/C | rs137853287\|rs193922103 | p.M41V\|p.M658V\|p.M769V |
| 13:108863591 | LIG4 | 0 | - | G/A | rs1805388 | p.T9I |
| 13:108863609 | LIG4 | 0 | - | G/A | rs1805389 | p.A3V |
| 14:024724663 | TGM1 | 0.02 | - | C/T | rs35312232 | p.V209M\|p.V518M\|p.V76M |
| 14:024731434 | TGM1 | 0.72 | - | G/T | rs41295338 | p.S42Y |
| 14:088452941 | GALC | 0.21 | - | T/C | rs147313927 | p.T112A\|p.T56A\|p.T86A\|p.T89 |
| | | | | | | A |
| 15:072638893 | HEXA | 0.58 | - | G/A | rs587779406 | p.Y262=\|p.Y435=\|p.Y446= |
| 15:072641434 | HEXA | 0.64 | - | A/T | rs28942072 | p.V324=\|p.V324V |
| 15:080472526 | FAH | 0 | + | C/T | rs11555096 | p.R271W\|p.R341W\|p.R41W |
| 15:089865073 | POLG | 0.01 | - | T/C | rs41549716 | p.Y831C |
| 16:000222980 | HBA2 | 0.33 | + | C/T | rs63751457 | p.G22G\|p.G23= |
| 16:003293257 | MEFV | 0.14 | - | C/A | rs61732874 | p.A533S\|p.A564S\|p.A744S |
| 16:003293310 | MEFV | 0.01 | - | A/G | rs28940579 | p.V515A\|p.V546A\|p.V726A |
| 16:003293403 | MEFV | 0 | - | T/C | rs104895094 | p.K484R\|p.K515R\|p.K695R |
| 16:003304626 | MEFV | 0 | - | C/G | rs3743930 | p.E148Q\|p.M694I |
| 16:023200921 | SCNN1G | 0 | + | G/A | rs5736 | p.G1835 |
| 16:023200963 | SCNN1G | 0 | + | G/A | rs5738 | p.E197K |
| 16:023360165 | SCNN1B | 0.54 | + | C/G | rs35731153 | p.S127C\|p.S82C |
| 16:053720436 | RPGRIP1L | 0 | - | C/T | rs61747071 | p.A229T |
| 16:088502971 | ZNF469 | 0.08 | + | G/- | rs281865162 | p.L3004_T3008del\|p.L3032_T |
| | | | | CTTCCCG | | 3036del |
| | | | | GGAACA | | |
| | | | | CC | | |
| 17:003550800 | CTNS | 0 | + | G/A | rs35086888 | p.V42I |
| 17:007123838 | ACADVL | 0 | + | C/T | rs28934585 | p.K247Q\|p.P65L\|p.P88L |
| 17:015134364 | PMP22 | 0.94 | - | G/A | rs104894619 | p.H58=\|p.T118M |
| 17:041063017 | G6PC | 0.27 | + | G/T | rs80356484 | p.L216= |
| 17:078078341 | GAA | 0.91 | + | T/G | rs199951626\|rs386834236 | . |
| 19:007125518 | INSR | 0 | - | C/T | rs1799816 | p.V1000M\|p.V1012M |
| 19:012917556 | RNASEH2A | 0.63 | + | G/A | rs397515480 | p.V23=\|p.V23V |
| 19:012917562 | RNASEH2A | 0.64 | + | C/T | rs397515479 | p.R25=\|p.R25R |
| 19:018710530 | CRLF1 | 0.5 | - | C/T | rs104894670 | p.L374R\|p.R81H |
| 19:036339044 | NPHS1 | 0.02 | - | C/T | rs28939695 | p.D819V\|p.E447K |
| 19:045860626 | ERCC2 | 0.81 | - | G/C | rs121913016 | p.L169V\|p.L383V\|p1437V\|p.L |
| | | | | | | 461V |
| 20:043255233 | ADA | 0.96 | - | G/A | rs121908736 | p.R76W |
| 20:043280227 | ADA | 0 | - | C/T | rs11555565\|rs73598374 | p.D8N |
| 22:050962500 | SCO2 | 0.34 | - | C/T | rs145100473 | p.R114H |
| 22:051064039 | ARSA | 0 | - | G/C | rs743616 | p.T16S\|p.T307S\|p.T391S\|p.T39 |
| | | | | | | 3S |
| 22:051064416 | ARSA | 0 | - | T/C | rs2071421 | p.N266S\|p.N350S\|p.N352S |

| Chr:bp | HGVSc | | | | | | | | | ClinVar entiries | |
|---|---|---|---|---|---|---|---|---|---|---|---|
| 01:043212925 | c.2055+12_2055+18delTCGAGCGinsTCGAGCA\|c.2067_2073delTCGAGCGinsTC | | | | | | | | | 5 | |
| | GAGCA\|c.2073G > A | | | | | | | | | | |
| 01:046655645 | c.*1335G > A\|c.1600G > A\|c.1666G > A | | | | | | | | | 01212551315 | |
| 01:063872032 | c.391T > C | | | | | | | | | 212551315 | |
| 01:076198337 | c.-259G > A\|c.118+4164G > A\|c.119-201G > A\|c.127G > A\|c.139G > A\|c.19G > A\|c.39G > | | | | | | | | | 2125515 | |
| | A | | | | | | | | | | |
| 01:097915614 | c.1905+1G > A | | | | | | | | | 1125515 | |
| 01:098348885 | c.39+37555C > T\|c.85C > T\|c.85T=\|c.85T > C | | | | | | | | | 01115 | |
| 01:100672060 | c.1150G= | | | | | | | | | 21315 | |
| 01:100672060 | c.1150A > G\|c.1150G=\|c.1150G > A | | | | | | | | | 212551315 | |
| 01:155204994 | c.1158G > C\|c.1236G > C\|c.1350G > C\|c.1497G > C | | | | | | | | | 215 | |
| 01:155205008 | c.1144G > C\|c.1222G > C\|c.1336G > C\|c.1483G > C | | | | | | | | | 215 | |
| 01:155206167 | c.1093G > A\|c.754G > A\|c.832G > A\|c.946G > A | | | | | | | | | 5 | |
| 01:156108401 | c.1821G > A | | | | | | | | | 115 | |
| 01:156108404 | c.1824C > T | | | | | | | | | 5 | |
| 01:156848918 | c.*402C > T\|c.1702C > T\|c.1792C > T\|c.1801C > T\|c.1810C > T | | | | | | | | | 215 | |
| 01:156848946 | c.*430G > T\|c.1730G > T\|c.1820G > T\|c.1829G > T\|c.1838G > T | | | | | | | | | 215 | |
| 01:216498841 | c.949C > A | | | | | | | | | 5 | |
| 01:227170648 | c.102+184C > T\|c.156C > T\|c.837C > T\|c.993C > T, EX8\|c.993C > T | | | | | | | | | 215 | |
| 01:241665852 | c.1127A > C | | | | | | | | | 2551315 | |
| 01:241675301 | c.521C > G | | | | | | | | | 2551315 | |
| 02:026461825 | c.157C > A\|c.30C > A | | | | | | | | | 5 | |
| 02:145156798 | c.1956C > T | | | | | | | | | 5 | |
| 02:215813331 | c.6139G > A\|c.7093G > A | | | | | | | | | 5 | |
| 02:219674479 | c.153G > T\|c.256-2466G > T\|c.435G > T | | | | | | | | | 5 | |
| 02:219678877 | c.1151C > T | | | | | | | | | 5 | |
| 02:219754966 | c.264-2530G > A\|c.637G > A | | | | | | | | | 5 | |
| 02:219755011 | c.264-2485T > A1c.682T > A | | | | | | | | | 5 | |
| 02:241808314 | c.32C > T | | | | | | | | | 2125515 | |
| 02:241817516 | c.1020A > G | | | | | | | | | 2125515 | |
| 03:014200382 | c.*454C > A\|c.1001C > A\|c.890C > A | | | | | | | | | 115 | |
| 03:015677019 | c.133G > A\|c.139G > A\|c.73G > A | | | | | | | | | 5 | |
| 03:015677098 | c.152T > C\|c.212T > C\|c.218T > C | | | | | | | | | 5 | |
| 03:015686243 | c.820A > G\|c.880A > G\|c.886A > G | | | | | | | | | 5 | |
| 03:015686331 | c.908A > G\|c.968A > G\|c.974A > G | | | | | | | | | 015 | |
| 03:015686534 | c.1111C > T\|c.1171C > T\|c.1177C > T | | | | | | | | | 215 | |
| 03:015686693 | c.1270G > C\|c.1330G > C\|c.1336G > C | | | | | | | | | 5 | |
| 03:128598490 | c.-44_-41dupTAAG\|c.-45delCinsCTAAG\|c.insTAAG | | | | | | | | | 5 | |
| 03:142275281 | c.2022A > G\|c.2101A-G | | | | | | | | | 5 | |
| 03:150645894 | c.104+13475T > G\|c.162+13475T > G\|c.300T > G\|c.528T > G\|c.567T > G | | | | | | | | | 5 | |
| 03:165491280 | c.*205G > A\|c.*89G > A\|c.1699G > A\|c.289G > A\|c.85G > A | | | | | | | | | 2125515 | |
| 03:165547569 | c.-98+7533G > T\|c.107+7533G > T\|c.1253G > T | | | | | | | | | 5 | |
| 03:165548529 | c.-98+6573A > G\|c.107+6573A > G\|c.293A > G | | | | | | | | | 5 | |
| 03:183966564 | c.154+6C > T\|c.158C > T\|c.159+6C > T\|c.160del37\|c.165C > T\|c.2106+104069G > A\|c.52 | | | | | | | | | 5 | |
| | +450C > T\|c.76+214C > T\|c.97C > T | | | | | | | | | | |
| 04:005755524 | c.1328G > A | | | | | | | | | 5 | |
| 04:187004074 | c.1042C > T\|c.1234C > T\|c.403C > T | | | | | | | | | 5 | |
| 05:073981270 | 215 | | | | | | | | | | |
| 05:073981270 | c.-376-3883T > C\|c.185C=\|c.185C > T\|c.185T > C | | | | | | | | | 215 | |
| 05:118792052 | c.-311C > T\|c.-37C > T\|c.101C > T\|c.111C > T\|c.58+3724C > T | | | | | | | | | 5 | |
| 05:131729380 | c.*195G > A\|c.*315G > A\|c.1463G > A\|c.1535G > A | | | | | | | | | 5 | |
| 06:007542236 | c.88G > A | | | | | | | | | 255131415 | |
| 06:032006858 | c.203-13C > G\|c.209C > G\|c.293-13C > G\|c.299C > G\|c.313C > G | | | | | | | | | 5 | |
| 06:161159625 | c.1858G > A | | | | | | | | | 5 | |
| 07:065432754 | c.*884C > T\|c.*997C > T\|c.1179C > T\|c.1464C > T\|c.1617C > T\|c.1642del38 | | | | | | | | | 5 | |
| 07:087060844 | c.1769G > A | | | | | | | | | 5 | |
| 07:087082273 | c.523A > G | | | | | | | | | 5 | |
| 07:117149147 | c.-20G > A\|c.224G > A | | | | | | | | | 0121315 | |
| 07:117175372 | c.560A > G\|c.650A > G | | | | | | | | | 5 | |
| 07:117188682 | c.1120-13_1120-11delGTTinsG\|c.1209+6520_1209+6522delGTTinsG\|c.1210-12[5] | | | | | | | | | 0125515 | |
| | \|c.1210-12_1210-6T[5]\|c.1210-13_1210-11delGTTinsG\|c.1210-7_1210-6delTT\|c | | | | | | | | | | |
| | .5T | | | | | | | | | | |
| 07:117227874 | c.1483A > G\|c.1576A > G\|c.1666A > G | | | | | | | | | 415 | |
| 07:117243663 | c.2552C > T\|c.2645C > T\|c.2735C > T | | | | | | | | | 215 | |
| 07:117251704 | c.3026G > A\|c.3119G > A\|c.3209G > A\|c.34G > A | | | | | | | | | 115 | |
| 07:117267556 | c.294-20T > C\|c.3286-20T > C\|c.3379-20T > C\|c.3469-20T > C\|c.3601, T-C, -20 | | | | | | | | | 5 | |
| 07:117304834 | c.182G > C\|c.3873G > C\|c.3966G > C\|c.4056G > C | | | | | | | | | 5 | |
| 08:022021460 | c.*127G > A\|c.*383G > A\|c.277-319G > A\|c.323G > A\|c.341G > A\|c.482G > A\|c.500G > A | | | | | | | | | 5 | |
| 08:086392989 | c.*341A > G\|c.754A > G | | | | | | | | | 5 | |
| 08:090983460 | c.*516C > T\|c.379C > T\|c.397C > T\|c.643C > T | | | | | | | | | 011125515 | |
| 08:100832259 | c.*4761A > G\|c.8903A > G\|c.8978A > G | | | | | | | | | 215 | |
| 09:034647661 | c.*145T > C\|c.*80T > C\|c.252+406T > C\|c.253-168T > C\|c.287T > C\|c.336T > C\|c.51-168 | | | | | | | | | 5 | |
| | T > C | | | | | | | | | | |
| 09:034648848 | c.777G > A | | | | | | | | | 5 | |
| 09:034649442 | c.*560A > G\|c.432+989A > G\|c.613A > G\|c.940A > G | | | | | | | | | 11212551315 | |
| 09:036217445 | c.1756G > A\|c.1864G > A\|c.2071G > A\|c.2086G > A\|c.2179G > A\|c.485+13269C > T | | | | | | | | | 5 | |
| 09:136301982 | c.*146C > G\|c.*210C > G\|c.*626C > G\|c.1249C > G\|c.1342C > G\|c.358C > G | | | | | | | | | 5 | |
| 09:136302063 | c.*227C > T\|c.*291C > T\|c.*707C > T\|c.1330C > T\|c.1423C > T\|c.439C > T | | | | | | | | | 5 | |
| 10:050678722 | c.1394C > G\|c.3284C > G | | | | | | | | | 5 | |
| 10:072358722 | c.755A > G | | | | | | | | | 5 | |
| 10:072360648 | c.11G > A | | | | | | | | | 5 | |
| 10:102749444 | c.1287C > T | | | | | | | | | 5 | |
| 11:005247873 | c.249G > Y | | | | | | | | | 25515 | |
| 11:005248029 | c.93G > T | | | | | | | | | 215 | |
| 11:005248177 | c.75T > A | | | | | | | | | 5 | |
| 11:017531093 | c.1192-7566C > G\|c.1211-7566C > G\|c.1228-7566C > G\|c.1285-7566C > G\|c.1823C > G | | | | | | | | | 315 | |
| 11:017548622 | EXP | | | | | | | | | 215 | |
| 11:017548878 | c.295G > A\|c.388G > A\|c.421G > A | | | | | | | | | 215 | |
| 11:017552978 | c.123G > A\|c.216G-A\|c.216G > A\|c.249G > A | | | | | | | | | 5 | |
| 11:036615075 | c.644C > T | | | | | | | | | 5 | |
| 11:064525266 | c.381G > A\|c.645G > A | | | | | | | | | 215 | |
| 11:066287196 | c.*158G > A\|c.*360G > A\|c.*403G > A\|c.*407G > A\|c.*489G > A\|c.364G > A\|c.427G > A\|c. | | | | | | | | | 2551315 | |
| | 433-1545G > A\|c.700G > A\|c.811G > A | | | | | | | | | | |
| 11:068562328 | c.79G > A\|c.823G > A | | | | | | | | | 215 | |
| 11:071906793 | c.493+2T > C | | | | | | | | | 5 | |
| 11:076869378 | c.872G > A\|c.905G > A | | | | | | | | | 215 | |
| 11:108143299 | c.3118A > G | | | | | | | | | 21315 | |
| 11:128709126 | c.1013T > C\|c.1070T > C | | | | | | | | | 5 | |
| 12:006458350 | c.*548T > C\|c.1439+143T > C\|c.1477T > C\|c.1543T > C\|c.1546T > C\|c.1654T > C\|c.577T > | | | | | | | | | 5 | |
| | C | | | | | | | | | | |
| 12:102164255 | c.1042A > C | | | | | | | | | 5 | |
| 12:121175678 | c.473-174C > T\|c.511C > T | | | | | | | | | 2551415 | |
| 12:121176083 | c.613G > A\|c.625G > A | | | | | | | | | 212551415 | |
| 12:122277904 | c.1005C > G\|c.888C > G | | | | | | | | | 5 | |
| 12:122295335 | c.-21A > G\|c.97A > G\|c.97G > A | | | | | | | | | 5 | |
| 13:020763234 | c.487A > G | | | | | | | | | 315 | |
| 13:020763553 | c.167delT | | | | | | | | | 5 | |
| 13:020763612 | 5 | | | | | | | | | | |
| 13:020763620 | c.101T > C | | | | | | | | | 0125515 | |
| 13:020763685 | c.35delG | | | | | | | | | 5 | |
| 13:020763710 | c.11G > A | | | | | | | | | 215 | |
| 13:052513266 | c.1253A > G\|c.2330A > G\|c.2999A > G\|c.3287A > G\|c.3386A > G\|c.3425A > G1c.3620A > G | | | | | | | | | 212551315 | |
| 13:052532497 | c.121A > G\|c.1286-8200A > G\|c.1870-754A > G\|c.1972A > G\|c.2122-754A > G1c.2305A | | | | | | | | | 215 | |
| | > G | | | | | | | | | | |
| 13:108863591 | c.26C > T | | | | | | | | | 215 | |
| 13:108863609 | c.8C > T | | | | | | | | | 215 | |
| 14:024724663 | c.1552G > A\|c.226G > A\|c.625G > A | | | | | | | | | 5 | |
| 14:024731434 | c.-130C > A\|c.125C > A | | | | | | | | | 5 | |
| 14:088452941 | c.*205A > G\|c.*83A > G\|c.166A > G\|c.256A > G\|c.265A > G1c.334A > G | | | | | | | | | 5 | |
| 15:072638893 | c.*110C > T\|c.*559-227C > T\|c.*715C > T\|c.*826C > T\|c.1305C > T\|c.1338C > T\|c.498-22 | | | | | | | | | 5 | |
| | 7C > T\|c.786C > T\|c.946-227C > T | | | | | | | | | | |
| 15:072641434 | c.972T > A | | | | | | | | | 5 | |
| 15:080472526 | c.1021C > T\|c.119C > T\|c.811C > T | | | | | | | | | 5 | |
| 15:089865073 | c.185+900A > G\|.196-582A > G\|c.2492A > G | | | | | | | | | 215 | |
| 16:000222980 | c.69C > T | | | | | | | | | 5 | |
| 16:003293257 | c.*434G > T\|c.*506G > T\|c.*614G > T\|c.*714G > T\|c.*747G > T\|c.*855G > T\|c.*863G > T\|c. | | | | | | | | | 0125515 | |
| | 1597G > T\|c.1690G > T\|c.2230G > T | | | | | | | | | | |
| 16:003293310 | c.*381T > C\|c.*453T > C\|c.*561T > C\|c.*661T > C\|c.*694T > C\|c.*802T > C\|c.*810T > C\|c.1 | | | | | | | | | 5 | |
| | 544T > C\|c.1637T > C\|c.2177T > C | | | | | | | | | | |
| 16:003293403 | c.*288A > G\|c.*360A > G\|c.*468A > G\|c.*568A > G\|c.*601A > G\|c.*709A > G\|c.*717A > G\| | | | | | | | | | 5 | |
| | c.1451A > G\|c.1544A > G\|c.2084A > G | | | | | | | | | | |
| 16:003304626 | c.277+1685G > C\|c.442G > C | | | | | | | | | 015 | |
| 16:023200921 | c.547G > A | | | | | | | | | 5 | |
| 16:023200963 | c.589G > A | | | | | | | | | 5 | |
| 16:023360165 | c.245C > G\|c.380C > G | | | | | | | | | 5 | |
| 16:053720436 | c.685G > A | | | | | | | | | 212551315 | |
| 16:088502971 | c.9010_9024delCTTCCCGGGAACACC\|c.9094_9108delCTTCCCGGGAACACC | | | | | | | | | 5 | |
| 17:003550800 | c.-113+7239G > A\|c.-216-7492G > A\|c.124G > A\|c.5+7239G > A | | | | | | | | | 5 | |
| 17:007123838 | c.*149C > T\|c.139-85C > T\|c.194C > T\|c.263C > T | | | | | | | | | 215 | |
| 17:015134364 | c.*62C > T\|c.174C > T\|c.353C > T | | | | | | | | | 5 | |
| 17:041063017 | c.*40G > T\|c.648G > T | | | | | | | | | 5 | |
| 17:078078341 | c.-32-13T > G | | | | | | | | | 5 | |
| 19:007125518 | c.2998G > A\|c.3034G > A | | | | | | | | | 415 | |
| 19:012917556 | c.69G > A | | | | | | | | | 5 | |
| 19:012917562 | c.75C > T | | | | | | | | | 5 | |
| 19:018710530 | c.242G > A | | | | | | | | | 215 | |
| 19:036339044 | c.1338_1339delTGinsTA\|c.1339G > A | | | | | | | | | 315 | |
| 19:045860626 | c.1147C > G\|c.1309C > G\|c.1381C > G\|c.504C > G | | | | | | | | | 115 | |
| 20:043255233 | c.218+2455C > T\|c.226C > T | | | | | | | | | 115 | |
| 20:043280227 | c.22G > A | | | | | | | | | 5 | |
| 22:050962500 | c.341G > A | | | | | | | | | 5 | |
| 22:051064039 | c.1172C > G\|c.1178C > G\|c.46C > G\|c.920C > G | | | | | | | | | 215 | |
| 22:051064416 | c.1049A > G\|c.1055A > G\|c.797A > G | | | | | | | | | 11215 | |
| | | | | | | | Popu- | | | | |
| | | | | | | | lation | | | | |
| | | | | | | | with | | | Number | |
| | | Poly | | | | Maximum | highest | | | of | |
| | | Phen- | CADD | PROVEAN | | population | fre- | | | homo- | Variant |
| Chr:bp | Variant type | 2 | (adj) | (adj) | VEST | frequency | quency | AN | Carriers | zygotes | source |
| 01:043212925 | Synonymous | . | 7.309 | . | . | 1.5051173 | NFE | 121028 | 1 | 0 | ExAC and |
| | | | | | | 9916e-05 | | | | | ClinVar |
| 01:046655645 | Missense | 0.997 | 23.1 | -3.12 | 0.148 | 0.0126391 | NFE | 118596 | 1064 | 9 | ExAC and |
| | | | | | | 218174 | | | | | ClinVar |
| 01:063872032 | Missense | 0.014 | 20.3 | -3.57 | 0.315 | 0.0397375 | NFE | 121148 | 3318 | 79 | ExAC and |
| | | | | | | 848196 | | | | | ClinVar |
| 01:076198337 | Missense | 0.422 | 26.9 | -2.69 | 0.744 | 0.0033676 | NFE | 120842 | 270 | 1 | ExAC and |
| | | | | | | 1080041 | | | | | ClinVar |
| 01:097915614 | Essential | . | 22.6 | . | . | 0.0058331 | NFE | 12125 | 624 | 5 | ExAC and |
| | splice site | | | | | 3339731 | | | | | ClinVar |
| | (intron) | | | | | | | | | | |
| 01:098348885 | Missense | . | 23.8 | . | 0.201 | 0.4163783 | AFR | 121114 | 2094 | 3749 | ExAC and |
| | | | | | | 16032 | | | 3 | | ClinVar |
| 01:100672060 | Missense | . | 22.8 | -2.05 | 0.463 | 0 | . | . | . | . | ClinVar |
| 01:100672060 | Missense | . | 7.686 | . | 0.158 | 0.2358254 | AFR | 121412 | 9188 | 641 | ExAC and |
| | | | | | | 85297 | | | | | ClinVar |
| 01:155204994 | Synonymous | . | 14.73 | | | 0.0010399 | EAS | 121388 | 35 | 0 | ExAC and |
| | | | | | | 8151144 | | | | | ClinVar |
| 01:155205008 | Missense | 0.037 | 13.26 | -1.51 | 0.831 | 0.0003845 | AFR | 121380 | 14 | 0 | ExAC, |
| | | | | | | 4143434 | | | | | ClinVar, |
| | | | | | | | | | | | and |
| | | | | | | | | | | | VariBench |
| 01:155206167 | Missense | 0.015 | 21.7 | -1.2 | 0.402 | 0.0119640 | NFE | 121320 | 1164 | 12 | ExAC, |
| | | | | | | 17991 | | | | | ClinVar, |
| | | | | | | | | | | | and |
| | | | | | | | | | | | VariBench |
| 01:156108401 | Synonymous | . | 15.94 | . | . | 0 | . | . | . | . | ClinVar |
| 01:156108404 | Synonymous | . | 18.65 | . | . | 0 | . | . | . | . | ClinVar |
| 01:156848918 | Missense | 0.986 | 28.2 | -5.34 | 0.727 | 0.0550196 | NFE | 120020 | 4832 | 136 | ExAC and |
| | | | | | | 552767 | | | | | ClinVar |
| 01:156848946 | Missense | 0.379 | 22.3 | -2.72 | 0.463 | 0.0549432 | NFE | 120296 | 4825 | 138 | ExAC and |
| | | | | | | 298587 | | | | | ClinVar |
| 01:216498841 | Synonymous | . | 10.51 | . | . | 2.9992202 | NFE | 121146 | 2 | 0 | ExAC and |
| | | | | | | 0275e-05 | | | | | ClinVar |
| 01:227170648 | Synonymous | . | 17.34 | . | . | 0.0286132 | NFE | 85834 | 1729 | 33 | ExAC and |
| | | | | | | 439656 | | | | | ClinVar |
| 01:241665852 | Missense | 1.0 | 33.0 | -5.97 | 0.95 | 0.0001499 | NFE | 121332 | 11 | 0 | ExAC and |
| | | | | | | 02563334 | | | | | ClinVar |
| 01:241675301 | Missense | 0.999 | 27.9 | -7.67 | 0.919 | 2.9967934 | NFE | 121408 | 2 | 0 | ExAC and |
| | | | | | | 3103e-05 | | | | | ClinVar |
| 02:026461825 | Synonymous | . | 22.8 | . | . | 0.0001648 | NFE | 121300 | 11 | 0 | ExAC and |
| | | | | | | 97763387 | | | | | ClinVar |
| 02:145156798 | Synonymous | . | 0.067 | . | . | 0 | . | . | . | . | ClinVar |
| 02:215813331 | Missense | 0.908 | 21.1 | -1.2 | 0.016 | 0.0321069 | AMR | 121124 | 3203 | 81 | ExAC, |
| | | | | | | 073238 | | | | | ClinVar, |
| | | | | | | | | | | | and |
| | | | | | | | | | | | VariBench |
| 02:219674479 | Synonymous | . | | | 6.272 | 0.0006939 | EAS | 120044 | 6 | 0 | ExAC and |
| | | | | | | 62526024 | | | | | ClinVar |
| 02:219678877 | Missense | 1.0 | 20.7 | -8.96 | 0.327 | 0.0304626 | SAS | 121374 | 2205 | 37 | ExAC and |
| | | | | | | 937984 | | | | | ClinVar |
| 02:219754966 | Missense | 1.0 | 33.0 | -5.2 | 0.982 | 0.0237364 | EAS | 118674 | 200 | 4 | ExAC and |
| | | | | | | 194615 | | | | | ClinVar |
| 02:219755011 | Missense | 0.999 | 31.0 | -4.95 | 0.843 | 0.0200445 | NFE | 117764 | 1471 | 14 | ExAC and |
| | | | | | | 0901 | | | | | ClinVar |
| 02:241808314 | Missense | 1.0 | 12.92 | -8.95 | 0.523 | 0.2126775 | NFE | 114234 | 1423 | 1706 | ExAC and |
| | | | | | | 36463 | | | 8 | | ClinVar |
| 02:241817516 | Missense | 0.0 | 0.003 | 3.24 | 0.095 | 0.2104424 | NFE | 120178 | 1606 | 1897 | ExAC, |
| | | | | | | 61763 | | | 7 | | ClinVar, |
| | | | | | | | | | | | and |
| | | | | | | | | | | | VariBench |
| 03:014200382 | Missense | 0.855 | 9.104 | -1.23 | 0.075 | 0.0250665 | AFR | 120636 | 341 | 2 | ExAC and |
| | | | | | | 029671 | | | | | ClinVar |
| 03:015677019 | Missense | 0.001 | 10.21 | -0.39 | 0.078 | 0.0133503 | NFE | 121412 | 1186 | 11 | ExAC, |
| | | | | | | 146539 | | | | | ClinVar, |
| | | | | | | | | | | | and |
| | | | | | | | | | | | VariBench |
| 03:015677098 | Missense | 0.0 | 6.242 | 3.27 | 0.634 | 0.0116279 | SAS | 121412 | 177 | 8 | ExAC and |
| | | | | | | 069767 | | | | | ClinVar |
| 03:015686243 | Missense | 0.0 | 0.001 | 0.64 | 0.018 | 0.0426758 | AFR | 121398 | 457 | 12 | ExAC, |
| | | | | | | 93887 | | | | | ClinVar, |
| | | | | | | | | | | | and |
| | | | | | | | | | | | VariBench |
| 03:015686331 | Missense | 1.0 | 25.3 | -5.52 | 0.877 | 0.0164728 | SAS | 121162 | 275 | 3 | ExAC and |
| | | | | | | 682171 | | | | | ClinVar |
| 03:015686534 | Missense | 0.002 | 12.3 | -0.63 | 0.077 | 0.0210680 | NFE | 121406 | 1650 | 24 | ExAC, |
| | | | | | | 891872 | | | | | ClinVar, |
| | | | | | | | | | | | and |
| | | | | | | | | | | | VariBench |
| 03:015686693 | Missense | 1.0 | 21.0 | -5.48 | 0.817 | 0.0393802 | NFE | 121398 | 3678 | 83 | ExAC and |
| | | | | | | 259718 | | | | | ClinVar |
| 03:128598490 | 5′ | . | 9.008 | . | . | 0.1260629 | AFR | 117854 | 4457 | 220 | ExAC and |
| | untranslated | | | | | 2517 | | | | | ClinVar |
| 03:142275281 | Synonymous | . | 13.68 | . | . | 0 | . | . | . | | ClinVar |
| 03:150645894 | Nonsense | . | 13.83 | . | 0.9999 | 0.0004874 | GLOBAL | 21034 | 55 | 2 | ExAC and |
| | | | | | | 66331775 | | | | | ClinVar |
| 03:165491280 | Missense | 0.001 | 22.9 | -0.52 | 0.204 | 0.2109435 | NFE | 113876 | 1747 | 2029 | ExAC, |
| | | | | | | 9241 | | | 8 | | ClinVar, |
| | | | | | | | | | | | and |
| | | | | | | | | | | | VariBench |
| 03:165547569 | Missense | 0.088 | 21.9 | -1.96 | 0.295 | 0.0046787 | NFE | 121182 | 362 | 2 | ExAC, |
| | | | | | | 8351629 | | | | | ClinVar, |
| | | | | | | | | | | | and |
| | | | | | | | | | | | VariBench |
| 03:165548529 | Missense | 0.687 | 24.7 | -5.56 | 0.913 | 0.0176578 | NFE | 121188 | 1433 | 15 | ExAC, |
| | | | | | | 25252 | | | | | ClinVar, |
| | | | | | | | | | | | and |
| | | | | | | | | | | | VariBench |
| 03:183966564 | Synonymous | . | 22.4 | . | . | 3.3451528 | NFE | 107798 | 2 | 0 | ExAC and |
| | | | | | | 7349e-05 | | | | | ClinVar |
| 04:005755524 | Missense | 0.265 | 17.45 | -0.53 | 0.191 | 0.2250096 | AFR | 121404 | 1987 | 283 | ExAC, |
| | | | | | | 11688 | | | | | ClinVar, |
| | | | | | | | | | | | and |
| | | | | | | | | | | | VariBench |
| 04:187004074 | Missense | 1.0 | 23.9 | -3.54 | 0.256 | 0.3373521 | EAS | 120980 | 2318 | 4813 | ExAC and |
| | | | | | | 9105 | | | 4 | | ClinVar |
| 05:073981270 | Missense | . | 9.454 | -0.82 | 0.386 | 0 | . | . | . | . | ClinVar |
| 05:073981270 | Missense | . | 9.134 | . | 0.191 | 0.0379595 | NFE | 113532 | 3207 | 53 | ExAC and |
| | | | | | | 528849 | | | | | ClinVar |
| 05:118792052 | Synonymous | . | 23.0 | . | . | 0.0001155 | EAS | 121398 | 1 | 0 | ExAC and |
| 80212668 | | | | | | | | | | | ClinVar |
| 05:131729380 | Missense | 0.972 | 34.0 | -3.27 | 0.876 | 0.0057004 | AMR | 121410 | 388 | 4 | ExAC and |
| | | | | | | 664018 | | | | | ClinVar |
| 06:007542236 | Missense | 0.0 | 8.982 | 0.4 | 0.683 | 0.0057011 | SAS | 47946 | 147 | 1 | ExAC and |
| | | | | | | 4022805 | | | | | ClinVar |
| 06:032006858 | Intron | . | . | . | . | 0.0027881 | NFE | 87414 | 202 | 2 | ExAC and |
| | | | | | | 0408922 | | | | | ClinVar |
| 06:161159625 | Missense | 1.0 | 23.8 | -2.77 | 0.802 | 0.0181418 | EAS | 121404 | 155 | 3 | ExAC, |
| | | | | | | 996996 | | | | | ClinVar, |
| | | | | | | | | | | | and |
| | | | | | | | | | | | VariBench |
| 07:065432754 | Synonymous | . | 11.35 | . | . | 5.9943054 | NFE | 121400 | 4 | 0 | ExAC and |
| | | | | | | 0986e-05 | | | | | ClinVar |
| 07:087060844 | Missense | 0.999 | 36.0 | -3.62 | 0.732 | 0.0057402 | NFE | 121370 | 503 | 2 | ExAC, |
| | | | | | | 3560445 | | | | | ClinVar, |
| | | | | | | | | | | | and |
| | | | | | | | | | | | VariBench |
| 07:087082273 | Missense | 0.841 | 25.0 | -3.82 | 0.702 | 0.0132630 | SAS | 121306 | 1266 | 11 | ExAC and |
| | | | | | | 813953 | | | | | ClinVar |
| 07:117149147 | Missense | 1.0 | 27.2 | -2.23 | 0.914 | 0.0248140 | NFE | 121258 | 1807 | 28 | ExAC, |
| | | | | | | 81804 | | | | | ClinVar, |
| | | | | | | | | | | | and |
| | | | | | | | | | | | VariBench |
| 07:117175372 | Missense | 0.053 | 24.5 | -2.03 | 0.422 | 0.0102866 | EAS | 121392 | 454 | 11 | ExAC, |
| | | | | | | 389274 | | | | | ClinVar, |
| | | | | | | | | | | | and |
| | | | | | | | | | | | VariBench |
| 07:117188682 | Intron | . | 13.2 | . | . | 0.0651453 | AFR | 99808 | 2746 | 26 | ExAC and |
| | | | | | | 340133 | | | | | ClinVar |
| 07:117227874 | Missense | 0.334 | 22.0 | -0.07 | 0.582 | 0.0400603 | EAS | 119930 | 325 | 12 | ExAC and |
| | | | | | | 808639 | | | | | ClinVar |
| 07:117243663 | Missense | 0.055 | 9.102 | -0.1 | 0.717 | 0.0013193 | NFE | 121272 | 108 | 0 | ExAC and |
| | | | | | | 0077059 | | | | | ClinVar |
| 07:117251704 | Missense | 1.0 | 22.0 | -0.14 | 0.936 | 0.0053908 | SAS | 120098 | 93 | 2 | ExAC, |
| | | | | | | 3557951 | | | | | ClinVar, |
| | | | | | | | | | | | and |
| | | | | | | | | | | | VariBench |
| 07:117267556 | Intron | . | . | . | . | 0.0132863 | SAS | 118048 | 208 | 7 | ExAC and |
| | | | | | | 675689 | | | | | ClinVar |
| 07:117304834 | Missense | 1.0 | 26.0 | -4.19 | 0.969 | 0.0134259 | EAS | 121348 | 109 | 4 | ExAC and |
| | | | | | | 259259 | | | | | ClinVar |
| 08:022021460 | Missense | 0.424 | 5.102 | 0.16 | 0.046 | 0.0161023 | AFR | 119764 | 160 | 3 | ExAC and |
| | | | | | | 947151 | | | | | ClinVar |
| 08:086392989 | Missense | 0.0 | 13.24 | -1.41 | 0.039 | 0.0913110 | AFR | 121250 | 916 | 47 | ExAC, |
| | | | | | | 342176 | | | | | ClinVar, |
| | | | | | | | | | | | and |
| | | | | | | | | | | | VariBench |
| 08:090983460 | Missense | 1.0 | 26.2 | -4.1 | 0.743 | 0.0047084 | NFE | 120222 | 349 | 3 | ExAC and |
| | | | | | | 1155453 | | | | | ClinVar |
| 08:100832259 | Missense | 0.998 | 23.4 | -4.61 | 0.488 | 0.0049163 | NFE | 121316 | 394 | 0 | ExAC, |
| | | | | | | 618922 | | | | | ClinVar, |
| | | | | | | | | | | | and |
| | | | | | | | | | | | VariBench |
| 09:034647661 | Synonymous | . | 20.7 | . | . | 1.4990705 | NFE | 121364 | 1 | 0 | ExAC and |
| | | | | | | 7624e-05 | | | | | ClinVar |
| 09:034648848 | Synonymous | . | 17.3 | . | . | 0 | . | . | . | . | ClinVar |
| 09:034649442 | Missense | 0.0 | 16.87 | 0.59 | 0.381 | 0.1832444 | SAS | 121390 | 9785 | 692 | ExAC and |
| | | | | | | 87521 | | | | | ClinVar |
| 09:036217445 | Missense | 0.993 | 22.3 | -0.52 | 0.952 | 0.0140913 | SAS | 121022 | 229 | 3 | ExAC, |
| | | | | | | 50826 | | | | | ClinVar, |
| | | | | | | | | | | | and |
| | | | | | | | | | | | VariBench |
| 09:136301982 | Missense | 0.0 | 5.693 | 1.81 | 0.068 | 0.5108259 | AMR | 60658 | 1672 | 5376 | ExAC and |
| | | | | | | 82358 | | | 7 | | ClinVar |
| 09:136302063 | Missense | 0.024 | 7.79 | -0.58 | 0.097 | 0.0506594 | AMR | 52912 | 380 | 4 | ExAC and |
| | | | | | | 724221 | | | | | ClinVar |
| 10:050678722 | Missense | 0.0 | 7.757 | -0.45 | 0.101 | 0.0419150 | AFR | 121382 | 439 | 10 | ExAC, |
| | | | | | | 16343 | | | | | ClinVar, |
| | | | | | | | | | | | and |
| | | | | | | | | | | | VariBench |
| 10:072358722 | Missense | 0.0 | 1.583 | -0.59 | 0.007 | 0.0101883 | AFR | 121358 | 622 | 3 | ExAC, |
| | | | | | | 890811 | | | | | ClinVar, |
| | | | | | | | | | | | and |
| | | | | | | | | | | | VariBench |
| 10:072360648 | Missense | 0.0 | 9.447 | 1.06 | 0.052 | 0.1174863 | AFR | 48114 | 511 | 23 | ExAC, |
| | | | | | | 38798 | | | | | ClinVar, |
| | | | | | | | | | | | and |
| | | | | | | | | | | | VariBench |
| 10:102749444 | Synonymous | . | 19.24 | . | . | 0 | . | . | . | . | ClinVar |
| 11:005247873 | Synonymous | . | 19.62 | . | . | 0 | . | . | . | . | ClinVar |
| 11:005248029 | Essential | . | 18.98 | . | . | 0.0002804 | GLOBAL | 121244 | 34 | 0 | ExAC and |
| | splice site | | | | | 26247897 | | | | | ClinVar |
| | (exon) | | | | | | | | | | |
| 11:005248177 | Synonymous | . | 21.9 | . | . | 9.6098404 | AFR | 121356 | 2 | 0 | ExAC and |
| | | | | | | 7665e-05 | | | | | ClinVar |
| 11:017531093 | Missense | 0.984 | 24.3 | -1.82 | 0.727 | 0.0008456 | NFE | 108256 | 57 | 0 | ExAC and |
| | | | | | | 86996319 | | | | | ClinVar |
| 11:017548622 | Intron | . | . | . | . | 0 | . | . | . | . | ClinVar |
| 11:017548878 | Essential | . | 19.43 | . | . | 0.0462371 | AFR | 120926 | 473 | 10 | ExAC and |
| | splice site | | | | | 832076 | | | | | ClinVar |
| | (exon) | | | | | | | | | | |
| 11:017552978 | Synonymous | . | 19.46 | . | . | 7.5918615 | NFE | 119738 | 5 | 0 | ExAC and |
| | | | | | | 2445e-05 | | | | | ClinVar |
| 11:036615075 | Missense | 0.51 | 12.39 | -2.65 | 0.478 | 0.0249515 | SAS | 121360 | 422 | 6 | ExAC, |
| | | | | | | 503876 | | | | | ClinVar, |
| | | | | | | | | | | | and |
| | | | | | | | | | | | VariBench |
| 11:064525266 | Synonymous | . | 18.78 | . | . | 0.0067532 | AMR | 120960 | 528 | 3 | ExAC and |
| | | | | | | 4675325 | | | | | ClinVar |
| 11:066287196 | Missense | 0.99 | 22.3 | -2.29 | 0.432 | 0.1017194 | AFR | 120856 | 1017 | 55 | ExAC, |
| | | | | | | 74498 | | | | | ClinVar, |
| | | | | | | | | | | | and |
| | | | | | | | | | | | VariBench |
| 11:068562328 | Missense | 0.0 | 13.13 | -0.07 | 0.085 | 0.0896940 | NFE | 121408 | 7079 | 388 | ExAC and |
| | | | | | | 273907 | | | | | ClinVar |
| 11:071906793 | Essential | . | 12.06 | . | . | 0.0136870 | SAS | 121396 | 400 | 8 | ExAC and |
| | splice site | | | | | 155039 | | | | | ClinVar |
| | (intron) | | | | | | | | | | |
| 11:076869378 | Missense | 0.998 | 30.0 | -4.28 | 0.697 | 0.0058803 | NFE | 116632 | 409 | 2 | ExAC, |
| | | | | | | 5559946 | | | | | ClinVar, |
| | | | | | | | | | | | and |
| | | | | | | | | | | | VariBench |
| 11:108143299 | Missense | 0.0 | 0.141 | -0.14 | 0.12 | 0.0413846 | AFR | 121182 | 428 | 10 | ExAC, |
| | | | | | | 451363 | | | | | ClinVar, |
| | | | | | | | | | | | and |
| | | | | | | | | | | | VariBench |
| 11:128709126 | Missense | 0.004 | 12.33 | -0.1 | 0.213 | 0.0119918 | NFE | 121350 | 950 | 12 | ExAC, |
| | | | | | | 45545 | | | | | ClinVar, |
| | | | | | | | | | | | and |
| | | | | | | | | | | | VariBench |
| 12:006458350 | Missense | 1.0 | 22.1 | -12.93 | 0.939 | 0.0250121 | SAS | 121400 | 2158 | 28 | ExAC and |
| | | | | | | 124031 | | | | | ClinVar |
| 12:102164255 | Missense | 0.904 | 23.3 | -1.85 | 0.307 | 0.0415224 | AFR | 121328 | 447 | 4 | ExAC and |
| | | | | | | 913495 | | | | | ClinVar |
| 12:121175678 | Missense | 0.994 | 13.76 | -4.32 | 0.194 | 0.0445456 | NFE | 120756 | 3588 | 97 | ExAC and |
| | | | | | | 464698 | | | | | ClinVar |
| 12:121176083 | Essential | . | 19.66 | . | . | 0.3787721 | AMR | 121136 | 2232 | 4537 | ExAC and |
| | splice site | | | | | 12383 | | | 2 | | ClinVar |
| | (exon) | | | | | | | | | | |
| 12:122277904 | Missense | 0.978 | 18.43 | -2.78 | 0.888 | 0.0076308 | SAS | 121382 | 252 | 2 | ExAC and |
| | | | | | | 1395349 | | | | | ClinVar |
| 12:122295335 | Missense | . | 13.89 | . | 0.345 | 0.2803318 | AMR | 121370 | 1487 | 1653 | ExAC and |
| | | | | | | 35465 | | | 3 | | ClinVar |
| 13:020763234 | Missense | 0.91 | 17.93 | -2.16 | 0.95 | 0.0006068 | SAS | 121056 | 20 | 0 | ExAC and |
| | | | | | | 69765748 | | | | | ClinVar |
| 13:020763553 | Frameshift | . | 13.32 | . | 0.9985 | 0.0011397 | NFE | 121310 | 77 | 3 | ExAC and |
| | | | | | | 7204559 | | | | | ClinVar |
| 13:020763612 | Missense | 1.0 | 11.2 | -0.82 | 0.703 | 0.0724201 | EAS | 121300 | 721 | 39 | ExAC, |
| | | | | | | 758445 | | | | | ClinVar, |
| | | | | | | | | | | | and |
| | | | | | | | | | | | VariBench |
| 13:020763620 | Missense | 0.038 | 5.565 | -3.8 | 0.396 | 0.0122214 | NFE | 121354 | 1006 | 13 | ExAC and |
| | | | | | | 557778 | | | | | ClinVar |
| 13:020763685 | Frameshift | . | 12.64 | . | 0.9892 | 0.0087724 | NFE | 121352 | 727 | 3 | ExAC and |
| | | | | | | 5598776 | | | | | ClinVar |
| 13:020763710 | Missense | 0.088 | 1.016 | -2.15 | 0.091 | 0.0040453 | EAS | 121210 | 49 | 1 | ExAC and |
| | | | | | | 0744337 | | | | | ClinVar |
| 13:052513266 | Missense | 0.044 | 14.93 | -4.29 | 0.112 | 0.1607605 | AMR | 120728 | 2727 | 187 | ExAC and |
| | | | | | | 87727 | | | | | ClinVar |
| 13:052532497 | Missense | 1.0 | 25.9 | -3.23 | 0.894 | 0.0001049 | NFE | 120692 | 8 | 0 | ExAC, |
| | | | | | | 72707096 | | | | | ClinVar, |
| | | | | | | | | | | | and |
| | | | | | | | | | | | VariBench |
| 13:108863591 | Missense | 0.52 | 20.2 | -2.7 | 0.139 | 0.2335606 | AMR | 116530 | 1624 | 1962 | ExAC and |
| | | | | | | 85554 | | | 0 | | ClinVar |
| 13:108863609 | Missense | 0.376 | 23.2 | -0.54 | 0.237 | 0.1202397 | EAS | 115050 | 6277 | 234 | ExAC and |
| | | | | | | 7433 | | | | | ClinVar |
| 14:024724663 | Missense | 0.985 | 29.5 | -1.42 | 0.407 | 0.0156704 | NFE | 121200 | 1249 | 6 | ExAC, |
| | | | | | | 992345 | | | | | ClinVar, |
| | | | | | | | | | | | and |
| | | | | | | | | | | | VariBench |
| 14:024731434 | Missense | 0.978 | 22.7 | -0.33 | 0.823 | 0.0058144 | NFE | 120520 | 480 | 2 | ExAC, |
| | | | | | | 8169347 | | | | | ClinVar, |
| | | | | | | | | | | | and |
| | | | | | | | | | | | VariBench |
| 14:088452941 | Missense | 0.991 | 23.4 | -2.67 | 0.9 | 0.0036304 | NFE | 117482 | 302 | 2 | ExAC and |
| | | | | | | 3411377 | | | | | ClinVar |
| 15:072638893 | Synonymous | . | 19.85 | . | . | 0.0004319 | AMR | 121304 | 6 | 0 | ExAC and |
| | | | | | | 2812716 | | | | | ClinVar |
| 15:072641434 | Synonymous | . | 19.55 | . | . | 0 | . | . | . | . | ClinVar |
| 15:080472526 | Missense | 1.0 | 28.4 | -5.4 | 0.758 | 0.0229433 | NFE | 119604 | 1973 | 19 | ExAC, |
| | | | | | | 272395 | | | | | ClinVar, |
| | | | | | | | | | | | and |
| | | | | | | | | | | | VariBench |
| 15:089865073 | Missense | 0.995 | 17.7 | -2.82 | 0.764 | 0.0089165 | NFE | 121386 | 756 | 3 | ExAC, |
| | | | | | | 2929717 | | | | | ClinVar, |
| | | | | | | | | | | | and |
| | | | | | | | | | | | VariBench |
| 16:000222980 | Synonymous | . | 2.648 | . | . | 0 | . | . | . | . | ClinVar |
| 16:003293257 | Missense | 0.0 | 0.003 | 1.05 | 0.492 | 0.0021430 | NFE | 121396 | 176 | 2 | ExAC, |
| | | | | | | 2841386 | | | | | ClinVar, |
| | | | | | | | | | | | and |
| | | | | | | | | | | | VariBench |
| 16:003293310 | Missense | 0.001 | 0.001 | 1.93 | 0.514 | 0.0032516 | NFE | 121408 | 212 | 6 | ExAC, |
| | | | | | | 1831695 | | | | | ClinVar, |
| | | | | | | | | | | | and |
| | | | | | | | | | | | VariBench |
| 16:003293403 | Missense | 0.939 | 1.747 | -0.95 | 0.218 | 0.0079112 | NFE | 121410 | 660 | 4 | ExAC, |
| | | | | | | 9757267 | | | | | ClinVar, |
| | | | | | | | | | | | and |
| | | | | | | | | | | | VariBench |
| 16:003304626 | Missense | 0.995 | 23.3 | -1.3 | 0.386 | 0.3150096 | EAS | 92068 | 6211 | 1038 | ExAC, |
| | | | | | | 92606 | | | | | ClinVar, |
| | | | | | | | | | | | and |
| | | | | | | | | | | | VariBench |
| 16:023200921 | Missense | 0.001 | 0.114 | 0.92 | 0.221 | 0.0373822 | AFR | 121392 | 561 | 8 | ExAC and |
| | | | | | | 794542 | | | | | ClinVar |
| 16:023200963 | Missense | 0.004 | 14.77 | -0.82 | 0.741 | 0.0074396 | NFE | 121330 | 566 | 4 | ExAC and |
| | | | | | | 280186 | | | | | ClinVar |
| 16:023360165 | Missense | 0.997 | 16.78 | -4.04 | 0.805 | 0.0068986 | NFE | 121310 | 575 | 3 | ExAC and |
| | | | | | | 2027594 | | | | | ClinVar |
| 16:053720436 | Missense | 0.005 | 17.44 | -0.19 | 0.081 | 0.1036573 | AFR | 121106 | 4265 | 138 | ExAC and |
| | | | | | | 62849 | | | | | ClinVar |
| 16:088502971 | Inframe | . | 17.04 | -5.38 | . | 0.0059508 | SAS | 17306 | 76 | 2 | ExAC and |
| | indel | | | | | 736389 | | | | | ClinVar |
| 17:003550800 | Missense | 0.007 | 12.64 | -0.02 | 0.351 | 0.0596914 | AFR | 121160 | 634 | 13 | ExAC and |
| | | | | | | 175506 | | | | | ClinVar |
| 17:007123838 | Missense | 0.019 | 8.442 | -1.95 | 0.113 | 0.1135270 | AFR | 121338 | 1136 | 68 | ExAC, |
| | | | | | | 34828 | | | | | ClinVar, |
| | | | | | | | | | | | and |
| | | | | | | | | | | | VariBench |
| 17:015134364 | Missense | 1.0 | 29.0 | -5.48 | 0.926 | 0.0073593 | NFE | 120452 | 560 | 2 | ExAC, |
| | | | | | | 9913383 | | | | | ClinVar, |
| | | | | | | | | | | | and |
| | | | | | | | | | | | VariBench |
| 17:041063017 | Synonymous | . | 7.36 | . | . | 0.0012710 | EAS | 121406 | 11 | 0 | ExAC and |
| | | | | | | 8851398 | | | | | ClinVar |
| 17:078078341 | Intron | . | . | . | . | 0.0053133 | NFE | 101332 | 359 | 2 | ExAC and |
| | | | | | | 6583313 | | | | | ClinVar |
| 19:007125518 | Missense | 0.992 | 34.0 | -2.42 | 0.662 | 0.0225317 | SAS | 121148 | 1068 | 11 | ExAC and |
| | | | | | | 989098 | | | | | ClinVar |
| 19:012917556 | Synonymous | . | 19.19 | . | . | 0.0 | NFE | 28346 | 0 | 0 | ExAC and |
| | | | | | | | | | | | ClinVar |
| 19:012917562 | Synonymous | . | 21.2 | . | . | 0 | . | . | . | . | ClinVar |
| 19:018710530 | Missense | 0.004 | 16.56 | -1.9 | 0.436 | 0.0005323 | AFR | 91230 | 5 | 0 | ExAC, |
| | | | | | | 39632686 | | | | | ClinVar, |
| | | | | | | | | | | | and |
| | | | | | | | | | | | VariBench |
| 19:036339044 | Missense | 0.996 | 30.0 | -1.73 | 0.738 | 0.0390278 | EAS | 119744 | 327 | 10 | ExAC, |
| | | | | | | 10236 | | | | | ClinVar, |
| | | | | | | | | | | | and |
| | | | | | | | | | | | VariBench |
| 19:045860626 | Missense | 0.982 | 25.3 | -2.43 | 0.934 | 0.0071038 | SAS | 120472 | 158 | 2 | ExAC, |
| | | | | | | 2513661 | | | | | ClinVar, |
| | | | | | | | | | | | and |
| | | | | | | | | | | | VariBench |
| 20:043255233 | Missense | 1.0 | 22.3 | -5.52 | 0.941 | 0.0025961 | AFR | 121358 | 38 | 2 | ExAC, |
| | | | | | | 5384615 | | | | | ClinVar, |
| | | | | | | | | | | | and |
| | | | | | | | | | | | VariBench |
| 20:043280227 | Missense | 0.001 | 23.1 | 0.28 | 0.038 | 0.1385834 | SAS | 13566 | 1628 | 80 | ExAC and |
| | | | | | | 82485 | | | | | ClinVar |
| 22:050962500 | Missense | 1.0 | 16.29 | -3.47 | 0.847 | 0.0023425 | AMR | 119592 | 89 | 2 | ExAC and |
| | | | | | | 2993233 | | | | | ClinVar |
| 22:051064039 | Missense | 0.0 | 8.571 | 0.13 | 0.106 | 0.5392830 | NFE | 120940 | 2910 | 14735 | ExAC and |
| | | | | | | 24552 | | | 2 | | ClinVar |
| 22:051064416 | Missense | 0.134 | 17.91 | -1.17 | 0.126 | 0.3947811 | AMR | 40070 | 7001 | 839 | ExAC and |
| | | | | | | 44781 | | | | | ClinVar |

TABLE 5
Summary VGD scores stratified by ClinVar and VariBench category

| | | | | | | | | Median max | |
|---|---|---|---|---|---|---|---|---|---|
| | | VGD | | VGD | | Median # of | | population | |
| | # of | Median | | mean | | homozygotes | | frequency | |
| | Variants | (IQR) | | (sd) | | (IQR) | | (IQR) | |
| ClinVar 2 | 2087 | 0 | (0, 0.01) | 0.0496 | (0.158) | 8 | (1, 211) | 0.0211 | (0.00377, 0.118) |
| ClinVar 3 | 1597 | 0 | (0, 0.04) | 0.0624 | (0.142) | 5 | (0, 166) | 0.0148 | (0.000606, 0.102) |
| ClinVar 4 | 1280 | 0.99 | (0.94, 1) | 0.921 | (0.172) | 0 | (0, 0) | 0 | (0, 1.5e−05) |
| ClinVar 5 | 6793 | 1 | (0.99, 1) | 0.975 | (0.129) | 0 | (0, 0) | 0 | (0, 4.51e−05) |
| ClinVar 5.1 | 6653 | 1 | (1, 1) | 0.991 | (0.0575) | 0 | (0, 0) | 0 | (0, 3.02e−05) |
| ClinVar 5.2 | 140 | 0.02 | (0, 0.508) | 0.249 | (0.33) | 6 | (2, 38) | 0.0118 | (0.00111, 0.0416) |
| Other or | 6406 | 0.755 | (0.3, 0.97) | 0.631 | (0.364) | 0 | (0, 0) | 0 | (0, 0.000108) |
| unkown | | | | | | | | | |
| variants | | | | | | | | | |
| All ClinVar | 17748 | 0.97 | (0.14, 1) | 0.661 | (0.42) | 0 | (0, 3) | 0 | (0, 0.000405) |
| variants | | | | | | | | | |
| VariBench | 753 | 0 | (0, 0.29) | 0.193 | (0.312) | 8 | (0, 135) | 0.0159 | (0.000207, 0.0865) |
| Benign | | | | | | | | | |
| VariBench | 5521 | 0.97 | (0.83, 0.99) | 0.869 | (0.216) | 0 | (0, 0) | 0 | (0, 2.2e−05) |
| Pathogenic | | | | | | | | | |
| VariBench | 5431 | 0.98 | (0.85, 0.99) | 0.883 | (0.189) | 0 | (0, 0) | 0 | (0, 1.53e−05) |
| Pathogenic | | | | | | | | | |
| group 1 | | | | | | | | | |
| VariBench | 90 | 0 | (0, 0.02) | 0.0649 | (0.19) | 9.5 | (3, 28) | 0.0186 | (0.00906, 0.0468) |
| Pathogenic | | | | | | | | | |
| group 2 | | | | | | | | | |
| All VariBench | 6274 | 0.96 | (0.75, 0.99) | 0.788 | (0.318) | 0 | (0, 0) | 0 | (0, 8.7e−05) |
| variants | | | | | | | | | |
| All ExAC | 242782 | 0.32 | (0.03, 0.77) | 0.403 | (0.372) | 0 | (0, 0) | 8.66e−05 | (1.65e−05, 0.000176) |
| variants | | | | | | | | | |

| | PP2 | | CADD | | PROVEAN | | VEST | |
|---|---|---|---|---|---|---|---|---|
| | median | | median | | median | | median | |
| | (IQR) | | (IQR) | | (IQR) | | (IQR) | |
| ClinVar 2 | 0.141 | (0.002, 0.972) | 12.6 | (6.68, 18.6) | −0.99 | (−2.35, −0.26) | 0.144 | (0.063, 0.35) |
| ClinVar 3 | 0.073 | (0.003, 0.937) | 12.9 | (7.34, 18.2) | −1.08 | (−2.2, −0.345) | 0.144 | (0.0632, 0.326) |
| ClinVar 4 | 1 | (0.982, 1) | 23.8 | (19.6, 33) | −4.72 | (−6.8, −2.97) | 0.925 | (0.778, 0.974) |
| ClinVar 5 | 1 | (0.985, 1) | 22.8 | (18.6, 29.8) | −4.76 | (−6.67, −3.2) | 0.941 | (0.847, 0.981) |
| ClinVar 5.1 | 1 | (0.988, 1) | 22.8 | (18.8, 30) | −4.82 | (−6.72, −3.28) | 0.945 | (0.855, 0.982) |
| ClinVar 5.2 | 0.841 | (0.005, 0.998) | 18.7 | (11.9, 23) | −1.96 | (−3.66, −0.503) | 0.463 | (0.191, 0.814) |
| Other or | 0.979 | (0.151, 1) | 21.1 | (14, 25) | −2.75 | (−4.73, −1.22) | 0.738 | (0.364, 0.923) |
| unkown variants | | | | | | | | |
| All ClinVar | 0.995 | (0.329, 1) | 21.2 | (13.8, 25.6) | −3.33 | (−5.49, −1.44) | 0.834 | (0.381, 0.956) |
| variants | | | | | | | | |
| VariBench | 0.243 | (0.003, 0.985) | 17.2 | (9.73, 23) | −1.31 | (−2.8, −0.39) | 0.183 | (0.07, 0.461) |
| Benign | | | | | | | | |
| VariBench | 1 | (0.975, 1) | 22.8 | (18.4, 27.3) | −4.71 | (−6.51, −2.97) | 0.935 | (0.828, 0.977) |
| Pathogenic | | | | | | | | |
| VariBench | 1 | (0.978, 1) | 22.8 | (18.4, 27.4) | −4.75 | (−6.53, −3.03) | 0.937 | (0.836, 0.977) |
| Pathogenic | | | | | | | | |
| group 1 | | | | | | | | |
| VariBench | 0.601 | (0.00525, 0.992) | 21.4 | (11, 24.4) | −1.58 | (−2.88, −0.5) | 0.402 | (0.149, 0.728) |
| Pathogenic | | | | | | | | |
| group 2 | | | | | | | | |
| All VariBench | 0.999 | (0.892, 1) | 22.6 | (17.5, 26.8) | −4.34 | (−6.25, −2.51) | 0.918 | (0.739, 0.973) |
| variants | | | | | | | | |
| All ExAC | 0.771 | (0.023, 0.998) | 16.2 | (9.85, 22.7) | −1.91 | (−3.59, −0.76) | 0.408 | (0.166, 0.737) |
| variants | | | | | | | | |

TABLE 6
Summary VGD scores stratified by mutation type

| | | | VGD | Median # of | Median max | |
|---|---|---|---|---|---|---|
| | # of | VGD Median | mean | homozygotes | population | PP2 median |
| | Variants | (IQR) | (sd) | (IQR) | frequency (IQR) | (IQR) |
| Unknown variants | 331 | 0 (0, 0) | 0.0181 | 0 (0, 0) | 0.000129 | NA (NA, NA) |
| | | | (0.113) | | (3.29e−05, 0.00043) | |
| Non-coding change | 360 | 0.02 (0, 0.07) | 0.116 | 0 (0, 0) | 0.000135 | NA (NA, NA) |
| variants | | | (0.243) | | (6.15e−05, 0.000443) | |
| Coding unknown | 3 | 0.02 (0.01, 0.07) | 0.0467 | 0 (0, 48.5) | 0.000123 | NA (NA, NA) |
| variants | | | (0.0643) | | (7.67e−05, 0.039) | |
| Intergenic | 66 | 0 (0, 0) | 0.0395 | 0 (0, 0) | 0.000207 | NA (NA, NA) |
| | | | (0.145) | | (0.000133, 0.000638) | |
| Intronic variants | 7586 | 0 (0, 0.01) | 0.0454 | 0 (0, 0) | 9.02e−05 | NA (NA, NA) |
| | | | (0.141) | | (2.09e−05, 0.000209) | |
| Synonymous | 63473 | 0.02 (0, 0.15) | 0.0981 | 0 (0, 0) | 9.61e−05 | NA (NA, NA) |
| variants | | | (0.146) | | (3e−05, 0.000219) | |
| Non-essential splice | 13915 | 0.02 (0, 0.11) | 0.116 | 0 (0, 0) | 8.7e−05 | NA (NA, NA) |
| site varaints | | | (0.214) | | (1.69e−05, 0.000184) | |
| 3′ variants | 2984 | 0 (0, 0) | 0.0109 | 0 (0, 0) | 0.000133 | NA (NA, NA) |
| | | | (0.0646) | | (6.06e−05, 0.000459) | |
| 5′ variants | 1657 | 0 (0, 0) | 0.0214 | 0 (0, 0) | 9.94e−05 | NA (NA, NA) |
| | | | (0.107) | | (1.98e−05, 0.000258) | |
| Stoploss variants | 105 | 0.36 (0.05, 0.83) | 0.422 | 0 (0, 0) | 6.07e−05 | NA (NA, NA) |
| | | | (0.364) | | (1.67e−05, 0.000107) | |
| Missense variants | 132248 | 0.57 (0.23, 0.83) | 0.534 | 0 (0, 0) | 8.64e−05 | 0.771 |
| | | | (0.323) | | (1.6e−05, 0.000172) | (0.023, 0.998) |
| Inframe indel | 2353 | 0.82 (0.53, 0.97) | 0.704 | 0 (0, 0) | 7.57e−05 | NA (NA, NA) |
| | | | (0.311) | | (1.54e−05, 0.000125) | |
| Startloss variants | 277 | 0.98 (0.98, 0.99) | 0.943 | 0 (0, 0) | 7.69e−05 | NA (NA, NA) |
| | | | (0.167) | | (1.9e−05, 0.000136) | |
| Essential splice site | 4704 | 0.99 (0.99, 1) | 0.954 | 0 (0, 0) | 8.64e−05 | NA (NA, NA) |
| variants (exon) | | | (0.167) | | (1.59e−05, 0.000131) | |
| Essential splice site | 3276 | 1 (0.99, 1) | 0.958 | 0 (0, 0) | 6.06e−05 | NA (NA, NA) |
| variants (intron) | | | (0.173) | | (1.51e−05, 0.000116) | |
| Nonsense variants | 3812 | 1(0.99, 1) | 0.99 | 0 (0, 0) | 6.06e−05 | NA (NA, NA) |
| | | | (0.064) | | (1.51e−05, 0.000102) | |
| Frameshift | 5632 | 1 (0.99, 1) | 0.986 | 0 (0, 0) | 6.04e−05 | NA (NA, NA) |
| | | | (0.0825) | | (1.5e−05, 9.93e−05) | |
| All ExAC variants | 242782 | 0.32 (0.03, 0.77) | 0.403 | 0 (0, 0) | 8.66e−05 | 0.771 |
| | | | (0.372) | | (1.65e−05, 0.000176) | (0.023, 0.998) |

| | CADD median | PROVEAN | VEST median |
|---|---|---|---|
| | (IQR) | median (IQR) | (IQR) |
| Unknown variants | 8.04 (4.17, 14.1) | NA (NA, NA) | NA (NA, NA) |
| Non-coding change | 10.1 (5.25, 12.8) | NA (NA, NA) | NA (NA, NA) |
| variants | | | |
| Coding unknown | 10 (8.7, 11.8) | NA (NA, NA) | NA (NA, NA) |
| variants | | | |
| Intergenic | 4.01 (2.02, 6.76) | NA (NA, NA) | NA (NA, NA) |
| Intronic variants | 6.72 (3.01, 11.5) | NA (NA, NA) | NA (NA, NA) |
| Synonymous | 11.6 (6.54, 16.2) | NA (NA, NA) | NA (NA, NA) |
| variants | | | |
| Non-essential splice | 9.95 (5.67, 13.6) | NA (NA, NA) | NA (NA, NA) |
| site varaints | | | |
| 3′ variants | 5.61 (2.74, 8.84) | NA (NA, NA) | NA (NA, NA) |
| 5′ variants | 11 (8.43, 15.3) | NA (NA, NA) | NA (NA, NA) |
| Stoploss variants | 14.5 (11.3, 19.5) | NA (NA, NA) | 1 (1, 1) |
| Missense variants | 20.3 (13, 24) | −1.88 (−3.53, −0.75) | 0.403 (0.164, 0.729) |
| Inframe indel | 19.2 (14.8, 22.4) | −5.9 (−10, −2.27) | 1 (0.999, 1) |
| Startloss variants | 16.9 (12.5, 21.9) | NA (NA, NA) | 0.86 (0.645, 0.936) |
| Essential splice site | 19.5 (13.7, 23.5) | NA (NA, NA) | 1 (0.998, 1) |
| variants (exon) | | | |
| Essential splice site | 22 (15.9, 23.8) | NA (NA, NA) | NA (NA, NA) |
| variants (intron) | | | |
| Nonsense variants | 32.5 (19.4, 39) | NA (NA, NA) | 1 (1, 1) |
| Frameshift | 23 (17.6, 35) | −2.92 (−6.68, −0.408) | 1 (0.999, 1) |
| All ExAC variants | 16.2 (9.85, 22.7) | −1.91 (−3.59, −0.76) | 0.408 (0.166, 0.737) |

TABLE 7
Bootstrap 99% one-sided confidence interval thresholds for each gene

| | # of | | | | # of |
|---|---|---|---|---|---|
| Gene | ClinVar 5 | Average | 99% | 99.9% | ClinVar 5.1 |
| Name | variants | VGD naive | cutoff | cutoff | variants |
| PEX10 | 10 | 0.98 | 0.967 | 0.963 | 10 |
| NPHP4 | 8 | NA | NA | NA | 8 |
| PLEKHG5 | 7 | NA | NA | NA | 7 |
| PLOD1 | 5 | NA | NA | NA | 5 |
| ALPL | 22 | 0.934545454545455 | 0.893636363636364 | 0.878180909090909 | 22 |
| HSPG2 | 1 | NA | NA | NA | 1 |
| HMGCL | 5 | NA | NA | NA | 5 |
| FUCA1 | 8 | NA | NA | NA | 8 |
| SEPN1 | 9 | NA | NA | NA | 9 |
| NDUFS5 | 0 | NA | NA | NA | 0 |
| PPT1 | 8 | NA | NA | NA | 8 |
| ZMPSTE24 | 9 | NA | NA | NA | 9 |
| CLDN19 | 3 | NA | NA | NA | 3 |
| P3H1 | 9 | NA | NA | NA | 8 |
| MPL | 9 | NA | NA | NA | 9 |
| ST3GAL3 | 1 | NA | NA | NA | 1 |
| MMACHC | 13 | 0.98 | 0.964615384615385 | 0.958461538461538 | 13 |
| POMGNT1 | 20 | 0.936 | 0.7965 | 0.736 | 19 |
| CPT2 | 18 | 0.919444444444444 | 0.863333333333333 | 0.842777777777778 | 18 |
| DHCR24 | 7 | NA | NA | NA | 7 |
| ALG6 | 7 | NA | NA | NA | 6 |
| SLC35D1 | 3 | NA | NA | NA | 3 |
| ACADM | 19 | 0.878421052631579 | 0.766842105263158 | 0.718947368421053 | 18 |
| DPYD | 9 | NA | NA | NA | 7 |
| AGL | 16 | 0.938125 | 0.755 | 0.693125 | 16 |
| DBT | 24 | 0.905 | 0.782083333333333 | 0.72916625 | 22 |
| TSHB | 3 | NA | NA | NA | 3 |
| HSD3B2 | 10 | 0.968 | 0.932 | 0.920999 | 10 |
| HFE2 | 8 | NA | NA | NA | 8 |
| CTSK | 7 | NA | NA | NA | 7 |
| HAX1 | 8 | NA | NA | NA | 8 |
| GBA | 68 | 0.821323529411765 | 0.759704411764706 | 0.734116470588235 | 65 |
| PKLR | 8 | NA | NA | NA | 8 |
| LMNA | 66 | 0.78969696969697 | 0.714392424242424 | 0.691969545454546 | 64 |
| NTRK1 | 15 | 0.662 | 0.395333333333333 | 0.295997333333333 | 13 |
| MPZ | 50 | 0.8368 | 0.78 | 0.7601984 | 50 |
| CD247 | 6 | NA | NA | NA | 6 |
| FASLG | 3 | NA | NA | NA | 3 |
| NPHS2 | 11 | 0.874545454545455 | 0.632727272727273 | 0.538165454545455 | 11 |
| LAMC2 | 4 | NA | NA | NA | 4 |
| LAMB3 | 14 | 0.84 | 0.66 | 0.592142857142857 | 14 |
| USH2A | 87 | 0.963103448275862 | 0.92321724137931 | 0.905860459770115 | 86 |
| RAB3GAP2 | 5 | NA | NA | NA | 5 |
| LBR | 7 | NA | NA | NA | 7 |
| ADCK3 | 14 | 0.890714285714286 | 0.696428571428571 | 0.634278571428571 | 13 |
| GJC2 | 8 | NA | NA | NA | 8 |
| TBCE | 1 | NA | NA | NA | 1 |
| LYST | 47 | 0.990212765957447 | 0.976170212765957 | 0.970425531914894 | 47 |
| FH | 26 | 0.943076923076923 | 0.869226923076923 | 0.846536923076923 | 24 |
| HADHA | 9 | NA | NA | NA | 8 |
| HADHB | 5 | NA | NA | NA | 5 |
| MPV17 | 26 | 0.971153846153846 | 0.955 | 0.947692307692308 | 26 |
| SRD5A2 | 14 | 0.853571428571429 | 0.758571428571429 | 0.722854285714286 | 14 |
| LRPPRC | 1 | NA | NA | NA | 1 |
| LHCGR | 24 | 0.849583333333333 | 0.8125 | 0.799582083333333 | 24 |
| PEX13 | 2 | NA | NA | NA | 2 |
| ALMS1 | 6 | NA | NA | NA | 6 |
| DGUOK | 4 | NA | NA | NA | 4 |
| MOGS | 3 | NA | NA | NA | 3 |
| SUCLG1 | 3 | NA | NA | NA | 3 |
| SFTPB | 1 | NA | NA | NA | 1 |
| ST3GAL5 | 2 | NA | NA | NA | 2 |
| EIF2AK3 | 2 | NA | NA | NA | 2 |
| NPHP1 | 4 | NA | NA | NA | 4 |
| IL1RN | 2 | NA | NA | NA | 2 |
| ERCC3 | 4 | NA | NA | NA | 4 |
| RAB3GAP1 | 8 | NA | NA | NA | 8 |
| ZEB2 | 62 | 0.952258064516129 | 0.896612903225806 | 0.872901935483871 | 61 |
| NEB | 7 | NA | NA | NA | 7 |
| ABCB11 | 7 | NA | NA | NA | 7 |
| LRP2 | 19 | 0.948947368421053 | 0.851052631578947 | 0.818421052631579 | 19 |
| ITGA6 | 0 | NA | NA | NA | 0 |
| CHRNA1 | 14 | 0.86 | 0.79 | 0.769997857142857 | 14 |
| AGPS | 4 | NA | NA | NA | 4 |
| HIBCH | 1 | NA | NA | NA | 1 |
| STAT1 | 24 | 0.791666666666667 | 0.684583333333333 | 0.650411666666667 | 24 |
| CASP10 | 6 | NA | NA | NA | 6 |
| ALS2 | 23 | 0.996086956521739 | 0.990869565217391 | 0.988695652173913 | 23 |
| ICOS | 0 | NA | NA | NA | 0 |
| FASTKD2 | 1 | NA | NA | NA | 1 |
| ACADL | 0 | NA | NA | NA | 0 |
| CPS1 | 5 | NA | NA | NA | 5 |
| ABCA12 | 13 | 0.908461538461538 | 0.688453846153846 | 0.602307692307692 | 12 |
| BCS1L | 13 | 0.913076923076923 | 0.856923076923077 | 0.834613076923077 | 13 |
| CYP27A1 | 55 | 0.922363636363636 | 0.846905454545455 | 0.818545272727273 | 53 |
| WNT10A | 8 | NA | NA | NA | 6 |
| NHEJ1 | 3 | NA | NA | NA | 3 |
| COL4A4 | 6 | NA | NA | NA | 6 |
| COL4A3 | 6 | NA | NA | NA | 6 |
| SP110 | 12 | 0.926666666666667 | 0.8275 | 0.763333333333333 | 12 |
| CHRND | 8 | NA | NA | NA | 8 |
| CHRNG | 5 | NA | NA | NA | 5 |
| COL6A3 | 8 | NA | NA | NA | 8 |
| AGXT | 16 | 0.8275 | 0.61936875 | 0.53187375 | 14 |
| WNT7A | 5 | NA | NA | NA | 5 |
| XPC | 2 | NA | NA | NA | 1 |
| BTD | 161 | 0.839813664596273 | 0.788941614906832 | 0.770683167701863 | 155 |
| GLB1 | 42 | 0.97 | 0.947140476190476 | 0.935714285714286 | 42 |
| CRTAP | 5 | NA | NA | NA | 5 |
| MYD88 | 3 | NA | NA | NA | 3 |
| PTH1R | 9 | NA | NA | NA | 9 |
| TREX1 | 23 | 0.904782608695652 | 0.830869565217391 | 0.80608347826087 | 23 |
| COL7A1 | 36 | 0.952777777777778 | 0.917222222222222 | 0.903333333333333 | 36 |
| SLC25A20 | 9 | NA | NA | NA | 9 |
| LAMB2 | 9 | NA | NA | NA | 9 |
| AMT | 7 | NA | NA | NA | 7 |
| RFT1 | 1 | NA | NA | NA | 1 |
| HESX1 | 8 | NA | NA | NA | 8 |
| GBE1 | 16 | 0.95 | 0.820625 | 0.77625 | 16 |
| POU1F1 | 17 | 0.974117647058824 | 0.933523529411765 | 0.919998235294118 | 17 |
| HGD | 17 | 0.985882352941176 | 0.971176470588235 | 0.965882352941176 | 17 |
| IQCB1 | 10 | 0.995 | 0.98 | 0.975 | 10 |
| ACAD9 | 6 | NA | NA | NA | 5 |
| NPHP3 | 9 | NA | NA | NA | 9 |
| PCCB | 21 | 0.96 | 0.887619047619048 | 0.858571428571429 | 21 |
| MRPS22 | 2 | NA | NA | NA | 2 |
| ATR | 4 | NA | NA | NA | 3 |
| CLRN1 | 10 | 0.909 | 0.823 | 0.791 | 9 |
| GFM1 | 4 | NA | NA | NA | 4 |
| IFT80 | 5 | NA | NA | NA | 5 |
| BCHE | 11 | 0.592727272727273 | 0.325454545454545 | 0.251811818181818 | 8 |
| DNAJC19 | 3 | NA | NA | NA | 3 |
| ALG3 | 5 | NA | NA | NA | 4 |
| CLDN1 | 0 | NA | NA | NA | 0 |
| IDUA | 30 | 0.726333333333333 | 0.572666666666667 | 0.535330666666667 | 30 |
| DOK7 | 12 | 0.970833333333333 | 0.895 | 0.87 | 12 |
| EVC2 | 6 | NA | NA | NA | 6 |
| EVC | 5 | NA | NA | NA | 4 |
| SGCB | 7 | NA | NA | NA | 7 |
| SRD5A3 | 6 | NA | NA | NA | 6 |
| GNRHR | 18 | 0.798333333333333 | 0.68555 | 0.646665 | 18 |
| FRAS1 | 4 | NA | NA | NA | 4 |
| ANTXR2 | 8 | NA | NA | NA | 8 |
| COQ2 | 4 | NA | NA | NA | 4 |
| DMP1 | 4 | NA | NA | NA | 4 |
| HADH | 4 | NA | NA | NA | 4 |
| PRSS12 | 0 | NA | NA | NA | 0 |
| MFSD8 | 8 | NA | NA | NA | 8 |
| MMAA | 4 | NA | NA | NA | 4 |
| FGA | 9 | NA | NA | NA | 9 |
| ETFDH | 11 | 0.97 | 0.943636363636364 | 0.933636363636364 | 11 |
| AGA | 8 | NA | NA | NA | 8 |
| TLR3 | 1 | NA | NA | NA | 0 |
| F11 | 13 | 0.857692307692308 | 0.680769230769231 | 0.598460769230769 | 13 |
| NDUFS6 | 1 | NA | NA | NA | 1 |
| NSUN2 | 3 | NA | NA | NA | 3 |
| AMACR | 2 | NA | NA | NA | 2 |
| LIFR | 2 | NA | NA | NA | 2 |
| OXCT1 | 7 | NA | NA | NA | 7 |
| MOCS2 | 14 | 0.926428571428571 | 0.712857142857143 | 0.640714285714286 | 14 |
| NDUFS4 | 4 | NA | NA | NA | 4 |
| ERCC8 | 5 | NA | NA | NA | 5 |
| NDUFAF2 | 1 | NA | NA | NA | 1 |
| SMN2 | 0 | NA | NA | NA | 0 |
| SMN1 | 18 | 0.798888888888889 | 0.62 | 0.542776666666667 | 18 |
| HEXB | 13 | 0.832307692307692 | 0.609984615384615 | 0.53692 | 11 |
| AP3B1 | 5 | NA | NA | NA | 5 |
| ARSB | 18 | 0.94 | 0.809444444444444 | 0.762221111111111 | 18 |
| ADGRV1 | 19 | 0.945789473684211 | 0.787894736842105 | 0.735263157894737 | 19 |
| HSD17B4 | 11 | 0.910909090909091 | 0.779090909090909 | 0.735451818181818 | 10 |
| ALDH7A1 | 7 | NA | NA | NA | 7 |
| SLC22A5 | 24 | 0.861666666666667 | 0.72375 | 0.674999166666667 | 23 |
| UQCRQ | 1 | NA | NA | NA | 1 |
| SIL1 | 4 | NA | NA | NA | 4 |
| SLC26A2 | 14 | 0.976428571428571 | 0.959285714285714 | 0.953571428571429 | 14 |
| IL12B | 1 | NA | NA | NA | 1 |
| NSD1 | 130 | 0.986230769230769 | 0.964230769230769 | 0.956230769230769 | 130 |
| PROP1 | 15 | 0.992666666666667 | 0.986666666666667 | 0.984666666666667 | 15 |
| DSP | 20 | 0.9185 | 0.783 | 0.7334965 | 19 |
| NHLRC1 | 8 | NA | NA | NA | 8 |
| ALDH5A1 | 3 | NA | NA | NA | 3 |
| NEU1 | 13 | 0.864615384615385 | 0.781530769230769 | 0.753845384615385 | 13 |
| CYP21A1P | 0 | NA | NA | NA | 0 |
| CYP21A2 | 19 | 0.702631578947368 | 0.539468421052632 | 0.498941578947368 | 18 |
| MOCS1 | 7 | NA | NA | NA | 7 |
| MUT | 15 | 0.962666666666667 | 0.908 | 0.886665333333333 | 15 |
| PKHD1 | 38 | 0.959736842105263 | 0.900263157894737 | 0.878420526315789 | 38 |
| RAB23 | 1 | NA | NA | NA | 1 |
| SLC17A5 | 8 | NA | NA | NA | 8 |
| BCKDHB | 25 | 0.986 | 0.974 | 0.9695996 | 25 |
| SLC35A1 | 0 | NA | NA | NA | 0 |
| NDUFAF4 | 1 | NA | NA | NA | 1 |
| GRIK2 | 0 | NA | NA | NA | 0 |
| PDSS2 | 2 | NA | NA | NA | 2 |
| OSTM1 | 1 | NA | NA | NA | 1 |
| TSPYL1 | 0 | NA | NA | NA | 0 |
| LAMA2 | 34 | 0.980882352941176 | 0.925588235294118 | 0.906764705882353 | 34 |
| ENPP1 | 13 | 0.980769230769231 | 0.96 | 0.951537692307692 | 13 |
| AHI1 | 15 | 0.994 | 0.988 | 0.985333333333333 | 15 |
| PEX7 | 14 | 0.808571428571429 | 0.5757 | 0.51357 | 14 |
| IFNGR1 | 14 | 0.962857142857143 | 0.920714285714286 | 0.904285714285714 | 14 |
| STX11 | 3 | NA | NA | NA | 3 |
| EPM2A | 7 | NA | NA | NA | 7 |
| GTF2H5 | 2 | NA | NA | NA | 2 |
| PLG | 11 | 0.837272727272727 | 0.610909090909091 | 0.509999090909091 | 10 |
| FAM20C | 1 | NA | NA | NA | 1 |
| FAM126A | 3 | NA | NA | NA | 3 |
| DDC | 6 | NA | NA | NA | 6 |
| GUSB | 19 | 0.887894736842105 | 0.754731578947368 | 0.709472105263158 | 18 |
| ASL | 11 | 0.905454545454546 | 0.764545454545455 | 0.701817272727273 | 11 |
| SBDS | 13 | 0.972307692307692 | 0.934615384615385 | 0.919999230769231 | 13 |
| POR | 8 | NA | NA | NA | 8 |
| ABCB4 | 17 | 0.878235294117647 | 0.698817647058824 | 0.597058235294118 | 15 |
| PEX1 | 11 | 0.921818181818182 | 0.846363636363636 | 0.824545454545455 | 11 |
| COL1A2 | 26 | 0.962307692307692 | 0.929615384615385 | 0.91846 | 26 |
| RELN | 4 | NA | NA | NA | 4 |
| SLC26A4 | 46 | 0.940217391304348 | 0.888910869565217 | 0.872391086956522 | 46 |
| DLD | 11 | 0.969090909090909 | 0.941818181818182 | 0.932724545454545 | 11 |
| CFTR | 255 | 0.904862745098039 | 0.870509803921569 | 0.858862470588235 | 247 |
| CLN8 | 5 | NA | NA | NA | 5 |
| TUSC3 | 2 | NA | NA | NA | 2 |
| SFTPC | 6 | NA | NA | NA | 5 |
| ESCO2 | 32 | 0.9340625 | 0.8096875 | 0.7778125 | 32 |
| STAR | 13 | 0.871538461538461 | 0.666923076923077 | 0.575383076923077 | 13 |
| HGSNAT | 12 | 0.974166666666667 | 0.9375 | 0.925833333333333 | 12 |
| TTPA | 16 | 0.9125 | 0.804375 | 0.761868125 | 16 |
| GDAP1 | 17 | 0.857647058823529 | 0.758235294117647 | 0.72411705882353 | 17 |
| CA2 | 5 | NA | NA | NA | 4 |
| CNGB3 | 8 | NA | NA | NA | 8 |
| NBN | 34 | 0.9629411876470588 | 0.891467647058824 | 0.869705882352941 | 33 |
| TMEM67 | 12 | 0.960833333333333 | 0.916666666666667 | 0.902498333333333 | 12 |
| PDP1 | 1 | NA | NA | NA | 1 |
| UQCRB | 0 | NA | NA | NA | 0 |
| VPS13B | 37 | 0.961351351351351 | 0.895405405405405 | 0.867837837837838 | 36 |
| RRM2B | 37 | 0.944054054054054 | 0.885672972972973 | 0.866756486486487 | 37 |
| TNFRSF11B | 2 | NA | NA | NA | 2 |
| TRAPPC9 | 3 | NA | NA | NA | 3 |
| CYP11B1 | 10 | 0.905 | 0.822 | 0.797999 | 10 |
| PLEC | 5 | NA | NA | NA | 5 |
| DOCK8 | 1 | NA | NA | NA | 1 |
| VLDLR | 5 | NA | NA | NA | 5 |
| GLDC | 10 | 0.973 | 0.956 | 0.950999 | 10 |
| APTX | 7 | NA | NA | NA | 7 |
| B4GALT1 | 0 | NA | NA | NA | 0 |
| GALT | 231 | 0.858484848484849 | 0.822897835497835 | 0.810085454545455 | 228 |
| RMRP | 13 | 0.509230769230769 | 0.236923076923077 | 0.168461538461538 | 13 |
| GNE | 17 | 0.752352941176471 | 0.597052941176471 | 0.548234117647059 | 16 |
| GRHPR | 3 | NA | NA | NA | 3 |
| AUH | 8 | NA | NA | NA | 8 |
| FANCC | 17 | 0.977647058823529 | 0.935882352941176 | 0.919997647058824 | 17 |
| HSD17B3 | 9 | NA | NA | NA | 9 |
| XPA | 4 | NA | NA | NA | 4 |
| ALG2 | 3 | NA | NA | NA | 3 |
| INVS | 4 | NA | NA | NA | 4 |
| ALDOB | 16 | 0.945625 | 0.88124375 | 0.856874375 | 16 |
| FKTN | 18 | 0.928333333333333 | 0.860555555555556 | 0.832776111111111 | 18 |
| IKBKAP | 3 | NA | NA | NA | 3 |
| NR5A1 | 23 | 0.79695652173913 | 0.635652173913044 | 0.58347652173913 | 23 |
| GLE1 | 4 | NA | NA | NA | 4 |
| DOLK | 5 | NA | NA | NA | 5 |
| ASS1 | 22 | 0.887727272727273 | 0.781363636363636 | 0.739089090909091 | 22 |
| POMT1 | 18 | 0.911666666666667 | 0.786111111111111 | 0.733331111111111 | 18 |
| SURF1 | 4 | NA | NA | NA | 4 |
| ADAMTS13 | 23 | 0.84695652173913 | 0.693039130434783 | 0.635646086956522 | 21 |
| ADAMTSL2 | 7 | NA | NA | NA | 7 |
| LHX3 | 7 | NA | NA | NA | 7 |
| DCLRE1C | 4 | NA | NA | NA | 4 |
| PDSS1 | 1 | NA | NA | NA | 1 |
| ERCC6 | 12 | 0.7825 | 0.504166666666667 | 0.403333333333333 | 11 |
| PCDH15 | 18 | 0.991111111111111 | 0.98 | 0.976110555555556 | 18 |
| EGR2 | 9 | NA | NA | NA | 9 |
| NEUROG3 | 2 | NA | NA | NA | 2 |
| PRF1 | 12 | 0.738333333333333 | 0.48 | 0.386665833333333 | 10 |
| CDH23 | 34 | 0.893823529411765 | 0.785585294117647 | 0.736762352941177 | 34 |
| PSAP | 8 | NA | NA | NA | 8 |
| MRPS16 | 1 | NA | NA | NA | 1 |
| PTEN | 60 | 0.949166666666667 | 0.903996666666667 | 0.886333333333333 | 60 |
| FAS | 21 | 0.780952380952381 | 0.614285714285714 | 0.56142619047619 | 21 |
| PLCE1 | 8 | NA | NA | NA | 8 |
| COX15 | 2 | NA | NA | NA | 2 |
| C10orf2 | 16 | 0.811875 | 0.698125 | 0.650623125 | 15 |
| CYP17A1 | 23 | 0.807826086956522 | 0.727391304347826 | 0.69478 | 23 |
| COL17A1 | 6 | NA | NA | NA | 6 |
| UROS | 14 | 0.945714285714286 | 0.905 | 0.892142857142857 | 14 |
| SLC25A22 | 3 | NA | NA | NA | 3 |
| CTSD | 3 | NA | NA | NA | 3 |
| TH | 9 | NA | NA | NA | 9 |
| STIM1 | 9 | NA | NA | NA | 9 |
| HBB | 140 | 0.888071428571429 | 0.8415 | 0.825142 | 137 |
| SMPD1 | 30 | 0.936333333333333 | 0.897996666666667 | 0.882333 | 30 |
| TPP1 | 10 | 0.879 | 0.781 | 0.742999 | 10 |
| ABCC8 | 34 | 0.837647058823529 | 0.744117647058824 | 0.707939705882353 | 34 |
| USH1C | 10 | 0.805 | 0.53595 | 0.446998 | 6 |
| PDHX | 7 | NA | NA | NA | 7 |
| RAG1 | 19 | 0.872631578947368 | 0.823684210526316 | 0.805256842105263 | 19 |
| RAG2 | 9 | NA | NA | NA | 8 |
| SLC35C1 | 4 | NA | NA | NA | 4 |
| DDB2 | 4 | NA | NA | NA | 4 |
| RAPSN | 9 | NA | NA | NA | 9 |
| NDUFS3 | 2 | NA | NA | NA | 2 |
| TMEM216 | 4 | NA | NA | NA | 4 |
| FERMT3 | 4 | NA | NA | NA | 4 |
| PYGM | 23 | 0.943913043478261 | 0.816086956521739 | 0.769997391304348 | 22 |
| RNASEH2C | 2 | NA | NA | NA | 2 |
| EFEMP2 | 12 | 0.975 | 0.955833333333333 | 0.9475 | 12 |
| BBS1 | 10 | 0.648 | 0.315 | 0.193995 | 9 |
| PC | 14 | 0.889285714285714 | 0.790714285714286 | 0.755712857142857 | 14 |
| NDUFV1 | 3 | NA | NA | NA | 3 |
| UNC93B1 | 0 | NA | NA | NA | 0 |
| NDUFS8 | 7 | NA | NA | NA | 7 |
| TCIRG1 | 4 | NA | NA | NA | 4 |
| CPT1A | 34 | 0.899705882352941 | 0.775882352941176 | 0.720293529411765 | 33 |
| IGHMBP2 | 8 | NA | NA | NA | 8 |
| DHCR7 | 24 | 0.888333333333333 | 0.782495833333333 | 0.736664583333333 | 24 |
| FOLR1 | 3 | NA | NA | NA | 2 |
| MYO7A | 63 | 0.982063492063492 | 0.960952380952381 | 0.951111111111111 | 62 |
| ALG8 | 5 | NA | NA | NA | 5 |
| DYNC2H1 | 21 | 0.932857142857143 | 0.842371428571429 | 0.801428571428571 | 21 |
| ACAT1 | 19 | 0.932631578947368 | 0.869473684210526 | 0.841052105263158 | 19 |
| ATM | 143 | 0.956713286713287 | 0.919300699300699 | 0.906713076923077 | 142 |
| ALG9 | 2 | NA | NA | NA | 2 |
| CD3E | 3 | NA | NA | NA | 3 |
| CD3D | 3 | NA | NA | NA | 3 |
| CD3G | 4 | NA | NA | NA | 4 |
| SLC37A4 | 12 | 0.921666666666667 | 0.829158333333333 | 0.7858325 | 12 |
| DPAGT1 | 12 | 0.889166666666667 | 0.819166666666667 | 0.794165833333333 | 12 |
| SC5D | 3 | NA | NA | NA | 3 |
| KCNJ1 | 8 | NA | NA | NA | 7 |
| SCNN1A | 5 | NA | NA | NA | 4 |
| PEX5 | 2 | NA | NA | NA | 2 |
| FGD4 | 10 | 0.982 | 0.965 | 0.959 | 10 |
| VDR | 14 | 0.947142857142857 | 0.89 | 0.866427142857143 | 14 |
| TUBA1A | 12 | 0.855833333333333 | 0.800825 | 0.776666666666667 | 12 |
| AAAS | 7 | NA | NA | NA | 7 |
| SUOX | 3 | NA | NA | NA | 3 |
| ERBB3 | 0 | NA | NA | NA | 0 |
| CYP27B1 | 16 | 0.941875 | 0.860625 | 0.82875 | 16 |
| TSFM | 4 | NA | NA | NA | 4 |
| BBS10 | 12 | 0.895833333333333 | 0.781666666666667 | 0.739166666666667 | 12 |
| CEP290 | 23 | 0.909565217391304 | 0.744347826086956 | 0.697391304347826 | 23 |
| GNPTAB | 188 | 0.954627659574468 | 0.926755319148936 | 0.91664829787234 | 187 |
| PAH | 78 | 0.91051282051282 | 0.858202564102564 | 0.841282051282051 | 78 |
| MMAB | 3 | NA | NA | NA | 3 |
| MVK | 17 | 0.907058823529412 | 0.804117647058823 | 0.761760588235294 | 17 |
| ACADS | 17 | 0.798235294117647 | 0.605294117647059 | 0.532939411764706 | 15 |
| ORAI1 | 2 | NA | NA | NA | 2 |
| HPD | 7 | NA | NA | NA | 5 |
| ATP6V0A2 | 11 | 0.998181818181818 | 0.995454545454545 | 0.993636363636364 | 11 |
| GJB2 | 73 | 0.808219178082192 | 0.744928767123288 | 0.721232602739726 | 67 |
| SACS | 11 | 0.952727272727273 | 0.894545454545455 | 0.873635454545455 | 11 |
| FREM2 | 2 | NA | NA | NA | 2 |
| SLC25A15 | 17 | 0.944705882352941 | 0.9 | 0.879998235294118 | 17 |
| SUCLA2 | 4 | NA | NA | NA | 4 |
| RNASEH2B | 2 | NA | NA | NA | 2 |
| ATP7B | 36 | 0.894722222222222 | 0.789997222222222 | 0.748608888888889 | 34 |
| CLN5 | 8 | NA | NA | NA | 8 |
| EDNRB | 4 | NA | NA | NA | 4 |
| PCCA | 8 | NA | NA | NA | 8 |
| ERCC5 | 10 | 0.983 | 0.964 | 0.956 | 10 |
| LIG4 | 7 | NA | NA | NA | 5 |
| TGM1 | 38 | 0.904736842105263 | 0.824207894736842 | 0.793419473684211 | 36 |
| FOXG1 | 16 | 0.99 | 0.965 | 0.95625 | 16 |
| MGAT2 | 4 | NA | NA | NA | 4 |
| NPC2 | 16 | 0.9125 | 0.768125 | 0.712496875 | 16 |
| POMT2 | 19 | 0.969473684210526 | 0.935263157894737 | 0.922631052631579 | 19 |
| VIPAS39 | 4 | NA | NA | NA | 4 |
| GALC | 9 | NA | NA | NA | 8 |
| FBLN5 | 12 | 0.833333333333333 | 0.649991666666667 | 0.5883325 | 12 |
| UBE3A | 150 | 0.975466666666667 | 0.9604 | 0.955733066666667 | 150 |
| SLC12A6 | 14 | 1 | 1 | 1 | 14 |
| IVD | 8 | NA | NA | NA | 8 |
| UBR1 | 2 | NA | NA | NA | 2 |
| BLOC1S6 | 1 | NA | NA | NA | 1 |
| SLC12A1 | 3 | NA | NA | NA | 3 |
| MYO5A | 0 | NA | NA | NA | 0 |
| RAB27A | 5 | NA | NA | NA | 5 |
| CLN6 | 13 | 0.871538461538462 | 0.77 | 0.73923 | 13 |
| HEXA | 49 | 0.952040816326531 | 0.903061224489796 | 0.886325714285714 | 47 |
| STRA6 | 18 | 0.908333333333333 | 0.832222222222222 | 0.809439444444444 | 18 |
| CYP11A1 | 6 | NA | NA | NA | 6 |
| MPI | 4 | NA | NA | NA | 4 |
| ETFA | 3 | NA | NA | NA | 3 |
| FAH | 16 | 0.798125 | 0.575625 | 0.487499375 | 15 |
| POLG | 25 | 0.8424 | 0.707996 | 0.6575992 | 24 |
| BLM | 45 | 0.987555555555556 | 0.964 | 0.954221333333333 | 45 |
| VPS33B | 6 | NA | NA | NA | 6 |
| HBA2 | 17 | 0.811176470588235 | 0.637052941176471 | 0.577646470588235 | 16 |
| HBA1 | 3 | NA | NA | NA | 3 |
| CLCN7 | 7 | NA | NA | NA | 7 |
| ABCA3 | 6 | NA | NA | NA | 6 |
| MEFV | 18 | 0.251111111111111 | 0.121666666666667 | 0.0894438888888889 | 14 |
| ALG1 | 8 | NA | NA | NA | 8 |
| PMM2 | 24 | 0.867916666666667 | 0.812083333333333 | 0.792916666666667 | 24 |
| ERCC4 | 11 | 0.984545454545455 | 0.966363636363636 | 0.959090909090909 | 11 |
| SCNN1G | 3 | NA | NA | NA | 1 |
| SCNN1B | 12 | 0.861666666666667 | 0.719166666666667 | 0.6733325 | 11 |
| COG7 | 0 | NA | NA | NA | 0 |
| CLN3 | 4 | NA | NA | NA | 4 |
| TUFM | 1 | NA | NA | NA | 1 |
| CD19 | 0 | NA | NA | NA | 0 |
| RPGRIP1L | 13 | 0.897692307692308 | 0.684607692307692 | 0.609215384615385 | 12 |
| COQ9 | 1 | NA | NA | NA | 1 |
| TK2 | 36 | 0.906111111111111 | 0.849166666666667 | 0.8313875 | 36 |
| HSD11B2 | 9 | NA | NA | NA | 9 |
| COG8 | 1 | NA | NA | NA | 1 |
| TAT | 5 | NA | NA | NA | 5 |
| GOSH | 0 | NA | NA | NA | 0 |
| ZNF469 | 21 | 0.348095238095238 | 0.214761904761905 | 0.185237619047619 | 20 |
| ASPA | 9 | NA | NA | NA | 9 |
| CTNS | 19 | 0.898421052631579 | 0.752105263157895 | 0.69841 | 18 |
| ACADVL | 25 | 0.8488 | 0.702392 | 0.6483972 | 24 |
| MPDU1 | 4 | NA | NA | NA | 4 |
| SCO1 | 3 | NA | NA | NA | 3 |
| COX10 | 6 | NA | NA | NA | 6 |
| PMP22 | 16 | 0.963125 | 0.93936875 | 0.929999375 | 15 |
| ALDH3A2 | 11 | 0.976363636363636 | 0.941818181818182 | 0.929090909090909 | 11 |
| FOXN1 | 1 | NA | NA | NA | 1 |
| PEX12 | 9 | NA | NA | NA | 9 |
| NAGLU | 18 | 0.891666666666667 | 0.836111111111111 | 0.819443333333333 | 18 |
| G6PC | 23 | 0.900869565217391 | 0.775652173913043 | 0.730433913043478 | 22 |
| NAGS | 8 | NA | NA | NA | 8 |
| G6PC3 | 6 | NA | NA | NA | 6 |
| WNT3 | 1 | NA | NA | NA | 1 |
| PNPO | 3 | NA | NA | NA | 3 |
| SGCA | 8 | NA | NA | NA | 8 |
| COL1A1 | 55 | 0.937090909090909 | 0.907636363636364 | 0.897272181818182 | 55 |
| MKS1 | 8 | NA | NA | NA | 8 |
| TRIM37 | 4 | NA | NA | NA | 4 |
| COG1 | 0 | NA | NA | NA | 0 |
| USH1G | 7 | NA | NA | NA | 7 |
| TSEN54 | 9 | NA | NA | NA | 9 |
| ITGB4 | 9 | NA | NA | NA | 9 |
| GALK1 | 5 | NA | NA | NA | 5 |
| UNC13D | 3 | NA | NA | NA | 3 |
| ACOX1 | 7 | NA | NA | NA | 7 |
| GAA | 29 | 0.910689655172414 | 0.839996551724138 | 0.816895862068965 | 28 |
| SGSH | 10 | 0.887 | 0.803 | 0.774 | 10 |
| NPC1 | 46 | 0.922173913043478 | 0.852391304347826 | 0.824782173913043 | 46 |
| LAMA3 | 4 | NA | NA | NA | 4 |
| TCF4 | 24 | 0.991666666666667 | 0.982916666666667 | 0.979583333333333 | 24 |
| ATP8B1 | 12 | 0.820833333333333 | 0.6225 | 0.5458325 | 12 |
| NDUFS7 | 2 | NA | NA | NA | 2 |
| GAMT | 6 | NA | NA | NA | 6 |
| INSR | 31 | 0.910645161290323 | 0.814516129032258 | 0.773225483870968 | 30 |
| MCOLN1 | 5 | NA | NA | NA | 5 |
| STXBP2 | 3 | NA | NA | NA | 3 |
| NDUFA7 | 0 | NA | NA | NA | 0 |
| TYK2 | 0 | NA | NA | NA | 0 |
| MAN2B1 | 12 | 0.975 | 0.936666666666667 | 0.9233325 | 12 |
| RNASEH2A | 12 | 0.813333333333333 | 0.689158333333333 | 0.6349975 | 10 |
| GCDH | 11 | 0.883636363636364 | 0.773636363636364 | 0.726362727272727 | 11 |
| JAK3 | 5 | NA | NA | NA | 5 |
| IL12RB1 | 5 | NA | NA | NA | 5 |
| CRLF1 | 18 | 0.932222222222222 | 0.829444444444444 | 0.781665 | 17 |
| HAMP | 1 | NA | NA | NA | 1 |
| COX6B1 | 1 | NA | NA | NA | 1 |
| NPHS1 | 9 | NA | NA | NA | 8 |
| DLL3 | 3 | NA | NA | NA | 3 |
| PRX | 10 | 0.966 | 0.875 | 0.844 | 10 |
| BCKDHA | 32 | 0.953125 | 0.916875 | 0.9021875 | 32 |
| ETHE1 | 5 | NA | NA | NA | 5 |
| ERCC2 | 12 | 0.880833333333333 | 0.7725 | 0.723333333333333 | 11 |
| OPA3 | 7 | NA | NA | NA | 7 |
| FKRP | 20 | 0.7825 | 0.626995 | 0.557499 | 20 |
| NUP62 | 1 | NA | NA | NA | 1 |
| ETFB | 2 | NA | NA | NA | 2 |
| SLC4A11 | 16 | 0.92625 | 0.85875 | 0.8375 | 16 |
| PANK2 | 12 | 0.914166666666667 | 0.805833333333333 | 0.765 | 12 |
| DNMT3B | 9 | NA | NA | NA | 9 |
| GSS | 6 | NA | NA | NA | 6 |
| SAMHD1 | 19 | 0.985789473684211 | 0.966315789473684 | 0.95999947368421 | 19 |
| ADA | 26 | 0.841538461538462 | 0.725 | 0.675766538461538 | 24 |
| DPM1 | 4 | NA | NA | NA | 4 |
| EDN3 | 4 | NA | NA | NA | 4 |
| IFNGR2 | 5 | NA | NA | NA | 5 |
| HLCS | 9 | NA | NA | NA | 9 |
| CBS | 16 | 0.84625 | 0.78 | 0.753123125 | 16 |
| CSTB | 9 | NA | NA | NA | 9 |
| AIRE | 11 | 0.984545454545455 | 0.969090909090909 | 0.963636363636364 | 11 |
| COL6A1 | 10 | 0.92 | 0.779 | 0.707999 | 10 |
| COL6A2 | 21 | 0.842857142857143 | 0.7 | 0.642849047619048 | 21 |
| PEX26 | 7 | NA | NA | NA | 7 |
| SNAP29 | 1 | NA | NA | NA | 1 |
| LARGE | 4 | NA | NA | NA | 4 |
| PLA2G6 | 37 | 0.938378378378378 | 0.877024324324324 | 0.847566216216216 | 37 |
| ALG12 | 6 | NA | NA | NA | 6 |
| MLC1 | 12 | 0.890833333333333 | 0.660833333333333 | 0.561665 | 12 |
| SCO2 | 8 | NA | NA | NA | 7 |
| TYMP | 9 | NA | NA | NA | 9 |
| ARSA | 58 | 0.867241379310345 | 0.783443103448276 | 0.752068103448276 | 56 |
| All | 6793 | 0.902891211541292 | 0.897057205947299 | 0.89538492713087 | 6653 |

| | Average VGD | 99% cutoff | 99.9% cutoff |
|---|---|---|---|
| | naive (only | (only using | (only using |
| Gene Name | using CV 5.1) | CV 5.1) | CV 5.1) |
| PEX10 | 0.98 | 0.967 | 0.962 |
| NPHP4 | NA | NA | NA |
| PLEKHG5 | NA | NA | NA |
| PLOD1 | NA | NA | NA |
| ALPL | 0.934545454545455 | 0.893636363636364 | 0.874545 |
| HSPG2 | NA | NA | NA |
| HMGCL | NA | NA | NA |
| FUCA1 | NA | NA | NA |
| SEPN1 | NA | NA | NA |
| NDUFS5 | NA | NA | NA |
| PPT1 | NA | NA | NA |
| ZMPSTE24 | NA | NA | NA |
| CLDN19 | NA | NA | NA |
| P3H1 | NA | NA | NA |
| MPL | NA | NA | NA |
| ST3GAL3 | NA | NA | NA |
| MMACHC | 0.98 | 0.963846153846154 | 0.956923076923077 |
| POMGNT1 | 0.985263157894737 | 0.948421052631579 | 0.934210526315789 |
| CPT2 | 0.919444444444444 | 0.864444444444444 | 0.844998888888889 |
| DHCR24 | NA | NA | NA |
| ALG6 | NA | NA | NA |
| SLC35D1 | NA | NA | NA |
| ACADM | 0.911666666666667 | 0.836661111111111 | 0.805554444444445 |
| DPYD | NA | NA | NA |
| AGL | 0.938125 | 0.755 | 0.693125 |
| DBT | 0.954545454545455 | 0.905909090909091 | 0.887271363636364 |
| TSHB | NA | NA | NA |
| HSD3B2 | 0.968 | 0.935 | 0.920999 |
| HFE2 | NA | NA | NA |
| CTSK | NA | NA | NA |
| HAX1 | NA | NA | NA |
| GBA | 0.845230769230769 | 0.790921538461538 | 0.769383846153846 |
| PKLR | NA | NA | NA |
| LMNA | 0.80421875 | 0.7328125 | 0.710624375 |
| NTRK1 | 0.762307692307692 | 0.491538461538462 | 0.396920769230769 |
| MPZ | 0.8368 | 0.779198 | 0.7575998 |
| CD247 | NA | NA | NA |
| FASLG | NA | NA | NA |
| NPHS2 | 0.874545454545455 | 0.627254545454546 | 0.515454545454545 |
| LAMC2 | NA | NA | NA |
| LAMB3 | 0.84 | 0.652142857142857 | 0.597850714285714 |
| USH2A | 0.974186046511628 | 0.945581395348837 | 0.932790232558139 |
| RAB3GAP2 | NA | NA | NA |
| LBR | NA | NA | NA |
| ADCK3 | 0.959230769230769 | 0.905384615384615 | 0.883074615384615 |
| GJC2 | NA | NA | NA |
| TBCE | NA | NA | NA |
| LYST | 0.990212765957447 | 0.976168085106383 | 0.970425106382979 |
| FH | 0.939583333333333 | 0.8625 | 0.831665833333333 |
| HADHA | NA | NA | NA |
| HADHB | NA | NA | NA |
| MPV17 | 0.971153846153846 | 0.954230769230769 | 0.947307692307692 |
| SRD5A2 | 0.853571428571429 | 0.755707142857143 | 0.717857142857143 |
| LRPPRC | NA | NA | NA |
| LHCGR | 0.849583333333333 | 0.812495833333333 | 0.80124875 |
| PEX13 | NA | NA | NA |
| ALMS1 | NA | NA | NA |
| DGUOK | NA | NA | NA |
| MOGS | NA | NA | NA |
| SUCLG1 | NA | NA | NA |
| SFTPB | NA | NA | NA |
| ST3GAL5 | NA | NA | NA |
| EIF2AK3 | NA | NA | NA |
| NPHP1 | NA | NA | NA |
| IL1RN | NA | NA | NA |
| ERCC3 | NA | NA | NA |
| RAB3GAP1 | NA | NA | NA |
| ZEB2 | 0.967868852459016 | 0.92917868852459 | 0.91295 |
| NEB | NA | NA | NA |
| ABCB11 | NA | NA | NA |
| LRP2 | 0.948947368421053 | 0.851578947368421 | 0.803157894736842 |
| ITGA6 | NA | NA | NA |
| CHRNA1 | 0.86 | 0.792142857142857 | 0.768571428571429 |
| AGPS | NA | NA | NA |
| HIBCH | NA | NA | NA |
| STAT1 | 0.791666666666667 | 0.687495833333333 | 0.65583125 |
| CASP10 | NA | NA | NA |
| ALS2 | 0.996086956521739 | 0.990869565217391 | 0.988695652173913 |
| ICOS | NA | NA | NA |
| FASTKD2 | NA | NA | NA |
| ACADL | NA | NA | NA |
| CPS1 | NA | NA | NA |
| ABCA12 | 0.984166666666667 | 0.950833333333333 | 0.938333333333333 |
| BCS1L | 0.913076923076923 | 0.856923076923077 | 0.839999230769231 |
| CYP27A1 | 0.957169811320755 | 0.911884905660377 | 0.894904528301887 |
| WNT10A | NA | NA | NA |
| NHEJ1 | NA | NA | NA |
| COL4A4 | NA | NA | NA |
| COL4A3 | NA | NA | NA |
| SP110 | 0.926666666666667 | 0.8275 | 0.795 |
| CHRND | NA | NA | NA |
| CHRNG | NA | NA | NA |
| COL6A3 | NA | NA | NA |
| AGXT | 0.945 | 0.898571428571429 | 0.877142857142857 |
| WNT7A | NA | NA | NA |
| XPC | NA | NA | NA |
| BTD | 0.871935483870968 | 0.830255483870968 | 0.81419335483871 |
| GLB1 | 0.97 | 0.947140476190476 | 0.937618571428571 |
| CRTAP | NA | NA | NA |
| MYD88 | NA | NA | NA |
| PTH1R | NA | NA | NA |
| TREX1 | 0.904782608695652 | 0.832169565217391 | 0.800869130434783 |
| COL7A1 | 0.952777777777778 | 0.916388888888889 | 0.903333055555556 |
| SLC25A20 | NA | NA | NA |
| LAMB2 | NA | NA | NA |
| AMT | NA | NA | NA |
| RFT1 | NA | NA | NA |
| HESX1 | NA | NA | NA |
| GBE1 | 0.95 | 0.820625 | 0.775625 |
| POU1F1 | 0.974117647058824 | 0.932352941176471 | 0.917058823529412 |
| HGD | 0.985882352941176 | 0.971176470588235 | 0.964705882352941 |
| IQCB1 | 0.995 | 0.98 | 0.975 |
| ACAD9 | NA | NA | NA |
| NPHP3 | NA | NA | NA |
| PCCB | 0.96 | 0.887142857142857 | 0.861428095238095 |
| MRPS22 | NA | NA | NA |
| ATR | NA | NA | NA |
| CLRN1 | NA | NA | NA |
| GFM1 | NA | NA | NA |
| IFT80 | NA | NA | NA |
| BCHE | NA | NA | NA |
| DNAJC19 | NA | NA | NA |
| ALG3 | NA | NA | NA |
| CLDN1 | NA | NA | NA |
| IDUA | 0.726333333333333 | 0.579663333333333 | 0.525664 |
| DOK7 | 0.970833333333333 | 0.895 | 0.868333333333333 |
| EVC2 | NA | NA | NA |
| EVC | NA | NA | NA |
| SGCB | NA | NA | NA |
| SRD5A3 | NA | NA | NA |
| GNRHR | 0.798333333333333 | 0.685555555555556 | 0.646666111111111 |
| FRAS1 | NA | NA | NA |
| ANTXR2 | NA | NA | NA |
| COQ2 | NA | NA | NA |
| DMP1 | NA | NA | NA |
| HADH | NA | NA | NA |
| PRSS12 | NA | NA | NA |
| MFSD8 | NA | NA | NA |
| MMAA | NA | NA | NA |
| FGA | NA | NA | NA |
| ETFDH | 0.97 | 0.943636363636364 | 0.934545454545455 |
| AGA | NA | NA | NA |
| TLR3 | NA | NA | NA |
| F11 | 0.857692307692308 | 0.683846153846154 | 0.616152307692308 |
| NDUFS6 | NA | NA | NA |
| NSUN2 | NA | NA | NA |
| AMACR | NA | NA | NA |
| LIFR | NA | NA | NA |
| OXCT1 | NA | NA | NA |
| MOCS2 | 0.926428571428571 | 0.712857142857143 | 0.641428571428571 |
| NDUFS4 | NA | NA | NA |
| ERCC8 | NA | NA | NA |
| NDUFAF2 | NA | NA | NA |
| SMN2 | NA | NA | NA |
| SMN1 | 0.798888888888889 | 0.612216666666667 | 0.55222 |
| HEXB | 0.955454545454545 | 0.893627272727273 | 0.871817272727273 |
| AP3B1 | NA | NA | NA |
| ARSB | 0.94 | 0.811111111111111 | 0.760555 |
| ADGRV1 | 0.945789473684211 | 0.788421052631579 | 0.736315263157895 |
| HSD17B4 | 0.952 | 0.854 | 0.819999 |
| ALDH7A1 | NA | NA | NA |
| SLC22A5 | 0.898695652173913 | 0.796517391304348 | 0.763912608695652 |
| UQCRQ | NA | NA | NA |
| SIL1 | NA | NA | NA |
| SLC26A2 | 0.976428571428571 | 0.959285714285714 | 0.952142857142857 |
| IL12B | NA | NA | NA |
| NSD1 | 0.986230769230769 | 0.964306923076923 | 0.951999615384615 |
| PROP1 | 0.992666666666667 | 0.986666666666667 | 0.984666666666667 |
| DSP | 0.965263157894737 | 0.926842105263158 | 0.911052105263158 |
| NHLRC1 | NA | NA | NA |
| ALDH5A1 | NA | NA | NA |
| NEU1 | 0.864615384615385 | 0.781538461538462 | 0.750768461538462 |
| CYP21A1P | NA | NA | NA |
| CYP21A2 | 0.687777777777778 | 0.517772222222222 | 0.460550555555556 |
| MOCS1 | NA | NA | NA |
| MUT | 0.962666666666667 | 0.907333333333333 | 0.884666666666667 |
| PKHD1 | 0.959736842105263 | 0.898684210526316 | 0.874736578947368 |
| RAB23 | NA | NA | NA |
| SLC17A5 | NA | NA | NA |
| BCKDHB | 0.986 | 0.9748 | 0.97 |
| SLC35A1 | NA | NA | NA |
| NDUFAF4 | NA | NA | NA |
| GRIK2 | NA | NA | NA |
| PDSS2 | NA | NA | NA |
| OSTM1 | NA | NA | NA |
| TSPYL1 | NA | NA | NA |
| LAMA2 | 0.980882352941176 | 0.925585294117647 | 0.906470588235294 |
| ENPP1 | 0.980769230769231 | 0.960769230769231 | 0.953076923076923 |
| AHI1 | 0.994 | 0.988 | 0.985333333333333 |
| PEX7 | 0.808571428571429 | 0.572135714285714 | 0.498568571428571 |
| IFNGR1 | 0.962857142857143 | 0.918571428571429 | 0.899999285714286 |
| STX11 | NA | NA | NA |
| EPM2A | NA | NA | NA |
| GTF2H5 | NA | NA | NA |
| PLG | 0.917 | 0.805 | 0.769 |
| FAM20C | NA | NA | NA |
| FAM126A | NA | NA | NA |
| DDC | NA | NA | NA |
| GUSB | 0.933333333333333 | 0.891666666666667 | 0.880555555555556 |
| ASL | 0.905454545454546 | 0.764545454545455 | 0.70818 |
| SBDS | 0.972307692307692 | 0.934615384615385 | 0.920766153846154 |
| POR | NA | NA | NA |
| ABCB4 | 0.988666666666667 | 0.974 | 0.967333333333333 |
| PEX1 | 0.921818181818182 | 0.85 | 0.820906363636364 |
| COL1A2 | 0.962307692307692 | 0.93 | 0.916537692307692 |
| RELN | NA | NA | NA |
| SLC26A4 | 0.940217391304348 | 0.888258695652174 | 0.869346086956522 |
| DLD | 0.969090909090909 | 0.940909090909091 | 0.93 |
| CFTR | 0.932186234817814 | 0.906760728744939 | 0.898137044534413 |
| CLN8 | NA | NA | NA |
| TUSC3 | NA | NA | NA |
| SFTPC | NA | NA | NA |
| ESCO2 | 0.9340625 | 0.810309375 | 0.7778121875 |
| STAR | 0.871538461538461 | 0.668461538461538 | 0.577689230769231 |
| HGSNAT | 0.974166666666667 | 0.938333333333333 | 0.9233275 |
| TTPA | 0.9125 | 0.801875 | 0.764374375 |
| GDAP1 | 0.857647058823529 | 0.759411764705882 | 0.72529294117647 |
| CA2 | NA | NA | NA |
| CNGB3 | NA | NA | NA |
| NBN | 0.98030303030303 | 0.922121212121212 | 0.90272696969697 |
| TMEM67 | 0.960833333333333 | 0.918333333333333 | 0.906665833333333 |
| PDP1 | NA | NA | NA |
| UQCRB | NA | NA | NA |
| VPS13B | 0.983055555555556 | 0.961666666666667 | 0.952221666666667 |
| RRM2B | 0.944054054054054 | 0.886751351351351 | 0.863512972972973 |
| TNFRSF11B | NA | NA | NA |
| TRAPPC9 | NA | NA | NA |
| CYP11B1 | 0.905 | 0.824 | 0.804999 |
| PLEC | NA | NA | NA |
| DOCK8 | NA | NA | NA |
| VLDLR | NA | NA | NA |
| GLDC | 0.973 | 0.957 | 0.950999 |
| APTX | NA | NA | NA |
| B4GALT1 | NA | NA | NA |
| GALT | 0.866798245614035 | 0.833113596491228 | 0.820482456140351 |
| RMRP | 0.509230769230769 | 0.234615384615385 | 0.168458461538462 |
| GNE | 0.795625 | 0.688125 | 0.65499875 |
| GRHPR | NA | NA | NA |
| AUH | NA | NA | NA |
| FANCC | 0.977647058823529 | 0.937052941176471 | 0.918235294117647 |
| HSD17B3 | NA | NA | NA |
| XPA | NA | NA | NA |
| ALG2 | NA | NA | NA |
| INVS | NA | NA | NA |
| ALDOB | 0.945625 | 0.88 | 0.854375 |
| FKTN | 0.928333333333333 | 0.86 | 0.841665555555556 |
| IKBKAP | NA | NA | NA |
| NR5A1 | 0.79695652173913 | 0.632608695652174 | 0.574779130434783 |
| GLE1 | NA | NA | NA |
| DOLK | NA | NA | NA |
| ASS1 | 0.887727272727273 | 0.783636363636364 | 0.753174090909091 |
| POMT1 | 0.911666666666667 | 0.779438888888889 | 0.729442777777778 |
| SURF1 | NA | NA | NA |
| ADAMTS13 | 0.927619047619048 | 0.88 | 0.863332857142857 |
| ADAMTSL2 | NA | NA | NA |
| LHX3 | NA | NA | NA |
| DCLRE1C | NA | NA | NA |
| PDSS1 | NA | NA | NA |
| ERCC6 | 0.853636363636364 | 0.635454545454545 | 0.497272727272727 |
| PCDH15 | 0.991111111111111 | 0.980555555555556 | 0.976111111111111 |
| EGR2 | NA | NA | NA |
| NEUROG3 | NA | NA | NA |
| PRF1 | 0.886 | 0.785 | 0.754999 |
| CDH23 | 0.893823529411765 | 0.783235294117647 | 0.744115588235294 |
| PSAP | NA | NA | NA |
| MRPS16 | NA | NA | NA |
| PTEN | 0.949166666666667 | 0.905833333333333 | 0.889665666666667 |
| FAS | 0.780952380952381 | 0.618090476190476 | 0.564284285714286 |
| PLCE1 | NA | NA | NA |
| COX15 | NA | NA | NA |
| C10orf2 | 0.841333333333333 | 0.74466 | 0.714665333333333 |
| CYP17A1 | 0.807826086956522 | 0.724347826086956 | 0.694781739130435 |
| COL17A1 | NA | NA | NA |
| UROS | 0.945714285714286 | 0.905 | 0.889999285714286 |
| SLC25A22 | NA | NA | NA |
| CTSD | NA | NA | NA |
| TH | NA | NA | NA |
| STIM1 | NA | NA | NA |
| HBB | 0.891240875912409 | 0.842260583941606 | 0.825764890510949 |
| SMPD1 | 0.936333333333333 | 0.897333333333333 | 0.879666 |
| TPP1 | 0.879 | 0.78399 | 0.752 |
| ABCC8 | 0.837647058823529 | 0.743526470588235 | 0.708529117647059 |
| USH1C | NA | NA | NA |
| PDHX | NA | NA | NA |
| RAG1 | 0.872631578947368 | 0.823684210526316 | 0.806841052631579 |
| RAG2 | NA | NA | NA |
| SLC35C1 | NA | NA | NA |
| DDB2 | NA | NA | NA |
| RAPSN | NA | NA | NA |
| NDUFS3 | NA | NA | NA |
| TMEM216 | NA | NA | NA |
| FERMT3 | NA | NA | NA |
| PYGM | 0.986363636363636 | 0.973636363636364 | 0.966817727272727 |
| RNASEH2C | NA | NA | NA |
| EFEMP2 | 0.975 | 0.956666666666667 | 0.950833333333333 |
| BBS1 | NA | NA | NA |
| PC | 0.889285714285714 | 0.794285714285714 | 0.757849285714286 |
| NDUFV1 | NA | NA | NA |
| UNC93B1 | NA | NA | NA |
| NDUFS8 | NA | NA | NA |
| TCIRG1 | NA | NA | NA |
| CPT1A | 0.926969696969697 | 0.811509090909091 | 0.765443333333333 |
| IGHMBP2 | NA | NA | NA |
| DHCR7 | 0.888333333333333 | 0.783745833333333 | 0.740827916666667 |
| FOLR1 | NA | NA | NA |
| MYO7A | 0.988387096774194 | 0.976774193548387 | 0.971935322580645 |
| ALG8 | NA | NA | NA |
| DYNC2H1 | 0.932857142857143 | 0.840471428571429 | 0.80666619047619 |
| ACAT1 | 0.932631578947368 | 0.867894736842105 | 0.845262105263158 |
| ATM | 0.963450704225352 | 0.926688732394366 | 0.912183098591549 |
| ALG9 | NA | NA | NA |
| CD3E | NA | NA | NA |
| CD3D | NA | NA | NA |
| CD3G | NA | NA | NA |
| SLC37A4 | 0.921666666666667 | 0.828333333333333 | 0.784996666666667 |
| DPAGT1 | 0.889166666666667 | 0.819166666666667 | 0.791665833333333 |
| SC5D | NA | NA | NA |
| KCNJ1 | NA | NA | NA |
| SCNN1A | NA | NA | NA |
| PEX5 | NA | NA | NA |
| FGD4 | 0.982 | 0.965 | 0.959999 |
| VDR | 0.947142857142857 | 0.888571428571429 | 0.867141428571429 |
| TUBA1A | 0.855833333333333 | 0.800833333333333 | 0.780833333333333 |
| AAAS | NA | NA | NA |
| SUOX | NA | NA | NA |
| ERBB3 | NA | NA | NA |
| CYP27B1 | 0.941875 | 0.8625 | 0.8318725 |
| TSFM | NA | NA | NA |
| BBS10 | 0.895833333333333 | 0.779166666666667 | 0.740833333333333 |
| CEP290 | 0.909565217391304 | 0.743473913043478 | 0.696956086956522 |
| GNPTAB | 0.959732620320856 | 0.93475935828877 | 0.925881818181818 |
| PAH | 0.91051282051282 | 0.860512820512821 | 0.839614230769231 |
| MMAB | NA | NA | NA |
| MVK | 0.907058823529412 | 0.806470588235294 | 0.768235294117647 |
| ACADS | 0.904666666666667 | 0.842666666666667 | 0.819999333333333 |
| ORAI1 | NA | NA | NA |
| HPD | NA | NA | NA |
| ATP6V0A2 | 0.998181818181818 | 0.995454545454545 | 0.993636363636364 |
| GJB2 | 0.832537313432836 | 0.779550746268657 | 0.761192388059701 |
| SACS | 0.952727272727273 | 0.892727272727273 | 0.878180909090909 |
| FREM2 | NA | NA | NA |
| SLC25A15 | 0.944705882352941 | 0.899411764705882 | 0.881764705882353 |
| SUCLA2 | NA | NA | NA |
| RNASEH2B | NA | NA | NA |
| ATP7B | 0.919117647058823 | 0.827352941176471 | 0.785 |
| CLN5 | NA | NA | NA |
| EDNRB | NA | NA | NA |
| PCCA | NA | NA | NA |
| ERCC5 | 0.983 | 0.963 | 0.954 |
| LIG4 | NA | NA | NA |
| TGM1 | 0.935555555555556 | 0.894722222222222 | 0.881109166666667 |
| FOXG1 | 0.99 | 0.96375 | 0.95625 |
| MGAT2 | NA | NA | NA |
| NPC2 | 0.9125 | 0.766875 | 0.69874875 |
| POMT2 | 0.969473684210526 | 0.934736842105263 | 0.923683684210526 |
| VIPAS39 | NA | NA | NA |
| GALC | NA | NA | NA |
| FBLN5 | 0.833333333333333 | 0.654166666666667 | 0.5833325 |
| UBE3A | 0.975466666666667 | 0.961066 | 0.9551996 |
| SLC12A6 | 1 | 1 | 1 |
| IVD | NA | NA | NA |
| UBR1 | NA | NA | NA |
| BLOC1S6 | NA | NA | NA |
| SLC12A1 | NA | NA | NA |
| MYO5A | NA | NA | NA |
| RAB27A | NA | NA | NA |
| CLN6 | 0.871538461538462 | 0.768453846153846 | 0.727692307692308 |
| HEXA | 0.974042553191489 | 0.944468085106383 | 0.932126595744681 |
| STRA6 | 0.908333333333333 | 0.833333333333333 | 0.804443888888889 |
| CYP11A1 | NA | NA | NA |
| MPI | NA | NA | NA |
| ETFA | NA | NA | NA |
| FAH | 0.851333333333333 | 0.651326666666667 | 0.579328666666667 |
| POLG | 0.877083333333333 | 0.774995833333333 | 0.731662083333333 |
| BLM | 0.987555555555556 | 0.964666666666667 | 0.954888666666667 |
| VPS33B | NA | NA | NA |
| HBA2 | 0.861875 | 0.736875 | 0.6881225 |
| HBA1 | NA | NA | NA |
| CLCN7 | NA | NA | NA |
| ABCA3 | NA | NA | NA |
| MEFV | 0.318571428571429 | 0.175714285714286 | 0.140713571428571 |
| ALG1 | NA | NA | NA |
| PMM2 | 0.867916666666667 | 0.811666666666667 | 0.795833333333333 |
| ERCC4 | 0.984545454545455 | 0.965454545454545 | 0.960908181818182 |
| SCNN1G | NA | NA | NA |
| SCNN1B | 0.892727272727273 | 0.761818181818182 | 0.700906363636364 |
| COG7 | NA | NA | NA |
| CLN3 | NA | NA | NA |
| TUFM | NA | NA | NA |
| CD19 | NA | NA | NA |
| RPGRIP1L | 0.9725 | 0.905 | 0.88 |
| COQ9 | NA | NA | NA |
| TK2 | 0.906111111111111 | 0.850555555555556 | 0.831666666666667 |
| HSD11B2 | NA | NA | NA |
| COG8 | NA | NA | NA |
| TAT | NA | NA | NA |
| GOSH | NA | NA | NA |
| ZNF469 | 0.362 | 0.226995 | 0.1899995 |
| ASPA | NA | NA | NA |
| CTNS | 0.948333333333333 | 0.89 | 0.869999444444444 |
| ACADVL | 0.884166666666667 | 0.760416666666667 | 0.7070825 |
| MPDU1 | NA | NA | NA |
| SCO1 | NA | NA | NA |
| COX10 | NA | NA | NA |
| PMP22 | 0.965333333333333 | 0.939993333333333 | 0.930666 |
| ALDH3A2 | 0.976363636363636 | 0.941809090909091 | 0.929090909090909 |
| FOXN1 | NA | NA | NA |
| PEX12 | NA | NA | NA |
| NAGLU | 0.891666666666667 | 0.836666666666667 | 0.818332777777778 |
| G6PC | 0.941818181818182 | 0.903181818181818 | 0.887727272727273 |
| NAGS | NA | NA | NA |
| G6PC3 | NA | NA | NA |
| WNT3 | NA | NA | NA |
| PNPO | NA | NA | NA |
| SGCA | NA | NA | NA |
| COL1A1 | 0.937090909090909 | 0.906363636363636 | 0.895272545454545 |
| MKS1 | NA | NA | NA |
| TRIM37 | NA | NA | NA |
| COG1 | NA | NA | NA |
| USH1G | NA | NA | NA |
| TSEN54 | NA | NA | NA |
| ITGB4 | NA | NA | NA |
| GALK1 | NA | NA | NA |
| UNC13D | NA | NA | NA |
| ACOX1 | NA | NA | NA |
| GAA | 0.910714285714286 | 0.835 | 0.810356785714286 |
| SGSH | 0.887 | 0.802 | 0.779 |
| NPC1 | 0.922173913043478 | 0.85304347826087 | 0.821521521739131 |
| LAMA3 | NA | NA | NA |
| TCF4 | 0.991666666666667 | 0.983329166666667 | 0.979166666666667 |
| ATP8B1 | 0.820833333333333 | 0.615 | 0.55 |
| NDUFS7 | NA | NA | NA |
| GAMT | NA | NA | NA |
| INSR | 0.941 | 0.898666666666667 | 0.882999666666667 |
| MCOLN1 | NA | NA | NA |
| STXBP2 | NA | NA | NA |
| NDUFA7 | NA | NA | NA |
| TYK2 | NA | NA | NA |
| MAN2B1 | 0.975 | 0.9375 | 0.920833333333333 |
| RNASEH2A | 0.881 | 0.801 | 0.773999 |
| GCDH | 0.883636363636364 | 0.769090909090909 | 0.731817272727273 |
| JAK3 | NA | NA | NA |
| IL12RB1 | NA | NA | NA |
| CRLF1 | 0.964705882352941 | 0.911764705882353 | 0.890588235294118 |
| HAMP | NA | NA | NA |
| COX6B1 | NA | NA | NA |
| NPHS1 | NA | NA | NA |
| DLL3 | NA | NA | NA |
| PRX | 0.966 | 0.875 | 0.843 |
| BCKDHA | 0.953125 | 0.915625 | 0.9012490625 |
| ETHE1 | NA | NA | NA |
| ERCC2 | 0.888181818181818 | 0.770909090909091 | 0.728180909090909 |
| OPA3 | NA | NA | NA |
| FKRP | 0.7825 | 0.6205 | 0.5534975 |
| NUP62 | NA | NA | NA |
| ETFB | NA | NA | NA |
| SLC4A11 | 0.92625 | 0.85875 | 0.83375 |
| PANK2 | 0.914166666666667 | 0.805825 | 0.7658325 |
| DNMT3B | NA | NA | NA |
| GSS | NA | NA | NA |
| SAMHD1 | 0.985789473684211 | 0.966836842105263 | 0.956842105263158 |
| ADA | 0.872083333333333 | 0.803333333333333 | 0.782914583333333 |
| DPM1 | NA | NA | NA |
| EDN3 | NA | NA | NA |
| IFNGR2 | NA | NA | NA |
| HLCS | NA | NA | NA |
| CBS | 0.84625 | 0.77875 | 0.753125 |
| CSTB | NA | NA | NA |
| AIRE | 0.984545454545455 | 0.969090909090909 | 0.963636363636364 |
| COL6A1 | 0.92 | 0.773 | 0.720999 |
| COL6A2 | 0.842857142857143 | 0.702371428571429 | 0.64238 |
| PEX26 | NA | NA | NA |
| SNAP29 | NA | NA | NA |
| LARGE | NA | NA | NA |
| PLA2G6 | 0.938378378378378 | 0.877564864864865 | 0.852159189189189 |
| ALG12 | NA | NA | NA |
| MLC1 | 0.890833333333333 | 0.660833333333333 | 0.5625 |
| SCO2 | NA | NA | NA |
| TYMP | NA | NA | NA |
| ARSA | 0.898214285714286 | 0.8275 | 0.80232125 |
| All | 0.917565008266947 | 0.912709950398317 | 0.911042912971592 |

1. A computer-implemented method, comprising:
generating a plurality of virtual progenies from a plurality of first virtual gametes and a plurality of second virtual gametes, each virtual progeny combining from one of the first virtual gametes and one of the second virtual gametes;
inputting, for each virtual progeny, data associated with the first virtual gamete of the virtual progeny to a machine learning model to determine a first variant-specific gene dysfunction score corresponding to a target allele site;
inputting, for each virtual progeny, data associated with the second virtual gamete of the virtual progeny to the machine learning model to determine a second variant-specific gene dysfunction score corresponding to the target allele site;
deriving, for each virtual progeny, a dysfunction likelihood score of the target allele site from the first variant-specific gene dysfunction score and the second variant-specific gene dysfunction score; and
generating a distribution of dysfunction likelihood of the target allele site based on a plurality of the dysfunction likelihood scores determined from the plurality of virtual progenies.

2. The computer-implemented method of claim 1, wherein, for at least one of the virtual progenies, the dysfunction likelihood score of the target allele corresponds to a geometric mean of the first variant-specific gene dysfunction score and the second variant-specific gene dysfunction score.

3. The computer-implemented method of claim 1, wherein, for at least one of the virtual progenies, the dysfunction likelihood score has more than two possible values.

4. The computer-implemented method of claim 1, wherein, for at least one of the virtual progenies, the dysfunction likelihood score corresponds to a continuous trait model that is normalizable to a range, a first end of the range represents a certainty of allele dysfunction and a second end of the range represents no likelihood of allele dysfunction.

5. The computer-implemented method of claim 1, wherein, for at least one of the virtual progenies, the dysfunction likelihood score, φ_{g}, is derived based on:
$${ϕ}_{g} ( D ) = 1 - [ \frac{( 1 + β ) \times {( 1 - D )}^{{η}_{g}}}{β + {( 1 - D )}^{{η}_{g}}} ]$$
and wherein D represents a diploid gene dysfunction score derived from the first variant-specific gene dysfunction score and the second variant-specific gene dysfunction score, β is a constant, and η_{g} is a gene-specific parameter corresponding to a gene to which the target allele site belongs.

6. The computer-implemented method of claim 1, further comprising:
predicting an expression of a target phenotype based at least on the distribution of dysfunction likelihood of the target allele site.

7. The computer-implemented method of claim 6, wherein predicting the expression of the target phenotype is further based on one or more other allele sites different from the target allele site.

8. The computer-implemented method of claim 6, wherein the target phenotype is an autosomal recessive disease.

9. The computer-implemented method of claim 1, wherein at least one of the first variant-specific gene dysfunction score or the second variant-specific gene dysfunction score is adjusted with a confliction coefficient in response to, based on clinical data, the target allele site expressing gene dysfunction for heterozygotes exceeding a first threshold portion and the target allele site expressing no gene dysfunction for homozygotes exceeding a second threshold portion.

10. The computer-implemented method of claim 1, wherein each of the first virtual gametes is a sequence of haploid alleles of a first potential parent, each haploid allele of the first potential parent selected from one of two alleles of the first potential parent, and each of the second virtual gametes is a sequence of haploid alleles of a second potential parent, each haploid allele of the second potential parent selected from one of two alleles of the second potential parent.

11. The computer-implemented method of claim 10, wherein selecting of each haploid allele from one of the two alleles of the first potential parent for the first virtual gamete or from one of the two alleles of the second potential parent for the second virtual gamete is based on linkage disequilibrium.

12. The computer-implemented method of claim 1, wherein the machine learning model is a neural network comprising multiple nodes, multiple gene-dysfunction metrics and multiple confidence weights, each node associated with one or more of the multiple gene-dysfunction metrics and each of the multiple gene-dysfunction metrics associated with one of the multiple confidence weights, wherein the neural network combines the multiple gene-dysfunction metrics according to the multiple confidence weights to generate one or more variant-specific gene dysfunction scores.

13. The computer-implemented method of claim 12, wherein at least a first one of the multiple nodes of neural network computes a first gene-dysfunction metrics based on clinical data, at least a second one of the multiple nodes of neural network computes a second gene-dysfunction metrics based on evolutionary data, and at least a third one of the multiple nodes of neural network computes a third gene-dysfunction metrics based on population selection.

14. A non-transitory computer readable medium for storing computer code comprising instructions, when executed by one or more processors, cause the one or more processors to perform a process comprising:
generating a plurality of virtual progenies from a plurality of first virtual gametes and a plurality of second virtual gametes, each virtual progeny combining from one of the first virtual gametes and one of the second virtual gametes;
inputting, for each virtual progeny, data associated with the first virtual gamete of the virtual progeny to a machine learning model to determine a first variant-specific gene dysfunction score corresponding to a target allele site;
inputting, for each virtual progeny, data associated with the second virtual gamete of the virtual progeny to the machine learning model to determine a second variant-specific gene dysfunction score corresponding to the target allele site;
deriving, for each virtual progeny, a dysfunction likelihood score of the target allele site from the first variant-specific gene dysfunction score and the second variant-specific gene dysfunction score; and
generating a distribution of dysfunction likelihood of the target allele site based on a plurality of the dysfunction likelihood scores determined from the plurality of virtual progenies.

15. The non-transitory computer readable medium of claim 14, wherein, for at least one of the virtual progenies, the dysfunction likelihood score of the target allele corresponds to a geometric mean of the first variant-specific gene dysfunction score and the second variant-specific gene dysfunction score.

16. The non-transitory computer readable medium of claim 14, wherein, for at least one of the virtual progenies, the dysfunction likelihood score corresponds to a continuous trait model that is normalizable to a range, a first end of the range represents a certainty of allele dysfunction and a second end of the range represents no likelihood of allele dysfunction.

17. The non-transitory computer readable medium of claim 14, wherein, for at least one of the virtual progenies, the dysfunction likelihood score, φ_{g}, is derived based on:
$${ϕ}_{g} ( D ) = 1 - [ \frac{( 1 + β ) \times {( 1 - D )}^{{η}_{g}}}{β + {( 1 - D )}^{{η}_{g}}} ]$$
and wherein D represents a diploid gene dysfunction score derived from the first variant-specific gene dysfunction score and the second variant-specific gene dysfunction score, β is a constant, and η_{g} is a gene-specific parameter corresponding to a gene to which the target allele site belongs.

18. The non-transitory computer readable medium of claim 14, wherein the process further comprises:
predicting an expression of a target phenotype based at least on the distribution of dysfunction likelihood of the target allele site.

19. The non-transitory computer readable medium of claim 18, wherein the target phenotype is an autosomal recessive disease.

20. A system comprising:
a processor; and
a memory configured to store instructions, the instructions, when executed by the processor, cause the processor to perform a process comprising:
generating a plurality of virtual progenies from a plurality of first virtual gametes and a plurality of second virtual gametes, each virtual progeny combining from one of the first virtual gametes and one of the second virtual gametes;
inputting, for each virtual progeny, data associated with the first virtual gamete of the virtual progeny to a machine learning model to determine a first variant-specific gene dysfunction score corresponding to a target allele site;
inputting, for each virtual progeny, data associated with the second virtual gamete of the virtual progeny to the machine learning model to determine a second variant-specific gene dysfunction score corresponding to the target allele site;
deriving, for each virtual progeny, a dysfunction likelihood score of the target allele site from the first variant-specific gene dysfunction score and the second variant-specific gene dysfunction score; and
generating a distribution of dysfunction likelihood of the target allele site based on a plurality of the dysfunction likelihood scores determined from the plurality of virtual progenies.
